(19) United States
(12) Patent Application Publication
(10) Pub. No.: US 2023/0392210 A1
(43) Pub. Date: Dec. 7, 2023

(54) METHODS AND COMPOSITIONS FOR CANCER IMMUNOTHERAPY

(71) Applicant: Genentech, Inc., South San Francisco, CA (US)

(72) Inventors: Soren MULLER, San Francisco, CA (US); Barzin Y. NABET, Philadelphia, PA (US); Namrata Srivastava PATIL, Sunnyvale, CA (US); Romain Francois BANCHEREAU, San Mateo, CA (US)

(21) Appl. No.: 18/062,780

(22) Filed: Dec. 7, 2022

Related U.S. Application Data
(63) Continuation of application No. PCT/US2021/037122, filed on Jun. 11, 2021.
(60) Provisional application No. 63/038,559, filed on Jun. 12, 2020.

(51) Int. Cl. C12Q 1/6886; G01N 33/574; G01N 33/569; C07K 16/28; A61P 35/00; A61K 45/06

(52) U.S. Cl. CPC C12Q 1/6886; G01N 33/57423; G01N 33/56972; C12Q 2600/158; A61P 35/00; A61K 45/06; C12Q 2600/106; C07K 16/2827

(57) ABSTRACT

The present invention provides diagnostic methods, therapeutic methods, and compositions for the treatment of cancer. The compositions and methods described herein can be used, for example, to identify a patient having a cancer who may benefit from treatment with a PD-L1 axis binding antagonist and to treat such patients accordingly. Using the compositions and methods of the disclosure, a patient, such as a human cancer patient, may be determined to be likely to benefit from treatment with a PD-L1 axis binding antagonist if the patient exhibits the presence or an elevated expression level of any of the biomarkers disclosed herein. Exemplary PD-L1 axis binding antagonists that may be used in conjunction with the compositions and methods of the disclosure are PD-L1 binding antagonists, such as anti-PD-L1 antibodies and antigen-binding fragments thereof, including atezolizumab, as well as PD-1 binding antagonists, such as anti-PD-1 antibodies and antigen-binding fragments thereof.

### CROSS-REFERENCE TO RELATED APPLICATIONS

This application is a continuation of International Patent Application No. PCT/US2021/037122, filed Jun. 11, 2021, which claims the benefit of priority to U.S. Provisional Application No. 63/038,559, filed on Jun. 12, 2020, the contents of which are incorporated herein by reference in their entirety.

### SEQUENCE LISTING

The instant application contains a Sequence Listing which has been submitted electronically in XML format and is hereby incorporated by reference in its entirety. Said XML copy, created on Dec. 6, 2022, is named 50474-209003_Sequence_Listing_12_6_22 and is 320,824 bytes in size.

### FIELD OF THE INVENTION

The present invention is directed to diagnostic and therapeutic methods for the treatment of cancer using PD-L1 axis binding antagonists. Also provided are related kits and compositions.

### BACKGROUND OF THE INVENTION

Cancer remains one of the deadliest threats to human health. In the U.S., cancer affects nearly 1.3 million new patients each year and is the second leading cause of death after heart disease, accounting for approximately 1 in 4 deaths. It is also predicted that cancer may surpass cardiovascular diseases as the number one cause of death within 5 years. Solid tumors are responsible for most of those deaths.

Studies in humans with immune checkpoint inhibitors have demonstrated the promise of harnessing the immune system to control and eradicate tumor growth. The programmed death 1 (PD-1) receptor and its ligand programmed death-ligand 1 (PD-L1) are immune checkpoint proteins that have been implicated in the suppression of immune system responses during chronic infections, pregnancy, tissue allografts, autoimmune diseases, and cancer. PD-L1 regulates the immune response by binding to the inhibitory receptor PD-1, which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Although there have been significant advances in the medical treatment of certain cancers, the overall 5-year survival rate for all cancers has improved only by about 10% in the past 20 years. Malignant solid tumors, in particular, metastasize and grow rapidly in an uncontrolled manner, making their timely detection and treatment extremely difficult.

Despite the significant advancement in the treatment of cancer, improved diagnostic and therapeutic methods and cancer therapies are still being sought.

### SUMMARY OF THE INVENTION

The present disclosure provides therapeutic and diagnostic methods and compositions for treating an individual having a cancer (e.g., lung cancer (e.g., non-small cell lung cancer (NSCLC)), endometrial cancer, colon adenocarcinoma, renal cell carcinoma, bladder cancer (e.g., urothelial carcinoma (UC)), kidney cancer (e.g., renal cell carcinoma (RCC)), and breast cancer (e.g., triple-negative breast cancer (TNBC)).

In one aspect, the invention features a method of identifying an individual having a cancer who may benefit from a treatment comprising a PD-L1 axis binding antagonist, the method comprising determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

In another aspect, the invention features a method of selecting a therapy for an individual having a cancer, the method comprising determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

In some aspects, the immune-score expression level of the two or more genes in the sample is above the reference immune-score expression level and the method further comprises administering to the individual an effective amount of a PD-L1 axis binding antagonist.

In another aspect, the invention features a method of treating an individual having a cancer, the method comprising: (a) determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and (b) administering an effective amount of a PD-L1 axis binding antagonist to the individual.

In another aspect, the invention features a method of treating cancer in an individual that has been determined to have an immune-score expression level of two or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes, the method comprising administering to the individual an effective amount of a PD-L1 axis binding antagonist.

In another aspect, the invention features a PD-L1 axis binding antagonist for use in a method of treating an individual having a cancer, the method comprising: (a) determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and (b) administering an effective amount of a PD-L1 axis binding antagonist to the individual.

In another aspect, the invention features a PD-L1 axis binding antagonist for treating cancer in an individual that has been determined to have an immune-score expression level of two or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes.

In some aspects, the immune-score reference expression level is an immune-score expression level of the two or more genes in a reference population.

In some aspects, the reference population is a population of individuals having the cancer.

In some aspects, the population of individuals comprises a first subset of individuals who have been treated with a PD-L1 axis binding antagonist and a second subset of individuals who have been treated with therapy that does not comprise a PD-L1 axis binding antagonist.

In some aspects, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

In some aspects, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

In some aspects, the therapy that does not comprise a PD-L1 axis binding antagonist comprises a chemotherapeutic agent.

In some aspects, the chemotherapeutic agent is docetaxel.

In some aspects, responsiveness to treatment comprises an extension in OS, an extension in progression-free survival (PFS), or an increase in best confirmed overall response (BOOR).

In some aspects, responsiveness to treatment comprises an extension in OS.

In some aspects, the reference immune-score expression level is a median of the expression level of each of the two or more genes in the reference population.

In some aspects, the genes comprise three or more of CD79A, CD19, BANK1, JCHAIN, SLAMF7, BTK, TNFRSF17, IGJ, IGLL5, RBPJ, and MZB1.

In some aspects, the genes comprise four or more of CD79A, CD19, BANK1, JCHAIN, SLAMF7, BTK, TNFRSF17, IGJ, IGLL5, RBPJ, and MZB1.

In some aspects, the genes comprise five or more of CD79A, CD19, BANK1, JCHAIN, SLAMF7, BTK, TNFRSF17, IGJ, IGLL5, RBPJ, and MZB1.

In some aspects, the genes comprise six or more of CD79A, CD19, BANK1, JCHAIN, SLAMF7, BTK, TNFRSF17, IGJ, IGLL5, RBPJ, and MZB1.

In some aspects, the genes comprise seven or more of CD79A, CD19, BANK1, JCHAIN, SLAMF7, BTK, TNFRSF17, IGJ, IGLL5, RBPJ, and MZB1.

In some aspects, the genes comprise CD79A, CD19, BANK1, JCHAIN, SLAMF7, BTK, TNFRSF17, IGJ, IGLL5, RBPJ, and MZB1.

In another aspect, the invention features a method of identifying an individual having a cancer who may benefit from a treatment comprising a PD-L1 axis binding antagonist, the method comprising determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's overall survival (OS) as compared to treatment without the PD-L1 axis binding antagonist.

In another aspect, the invention features a method of selecting a therapy for an individual having a cancer, the method comprising determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In some aspects, the immune-score expression level of the one or more genes in the sample is above the reference immune-score expression level and the method further comprises administering to the individual an effective amount of a PD-L1 axis binding antagonist.

In another aspect, the invention features a method of treating an individual having a cancer, the method comprising: (a) determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist to the individual.

In another aspect, the invention features a method of treating cancer in an individual that has been determined to have an immune-score expression level of one or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, the method comprising administering to the individual an effective amount of a PD-L1 axis binding antagonist, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another aspect, the invention features a PD-L1 axis binding antagonist for use in a method of treating an individual having a cancer, the method comprising: (a) determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist to the individual.

In another aspect, the invention features a PD-L1 axis binding antagonist for use in treating cancer in an individual that has been determined to have an immune-score expression level of one or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In some aspects, the immune-score expression level of one of genes CD79A, CD19, BANK1, JCHAIN, SLAMF7, BTK, TNFRSF17, IGJ, IGLL5, RBPJ, and MZB1 is determined.

In some aspects, the immune-score expression level of CD79A is determined.

In some aspects, the immune-score reference expression level is an immune-score expression level of the one or more genes in a reference population.

In some aspects, the reference population is a population of individuals having the cancer.

In some aspects, the population of individuals comprises a first subset of individuals who have been treated with a PD-L1 axis binding antagonist and a second subset of individuals who have been treated with therapy that does not comprise a PD-L1 axis binding antagonist.

In some aspects, the immune-score reference expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

In some aspects, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

In some aspects, the therapy that does not comprise a PD-L1 axis binding antagonist comprises a chemotherapeutic agent.

In some aspects, the chemotherapeutic agent is docetaxel.

In some aspects, responsiveness to treatment comprises an extension in OS, an extension in PFS, or an increase in BOOR.

In some aspects, responsiveness to treatment comprises an extension in OS.

In some aspects, the immune-score reference expression level is a median of the expression level of each of the one or more genes in the reference population.

In some aspects, the median expression level is the median of a mean Z score of the expression level of each of the two or more genes in the reference population.

In some aspects, the genes comprise two or more of CD79A, CD19, BANK1, JCHAIN, SLAMF7, BTK, TNFRSF17, IGJ, IGLL5, RBPJ, and MZB1.

In some aspects, the two or more genes comprise TNFRSF17 and IGJ.

In some aspects, the two genes consist of TNFRSF17 and IGJ.

In some aspects, the genes comprise three or more of CD79A, CD19, BANK1, JCHAIN, SLAMF7, BTK, TNFRSF17, IGJ, IGLL5, RBPJ, and MZB1.

In some aspects, the genes comprise four or more of CD79A, CD19, BANK1, JCHAIN, SLAMF7, BTK, TNFRSF17, IGJ, IGLL5, RBPJ, and MZB1.

In some aspects, the genes comprise five or more of CD79A, CD19, BANK1, JCHAIN, SLAMF7, BTK, TNFRSF17, IGJ, IGLL5, RBPJ, and MZB1.

In some aspects, the genes comprise six or more of CD79A, CD19, BANK1, JCHAIN, SLAMF7, BTK, TNFRSF17, IGJ, IGLL5, RBPJ, and MZB1.

In some aspects, the genes comprise seven or more of CD79A, CD19, BANK1, JCHAIN, SLAMF7, BTK, TNFRSF17, IGJ, IGLL5, RBPJ, and MZB1.

In some aspects, the genes comprise CD79A, CD19, BANK1, JCHAIN, SLAMF7, BTK, TNFRSF17, IGJ, IGLL5, RBPJ, and MZB1.

In another aspect, the invention features a method of identifying an individual having a cancer who may benefit from a treatment comprising a PD-L1 axis binding antagonist, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist.

In another aspect, the invention features a method of selecting a therapy for an individual having a cancer, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist.

In some embodiments of either of the preceding two aspects, the immune-score expression level of the one or more genes in the sample is above the reference immune-score expression level and the method further comprises administering to the individual an effective amount of a PD-L1 axis binding antagonist.

In a further aspect, the invention features a method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and
(b) administering an effective amount of a PD-L1 axis binding antagonist to the individual.

In another aspect, the invention features a method of treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, the method comprising administering to the individual an effective amount of a PD-L1 axis binding antagonist.

In some embodiments, the immune-score reference expression level is an immune-score expression level of the one or more genes in a reference population.

In some embodiments, the reference population is a population of individuals having the cancer.

In some embodiments, the population of individuals comprises a first subset of individuals who have been treated with a PD-L1 axis binding antagonist and a second subset of individuals who have been treated with therapy that does not comprise a PD-L1 axis binding antagonist.

In some embodiments, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

In some embodiments, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

In some embodiments, the therapy that does not comprise a PD-L1 axis binding antagonist comprises a chemotherapeutic agent.

In some embodiments, the chemotherapeutic agent is docetaxel.

In some embodiments, responsiveness to treatment comprises an extension in OS, an extension in PFS, or an increase in BOOR.

In some embodiments, responsiveness to treatment comprises an extension in OS.

In some embodiments, the reference immune-score expression level is a median of the expression level of each of the one or more genes in the reference population.

In some embodiments, the genes comprise two or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise three or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise four or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise five or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise six or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise seven or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise eight or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise nine or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 10 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 11 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 12 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 13 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In another aspect, the invention features a method of identifying an individual having a cancer who may benefit from a treatment comprising a PD-L1 axis binding antagonist, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another aspect, the invention features a method of selecting a therapy for an individual having a cancer, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In some embodiments of either of the two preceding aspects, the immune-score expression level of the one or more genes in the sample is above the reference immune-score expression level and the method further comprises administering to the individual an effective amount of a PD-L1 axis binding antagonist.

In another aspect, the invention features a method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and
(b) administering an effective amount of a PD-L1 axis binding antagonist to the individual.

In another aspect, the invention features a method of treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, the method comprising administering to the individual an effective amount of a PD-L1 axis binding antagonist, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In some embodiments, the immune-score reference expression level is an immune-score expression level of the one or more genes in a reference population.

In some embodiments, the reference population is a population of individuals having the cancer.

In some embodiments, the population of individuals comprises a first subset of individuals who have been treated with a PD-L1 axis binding antagonist and a second subset of individuals who have been treated with therapy that does not comprise a PD-L1 axis binding antagonist.

In some embodiments, the immune-score reference expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

In some embodiments, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

In some embodiments, the therapy that does not comprise a PD-L1 axis binding antagonist comprises a chemotherapeutic agent.

In some embodiments, the chemotherapeutic agent is docetaxel.

In some embodiments, responsiveness to treatment comprises an extension in OS, an extension in PFS, or an increase in BOOR.

In some embodiments, responsiveness to treatment comprises an extension in OS.

In some embodiments, the immune-score reference expression level is a median of the expression level of each of the one or more genes in the reference population.

In some embodiments, the median expression level is the median of a mean Z score of the expression level of each of the one or more genes in the reference population.

In some embodiments, the genes comprise two or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise three or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise four or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise five or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise six or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise seven or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise eight or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise nine or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 10 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 11 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 12 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise 13 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some embodiments, the genes comprise MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In another aspect, the invention features a method of identifying an individual having a cancer who may benefit from a treatment comprising a PD-L1 axis binding antagonist, the method comprising determining the presence of a tertiary lymphoid structure (TLS) in a tumor sample from the individual, wherein the presence of a TLS in the tumor sample identifies the individual as one who may benefit from a treatment comprising a PD-L1 axis binding antagonist.

In another aspect, the invention features a method of selecting a therapy for an individual having a cancer, the method comprising determining the presence of a TLS in a tumor sample from the individual, wherein the presence of a TLS in the tumor sample identifies the individual as one who may benefit from a treatment comprising a PD-L1 axis binding antagonist.

In some aspects, the sample from the individual is determined to have the presence of a TLS and the method further comprises administering to the individual an effective amount of a PD-L1 axis binding antagonist.

In another aspect, the invention features a method of treating an individual having a cancer, the method comprising: (a) determining the presence of a TLS in a tumor sample from the individual; and (b) administering an effective amount of a PD-L1 axis binding antagonist to the individual.

In another aspect, the invention features a method of treating cancer in an individual that has been determined to have the presence of a TLS in a tumor sample from the individual, the method comprising administering to the individual an effective amount of a PD-L1 axis binding antagonist.

In another aspect, the invention features a PD-L1 axis binding antagonist for use in a method of treating an individual having a cancer, the method comprising: (a) determining the presence of a TLS in a tumor sample from the individual; and (b) administering an effective amount of a PD-L1 axis binding antagonist to the individual.

In another aspect, the invention features a PD-L1 axis binding antagonist for use in treating cancer in an individual that has been determined to have the presence of a TLS in a tumor sample from the individual.

In some aspects, the presence of a TLS is determined by histological staining, immunohistochemistry (IHC), immunofluorescence, or gene expression analysis.

In some aspects, the histological staining comprises hematoxylin and eosin (H&E) staining.

In some aspects, the IHC or immunofluorescence comprises detecting CD62L, L-selectin, CD40, or CD8.

In some aspects, CD62L or L-selectin is detected using a MECA-79 antibody.

In some aspects, the gene expression analysis comprises determining the expression level of a TLS gene signature in the sample.

In some aspects, the TLS gene signature comprises one or more of genes CCL2, CCL3, CCL4, CCL5, CCL8, CCL18, CCL19, CCL21, CXCL9, CXCL10, CXCL11, and CXCL13.

In some aspects, the genes comprise two or more of CCL2, CCL3, CCL4, CCL5, CCL8, CCL18, CCL19, CCL21, CXCL9, CXCL10, CXCL11, and CXCL13.

In another aspect, the invention features a method of identifying an individual having a cancer who may benefit from a treatment comprising a PD-L1 axis binding antagonist, the method comprising determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

In another aspect, the invention features a method of selecting a therapy for an individual having a cancer, the method comprising determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

In some aspects, the immune-score expression level of the two or more genes in the sample is above the reference immune-score expression level and the method further comprises administering to the individual an effective amount of a PD-L1 axis binding antagonist.

In another aspect, the invention features a method of treating an individual having a cancer, the method comprising: (a) determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and (b) administering an effective amount of a PD-L1 axis binding antagonist to the individual.

In another aspect, the invention features a method of treating cancer in an individual that has been determined to have an immune-score expression level of two or more of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes, the method comprising administering to the individual an effective amount of a PD-L1 axis binding antagonist.

In another aspect, the invention features a PD-L1 axis binding antagonist for use in a method of treating an individual having a cancer, the method comprising: (a) determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and (b) administering an effective amount of a PD-L1 axis binding antagonist to the individual.

In another aspect, the invention features a PD-L1 axis binding antagonist for use in treating cancer in an individual that has been determined to have an immune-score expression level of two or more of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes.

In some aspects, the reference immune-score expression level is an immune-score expression level of the two or more genes in a reference population.

In some aspects, the reference population is a population of individuals having the cancer.

In some aspects, the population of individuals comprises a first subset of individuals who have been treated with a PD-L1 axis binding antagonist and a second subset of individuals who have been treated with therapy that does not comprise a PD-L1 axis binding antagonist.

In some aspects, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist.

In some aspects, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

In some aspects, the therapy that does not comprise a PD-L1 axis binding antagonist comprises a chemotherapeutic agent.

In some aspects, the chemotherapeutic agent is docetaxel.

In some aspects, responsiveness to treatment comprises an extension in OS, an extension in PFS, or an increase in BOOR.

In some aspects, responsiveness to treatment comprises an extension in OS.

In some aspects, the reference immune-score expression level is a median of the expression level of each of the two or more genes in the reference population.

In some aspects, the median expression level is the median of a mean Z score of the expression level of each of the two or more genes in the reference population.

In some aspects, the genes comprise three or more of CCL2, CCL3, CCL4, CCL5, CCL8, CCL18, CCL19, CCL21, CXCL9, CXCL10, CXCL11, and CXCL13.

In some aspects, the genes comprise four or more of CCL2, CCL3, CCL4, CCL5, CCL8, CCL18, CCL19, CCL21, CXCL9, CXCL10, CXCL11, and CXCL13.

In some aspects, the genes comprise five or more of CCL2, CCL3, CCL4, CCL5, CCL8, CCL18, CCL19, CCL21, CXCL9, CXCL10, CXCL11, and CXCL13.

In some aspects, the genes comprise six or more of CCL2, CCL3, CCL4, CCL5, CCL8, CCL18, CCL19, CCL21, CXCL9, CXCL10, CXCL11, and CXCL13.

In some aspects, the genes comprise seven or more of CCL2, CCL3, CCL4, CCL5, CCL8, CCL18, CCL19, CCL21, CXCL9, CXCL10, CXCL11, and CXCL13.

In some aspects, the genes comprise eight or more of CCL2, CCL3, CCL4, CCL5, CCL8, CCL18, CCL19, CCL21, CXCL9, CXCL10, CXCL11, and CXCL13.

In some aspects, the genes comprise nine or more of CCL2, CCL3, CCL4, CCL5, CCL8, CCL18, CCL19, CCL21, CXCL9, CXCL10, CXCL11, and CXCL13.

In some aspects, the genes comprise ten or more of CCL2, CCL3, CCL4, CCL5, CCL8, CCL18, CCL19, CCL21, CXCL9, CXCL10, CXCL11, and CXCL13.

In some aspects, the genes comprise eleven or more of CCL2, CCL3, CCL4, CCL5, CCL8, CCL18, CCL19, CCL21, CXCL9, CXCL10, CXCL11, and CXCL13.

In some aspects, the genes comprise CCL2, CCL3, CCL4, CCL5, CCL8, CCL18, CCL19, CCL21, CXCL9, CXCL10, CXCL11, and CXCL13.

In some aspects, the expression level is a nucleic acid expression level.

In some aspects, the nucleic acid expression level is an mRNA expression level.

In some aspects, the mRNA expression level is determined by RNA-seq, RT-qPCR, qPCR, multiplex qPCR or RT-qPCR, microarray analysis, SAGE, MassARRAY technique, FISH, or a combination thereof.

In some aspects, the mRNA expression level is detected using RNA-seq.

In some aspects, the expression level is a protein expression level.

In some aspects, the protein expression level is determined by IHC, immunofluorescence, mass spectrometry, flow cytometry, and Western blot, or a combination thereof.

In some aspects, the expression level is detected in tumor cells, tumor-infiltrating immune cells, stromal cells, normal adjacent tissue (NAT) cells, or a combination thereof.

In another aspect, the invention features a method of identifying an individual having a cancer who may benefit from a treatment comprising a PD-L1 axis binding antagonist, the method comprising determining the number of B cells in a tumor sample from the individual, wherein a number of B cells in the tumor sample that is above a reference number of B cells identifies the individual as one who may benefit from a treatment comprising a PD-L1 axis binding antagonist.

In another aspect, the invention features a method of selecting a therapy for an individual having a cancer, the method comprising determining the number of B cells in a tumor sample from the individual, wherein a number of B cells in the tumor sample that is above a reference number of B cells identifies the individual as one who may benefit from a treatment comprising a PD-L1 axis binding antagonist.

In some aspects, the number of B cells in the sample is above the reference number and the method further comprises administering to the individual an effective amount of a PD-L1 axis binding antagonist.

In another aspect, the invention features a method of treating an individual having a cancer, the method comprising: (a) determining the number of B cells in a tumor sample from the individual; and (b) administering an effective amount of a PD-L1 axis binding antagonist to the individual.

In another aspect, the invention features a method of treating cancer in an individual that has been determined to have a number of B cells in a tumor sample from the individual that is above a reference number of B cells, the method comprising administering to the individual an effective amount of a PD-L1 axis binding antagonist.

In another aspect, the invention features a PD-L1 axis binding antagonist for use in a method of treating an individual having a cancer, the method comprising: (a) determining the number of B cells in a tumor sample from the individual; and (b) administering an effective amount of a PD-L1 axis binding antagonist to the individual.

In another aspect, the invention features a PD-L1 axis binding antagonist for use in treating cancer in an individual that has been determined to have a number of B cells in a tumor sample from the individual that is above a reference number of B cells.

In some aspects, the B cells comprise CD79+ B cells, IgG+ B cells, and/or plasma cells.

In another aspect, the invention features a method of identifying an individual having a cancer who may benefit from a treatment comprising a PD-L1 axis binding antagonist, the method comprising determining whether the individual has clonally expanded B cells in a tumor sample from the individual, wherein clonally expanded B cells in the sample identify the individual as one who may benefit from a treatment comprising a PD-L1 axis binding antagonist.

In another aspect, the invention features a method of selecting a therapy for an individual having a cancer, the method comprising determining whether the individual has clonally expanded B cells in a tumor sample from the individual, wherein clonally expanded B cells in the sample identify the individual as one who may benefit from a treatment comprising a PD-L1 axis binding antagonist.

In some aspects, the tumor sample comprises clonally expanded B cells and the method further comprises administering to the individual an effective amount of a PD-L1 axis binding antagonist.

In another aspect, the invention features a method of treating an individual having a cancer, the method comprising: (a) determining that the individual has clonally expanded B cells in a tumor sample from the individual; and (b) administering an effective amount of a PD-L1 axis binding antagonist to the individual.

In another aspect, the invention features a method of treating cancer in an individual that has been determined to have clonally expanded B cells in a tumor sample from the individual, the method comprising administering to the individual an effective amount of a PD-L1 axis binding antagonist.

In another aspect, the invention features a PD-L1 axis binding antagonist for use in a method of treating an individual having a cancer, the method comprising: (a) determining that the individual has clonally expanded B cells in a tumor sample from the individual; and (b) administering an effective amount of a PD-L1 axis binding antagonist to the individual.

In another aspect, the invention features a PD-L1 axis binding antagonist for use in treating cancer in an individual that has been determined to have clonally expanded B cells in a tumor sample from the individual.

In some aspects, the clonally expanded B cells are clonally expanded plasma cells.

In some aspects, clonally expanded B cells are detected by measuring the diversity of the B cell receptor (BCR) gene repertoire in the tumor sample.

In some aspects, a Shannon Diversity Index (SDI) of the BCR gene repertoire in the tumor sample from the individual that is below a reference SDI identifies the individual as one who may benefit from a treatment comprising a PD-L1 axis binding antagonist.

In some aspects, the sample is a tissue sample, a cell sample, a whole blood sample, a plasma sample, a serum sample, or a combination thereof.

In some aspects, the tissue sample is a tumor tissue sample.

In some aspects, the tumor sample is a tumor tissue sample.

In some aspects, the tumor tissue sample comprises tumor cells, tumor-infiltrating immune cells, stromal cells, NAT cells, or a combination thereof.

In some aspects, the tumor tissue sample is a formalin-fixed and paraffin-embedded (FFPE) sample, an archival sample, a fresh sample, or a frozen sample.

In some aspects, the tumor tissue sample is an FFPE sample.

In some aspects, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy.

In some aspects, the cancer is a lung cancer, a kidney cancer, a bladder cancer, or a breast cancer.

In some aspects, the lung cancer is a non-small cell lung cancer (NSCLC).

In some aspects, the NSCLC is non-squamous NSCLC.

In some aspects, the NSCLC is squamous NSCLC.

In some aspects, the benefit comprises an extension in the individual's OS, an extension in the individual's PFS, and/or an improvement in the individual's BOOR, as compared to treatment without the PD-L1 axis binding antagonist.

In some aspects, the benefit comprises an extension in the individual's OS as compared to treatment without the PD-L1 axis binding antagonist.

In some aspects, the PD-L1 axis binding antagonist is selected from the group consisting of a PD-L1 binding antagonist, a PD-1 binding antagonist, and a PD-L2 binding antagonist.

In some aspects, the PD-L1 axis binding antagonist is a PD-L1 binding antagonist.

In some aspects, the PD-L1 binding antagonist inhibits the binding of PD-L1 to one or more of its ligand binding partners.

In some aspects, the PD-L1 binding antagonist inhibits the binding of PD-L1 to PD-1.

In some aspects, the PD-L1 binding antagonist inhibits the binding of PD-L1 to B7-1.

In some aspects, the PD-L1 binding antagonist inhibits the binding of PD-L1 to both PD-1 and

B7-1.

In some aspects, the PD-L1 binding antagonist is an antibody or antigen-binding fragment thereof.

In some aspects, the antibody is selected from the group consisting of atezolizumab, MDX-1105, MED14736 (durvalumab), and MSB0010718C (avelumab).

In some aspects, the antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

In some aspects, the antibody comprises a heavy chain variable region comprising the amino acid sequence of SEQ ID NO: 7 and a light chain variable region comprising the amino acid sequence of SEQ ID NO: 8.

In some aspects, the PD-L1 axis binding antagonist is a PD-1 binding antagonist.

In some aspects, the PD-1 binding antagonist inhibits the binding of PD-1 to one or more of its ligand binding partners.

In some aspects, the PD-1 binding antagonist inhibits the binding of PD-1 to PD-L1.

In some aspects, the PD-1 binding antagonist inhibits the binding of PD-1 to PD-L2.

In some aspects, the PD-1 binding antagonist inhibits the binding of PD-1 to both PD-L1 and PD-L2.

In some aspects, the PD-1 binding antagonist is an antibody or antigen-binding fragment thereof.

In some aspects, the antibody is selected from the group consisting of: MDX-1106 (nivolumab), MK-3475 (pembrolizumab), MEDI-0680 (AMP-514), PDR001, REGN2810, and BGB-108.

In some aspects, the PD-1 binding antagonist is an Fc-fusion protein.

In some aspects, the Fc-fusion protein is AMP-224.

In some aspects, the individual has not been previously treated for the cancer.

In some aspects, the individual has not been previously administered a PD-L1 axis binding antagonist.

In some aspects, the cancer is NSCLC, and wherein the individual has no EGFR or ALK genomic tumor aberrations.

In some aspects, the individual has previously been treated for the cancer.

In some aspects, the individual has previously been treated for the cancer by administration of a platinum-containing chemotherapeutic agent to the individual, and wherein the individual has failed to respond to the chemotherapeutic agent.

In some aspects, the PD-L1 axis binding antagonist is administered as a monotherapy.

In some aspects, the method further comprises administering an effective amount of one or more additional therapeutic agents.

In some aspects, the one or more additional therapeutic agents comprise an anti-neoplastic agent, a chemotherapeutic agent, a growth inhibitory agent, an anti-angiogenic agent, a radiation therapy, a cytotoxic agent, an immunomodulatory agent, or a combination thereof.

In some aspects, the individual is a human.

In another aspect, the invention features a kit comprising a PD-L1 axis binding antagonist and instructions to administer the PD-L1 axis binding antagonist to an individual who has been identified as one who may benefit from a treatment comprising the PD-L1 binding antagonist in accordance with any one of the methods disclosed herein.

In another aspect, the invention features a kit comprising a PD-L1 axis binding antagonist and instructions to administer the PD-L1 axis binding antagonist to an individual who has been selected for a treatment comprising the PD-L1 binding antagonist in accordance with any one of the methods disclosed herein.

In another aspect, the invention features a kit for identifying an individual having a cancer who may benefit from a treatment comprising a PD-L1 axis binding antagonist, the kit comprising reagents for determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

In another aspect, the invention features a kit for identifying an individual having a cancer who may benefit from a treatment comprising a PD-L1 axis binding antagonist, the kit comprising reagents for determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another aspect, the invention features a kit for identifying an individual having a cancer who may benefit from a treatment comprising a PD-L1 axis binding antagonist, the kit comprising reagents for determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another aspect, the invention features a PD-L1 axis binding antagonist for use in a method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and

(b) administering an effective amount of a PD-L1 axis binding antagonist to the individual.

In another aspect, the invention features a PD-L1 axis binding antagonist for treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes.

In another aspect, the invention features a PD-L1 axis binding antagonist for use in a method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and
(b) administering an effective amount of a PD-L1 axis binding antagonist to the individual.

In another aspect, the invention features a PD-L1 axis binding antagonist for use in treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another aspect, the invention features a kit for identifying an individual having a cancer who may benefit from a treatment comprising a PD-L1 axis binding antagonist, the kit comprising reagents for determining the presence of a tertiary lymphoid structure (TLS) in a tumor sample from the individual, wherein the presence of a TLS in the tumor sample identifies the individual as one who may benefit from a treatment comprising a PD-L1 axis binding antagonist.

In another aspect, the invention features a kit for identifying an individual having a cancer who may benefit from a treatment comprising a PD-L1 axis binding antagonist, the kit comprising reagents for determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

In another aspect, the invention features a kit for identifying an individual having a cancer who may benefit from a treatment comprising a PD-L1 axis binding antagonist, the kit comprising reagents for determining the number of B cells in a tumor sample from the individual, wherein a number of B cells in the tumor sample that is above a reference number of B cells identifies the individual as one who may benefit from a treatment comprising a PD-L1 axis binding antagonist.

In another aspect, the invention features a kit for identifying an individual having a cancer who may benefit from a treatment comprising a PD-L1 axis binding antagonist, the kit comprising reagents for determining whether the individual has clonally expanded B cells in a tumor sample from the individual, wherein clonally expanded B cells in the sample identify the individual as one who may benefit from a treatment comprising a PD-L1 axis binding antagonist.

### BRIEF DESCRIPTION OF THE DRAWINGS

FIGS. 1A-1C show B cell gene signature and CD79A associate with atezolizumab mediated survival benefits in the POPLAR Phase 2 study. FIG. 1A: Differential gene expression analysis showing enrichment of B cell gene signature when comparing patients with OS 6 months (n=24) versus OS 2 months (n=43). Yellow circles indicate B cell gene transcripts and redcircles indicate T_{eff} gene transcripts. Kaplan Meier (KM) curves comparing probability of survival in patients enriched for (FIG. 1B) B cell gene signature (>median docetaxel=11.07, <median docetaxel=9.23, <median atezolizumab=8.44 months), and (FIG. 1C) CD79A gene (>median docetaxel=12.45, <median docetaxel=9.23, <median atezolizumab=8.44 months) (n=194).

FIGS. 2A-2D show B cell gene signature high patients associate with atezolizumab provoked tumor responses in multiple phase 3 studies. KM curves comparing probability of survival in patients enriched for B cell gene signature (both high (>median) & low (<median) as indicated) in (FIG. 2A) OAK trial (>median docetaxel=12.39, <median docetaxel=8.8, >median atezolizumab=18.04, <median atezolizumab=7.79 months) (n=727) and (FIG. 2B) BIRCH trial (>median atezolizumab=17.74, <median atezolizumab=14.09 months) (n=591). FIG. 2C: Comparison of BCOR for enrichment of B cell gene signature across docetaxel and atezolizumab arm in patient population classified as complete response (CR), partial response (PR), progressive disease (PD), stable disease (SD) according to RECIST V1.1 classification. p values for docetaxel group 0.34 and atezolizumab group 0.00094 (Kruskal-Wallis test). FIG. 2D: Progression-free Survival (PFS) curves in OAK trial (n=727) (>median docetaxel=4.17, <median docetaxel=2.83, >median atezolizumab=2.99, <median atezolizumab=1.64 months).

FIGS. 3A-3E show that patients responsive to atezolizumab have infiltration of B cells and TLS into tumors showing association with survival benefits. FIG. 3A: An immunofluorescence representative image of pre-treatment lung adenocarcinoma sample showing high B cell gene signature and classified as atezolizumab responder: CD79A in green, CD8 in red and Ki67 in blue. Scale Bar: 100 mm. FIG. 3B: Representative H&E stain of a lung adenocarcinoma from atezolizumab responsive patient showing presence of TLS as shown by marking. Scale Bar: 500 mm. FIG. 3C: Association of CD79a gene expression comparing patient tissue showing presence or absence of TLS (***: p<0.001, paired t test). FIG. 3D: Association of CD3D gene expression comparing patient tissue showing presence or absence of TLS (**: p<0.01, paired t test). FIG. 3E: Association of overall survival (OS in months) to presence/absence of TLS in both docetaxel and atezolizumab arm in POPLAR trial (*:p<0.05, paired t test) (n=194).

FIGS. 4A-4D show immuno-staining of TLS. An IHC representative image of a lung adenocarcinoma tissue from a patient showing high B cell gene signature and classified as atezolizumab responder, showing (FIG. 4A) hematoxylin & eosin (H&E) staining for presence of TLS as indicated by the white circle, (FIG. 4B) panCK and CD8 (red) staining, (FIG. 4C) PNAd (peripheral lymph node addressins) (FIG. 4D) CD40 staining. Scale bar is 20 μm.

FIGS. 5A-5D are KM curves comparing probability of survival in patients enriched for TLS gene signature (FIG. 5A) in POPLAR trial (>median docetaxel=10.63, <median docetaxel=9.9, >median atezolizumab=15.47, <median atezolizumab=8.54 months) (n=194) and (FIG. 5B) in OAK (>median docetaxel=10.28, <median docetaxel=10.28, >median atezolizumab=14.32, <median atezolizumab=11.76 months) (n=727). KM curves comparing probability of survival in patients enriched for germinal center gene signature (FIG. 5C) in POPLAR trial ((>median docetaxel=8.87, <median docetaxel=10.05, <median atezolizumab=9.72 months) (n=194) and (FIG. 5D) in OAK trial ((>median docetaxel=11.43, <median docetaxel=8.9, >median atezolizumab=16.26, <median atezolizumab=9.95 months) (n=727).

FIGS. 6A-6E show B cell repertoire is enriched in patients with atezolizumab mediated benefit. FIG. 6A: KM curves comparing probability of survival in patients enriched for plasma B cell 2-gene signature (>median docetaxel=11.07, <median docetaxel=9.53, >median atezolizumab=16.43, <median atezolizumab=7.82 months) (n=727). BCR sequencing done from limited patient samples as indicated indicating diversity of clonality using Shannon Index for (FIG. 6B) control patients (n=3) (FIG. 6C) PR (red) and SD (blue) patients before and after atezolizumab treatment (n=8), (FIG. 6D) PD patients (n=3) and (FIG. 6E) summary plots showing change in Shannon Index FIG. 7 shows enrichment of genes in the POPLAR trial and provides a list of genes enriched in atezolizumab responders along with their HR and p values.

FIGS. 8A and 8B show the association of B cell with PD-L1 status. Plots quantifying association of B cells gene signature with (FIG. 8A) immune cell PD-L1 levels where 100=0%, IC1=1-5%, 102=5-49%, 103=>50% (FIG. 8B) immune & tumor cell PD-L1 determined by SP142 PD-L1 assay (Wilcoxon paired analysis).

FIGS. 9A-9F show TLS prevalence and its association. FIG. 9A: Distribution of TLS (with germinal center) and lymphoid aggregate (without germinal center) based on histology. FIG. 9B: Distribution of TLS in biopsy and resection samples. Association of TLS presence identified by IHC with RNA sequencing based gene signatures for (FIG. 9C) B cell, (FIG. 9D) Ten, and (FIG. 9E) TLS (Wilcoxon paired analysis). FIG. 9F: Association of overall survival (OS in months) to presence/absence of TLS in both docetaxel and atezolizumab arm in OAK trial (***:p<0.001, paired t test).

FIGS. 10A-10D show the association of B cell and TLS gene signatures with other biomarkers. Plots quantifying association of B cells gene signature with (FIG. 10A) tumor mutation burden (TMB) and (FIG. 10B) STK11 mutation status. Plots showing association of TLS gene signature with (FIG. 100) TMB and (FIG. 10D) STK11 mutation status (Wilcoxon paired analysis).

FIGS. 11A-11C show the association of survival benefit with B cell immunophenotypes. KM curves comparing probability of survival in patients enriched for (FIG. 11A) naïve B cell gene signature (>median docetaxel=10.71, <median docetaxel=9.9, >median atezolizumab=13.47, <median atezolizumab=11.79 months), (FIG. 11B) memory B cell signature (>median docetaxel=12.39, <median docetaxel=8.8, >median atezolizumab=17.64, <median atezolizumab=8.9 months), and (FIG. 11C) plasma B cell (>median docetaxel=11.53, <median docetaxel=9.72, >median atezolizumab=15.49, <median atezolizumab=9.72 months) (n=727).

FIGS. 12A-12E show the enrichment of IgG subtype plasma cells in atezolizumab responders. FIG. 12A shows % IgG; FIG. 12B shows the ratio of IgG to IgM; FIG. 12C shows % IgM; FIG. 12D shows the relative amount of IgG as compared to total IgG and IgM content; and FIG. 12E shows % IgA. Summary plots of BCR sequencing for different Ig domains were compiled from limited patient samples classified as PR (brown), SD (blue) and PD (red) patients according to RECIST v1.1 before and after atezolizumab treatment (n=17).

FIGS. 13A-13D show the association of survival benefit with B cell immunophenotypes. KM curves comparing probability of survival in patients enriched for (FIG. 13A) T cell effector gene signature along with CD79A in OAK trial (>median docetaxel=12.97, <median docetaxel=9.12, >median atezolizumab=15.9, <median atezolizumab=9.48 months); (FIG. 13B) T cell effector gene signature along with CD79A in POPLAR trial (>median docetaxel=9.63, <median docetaxel=9.35, <median atezolizumab=8.54 months); (FIG. 13C) T cell effector gene signature alone in OAK trial (>median docetaxel=11.1, <median docetaxel=9.82, >median atezolizumab=15.34, <median atezolizumab=months); and (FIG. 13D) T cell effector gene signature alone in POPLAR trial (>median docetaxel=9.72, <median docetaxel=9.23, >median atezolizumab=15.47, <median atezolizumab=9.72 months).

FIGS. 14A-14C show the association of B cell signature with OS benefit is consistent across major subgroups. High B cell signature is associated with atezolizumab mediated OS benefit across major subgroups: (FIG. 14A) squamous vs non-squamous, (FIG. 14B) biopsy vs resection, and (FIG. 14C) lung tumors vs lymph node metastases.

FIGS. 15A-15G show that intratumoral B cells associate with increased OS in NSCLC patients treated with atezolizumab. (FIG. 15A) Volcano plot depicting differentially expressed genes (FDR P<0.05, absolute logFC>=0.5) between patients from OAK with OS<6 months (n=205) versus OS>12 months (n=205) after treatment with atezolizumab. (FIG. 15B) Same as FIG. 15A, in patients treated with docetaxel. (FIGS. 15C-15F) Kaplan Meier (KM) curves comparing the probability of survival in patients enriched for CD79A, CD19, IFNG and the IFN inducible chemokine CXCL10. Gene expression was dichotomized as high (top tertile T3) or low/intermediate (tertiles T1 and T2). (FIG. 15G) Representative immunofluorescence images of pre-treatment lung adenocarcinoma tumor from two patients responsive to atezolizumab (left panels) and two patients non-responsive to atezolizumab. (Scale Bar: 100 μm).

FIGS. 16A-16D show the identification of three B cell subsets in NSCLC tumors. (FIG. 16A) Left: UMAP dimensionality reduction of 20,362 cells (dots). The same UMAP is given on the top right. Bottom right: The fraction of cells in each cluster from metastatic lymph node (mLN), non-metastatic lymph node (nLN), normal adjacent lung tissue (nLung), tumor biopsy (Tbio) and tumor resection (T res). Bottom center: Relative average expression of indicated markers in clusters from FIG. 16A. (FIG. 16B) Left: The fraction of cells from each patient (rows) for clusters given in FIG. 16A. Right: Absolute numbers of cells from each patient for clusters in FIG. 16A. (FIG. 16C) Violin plots indicating the expression of marker genes in clusters from FIG. 16A. (FIG. 16D) UMAPs of B cell subsets from six procured fresh NSCLC tumors analyzed by CyTOF, recapitulating the presence of follicular B cells (HLA-DR+, CD38−), germinal center (GC) B cells (HLA-DR+, CD38+Ki67+) and plasma cells (HLA-DR−, CD38++).

FIGS. 17A and 17B show B cell subset signatures in bulk RNAseq profiles. (FIG. 17A) Hierarchical clustering of the three B cell signatures identified from the scRNA-seq data in OAK. (FIG. 17B) Scatter plots showing the correlations between plasma cell, germinal center B cell and follicular B cell signatures. The Pearson R value is reported.

FIGS. 18A-18F show that plasma cell signature independently predicts response to atezolizumab. (FIGS. 18A-18C) Kaplan-Meier curves of OS for each of the three signatures, dichotomized as T3 (top tertile) vs T1-T2 (low/median tertiles). The log-rank p-value is reported. (FIG. 18D) Heatmap depicting the results from Cox proportional hazard models testing hazard ratios within and across arms. Dots represent statistically significant HRs (p<0.05). (FIG. 18E) Forest plot depicting the significance of the three B cell signatures and a previously reported 8-gene T-effector signature (tGE8) in univariate interaction models, where the interaction between signature score and treatment arm is considered. (FIG. 18F) Forest plots depicting the significance of the four signatures shown in FIG. 18E in multivariate analysis for atezolizumab (left panel) and docetaxel (right panel) arms. Signatures are dichotomized as T3 vs. T1-T2 in all models.

FIGS. 19A-19C show that patients with TLS/LA+ tumors exhibit improved OS with atezolizumab. (FIG. 19A) H&E staining depicting tumors with tertiary lymphoid structures (TLS, left panel), lymphoid aggregates only (center panel) or neither (right panel) in representative samples from POPLAR. (FIG. 19B) Bar chart representing the proportion of tumors with TLS, lymphoid aggregates only (LA) or neither in each treatment arm in POPLAR. (FIG. 19C) Kaplan-Meier curve representing OS for tumors with TLS or LA vs. those with neither, by treatment arm.

FIGS. 20A-20C show that TLS/LA+ tumors are enriched for plasma cells. (FIG. 20A) Hierarchical cluster of the three B cell subset signatures. Samples are ordered by TLS/LA status. (FIG. Volcano plot representing differentially expressed genes between tumors with TLS and/or LA vs. tumors with neither. Genes from the three B cell signatures are highlighted. (FIG. 20C) Violin plots depicting signature z-scores for the plasma cell, germinal center B cell and follicular B cells, grouped by TLS/LA status. Mann-Whitney p-values are reported.

FIGS. 21A-21F provide additional information on the data shown in FIGS. 15A-15G. (FIG. 21A) Volcano plot depicting differentially expressed genes (FDR P<0.05, absolute logFC>=0.5) between patients from POPLAR with OS<6 months (n=58) versus OS>12 months (n=87) after treatment with atezolizumab. (FIG. 21B) Same as FIG. 21A, in patients treated with docetaxel. FIGS. 21C-21F) Kaplan Meier (KM) curves comparing the probability of survival in patients enriched for CD79A, CD19, IFNG and the IFN-inducible chemokine CXCL10. Gene expression was dichotomized as high (top tertile T3) or low/intermediate (tertiles T1 and T2).

FIGS. 22A and 22B provide additional information on the data shown in FIGS. 16A-16D. (FIG. 22A) Expression of putative signature genes for follicular B cells, plasma cells, and GC B cells in non-B cell scRNA-seq compartments. Highlighted are candidate markers for bulk deconvolution, reasoning for removal of signature genes due to high background in bulk are indicated. (FIG. 22B) UMAP projection of scRNA-seq expression within B cells for CyTOF marker genes that delineate follicular B cells, plasma cells, and GC B cells recapitulating the CyTOF results where follicular B cells (HLA-DR+, CD38−), germinal center (GC) B cells (HLA-DR+, CD38+Ki67+) and plasma cells (HLADR−, CD38++).

FIG. 23 is a Pearson correlation of B cell subset signature genes across all samples in OAK, as described in Example 1, below.

FIGS. 24A-24E provide additional information on FIGS. 18A-18F. (FIG. 24A) Dichotomized plasma cell signature score by tertile stratifying best overall responses as objective responses or durable stable disease (SD with PFS 6 months) versus progressive disease or non-durable stable disease (SD with PFS<6 months) within each arm. P-value is a Fisher's Exact Test. (FIGS. 24B-24D) Kaplan-Meier curves of OS for each of the three signatures, dichotomized as T3 (top tertile) vs T1-T2 (low/median tertiles). The log-rank P-value is reported. (FIG. 24E) Kaplan-Meier curves of OS for the plasma cell signature, dichotomized as T3 vs. T1-2 in TOGA 365 LUAD/LUSC data. The hazard ratio for overall survival with high plasma signature and associated P-value is shown.

### DETAILED DESCRIPTION

The present disclosure provides diagnostic methods, therapeutic methods, and compositions for the treatment of cancer (e.g., lung cancer (e.g., non-small cell lung cancer (NSCLC)), bladder cancer (e.g., urothelial carcinoma (UC)), kidney cancer (e.g., renal cell carcinoma (RCC)), and breast cancer (e.g., triple-negative breast cancer (TNBC))).

The disclosure is based, at least in part, on the discovery that one or more of the biomarkers disclosed herein, e.g., the presence and/or expression level of any gene set forth in any one of Tables 1-17, the presence and/or expression level of a B cell signature (e.g., a plasma B cell signature), the presence of a tertiary lymphoid structure (TLS), the presence and/or expression level of a TLS signature, the presence and/or number of B cells, and/or the presence and/or number of clonally expanded B cells, can be used to identify and select individuals who are likely to benefit from treatment with a PD-L1 axis binding antagonist (e.g., a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

For example, as described in Example 1, below, the present disclosure demonstrates that elevated expression levels of CD79A and other B cell signature genes, including plasma B cell signature genes, were associated with improved overall survival (OS) in NSCLC patients receiving treatment with the anti-PD-L1 antibody atezolizumab. Similarly, the presence of tertiary lymphoid structures (TLS), as well as elevated expression levels of TLS signature genes, were also associated with improved OS in NSCLC patients receiving treatment with the anti-PD-L1 antibody atezolizumab. Thus, the biomarkers disclosed herein can be used, e.g., to identify individuals who are likely to benefit from treatment with a PD-L1 axis binding antagonist (e.g., a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), to select patients for an optimized cancer therapy, and to provide personalized treatment approaches for patients who are likely to benefit.

### I. DEFINITIONS

The term “about” as used herein refers to the usual error range for the respective value readily known to the skilled person in this technical field. Reference to “about” a value or parameter herein includes (and describes) embodiments that are directed to that value or parameter per se.

As used herein, “administering” is meant a method of giving a dosage of a compound (e.g., a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) or a composition (e.g., a pharmaceutical composition, e.g., a pharmaceutical composition including a PD-L1 axis binding antagonist) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in creams, or in lipid compositions. The method of administration can vary depending on various factors (e.g., the compound or composition being administered, and the severity of the condition, disease, or disorder being treated).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “antibody” herein is used in the broadest sense and encompasses various antibody structures, including, but not limited to, monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one embodiment, the extent of binding of an anti-PD-L1 antibody to an unrelated, non-PD-L1 protein is less than about 10% of the binding of the antibody to PD-L1 as measured, e.g., by a radioimmunoassay (RIA). In certain embodiments, an anti-PD-L1 antibody binds to an epitope of PD-L1 that is conserved among PD-L1 from different species. In certain embodiments, the anti-PD-L1 antibody is atezolizumab. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No. Q9NZQ7.1.

The term “anti-cancer therapy” refers to a therapy useful for treating a cancer (e.g., a lung cancer (e.g., non-small cell lung cancer (NSCLC), including non-squamous NSCLC and squamous NSCLC), a bladder cancer (e.g., a urothelial carcinoma (UC)), a kidney cancer (e.g., a renal cell carcinoma (RCC)), or a breast cancer (e.g., a triple-negative breast cancer (TNBC))). Examples of anti-cancer therapeutic agents include, but are limited to, e.g., PD-L1 axis binding antagonists (e.g., a PD-L1 binding antagonist (e.g., an anti-PD-L1 antibody, e.g., atezolizumab) or a PD-1 binding antagonist (e.g., an anti-PD-1 antibody)), chemotherapeutic agents, growth inhibitory agents, cytotoxic agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets: PDGFR-8, BlyS, APRIL, BCMA receptor(s), TRAIL/Apo2, other bioactive and organic chemical agents, and the like. Combinations thereof are also included in the invention.

An “article of manufacture” or a “kit,” as used interchangeably herein, refers to any manufacture (e.g., a package or container) or kit comprising at least one reagent, e.g., a medicament for treatment of a disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)), or a probe (e.g., a nucleic acid probe or an antibody) for specifically detecting a biomarker described herein. In certain embodiments, the manufacture or kit is promoted, distributed, or sold as a unit for performing the methods described herein.

The phrase “based on” when used herein means that the information about one or more biomarkers is used to inform a treatment decision, information provided on a package insert, or marketing/promotional guidance, etc.

The term “B cell,” as used herein, refers to a lymphocyte that matures within the bone marrow, and includes, without limitation, a naïve B cell, memory B cell, or a plasma B cell (also referred to as a plasma cell or an effector B cell). B cells are also known in the art as “B lymphocytes.” B cells, unlike other lymphocytes such as T cells or natural killer cells, can express B cell receptors (BCRs) on their plasma membrane.

A “B cell receptor” or “BCR” is a transmembrane receptor complex located on the plasma membrane of B cells. BCRs include a membrane-bound immunoglobulin (mIg) moiety (e.g., mIgA, mlgG, mIgE, mlgM, or mlgD) and a signal transduction moiety composed of a CD79A/CD79B heterodimer (also known as Ig-α/Ig13). Each member of the CD79A/CD79B heterodimer spans the plasma membrane and includes a cytoplasmic tail that includes an immunoreceptor tyrosine-based activation motif (ITAM).

A “blocking” antibody or an “antagonist” antibody is one which inhibits or reduces biological activity of the antigen it binds. Preferred blocking antibodies or antagonist antibodies substantially or completely inhibit the biological activity of the antigen.

By “binding domain” is meant a part of a compound or a molecule that specifically binds to a target epitope, antigen, ligand, or receptor. Binding domains include, but are not limited to, antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “biomarker” as used herein refers to an indicator, e.g., predictive, diagnostic, and/or prognostic, which can be detected in a sample (e.g., any gene set forth in any one of Tables 1-17, e.g., one or more of CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7). The biomarker may serve as an indicator of a particular subtype of a disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)) characterized by certain molecular, pathological, histological, and/or clinical features. In some embodiments, a biomarker is a gene. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, glycolipid-based molecular markers, cells (e.g., B cells), and/or histological structures (e.g., tertiary lymphoid structures).

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic (e.g., the immune-score expression level of one or more of any gene set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7). The biomarker signature may serve as an indicator of a particular subtype of a disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. The gene signature may be, e.g., a B cell gene signature (e.g., one or more of genes CD79A, CD19, BANK1, JCHAIN, SLAMF7, BTK, TNFRSF17, IGJ, IGLL5, RBPJ, and/or MZB1), a naïve B cell gene signature (e.g., one or more of genes ABCB4, BCL7A, BENDS, BRAF, IL4R, LINC00921, MEP1A, MICAL3, NIPSNAP3B, PSG2, SELL, TCL1A, UGT1A8, and/or ZNF286A), a memory B cell gene signature (e.g., one or more of genes AIM2, ALOX5, CLCA3P, FAM65B, IFNA10, IL7, NPIPB15, SP140, TNFRSF13B, TRAF4, and/or ZBTB32), a plasma cell gene signature (e.g., one or more of genes DERL3, JSRP1, TNFRSF17, SLAMF7, IGHG2, IGHGP, IGLV3-1, IGLV6-57, IGHA2, IGKV4-1, IGKV1-12, IGLC7, and/or IGLL5, and/or one or more of genes ABCB9, AMPD1, ANGPT4, ATXN8OS, C11, CCr10, HIST1H2AE, HIST1H2BG, IGHE, KCNA3, KCNG2, LOC100130100, MANI A1, MANEA, MAST1, MROH7, MZB1, PAX7, PDK1, RASGRP3, REN, SPAG4, ST6GALNAC4, TGM5, UGT2B17, ZBP1, and/or ZNF16), a TLS signature (e.g., one or more of genes CCL2, CCL3, CCL4, CCL5, CCL8, CCL18, CCL19, CCL21, CXCL9, CXCL10, CXCL11, and/or CXCL13), or a T effector signature (e.g., one or more of genes CD8A, EOMES, GZMA, TBX21, IFNG, GZMB, CXCL9, and/or CXCL10). In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The term “CD79A” as used herein refers to the cluster of differentiation CD79A gene, including any native CD79A from any vertebrate source, including mammals such as primates (e.g., humans) and rodents (e.g., mice and rats), unless otherwise indicated. CD79A is also known in the art as Ig-α, B-cell antigen receptor complex-associated protein alpha chain, and MB-1 membrane glycoprotein. The term encompasses “full-length,” unprocessed CD79A as well as any form of CD79A that results from processing in the cell. The term also encompasses naturally occurring variants of CD79A, e.g., splice variants or allelic variants. The nucleic acid sequence of an exemplary human CD79A is listed in SEQ ID NO: 13 (NCBI Reference Sequence: NM 001783.4). The amino acid sequence of an exemplary protein encoded by human CD79A is shown in SEQ ID NO: 14 (UNIPROT™ Accession No. P11912-1).

The term “SLAMF7” as used herein, refers to any native SLAMF7 (signaling lymphocytic activation molecule (SLAM) Family Member 7) from any vertebrate source, including mammals such as primates (e.g., humans) and rodents (e.g., mice and rats), unless otherwise indicated. The term encompasses “full-length,” unprocessed SLAMF7 as well as any form of SLAMF7 that results from processing in the cell. The term also encompasses naturally occurring variants of SLAMF7, e.g., splice variants or allelic variants. The nucleic acid sequence of an exemplary human SLAMF7 is listed in SEQ ID NO: 15 (NCBI Reference Sequence: NM 021181.5). The amino acid sequence of an exemplary protein encoded by human SLAMF7 is shown in SEQ ID NO: 16 (UNIPROT™ Accession No. Q9NQ25-1).

The term “BTK” as used herein, refers to any native BTK (Bruton's tyrosine kinase) from any vertebrate source, including mammals such as primates (e.g., humans) and rodents (e.g., mice and rats), unless otherwise indicated. The term encompasses “full-length,” unprocessed BTK as well as any form of BTK that results from processing in the cell. The term also encompasses naturally occurring variants of BTK, e.g., splice variants or allelic variants. The nucleic acid sequence of an exemplary human BTK is listed in SEQ ID NO: 17 (NCBI Reference Sequence: NM 000061.2). The amino acid sequence of an exemplary protein encoded by human BTK is shown in SEQ ID NO: 18 (UNIPROT™ Accession No. Q06187-1).

The term “TNFRSF17” as used herein, refers to any native TNFRSF17 (tumor necrosis factor receptor superfamily member 17) from any vertebrate source, including mammals such as primates (e.g., humans) and rodents (e.g., mice and rats), unless otherwise indicated. TNFRSF17 is also known in the art as B cell maturation antigen (BCMA). The term encompasses “full-length,” unprocessed TNFRSF17 as well as any form of TNFRSF17 that results from processing in the cell. The term also encompasses naturally occurring variants of TNFRSF17, e.g., splice variants or allelic variants. The nucleic acid sequence of an exemplary human TNFRSF17 is listed in SEQ ID NO: 19 (NCBI Reference Sequence: NM 001192.3). The amino acid sequence of an exemplary protein encoded by human TNFRSF17 is shown in SEQ ID NO: 20 (UNIPROT™ Accession No. Q02223-1).

The term “IGJ” as used herein, refers to any native IGJ (immunoglobulin J chain) from any vertebrate source, including mammals such as primates (e.g., humans) and rodents (e.g., mice and rats), unless otherwise indicated. The term encompasses “full-length,” unprocessed IGJ as well as any form of IGJ that results from processing in the cell. The term also encompasses naturally occurring variants of IGJ, e.g., splice variants or allelic variants. The nucleic acid sequence of an exemplary human IGJ is listed in SEQ ID NO: 21 (NCBI Reference Sequence: NM 144646.4). The amino acid sequence of an exemplary protein encoded by human IGJ is shown in SEQ ID NO: 22 (UNIPROT™ Accession No. P01591-1).

The term “IGLL5” as used herein, refers to any native IGLL5 (immunoglobulin lambda like polypeptide 5) from any vertebrate source, including mammals such as primates (e.g., humans) and rodents (e.g., mice and rats), unless otherwise indicated. IGLL5 is also known in the art as IGL, IGLV, and VL_MAR. The term encompasses “full-length,” unprocessed IGLL5 as well as any form of IGLL5 that results from processing in the cell. The term also encompasses naturally occurring variants of IGLL5, e.g., splice variants or allelic variants. The nucleic acid sequence of an exemplary human IGLL5 is listed in SEQ ID NO: 23 (NCBI Reference Sequence: NM 001178126.2). The amino acid sequence of an exemplary protein encoded by human IGLL5 is shown in SEQ ID NO: 24 (UNIPROT™ Accession No. B9A064-1).

The term “RBPJ” as used herein, refers to any native RBPJ (recombination signal binding protein for immunoglobulin kappa J region) from any vertebrate source, including mammals such as primates (e.g., humans) and rodents (e.g., mice and rats), unless otherwise indicated. RBPJ is also known in the art as CBF1 and recombining binding protein suppressor of hairless. The term encompasses “full-length,” unprocessed RBPJ as well as any form of RBPJ that results from processing in the cell. The term also encompasses naturally occurring variants of RBPJ, e.g., splice variants or allelic variants. The nucleic acid sequence of an exemplary human RBPJ is listed in SEQ ID NO: 25 (NCBI Reference Sequence: NM 005349.3). The amino acid sequence of an exemplary protein encoded by human RBPJ is shown in SEQ ID NO: 26 (UNIPROT™ Accession No. Q06330-1).

The term “MZB1” as used herein, refers to any native MZB1 (marginal zone B and B1 cell-specific protein) from any vertebrate source, including mammals such as primates (e.g., humans) and rodents (e.g., mice and rats), unless otherwise indicated. MZB1 is also known in the art as MEDA-7, PACAP, and pERp1. The term encompasses “full-length,” unprocessed MZB1 as well as any form of MZB1 that results from processing in the cell. The term also encompasses naturally occurring variants of MZB1, e.g., splice variants or allelic variants. The nucleic acid sequence of an exemplary human MZB1 is listed in SEQ ID NO: 27 (NCBI Reference Sequence: NM 016459.4). The amino acid sequence of an exemplary protein encoded by human MZB1 is shown in SEQ ID NO: 28 (UNIPROT™ Accession No. Q8WU39-1).

The term “CCL2” as used herein, refers to any native CCL2 (chemokine (C—C motif) ligand 2) from any vertebrate source, including mammals such as primates (e.g., humans) and rodents (e.g., mice and rats), unless otherwise indicated. CCL2 is also known in the art as monocyte chemoattractant protein 1 (MCP1) and small inducible cytokine A2. The term encompasses “full-length,” unprocessed CCL2 as well as any form of CCL2 that results from processing in the cell. The term also encompasses naturally occurring variants of CCL2, e.g., splice variants or allelic variants. The nucleic acid sequence of an exemplary human CCL2 is listed in SEQ ID NO: 29 (NCBI Reference Sequence: NM 002982.4). The amino acid sequence of an exemplary protein encoded by human CCL2 is shown in SEQ ID NO: 30 (UNIPROT™ Accession No. P13500-1).

The term “CCL3” as used herein, refers to any native CCL3 (chemokine (C—C motif) ligand 3) from any vertebrate source, including mammals such as primates (e.g., humans) and rodents (e.g., mice and rats), unless otherwise indicated. CCL3 is also known in the art as macrophage inflammatory protein 1-alpha (MIP-1-alpha). The term encompasses “full-length,” unprocessed CCL3 as well as any form of CCL3 that results from processing in the cell. The term also encompasses naturally occurring variants of CCL3, e.g., splice variants or allelic variants. The nucleic acid sequence of an exemplary human CCL3 is listed in SEQ ID NO: 31 (NCBI Reference Sequence: NM 002983.3). The amino acid sequence of an exemplary protein encoded by human CCL3 is shown in SEQ ID NO: 32 (UNIPROT™ Accession No. P10147-1).

The term “CCL4” as used herein, refers to any native CCL4 (chemokine (C—C motif) ligand 4) from any vertebrate source, including mammals such as primates (e.g., humans) and rodents (e.g., mice and rats), unless otherwise indicated. CCL4 is also known in the art as macrophage inflammatory protein 1-beta (MIP-1-beta). The term encompasses “full-length,” unprocessed CCL4 as well as any form of CCL4 that results from processing in the cell. The term also encompasses naturally occurring variants of CCL4, e.g., splice variants or allelic variants. The nucleic acid sequence of an exemplary human CCL4 is listed in SEQ ID NO: 33 (NCBI Reference Sequence: NM 002984.4). The amino acid sequence of an exemplary protein encoded by human CCL4 is shown in SEQ ID NO: 34 (UNIPROT™ Accession No. P13236-1).

The term “CCL5” as used herein, refers to any native CCL5 (chemokine (C—C motif) ligand 5) from any vertebrate source, including mammals such as primates (e.g., humans) and rodents (e.g., mice and rats), unless otherwise indicated. CCL5 is also known in the art as regulated on activation, normal T cell expressed and secreted (RANTES), SCYAS, SIS-delta, SISd, TCP228, and eoCP. The term encompasses “full-length,” unprocessed CCL5 as well as any form of CCL5 that results from processing in the cell. The term also encompasses naturally occurring variants of CCL5, e.g., splice variants or allelic variants. The nucleic acid sequence of an exemplary human CCL5 is listed in SEQ ID NO: 35 (European Nucleotide Archive Accession No. AF043341.1). The amino acid sequence of an exemplary protein encoded by human CCL5 is shown in SEQ ID NO: 36 (UNIPROT™ Accession No. P13501-1).

The term “CCL8” as used herein, refers to any native CCL8 (chemokine (C—C motif) ligand 8) from any vertebrate source, including mammals such as primates (e.g., humans) and rodents (e.g., mice and rats), unless otherwise indicated. CCL8 is also known in the art as monocyte chemoattractant protein 2 (MCP2). The term encompasses “full-length,” unprocessed CCL8 as well as any form of CCL8 that results from processing in the cell. The term also encompasses naturally occurring variants of CCL8, e.g., splice variants or allelic variants. The nucleic acid sequence of an exemplary human CCL8 is listed in SEQ ID NO: 37 (NCBI Reference Sequence: NM 005623.3). The amino acid sequence of an exemplary protein encoded by human CCL8 is shown in SEQ ID NO: 38 (UNIPROT™ Accession No. P80075-1).

The term “CCL18” as used herein, refers to any native CCL18 (chemokine (C—C motif) ligand 18) from any vertebrate source, including mammals such as primates (e.g., humans) and rodents (e.g., mice and rats), unless otherwise indicated. CCL18 is also known in the art as pulmonary and activation-regulated chemokine (PARC), dendritic cell (DC)-chemokine 1 (DC—CK1), alternative macrophage activation-associated CC chemokine-1 (AMAC-1), and macrophage inflammatory protein-4 (MIP-4). The term encompasses “full-length,” unprocessed CCL18 as well as any form of CCL18 that results from processing in the cell. The term also encompasses naturally occurring variants of CCL18, e.g., splice variants or allelic variants. The nucleic acid sequence of an exemplary human CCL18 is listed in SEQ ID NO: 39 (NCBI Reference Sequence: NM 002988.4). The amino acid sequence of an exemplary protein encoded by human CCL18 is shown in SEQ ID NO: 40 (UNIPROT™ Accession No. P55774-1).

The term “CCL19” as used herein, refers to any native CCL19 (chemokine (C—C motif) ligand 19) from any vertebrate source, including mammals such as primates (e.g., humans) and rodents (e.g., mice and rats), unless otherwise indicated. CCL19 is also known in the art as EBI1 ligand chemokine (ELC) and macrophage inflammatory protein-3-beta (MIP-3-beta). The term encompasses “full-length,” unprocessed CCL19 as well as any form of CCL19 that results from processing in the cell. The term also encompasses naturally occurring variants of CCL19, e.g., splice variants or allelic variants. The nucleic acid sequence of an exemplary human CCL19 is listed in SEQ ID NO: 41 (NCBI Reference Sequence: NM 006274.3). The amino acid sequence of an exemplary protein encoded by human CCL19 is shown in SEQ ID NO: 42 (UNIPROT™ Accession No. Q99731-1).

The term “CCL21” as used herein, refers to any native CCL21 (chemokine (C—C motif) ligand 21) from any vertebrate source, including mammals such as primates (e.g., humans) and rodents (e.g., mice and rats), unless otherwise indicated. CCL21 is also known in the art as 6Ckine, exodus-2, and secondary lymphoid-tissue chemokine (SLC). The term encompasses “full-length,” unprocessed CCL21 as well as any form of CCL21 that results from processing in the cell. The term also encompasses naturally occurring variants of CCL21, e.g., splice variants or allelic variants. The nucleic acid sequence of an exemplary human CCL21 is listed in SEQ ID NO: 43 (NCBI Reference Sequence: NM 002989.4). The amino acid sequence of an exemplary protein encoded by human CCL21 is shown in SEQ ID NO: 44 (UNIPROT™ Accession No. 000585-1).

The term “CXCL9” as used herein, refers to any native CXCL9 (chemokine (C—X-C motif) ligand 9) from any vertebrate source, including mammals such as primates (e.g., humans) and rodents (e.g., mice and rats), unless otherwise indicated. CXCL9 is also known in the art as monokine induced by gamma interferon (MIG). The term encompasses “full-length,” unprocessed CXCL9 as well as any form of CXCL9 that results from processing in the cell. The term also encompasses naturally occurring variants of CXCL9, e.g., splice variants or allelic variants. The nucleic acid sequence of an exemplary human CXCL9 is listed in SEQ ID NO: 45 (NCBI Reference Sequence: NM 002416.3). The amino acid sequence of an exemplary protein encoded by human CXCL9 is shown in SEQ ID NO: 46 (UNIPROT™ Accession No. Q07325-1).

The term “CXCL10” as used herein, refers to any native CXCL10 (C—X-C motif chemokine ligand from any vertebrate source, including mammals such as primates (e.g., humans) and rodents (e.g., mice and rats), unless otherwise indicated. CXCL10 is also known in the art as interferon gamma-induced protein 10 (IP-10) or small-inducible cytokine B10. The term encompasses “full-length,” unprocessed CXCL10 as well as any form of CXCL10 that results from processing in the cell. The term also encompasses naturally occurring variants of CXCL10, e.g., splice variants or allelic variants. The nucleic acid sequence of an exemplary human CXCL10 is listed in SEQ ID NO: 47 (NCBI Reference Sequence: NM 001565.4). The amino acid sequence of an exemplary protein encoded by human CXCL10 is shown in SEQ ID NO: 48 (UNIPROT™ Accession No. P02778-1).

The term “CXCL11” as used herein, refers to any native CXCL11 (C—X-C motif chemokine ligand 11) from any vertebrate source, including mammals such as primates (e.g., humans) and rodents (e.g., mice and rats), unless otherwise indicated. CXCL11 is also known in the art as interferon-inducible T-cell alpha chemoattractant (I-TAC) and Interferon-gamma-inducible protein 9 (IP-9). The term encompasses “full-length,” unprocessed CXCL11 as well as any form of CXCL11 that results from processing in the cell. The term also encompasses naturally occurring variants of CXCL11, e.g., splice variants or allelic variants. The nucleic acid sequence of an exemplary human CXCL11 is listed in SEQ ID NO: 49 (NCBI Reference Sequence: NM 005409.5). The amino acid sequence of an exemplary protein encoded by human CXCL11 is shown in SEQ ID NO: 50 (UNIPROT™ Accession No. 014625-1).

The term “CXCL13” as used herein, refers to any native CXCL13 (C—X-C motif chemokine ligand 13) from any vertebrate source, including mammals such as primates (e.g., humans) and rodents (e.g., mice and rats), unless otherwise indicated. CXCL13 is also known in the art as B lymphocyte chemoattractant (BLC) and B cell-attracting chemokine 1 (BCA-1). The term encompasses “full-length,” unprocessed CXCL13 as well as any form of CXCL13 that results from processing in the cell. The term also encompasses naturally occurring variants of CXCL13, e.g., splice variants or allelic variants. The nucleic acid sequence of an exemplary human CXCL13 is listed in SEQ ID NO: 51 (NCBI Reference Sequence: NM 006419.2). The amino acid sequence of an exemplary protein encoded by human CXCL13 is shown in SEQ ID NO: 52 (UNIPROT™ Accession No. 043927-1).

The term “CD8A” as used herein refers to the cluster of differentiation 8a gene, including any native CD8A from any vertebrate source, including mammals such as primates (e.g., humans) and rodents (e.g., mice and rats), unless otherwise indicated. The term encompasses “full-length,” unprocessed CD8A as well as any form of CD8A that results from processing in the cell. The term also encompasses naturally occurring variants of CD8A, e.g., splice variants or allelic variants. The nucleic acid sequence of an exemplary human CD8A is listed in SEQ ID NO: 53 (GENBANK™ Accession No. M12828.1). The amino acid sequence of an exemplary protein encoded by human CD8A is shown in SEQ ID NO: 54 (UNIPROT™ Accession No. P01732-1).

The term “EOMES” as used herein refers to the eomesodermin gene, including any native EOMES from any vertebrate source, including mammals such as primates (e.g., humans) and rodents (e.g., mice and rats), unless otherwise indicated. EOMES is also known in the art as T-box brain protein 2 (Tbr2). The term encompasses “full-length,” unprocessed EOMES as well as any form of EOMES that results from processing in the cell. The term also encompasses naturally occurring variants of EOMES, e.g., splice variants or allelic variants. The nucleic acid sequence of an exemplary human EOMES is listed in SEQ ID NO: 55 (NCBI Reference Sequence: NM 005442.4). The amino acid sequence of an exemplary protein encoded by human EOMES is shown in SEQ ID NO: 56 (UNIPROT™ Accession No. 095936-1).

The term “GZMA” as used herein refers to the granzyme A gene, including any native GZMA from any vertebrate source, including mammals such as primates (e.g., humans) and rodents (e.g., mice and rats), unless otherwise indicated. The term encompasses “full-length,” unprocessed GZMA as well as any form of GZMA that results from processing in the cell. The term also encompasses naturally occurring variants of GZMA, e.g., splice variants or allelic variants. The nucleic acid sequence of an exemplary human GZMA is listed in SEQ ID NO: 57 (GENBANK™ Accession No. BC015739). The amino acid sequence of an exemplary protein encoded by human GZMA is shown in SEQ ID NO: 58 (UNIPROT™ Accession No. P12544-1).

The term “TBX21” as used herein refers to the T-box transcription factor TBX21 gene, including any native TBX21 from any vertebrate source, including mammals such as primates (e.g., humans) and rodents (e.g., mice and rats), unless otherwise indicated. TBX21 is also known in the art as T-PET, T-bet, TBLYM, and T-box21. The term encompasses “full-length,” unprocessed TBX21 as well as any form of TBX21 that results from processing in the cell. The term also encompasses naturally occurring variants of TBX21, e.g., splice variants or allelic variants. The nucleic acid sequence of an exemplary human TBX21 is listed in SEQ ID NO: 59 (NCBI Reference Sequence: NM 013351.2). The amino acid sequence of an exemplary protein encoded by human TBX21 is shown in SEQ ID NO: 60 (UNIPROT™ Accession No. Q9UL17-1).

The term “IFNG” as used herein refers to the interferon gamma gene, including any native IFNG from any vertebrate source, including mammals such as primates (e.g., humans) and rodents (e.g., mice and rats), unless otherwise indicated. IFNG is also known in the art as type II interferon. The term encompasses “full-length,” unprocessed IFNG as well as any form of IFNG that results from processing in the cell. The term also encompasses naturally occurring variants of IFNG, e.g., splice variants or allelic variants. The nucleic acid sequence of an exemplary human IFNG is listed in SEQ ID NO: 61 (NCBI Reference Sequence: NM 000619.3). The amino acid sequence of an exemplary protein encoded by human IFNG is shown in SEQ ID NO: 62 (UNIPROT™ Accession No. P01579-1).

The term “GZMB” as used herein refers to the granzyme B gene, including any native GZMB from any vertebrate source, including mammals such as primates (e.g., humans) and rodents (e.g., mice and rats), unless otherwise indicated. The term encompasses “full-length,” unprocessed GZMB as well as any form of GZMB that results from processing in the cell. The term also encompasses naturally occurring variants of GZMB, e.g., splice variants or allelic variants. The nucleic acid sequence of an exemplary human GZMB is listed in SEQ ID NO: 63 (GENBANK™ Accession No. J03072). The amino acid sequence of an exemplary protein encoded by human GZMB is shown in SEQ ID NO: 64 (UNIPROT™ Accession No. P10144-1).

The term “DERL3” as used herein refers to the derlin-3 gene, including any native DERL3 from any vertebrate source, including mammals such as primates (e.g., humans) and rodents (e.g., mice and rats), unless otherwise indicated. The term encompasses “full-length,” unprocessed DERL3 as well as any form of DERL3 that results from processing in the cell. The term also encompasses naturally occurring variants of DERL3, e.g., splice variants or allelic variants. The nucleic acid sequence of an exemplary human DERL3 is listed in SEQ ID NO: 65 (European Nucleotide Archive Accession No. AK125830.1). The amino acid sequence of an exemplary protein encoded by human DERL3 is shown in SEQ ID NO: 66 (UNIPROT™ Accession No. Q96Q80-1).

The term “JSRP1” as used herein refers to the junctional sarcoplasmic reticulum protein 1 gene, including any native JSRP1 from any vertebrate source, including mammals such as primates (e.g., humans) and rodents (e.g., mice and rats), unless otherwise indicated. The term encompasses “full-length,” unprocessed JSRP1 as well as any form of JSRP1 that results from processing in the cell. The term also encompasses naturally occurring variants of JSRP1, e.g., splice variants or allelic variants. The nucleic acid sequence of an exemplary human JSRP1 is listed in SEQ ID NO: 67 (European Nucleotide Archive Accession No. BC021201.2). The amino acid sequence of an exemplary protein encoded by human JSRP1 is shown in SEQ ID NO: 68 (UNIPROT™ Accession No. Q96MG2-1).

The term “IGHG2” as used herein refers to the immunoglobulin heavy constant gamma 2 gene, including any native IGHG2 from any vertebrate source, including mammals such as primates (e.g., humans) and rodents (e.g., mice and rats), unless otherwise indicated. The term encompasses “full-length,”unprocessed IGHG2 as well as any form of IGHG2 that results from processing in the cell. The term also encompasses naturally occurring variants of IGHG2, e.g., splice variants or allelic variants. The nucleic acid sequence of an exemplary human IGHG2 is listed in SEQ ID NO: 69 (European Nucleotide Archive Accession No. AL928742.3). The amino acid sequence of an exemplary protein encoded by human IGHG2 is shown in SEQ ID NO: 70 (UNIPROT™ Accession No. P01859-1).

The term “IGHGP” as used herein refers to the immunoglobulin heavy constant gamma P gene, including any native IGHGP from any vertebrate source, including mammals such as primates (e.g., humans) and rodents (e.g., mice and rats), unless otherwise indicated. The term encompasses “full-length,” unprocessed IGHGP as well as any form of IGHGP that results from processing in the cell. The term also encompasses naturally occurring variants of IGHGP, e.g., splice variants or allelic variants. The nucleic acid sequence of an exemplary human IGHGP is listed in SEQ ID NO: 71 (NCBI Reference Sequence No. NG 001019.6).

The term “IGLV3-1” as used herein refers to the immunoglobulin lambda variable 3-1 gene, including any native IGLV3-1 from any vertebrate source, including mammals such as primates (e.g., humans) and rodents (e.g., mice and rats), unless otherwise indicated. The term encompasses “full-length,” unprocessed IGLV3-1 as well as any form of IGLV3-1 that results from processing in the cell. The term also encompasses naturally occurring variants of IGLV3-1, e.g., splice variants or allelic variants. The nucleic acid sequence of an exemplary human IGLV3-1 is listed in SEQ ID NO: 72 (European Nucleotide Archive Accession No. AC245028.2). The amino acid sequence of an exemplary protein encoded by human IGLV3-1 is shown in SEQ ID NO: 73 (UNIPROT™ Accession No. P01715-1).

The term “IGLV6-57” as used herein refers to the immunoglobulin lambda variable 6-57 gene, including any native IGLV6-57 from any vertebrate source, including mammals such as primates (e.g., humans) and rodents (e.g., mice and rats), unless otherwise indicated. The term encompasses “full-length,” unprocessed IGLV6-57 as well as any form of IGLV6-57 that results from processing in the cell. The term also encompasses naturally occurring variants of IGLV6-57, e.g., splice variants or allelic variants. The nucleic acid sequence of an exemplary human IGLV6-57 is listed in SEQ ID NO: 74 (European Nucleotide Archive Accession No. AC245060.1). The amino acid sequence of an exemplary protein encoded by human IGLV6-57 is shown in SEQ ID NO: 75 (UNIPROT™ Accession No. P01721-1).

The term “IGHA2” as used herein refers to the immunoglobulin heavy constant alpha 2 gene, including any native IGHA2 from any vertebrate source, including mammals such as primates (e.g., humans) and rodents (e.g., mice and rats), unless otherwise indicated. The term encompasses “full-length,” unprocessed IGHA2 as well as any form of IGHA2 that results from processing in the cell. The term also encompasses naturally occurring variants of IGHA2, e.g., splice variants or allelic variants. The nucleic acid sequence of an exemplary human IGHA2 is listed in SEQ ID NO: 76 (European Nucleotide Archive Accession No. AL928742.3). The amino acid sequence of an exemplary protein encoded by human IGHA2 is shown in SEQ ID NO: 77 (UNIPROT′ Accession No. P01877-1).

The term “IGKV4-1” as used herein refers to the immunoglobulin kappa variable 4-1 gene, including any native IGKV4-1 from any vertebrate source, including mammals such as primates (e.g., humans) and rodents (e.g., mice and rats), unless otherwise indicated. The term encompasses “full-length,” unprocessed IGKV4-1 as well as any form of IGKV4-1 that results from processing in the cell. The term also encompasses naturally occurring variants of IGKV4-1, e.g., splice variants or allelic variants. The nucleic acid sequence of an exemplary human IGKV4-1 is listed in SEQ ID NO: 78 (European Nucleotide Archive Accession No. X02990.1). The amino acid sequence of an exemplary protein encoded by human IGKV4-1 is shown in SEQ ID NO: 79 (UNIPROT™ Accession No. P06312-1).

The term “IGKV1-12” as used herein refers to the immunoglobulin kappa variable 1-12 gene, including any native IGKV1-12 from any vertebrate source, including mammals such as primates (e.g., humans) and rodents (e.g., mice and rats), unless otherwise indicated. The term encompasses “full-length,” unprocessed IGKV1-12 as well as any form of IGKV1-12 that results from processing in the cell. The term also encompasses naturally occurring variants of IGKV1-12, e.g., splice variants or allelic variants. The nucleic acid sequence of an exemplary human IGKV1-12 is listed in SEQ ID NO: 80 (European Nucleotide Archive Accession No. AC245015.2). The amino acid sequence of an exemplary protein encoded by human IGKV1-12 is shown in SEQ ID NO: 81 (UNIPROT™ Accession No. A0A0C4DH73-1).

The term “IGLC7” as used herein refers to the immunoglobulin lambda constant 7 gene, including any native IGLC7 from any vertebrate source, including mammals such as primates (e.g., humans) and rodents (e.g., mice and rats), unless otherwise indicated. The term encompasses “full-length,” unprocessed IGLC7 as well as any form of IGLC7 that results from processing in the cell. The term also encompasses naturally occurring variants of IGLC7, e.g., splice variants or allelic variants. The nucleic acid sequence of an exemplary human IGLC7 is listed in SEQ ID NO: 82 (European Nucleotide Archive Accession No. AC245028.2). The amino acid sequence of an exemplary protein encoded by human IGLC7 is shown in SEQ ID NO: 83 (UNIPROT™ Accession No. A0M8Q6-1).

The term “clonally expanded B cells” refers to B cells having a common antigen specificity, as assessed, for example, by sequence homology to the mlg moiety of the BCR. For instance, clonally expanded B cells may share at least 85% sequence identity (e.g., 85%, 86%, 87%, 88%, 89%, 90%, 91%, 92%, 93%, 94%, 95%, 96%, 97%, 98%, 99%, 99.9%, or 100% sequence identity) across the mlg moiety heavy and/or light chains (e.g., 85%, 86%, 87%, 88%, 89%, 90%, 91%, 92%, 93%, 94%, 95%, 96%, 97%, 98%, 99%, 99.9%, or 100% sequence identity across the CDR1, CDR2, and/or CDR3 regions of their mlg moiety heavy and/or light chains).

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, lung cancer, including small-cell lung cancer, NSCLC (e.g., non-squamous NSCLC and squamous NSCLC), adenocarcinoma of the lung, and squamous carcinoma of the lung; bladder cancer (e.g., urothelial carcinoma cancer (UC), muscle invasive bladder cancer (MIBC), and BCG-refractory non-muscle invasive bladder cancer (NMIBC)); kidney or renal cancer (e.g., renal cell carcinoma (RCC)); cancer of the urinary tract; breast cancer (e.g., HER2+ breast cancer and triple-negative breast cancer (TNBC), which are estrogen receptors (ER-), progesterone receptors (PR-), and HER2 (HER2-) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer; hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The terms “cell proliferative disorder” and “proliferative disorder” refer to disorders that are associated with some degree of abnormal cell proliferation. In one embodiment, the cell proliferative disorder is a cancer (e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). In another embodiment, the cell proliferative disorder is a tumor.

A “chemotherapeutic agent” is a chemical compound useful in the treatment of a cancer (e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1′ and calicheamicin w1′ (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET0), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA0), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (TARCEVA™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein also include “anti-hormonal agents” or “endocrine therapeutics” which act to regulate, reduce, block, or inhibit the effects of hormones that can promote the growth of cancer (e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® (toremifene); aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; vinorelbine and esperamicins (see U.S. Pat. No. 4,675,187), and pharmaceutically acceptable salts, acids or derivatives of any of the above; as well as combinations of two or more of the above.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

As used herein, “delaying progression” of a disorder or disease means to defer, hinder, slow, retard, stabilize, and/or postpone development of the disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). This delay can be of varying lengths of time, depending on the history of the disease and/or subject being treated. As is evident to one skilled in the art, a sufficient or significant delay can, in effect, encompass prevention, in that the subject does not develop the disease.

The terms “determination,” “determining,” “detection,” “detecting,” and grammatical variations thereof include any means of determining or detecting, including direct and indirect determination or detection.

A “disorder” or “disease” is any condition that would benefit from treatment including, but not limited to, chronic and acute disorders or diseases including those pathological conditions which predispose the mammal to the disorder in question (e.g., cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)).

The term “diagnosis” is used herein to refer to the identification or classification of a molecular or pathological state, disease or condition (e.g., cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by the genes)).

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down-regulation of cell surface receptors (e.g., PD-L1); and B cell activation.

An “effective amount” of a compound, for example, an PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), or a composition (e.g., pharmaceutical composition) thereof, is at least the minimum amount required to achieve the desired therapeutic or prophylactic result, such as a measurable increase in overall survival (OS), progression-free survival (PFS), or overall response (e.g., best confirmed overall response (BOOR)) of a particular disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). An effective amount herein may vary according to factors such as the disease state, age, sex, and weight of the individual, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may optionally be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. For example, an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) as a cancer treatment may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk et al., Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al., J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The word “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression level can be measured by methods known to one skilled in the art and also disclosed herein, including, for example, RT-qPCR and RNA-seq. The expression level assessed can be used to determine the response to the treatment.

The term “immune-score expression level” refers to a numerical value that reflects the expression level (e.g., a normalized expression level) of a single gene of interest, or an aggregated expression level for more than one gene of interest (e.g., at least two, at least three, at least four, at least five, or more genes of interest), related to immune response. An immune-score expression level for more than one gene of interest may be determined by aggregation methods known to one skilled in the art and also disclosed herein, including, for example, by calculating the median or mean of the expression levels of all of the genes of interest. Before aggregation, the expression level of each gene of interest may be normalized by using statistical methods known to one skilled in the art and also disclosed herein, including, for example, normalized to the expression level of one or more housekeeping genes, or normalized to a total library size, or normalized to the median or mean expression level value across all genes measured. In some instances, before aggregation across multiple genes of interest, the normalized expression level of each gene of interest may be standardized by calculating the Z-score of the normalized expression level of each gene of interest. In some instances, each gene of interest may have an assigned weight score, and the immune-score expression level of multiple genes of interest may be calculated by incorporating the weight score to determine the mean of the weighted expression levels of all of the genes of interest. For example, an immune-score expression level may refer to a numerical value that reflects the normalized expression level of a single gene selected from any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7. Alternatively, an immune-score expression level may, for example, refer to a numerical value that reflects the aggregated normalized expression level (e.g., median of the normalized expression levels, or mean of the normalized expression levels) for at least two, at least three, at least four, at least five, at least six, at least seven, at least eight, at least nine, at least 10, at least 11, at least 12, at least 13, at least 14, at least 15, at least 16, at least 17, at least 18, at least 19, or more, of genes set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7, or combinations thereof. In some instances, an immune-score expression level may, for example, refer to a numerical value that reflects the aggregated Z-score expression level (e.g., mean of the Z-score normalized expression level, or median of the Z-score normalized expression level) for at least two, at least three, at least four, at least five, at least six, at least seven, at least eight, at least nine, at least 10, at least 11, at least 12, at least 13, at least 14, at least 15, at least 16, at least 17, at least 18, at least 19, or more, of genes set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7, or combinations thereof.

As used herein, the term “reference immune-score expression level” refers to an immune-score expression level against which another immune-score expression level (e.g., for one or more of genes set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7) is compared, e.g., to make a diagnostic, predictive, prognostic, and/or therapeutic determination. For example, the reference immune-score expression level may be derived from expression levels (e.g., for one or more of genes set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7) in a reference sample, a reference population, and/or a pre-assigned value (e.g., a cut-off value which was previously determined to significantly (e.g., statistically significantly) separate a first subset of individuals who have been treated with a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above the cut-off value and/or below the cut-off value, wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy above the cut-off value and/or the individual's responsiveness to treatment with the non-PD-L1 axis binding antagonist therapy is significantly (e.g., statistically significantly) improved relative to the individual's responsiveness to treatment with the PD-L1 axis binding antagonist therapy below the cut-off value). It will be appreciated by one skilled in the art that the numerical value for the reference immune-score expression level may vary depending on the indication (e.g., a cancer (e.g., a breast cancer, a lung cancer (e.g., NSCLC, including non-squamous NSCLC and squamous NSCLC), a kidney cancer, or a bladder cancer), the methodology used to detect expression levels (e.g., RNA-seq or RT-qPCR), the statistical methods used to generate an immune-score, and/or the specific combinations of genes examined.

“Elevated expression,” “elevated expression levels,” or “elevated levels” refers to an increased expression or increased levels of a gene or combination of genes (e.g., for one or more of genes set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7) in a subject relative to a control, such as a subject or subjects who are not suffering from the disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)) or an internal control (e.g., housekeeping gene), or a reference level, such as a reference immune-score expression level.

“Reduced expression,” “reduced expression levels,” or “reduced levels” refers to a decrease expression or decreased levels of a gene or combination of genes (e.g., for one or more of genes set forth in any one of Tables 1-17, e.g., CD79A, CD19, BANK1, JCHAIN, SLAMF7, BTK, TNFRSF17, IGJ, IGLL5, RBPJ, MZB1, CCL2, CCL3, CCL4, CCL5, CCL8, CCL18, CCL19, CCL21, CXCL9, CXCL10, CXCL11, CXCL13, DERL3, JSRP1, IGHG2, IGHGP, IGLV3-1, IGLV6-57, IGHA2, IGKV4-1, IGKV1-12, and IGLC7) in a subject relative to a control, such as a subject or subjects who are not suffering from the disease or disorder (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)) or an internal control (e.g., housekeeping gene), or a reference level, such as a reference immune-score expression level. In some embodiments, reduced expression is little or no expression.

A “reference gene” as used herein, refers to a gene or group of genes (e.g., one, two, three, four, five, or six or more genes) that is used for comparison purposes, such as a housekeeping gene. A “housekeeping gene” refers herein to a gene or group of genes (e.g., one, two, three, four, five, or six or more genes) which encode proteins whose activities are essential for the maintenance of cell function and which are typically similarly present in all cell types.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (Δ), based on the amino acid sequence of its constant domain.

The term “oligonucleotide” refers to a relatively short polynucleotide (e.g., less than about 250 nucleotides in length), including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The terms “Programmed Death Ligand 1” and “PD-L1” refer herein to a native sequence PD-L1 polypeptide, polypeptide variants, and fragments of a native sequence polypeptide and polypeptide variants (which are further defined herein). The PD-L1 polypeptide described herein may be that which is isolated from a variety of sources, such as from human tissue types or from another source, or prepared by recombinant or synthetic methods.

“PD-L1 polypeptide variant”, or variations thereof, means a PD-L1 polypeptide, generally an active PD-L1 polypeptide, as defined herein having at least about 80% amino acid sequence identity with any of the native sequence PD-L1 polypeptide sequences as disclosed herein. Such PD-L1 polypeptide variants include, for instance, PD-L1 polypeptides wherein one or more amino acid residues are added, or deleted, at the N- or C-terminus of a native amino acid sequence. Ordinarily, a PD-L1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence PD-L1 polypeptide sequence as disclosed herein. Ordinarily, PD-L1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 281, 282, 283, 284, 285, 286, 287, 288, 289 amino acids in length, or more. Optionally, PD-L1 variant polypeptides will have no more than one conservative amino acid substitution as compared to a native PD-L1 polypeptide sequence, alternatively no more than 2, 3, 4, 5, 6, 7, 8, 9, or conservative amino acid substitution as compared to the native PD-L1 polypeptide sequence.

A “native sequence PD-L1 polypeptide” comprises a polypeptide having the same amino acid sequence as the corresponding PD-L1 polypeptide derived from nature.

The term “PD-L1 axis binding antagonist” refers to a molecule that inhibits the interaction of a PD-L1 axis binding partner with one or more of its binding partners, so as to remove T cell dysfunction resulting from signaling on the PD-1 signaling axis, with a result being restored or enhanced T cell function. As used herein, a PD-L1 axis binding antagonist includes a PD-L1 binding antagonist and a PD-1 binding antagonist, as well as molecules that interfere with the interaction between PD-L1 and PD-1 (e.g., a PD-L2-Fc fusion).

The term “PD-L1 binding antagonist” refers to a molecule that decreases, blocks, inhibits, abrogates, or interferes with signal transduction resulting from the interaction of PD-L1 with either one or more of its binding partners, such as PD-1 or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides, and other molecules that decrease, block, inhibit, abrogate, or interfere with signal transduction resulting from the interaction of PD-L1 with one or more of its binding partners, such as PD-1 or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific embodiment, the anti-PD-L1 antibody is atezolizumab (CAS Registry Number: 1422185-06-5), also known as MPDL3280A, and described herein. In another specific embodiment, the anti-PD-L1 antibody is YW243.55.570, described herein. In another specific embodiment, the anti-PD-L1 antibody is MDX-1105, described herein. In still another specific aspect, the anti-PD-L1 antibody is MED14736 (durvalumab), described herein. In still another specific aspect, the anti-PD-L1 antibody is MSB0010718C (avelumab), described herein.

As used herein, a “PD-1 binding antagonist” is a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti PD-1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-1 with PD-L1 and/or PD-L2. In one embodiment, a PD-1 binding antagonist reduces the negative signal mediated by or through cell surface proteins expressed on T lymphocytes, and other cells, mediated signaling through PD-1 or PD-L1 so as render a dysfunctional T cell less dysfunctional. In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is MEDI-0680 (AMP-514). In another specific aspect, a PD-1 binding antagonist is PDR001. In another specific aspect, a PD-1 binding antagonist is REGN2810. In another specific aspect, a PD-1 binding antagonist is BGB-108. In another specific aspect, a PD-1 binding antagonist is AMP-224.

“Polynucleotide” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

As used herein, the term “reverse transcriptase polymerase chain reaction” or “RT-PCR” refers to the replication and amplification of RNA sequences. In this method, reverse transcription is coupled to PCR, e.g., as described in U.S. Pat. No. 5,322,770, herein incorporated by reference in its entirety. In RT-PCR, the RNA template is converted to cDNA due to the reverse transcriptase activity of an enzyme, and then amplified using the polymerizing activity of the same or a different enzyme. Both thermostable and thermolabile reverse transcriptase and polymerase can be used. The “reverse transcriptase” (RT) may include reverse transcriptases from retroviruses, other viruses, as well as a DNA polymerase exhibiting reverse transcriptase activity.

As used herein, the term “reverse transcriptase quantitative polymerase chain reaction” or “RT-qPCR” is a form of PCR wherein the nucleic acid to be amplified is RNA that is first reverse transcribed into cDNA and the amount of PCR product is measured at each step in a PCR reaction.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “multiplex-PCR” refers to a single PCR reaction carried out on nucleic acid obtained from a single source (e.g., an individual) using more than one primer set for the purpose of amplifying two or more DNA sequences in a single reaction.

The term “RNA-seq,” also called “Whole Transcriptome Shotgun Sequencing (WTSS),” refers to the use of high-throughput sequencing technologies to sequence and/or quantify cDNA to obtain information about a sample's RNA content. Publications describing RNA-seq include: Wang et al., Nature Reviews Genetics 10(1): 57-63 (2009); Ryan et al. BioTechniques 45(1): 81-94 (2008); and Maher et al., Nature 458(7234): 97-101 (2009).

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

“Response to a treatment,” “responsiveness to treatment,” or “benefit from a treatment” can be assessed using any endpoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer); (6) increase or extend in the length of survival, including overall survival (OS HR<1) and progression free survival (PFS HR<1); and/or (9) decreased mortality at a given point of time following treatment (e.g., a treatment including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody))). As used herein, a patient that “fails to respond” to a particular form of treatment is one that fails to exhibit any or all of the above-described benefits following administration of the therapy of interest.

As used herein, “progression-free survival” or “PFS” refers to the length of time during and after treatment during which the disease being treated (e.g., cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)) does not progress or get worse. Progression-free survival may include the amount of time individuals have experienced a complete response or a partial response, as well as the amount of time individuals have experienced stable disease.

As used herein, “overall survival” or “OS” refers to the length of time during and after treatment that subjects are likely to be alive after a particular duration of time (e.g., 6 months, 1 year, 2 years, 3 years, 4 years, 5 years, 10 years, 15 years, 20 years, or more than 20 years from the time of diagnosis or treatment).

As used herein, “complete response” or “CR” refers to disappearance of all signs of cancer in response to treatment. This does not necessarily mean the cancer has been cured.

As used herein, “partial response” or “PR” refers to a decrease in the size of one or more tumors or lesions, or in the extent of cancer in the body, in response to treatment.

As used herein, “hazard ratio” or “HR” is a statistical definition for rates of events. For the purpose of the invention, hazard ratio is defined as representing the probability of an event (e.g., PFS or OS) in the experimental (e.g., treatment) group/arm divided by the probability of an event in the control group/arm at any specific point in time. An HR with a value of 1 indicates that the relative risk of an endpoint (e.g., death) is equal in both the “treatment” and “control” groups; a value greater than 1 indicates that the risk is greater in the treatment group relative to the control group; and a value less than 1 indicates that the risk is greater in the control group relative to the treatment group. “Hazard ratio” in progression-free survival analysis (i.e., PFS HR) is a summary of the difference between two progression-free survival curves, representing the reduction in the risk of death on treatment compared to control, over a period of follow-up. “Hazard ratio” in overall survival analysis (i.e., OS HR) is a summary of the difference between two overall survival curves, representing the reduction in the risk of death on treatment compared to control, over a period of follow-up.

By “extending survival” or an “extension in survival” is meant increasing overall survival or progression free survival in a treated individual relative to an untreated individual (i.e. relative to an individual not treated with the medicament), or relative to an individual who does not express a biomarker at the designated level, and/or relative to an individual treated with an approved anti-tumor agent. An objective response refers to a measurable response, including complete response (CR) or partial response (PR).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)), the presence or size of metastases, or the size of the primary tumor.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from the same subject or individual. In another embodiment, a reference sample is obtained from one or more individuals who are not the subject or individual. In either of the preceding embodiments, the one or more individuals from which the reference sample, reference cell, reference tissue, control sample, control cell, or control tissue is obtained has a cancer. In certain embodiments, the one or more individuals from which the reference sample, reference cell, reference tissue, control sample, control cell, or control tissue is obtained has a cancer and has been previously treated with an anti-cancer therapy (e.g., one or more doses of a PD-L1 axis binding antagonist). In other embodiments, the one or more individuals from which the reference sample, reference cell, reference tissue, control sample, control cell, or control tissue is obtained has a cancer and is treatment naïve. In any of the preceding embodiments, the subject/individual and the one or more individuals who are not the subject or individual have the same cancer. In yet another embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

As used herein, the terms “individual,” “patient,” and “subject” are used interchangeably and refer to any single animal, more preferably a mammal (including such non-human animals as, for example, cats, dogs, horses, rabbits, zoo animals, cows, pigs, sheep, and non-human primates) for which treatment is desired. In certain embodiments, the individual, patient, or subject is a human.

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of a disease (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)), alleviation of symptoms, diminishment of any direct or indirect pathological consequences of the disease, preventing metastasis, decreasing the rate of disease progression, amelioration or palliation of the disease state, and remission or improved prognosis. In some embodiments, the treatments described herein are used to delay development of a disease or to slow the progression of a disease (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)). In some instances, the treatment may increase overall survival (OS) (e.g., by about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the treatment may increase OS, e.g., by about 5% to about 500%, e.g., from about 10% to about 450%, e.g., from about 20% to about 400%, e.g., from about 25% to about 350%, e.g., from about 30% to about 400%, e.g., from about 35% to about 350%, e.g., from about 40% to about 300%, e.g., from about 45% to about 250%, e.g., from about 50% to about 200%, e.g., from about 55% to about 150%, e.g., from about 60% to about 100%, e.g., from about 65% to about 100%, e.g., from about 70% to about 100%, e.g., from about 75% to about 100%, e.g., from about 80% to about 100%, e.g., from about 85% to about 100%, e.g., from about 90% to about 100%, e.g., from about 95% to about 100%, e.g., from about 98% to about 100%. In some instances, the treatment may increase the progression-free survival (PFS) (e.g., by about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the treatment may increase PFS, e.g., by about 5% to about 500%, e.g., from about 10% to about 450%, e.g., from about 20% to about 400%, e.g., from about 25% to about 350%, e.g., from about 30% to about 400%, e.g., from about 35% to about 350%, e.g., from about 40% to about 300%, e.g., from about 45% to about 250%, e.g., from about 50% to about 200%, e.g., from about 55% to about 150%, e.g., from about 60% to about 100%, e.g., from about 65% to about 100%, e.g., from about 70% to about 100%, e.g., from about 75% to about 100%, e.g., from about 80% to about 100%, e.g., from about 85% to about 100%, e.g., from about 90% to about 100%, e.g., from about 95% to about 100%, e.g., from about 98% to about 100%.

By “tissue sample” or “cell sample” is meant a collection of similar cells obtained from a tissue of a subject or individual. The source of the tissue or cell sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease (e.g., a cancer, e.g., a lung cancer (e.g., NSCLC, including non-squamous NSCLC and squamous NSCLC), a bladder cancer (e.g., UC), a kidney cancer (e.g., RCC), or a breast cancer (e.g., TNBC)) tissue/organ. The tissue sample may contain compounds which are not naturally intermixed with the tissue in nature such as preservatives, anticoagulants, buffers, fixatives, nutrients, antibiotics, or the like.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

The terms “tertiary lymphoid structure” and “TLS” are used interchangeably herein to refer to an ectopic lymphoid-like structure that can develop in non-lymphoid tissues, e.g., at sites of chronic inflammation, including tumors. These terms can refer to structures of varying organization, e.g., from clusters of lymphocytes to segregated structures that are reminiscent of secondary lymphoid organs. For example, the terms encompass TLS-like structures. In some instances, a TLS may contain distinct T and B cell compartments, fibroblastic reticular cell (FRC) networks, peripheral node addressin (PNAd+) high endothelial venules (HEVs), follicular dendritic cells (FDCs), evidence for class switching and reactive germinal centers (GCs) in B cell zones, and/or expression of activation-induced cytidine deaminase (AID), an enzyme expressed in GC B cells involved in initiation of somatic hypermutation and immunoglobulin gene class switching. For a review of TLS in cancer, see Colbeck et al. Front. Immunol. 8:1830, 2017.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### II. DIAGNOSTIC METHODS

Provided herein are methods for identifying an individual having a cancer (e.g., a lung cancer (e.g., non-small cell lung cancer (NSCLC)), a bladder cancer (e.g., a urothelial carcinoma (UC)), a kidney cancer (e.g., a renal cell carcinoma (RCC)), or a breast cancer (e.g., triple-negative breast cancer (TNBC))) who may benefit from a treatment including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

Further provided herein are methods for selecting a therapy for an individual having a cancer (e.g., lung cancer (e.g., NSCLC), bladder cancer (e.g., UC), kidney cancer (e.g., RCC), or breast cancer (e.g., TNBC)); methods for determining whether an individual having a cancer is likely to respond to treatment including a PD-L1 axis binding antagonist; methods for predicting the responsiveness of an individual having a cancer to treatment comprising a PD-L1 axis binding antagonist; and methods for monitoring the response of an individual having a cancer to treatment including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

Any of the methods provided herein may include determining the presence and/or expression level of any biomarker disclosed herein. For example, the biomarker may include the presence and/or expression level of a biomarker set forth in any one of Tables 1-17 below in a sample obtained from an individual; the presence of a TLS in a sample obtained from an individual; the number of B cells in a sample obtained from an individual; the presence of clonally expanded B cells in a sample from the individual; and/or a combination thereof. Any suitable sample may be used, e.g., any sample type disclosed herein, including a tumor sample.

Any of the methods provided herein may further include selecting a therapy for the individual, e.g., a therapy comprising a PD-L1 axis binding antagonist (e.g., as described below in Section IV).

Any of the methods provided herein may further include administering to the individual a PD-L1 axis binding antagonist (e.g., as described below in Section IV) to the individual.

TABLE 1
Exemplary Biomarkers

CD79A
SLAMF7
BTK
TNFRSF17
IGJ
IGLL5
RBPJ
MZB1
CCL2
CCL3
CCL4
CCL5
CCL8
CCL18
CCL19
CCL21
CXCL9
CXCL10
CXCL11
CXCL13

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of one or more (e.g., 1, 2, 3, 4, 5, 6, 7, 8, 9, 10, 11, 12, 13, 14, 15, 16, 17, 18, 19, or 20) genes set forth in Table 1 in a sample from the individual, wherein an immune-score expression level of the one or more genes that is above a reference immune-score expression level of the one or more genes identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of one or more (e.g., 1, 2, 3, 4, 5, 6, 7, 8, 9, 10, 11, 12, 13, 14, 15, 16, 17, 18, 19, or 20) of genes set forth in Table 1 in a sample from the individual, wherein an immune-score expression level of the one or more genes that is above a reference immune-score expression level of the one or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the immune-score expression level of the one or more (e.g., 1, 2, 3, 4, 5, 6, 7, 8, 9, 10, 11, 12, 13, 14, 15, 16, 17, 18, 19, or 20) genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in progression-free survival (PFS), or an increase in best confirmed overall response (BOOR). In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is a formalin-fixed and paraffin-embedded (FFPE) sample, an archival sample, a fresh sample, or a frozen sample. In some instances, the tumor tissue sample is an FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is a non-small cell lung cancer (NSCLC). In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### A. B Cell Signatures

(i) Gene Signatures Associated with B Cells

In some aspects, the methods provided herein may involve determining an expression level of one or more genes in a B cell signature. Any suitable B cell signature may be used. For example, the B cell signature may include one or more (e.g., 1, 2, 3, 4, 5, 6, 7, 8, 9, 10, or 11) genes set forth in Table 2.

TABLE 2
Exemplary B Cell Signature Genes

CD79A
CD19
BANK1
JCHAIN
SLAMF7
BTK
TNFRSF17
IGJ
IGLL5
RBPJ
MZB1

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the method comprises determining the expression level of one of genes CD79A, CD19, BANK1, JCHAIN, SLAMF7, BTK, TNFRSF17, IGJ, IGLL5, RBPJ, and MZB1.

In some instances, the method comprises determining the expression level of CD79A.

In some instances, the method comprises determining the expression level of CD19.

In some instances, the method comprises determining the expression level of BANK1.

In some instances, the method comprises determining the expression level of JCHAIN.

In some instances, the method comprises determining the expression level of SLAMF7.

In some instances, the method comprises determining the expression level of BTK.

In some instances, the method comprises determining the expression level of TNFRSF17.

In some instances, the method comprises determining the expression level of IGJ.

In some instances, the method comprises determining the expression level of IGLL5.

In some instances, the method comprises determining the expression level of RBPJ.

In some instances, the method comprises determining the expression level of MZB1.

In some instances, the immune-score expression level of the one or more genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), wherein the benefit comprises an extension in the individual's OS as compared to treatment without the PD-L1 axis binding antagonist.

In some instances, the immune-score expression level of the one or more genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

In some instances, the genes comprise two or more of CD79A, CD19, BANK1, JCHAIN, SLAMF7, BTK, TNFRSF17, IGJ, IGLL5, RBPJ, and MZB1.

For example, provided herein is a method of identifying an individual having a cancer who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

In another example, provided herein is a method of selecting a therapy for an individual having a cancer, the method comprising determining the expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

In a further example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist, wherein the benefit comprises an extension in the individual's OS as compared to treatment without the PD-L1 axis binding antagonist.

In yet another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of two or more (e.g., 2, 3, 4, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), wherein the benefit comprises an extension in the individual's OS as compared to treatment without the PD-L1 axis binding antagonist.

In some instances, the immune-score expression level of the two or more genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

Any suitable PD-L1 axis binding antagonist may be administered, e.g., any PD-L1 axis binding antagonist provided herein (e.g., as described below in Section IV). Any combination of B cell signature genes may be determined. For example, the combination may include two genes selected from CD79A, CD19, BANK1, JCHAIN, SLAMF7, BTK, TNFRSF17, IGJ, IGLL5, RBPJ, and MZB1, e.g., any one of the combinations set forth in Table 3. In another example, the combination may include three genes selected from CD79A, CD19, BANK1, JCHAIN, SLAMF7, BTK, TNFRSF17, IGJ, IGLL5, RBPJ, and MZB1, e.g., any one of the combinations set forth in Table 4. In another example, the combination may include four genes selected from CD79A, CD19, BANK1, JCHAIN, SLAMF7, BTK, TNFRSF17, IGJ, IGLL5, RBPJ, and MZB1, e.g., any one of the combinations set forth in Table 5. In another example, the combination may include five genes selected from CD79A, CD19, BANK1, JCHAIN, SLAMF7, BTK, TNFRSF17, IGJ, IGLL5, RBPJ, and MZB1, e.g., any one of the combinations set forth in Table 6. In another example, the combination may include six genes selected from CD79A, CD19, BANK1, JCHAIN, SLAMF7, BTK, TNFRSF17, IGJ, IGLL5, RBPJ, and MZB1, e.g., any one of the combinations set forth in Table 7. In another example, the combination may include seven genes selected from CD79A, CD19, BANK1, JCHAIN, SLAMF7, BTK, TNFRSF17, IGJ, IGLL5, RBPJ, and MZB1, e.g., any one of the combinations set forth in Table 8.

TABLE 3
Exemplary Two-Gene Combinations of B Cell Signature Genes

CD79A and SLAMF7
CD79A and BTK
CD79A and TNFRSF17
CD79A and IGJ
CD79A and IGLL5
CD79A and RBPJ
CD79A and MZB1
SLAMF7 and BTK
SLAMF7 and TNFRSF17
SLAMF7 and IGJ
SLAMF7 and IGLL5
SLAMF7 and RBPJ
SLAMF7 and MZB1
BTK and TNFRSF17
BTK and IGJ
BTK and IGLL5
BTK and RBPJ
BTK and MZB1
TNFRSF17 and IGJ
TNFRSF17 and IGLL5
TNFRSF17 and RBPJ
TNFRSF17 and MZB1
IGJ and IGLL5
IGJ and RBPJ
IGJ and MZB1
IGLL5 and RBPJ
IGLL5 and MZB1
RBPJ and MZB1
CD19 and CD79A
CD19 and SLAMF7
CD19 and BTK
CD19 and TNFRSF17
CD19 and IGJ
CD19 and IGLL5
CD19 and RBPJ
CD19 and MZB1
CD19 and BANK1
CD19 and JCHAIN
BANK1 and CD79A
BANK1 and SLAMF7
BANK1 and BTK
BANK1 and TNFRSF17
BANK1 and IGJ
BANK1 and IGLL5
BANK1 and RBPJ
BANK1 and MZB1
BANK1 and JCHAIN
JCHAIN and CD79A
JCHAIN and SLAMF7
JCHAIN and BTK
JCHAIN and TNFRSF17
JCHAIN and IGJ
JCHAIN and IGLL5
JCHAIN and RBPJ
JCHAIN and MZB1

TABLE 4
Exemplary Three-Gene Combinations of B Cell Signature Genes

CD79A, SLAMF7, and BTK
CD79A, SLAMF7, and TNFRSF17
CD79A, SLAMF7, and IGJ
CD79A, SLAMF7, and IGLL5
CD79A, SLAMF7, and RBPJ
CD79A, SLAMF7, and MZB1
CD79A, BTK, and TNFRSF17
CD79A, BTK, and IGJ
CD79A, BTK, and IGLL5
CD79A, BTK, and RBPJ
CD79A, BTK, and MZB1
CD79A, TNFRSF17, and IGJ
CD79A, TNFRSF17, and IGLL5
CD79A, TNFRSF17, and RBPJ
CD79A, TNFRSF17, and MZB1
CD79A, IGJ, and IGLL5
CD79A, IGJ, and RBPJ
CD79A, IGJ, and MZB1
CD79A, IGLL5, and RBPJ
CD79A, IGLL5, and MZB1
CD79A, RBPJ, and MZB1
SLAMF7, BTK, and TNFRSF17
SLAMF7, BTK, and IGJ
SLAMF7, BTK, and IGLL5
SLAMF7, BTK, and RBPJ
SLAMF7, BTK, and MZB1
SLAMF7, TNFRSF17, and IGJ
SLAMF7, TNFRSF17, and IGLL5
SLAMF7, TNFRSF17, and RBPJ
SLAMF7, TNFRSF17, and MZB1
SLAMF7, IGJ, and IGLL5
SLAMF7, IGJ, and RBPJ
SLAMF7, IGJ, and MZB1
SLAMF7, IGLL5, and RBPJ
SLAMF7, IGLL5, and MZB1
SLAMF7, RBPJ, and MZB1
BTK, TNFRSF17, and IGJ
BTK, TNFRSF17, and IGLL5
BTK, TNFRSF17, and RBPJ
BTK, TNFRSF17, and MZB1
BTK, IGJ, and IGLL5
BTK, IGJ, and RBPJ
BTK, IGJ, and MZB1
BTK, IGLL5, and RBPJ
BTK, IGLL5, and MZB1
BTK, RBPJ, and MZB1
TNFRSF17, IGJ, and IGLL5
TNFRSF17, IGJ, and RBPJ
TNFRSF17, IGJ, and MZB1
TNFRSF17, IGLL5, and RBPJ
TNFRSF17, IGLL5, and MZB1
TNFRSF17, RBPJ, and MZB1
IGJ, IGLL5, and RBPJ
IGJ, IGLL5, and MZB1
IGJ, RBPJ, and MZB1
IGLL5, RBPJ, and MZB1
CD19, BANK1, and CD79A
CD19, BANK1, and JCHAIN
CD19, BANK1, and SLAMF7
CD19, BANK1, and BTK
CD19, BANK1, and TNFRSF17
CD19, BANK1, and IGJ
CD19, BANK1, and IGLL5
CD19, BANK1, and RBPJ
CD19, BANK1, and MZB1
CD19, JCHAIN, and CD79A
CD19, JCHAIN, and SLAMF7
CD19, JCHAIN, and BTK
CD19, JCHAIN, and TNFRSF17
CD19, JCHAIN, and IGJ
CD19, JCHAIN, and IGLL5
CD19, JCHAIN, and RBPJ
CD19, JCHAIN, and MZB1
CD19, SLAMF7, and CD79A
CD19, SLAMF7, and BTK
CD19, SLAMF7, and TNFRSF17
CD19, SLAMF7, and IGJ
CD19, SLAMF7, and IGLL5
CD19, SLAMF7, and RBPJ
CD19, SLAMF7, and MZB1
CD19, BTK, and CD79A
CD19, BTK, and TNFRSF17
CD19, BTK, and IGJ
CD19, BTK, and IGLL5
CD19, BTK, and RBPJ
CD19, BTK, and MZB1
CD19, TNFRSF17, and IGJ
CD19, TNFRSF17, and IGLL5
CD19, TNFRSF17, and RBPJ
CD19, TNFRSF17, and MZB1
CD19, IGJ, and IGLL5
CD19, IGJ, and RBPJ
CD19, IGJ, and MZB1
CD19, IGLL5, and RBPJ
CD19, IGLL5, and MZB1
CD19, RBPJ, and MZB1

TABLE 5
Exemplary Four-Gene Combinations of B Cell Signature Genes

CD79A, SLAMF7, BTK, and TNFRSF17
CD79A, SLAMF7, BTK, and IGJ
CD79A, SLAMF7, BTK, and IGLL5
CD79A, SLAMF7, BTK, and RBPJ
CD79A, SLAMF7, BTK, and MZB1
CD79A, SLAMF7, TNFRSF17, and IGJ
CD79A, SLAMF7, TNFRSF17, and IGLL5
CD79A, SLAMF7, TNFRSF17, and RBPJ
CD79A, SLAMF7, TNFRSF17, and MZB1
CD79A, SLAMF7, IGJ, and IGLL5
CD79A, SLAMF7, IGJ, and RBPJ
CD79A, SLAMF7, IGJ, and MZB1
CD79A, SLAMF7, IGLL5, and RBPJ
CD79A, SLAMF7, IGLL5, and MZB1
CD79A, SLAMF7, RBPJ, and MZB1
CD79A, BTK, TNFRSF17, and IGJ
CD79A, BTK, TNFRSF17, and IGLL5
CD79A, BTK, TNFRSF17, and RBPJ
CD79A, BTK, TNFRSF17, and MZB1
CD79A, BTK, IGJ, and IGLL5
CD79A, BTK, IGJ, and RBPJ
CD79A, BTK, IGJ, and MZB1
CD79A, BTK, IGLL5, and RBPJ
CD79A, BTK, IGLL5, and MZB1
CD79A, BTK, RBPJ, and MZB1
CD79A, TNFRSF17, IGJ, and IGLL5
CD79A, TNFRSF17, IGJ, and RBPJ
CD79A, TNFRSF17, IGJ, and MZB1
CD79A, TNFRSF17, IGLL5, and RBPJ
CD79A, TNFRSF17, IGLL5, and MZB1
CD79A, TNFRSF17, RBPJ, and MZB1
CD79A, IGJ, IGLL5, and RBPJ
CD79A, IGJ, IGLL5, and MZB1
CD79A, IGJ, RBPJ, and MZB1
CD79A, IGLL5, RBPJ, and MZB1
SLAMF7, BTK, TNFRSF17, and IGJ
SLAMF7, BTK, TNFRSF17, and IGLL5
SLAMF7, BTK, TNFRSF17, and RBPJ
SLAMF7, BTK, TNFRSF17, and MZB1
SLAMF7, BTK, IGJ, and IGLL5
SLAMF7, BTK, IGJ, and RBPJ
SLAMF7, BTK, IGJ, and MZB1
SLAMF7, BTK, IGLL5, and RBPJ
SLAMF7, BTK, IGLL5, and MZB1
SLAMF7, BTK, RBPJ, and MZB1
SLAMF7, TNFRSF17, IGJ, and IGLL5
SLAMF7, TNFRSF17, IGJ, and RBPJ
SLAMF7, TNFRSF17, IGJ, and MZB1
SLAMF7, TNFRSF17, IGLL5, and RBPJ
SLAMF7, TNFRSF17, IGLL5, and MZB1
SLAMF7, TNFRSF17, RBPJ, and MZB1
SLAMF7, IGJ, IGLL5, and RBPJ
SLAMF7, IGJ, IGLL5, and MZB1
SLAMF7, IGJ, RBPJ, and MZB1
SLAMF7, IGLL5, RBPJ, and MZB1
BTK, TNFRSF17, IGJ, and IGLL5
BTK, TNFRSF17, IGJ, and RBPJ
BTK, TNFRSF17, IGJ, and MZB1
BTK, TNFRSF17, IGLL5, and RBPJ
BTK, TNFRSF17, IGLL5, and MZB1
BTK, TNFRSF17, RBPJ, and MZB1
BTK, IGJ, IGLL5, and RBPJ
BTK, IGJ, IGLL5, and MZB1
BTK, IGJ, RBPJ, and MZB1
BTK, IGLL5, RBPJ, and MZB1
TNFRSF17, IGJ, IGLL5, and RBPJ
TNFRSF17, IGJ, IGLL5, and MZB1
TNFRSF17, IGJ, RBPJ, and MZB1
TNFRSF17, IGLL5, RBPJ, and MZB1
IGJ, IGLL5, RBPJ, and MZB1
CD19, JCHAIN, BANK1, and CD79A
CD19, JCHAIN, BANK1, and SLAMF7
CD19, JCHAIN, BANK1, and BTK
CD19, JCHAIN, BANK1, and TNFRSF17
CD19, JCHAIN, BANK1, and IGJ
CD19, JCHAIN, BANK1, and IGLL5
CD19, JCHAIN, BANK1, and RBPJ
CD19, JCHAIN, BANK1, and MZB1

TABLE 6
Exemplary Five-Gene Combinations of B Cell Signature Genes
CD79A, SLAMF7, BTK, TNFRSF17, and IGJ
CD79A, SLAMF7, BTK, TNFRSF17, and IGLL5
CD79A, SLAMF7, BTK, TNFRSF17, and RBPJ
CD79A, SLAMF7, BTK, TNFRSF17, and MZB1
CD79A, SLAMF7, BTK, IGJ, and IGLL5
CD79A, SLAMF7, BTK, IGJ, and RBPJ
CD79A, SLAMF7, BTK, IGJ, and MZB1
CD79A, SLAMF7, BTK, IGLL5, and RBPJ
CD79A, SLAMF7, BTK, IGLL5, and MZB1
CD79A, SLAMF7, BTK, RBPJ, and MZB1
CD79A, SLAMF7, TNFRSF17, IGJ, and IGLL5
CD79A, SLAMF7, TNFRSF17, IGJ, and RBPJ
CD79A, SLAMF7, TNFRSF17, IGJ, and MZB1
CD79A, SLAMF7, TNFRSF17, IGLL5, and RBPJ
CD79A, SLAMF7, TNFRSF17, IGLL5, and MZB1
CD79A, SLAMF7, TNFRSF17, RBPJ, and MZB1
CD79A, SLAMF7, IGJ, IGLL5, and RBPJ
CD79A, SLAMF7, IGJ, IGLL5, and MZB1
CD79A, SLAMF7, IGJ, RBPJ, and MZB1
CD79A, SLAMF7, IGLL5, RBPJ, and MZB1
CD79A, BTK, TNFRSF17, IGJ, and IGLL5
CD79A, BTK, TNFRSF17, IGJ, and RBPJ
CD79A, BTK, TNFRSF17, IGJ, and MZB1
CD79A, BTK, TNFRSF17, IGLL5, and RBPJ
CD79A, BTK, TNFRSF17, IGLL5, and MZB1
CD79A, BTK, TNFRSF17, RBPJ, and MZB1
CD79A, BTK, IGJ, IGLL5, and RBPJ
CD79A, BTK, IGJ, IGLL5, and MZB1
CD79A, BTK, IGJ, RBPJ, and MZB1
CD79A, BTK, IGLL5, RBPJ, and MZB1
CD79A, TNFRSF17, IGJ, IGLL5, and RBPJ
CD79A, TNFRSF17, IGJ, IGLL5, and MZB1
CD79A, TNFRSF17, IGJ, RBPJ, and MZB1
CD79A, TNFRSF17, IGLL5, RBPJ, and MZB1
CD79A, IGJ, IGLL5, RBPJ, and MZB1
SLAMF7, BTK, TNFRSF17, IGJ, and IGLL5
SLAMF7, BTK, TNFRSF17, IGJ, and RBPJ
SLAMF7, BTK, TNFRSF17, IGJ, and MZB1
SLAMF7, BTK, TNFRSF17, IGLL5, and RBPJ
SLAMF7, BTK, TNFRSF17, IGLL5, and MZB1
SLAMF7, BTK, TNFRSF17, RBPJ, and MZB1
SLAMF7, BTK, IGJ, IGLL5, and RBPJ
SLAMF7, BTK, IGJ, IGLL5, and MZB1
SLAMF7, BTK, IGJ, RBPJ, and MZB1
SLAMF7, BTK, IGLL5, RBPJ, and MZB1
SLAMF7, TNFRSF17, IGJ, IGLL5, and RBPJ
SLAMF7, TNFRSF17, IGJ, IGLL5, and MZB1
SLAMF7, TNFRSF17, IGJ, RBPJ, and MZB1
SLAMF7, TNFRSF17, IGLL5, RBPJ, and MZB1
SLAMF7, IGJ, IGLL5, RBPJ, and MZB1
BTK, TNFRSF17, IGJ, IGLL5, and RBPJ
BTK, TNFRSF17, IGJ, IGLL5, and MZB1
BTK, TNFRSF17, IGJ, RBPJ, and MZB1
BTK, TNFRSF17, IGLL5, RBPJ, and MZB1
BTK, IGJ, IGLL5, RBPJ, and MZB1
TNFRSF17, IGJ, IGLL5, RBPJ, and MZB1
CD19, BANK1, JCHAIN, SLAMF7, and BTK
CD19, BANK1, JCHAIN, SLAMF7, and TNFRSF17
CD19, BANK1, JCHAIN, SLAMF7, and IGJ
CD19, BANK1, JCHAIN, SLAMF7, and IGLL5
CD19, BANK1, JCHAIN, SLAMF7, and RBPJ
CD19, CD79A, JCHAIN, SLAMF7, and BTK
CD19, CD79A, JCHAIN, SLAMF7, and TNFRSF17
CD19, CD79A, JCHAIN, SLAMF7, and IGJ
CD19, CD79A, JCHAIN, SLAMF7, and IGLL5
CD19, CD79A, JCHAIN, SLAMF7, and RBPJ

TABLE 7
Exemplary Six-Gene Combinations of B Cell Signature Genes
CD79A, SLAMF7, BTK, TNFRSF17, IGJ, and IGLL5
CD79A, SLAMF7, BTK, TNFRSF17, IGJ, and RBPJ
CD79A, SLAMF7, BTK, TNFRSF17, IGJ, and MZB1
CD79A, SLAMF7, BTK, TNFRSF17, IGLL5, and RBPJ
CD79A, SLAMF7, BTK, TNFRSF17, IGLL5, and MZB1
CD79A, SLAMF7, BTK, TNFRSF17, RBPJ, and MZB1
CD79A, SLAMF7, BTK, IGJ, IGLL5, and RBPJ
CD79A, SLAMF7, BTK, IGJ, IGLL5, and MZB1
CD79A, SLAMF7, BTK, IGJ, RBPJ, and MZB1
CD79A, SLAMF7, BTK, IGLL5, RBPJ, and MZB1
CD79A, SLAMF7, TNFRSF17, IGJ, IGLL5, and RBPJ
CD79A, SLAMF7, TNFRSF17, IGJ, IGLL5, and MZB1
CD79A, SLAMF7, TNFRSF17, IGJ, RBPJ, and MZB1
CD79A, SLAMF7, TNFRSF17, IGLL5, RBPJ, and MZB1
CD79A, SLAMF7, IGJ, IGLL5, RBPJ, and MZB1
CD79A, BTK, TNFRSF17, IGJ, IGLL5, and RBPJ
CD79A, BTK, TNFRSF17, IGJ, IGLL5, and MZB1
CD79A, BTK, TNFRSF17, IGJ, RBPJ, and MZB1
CD79A, BTK, TNFRSF17, IGLL5, RBPJ, and MZB1
CD79A, BTK, IGJ, IGLL5, RBPJ, and MZB1
CD79A, TNFRSF17, IGJ, IGLL5, RBPJ, and MZB1
SLAMF7, BTK, TNFRSF17, IGJ, IGLL5, and RBPJ
SLAMF7, BTK, TNFRSF17, IGJ, IGLL5, and MZB1
SLAMF7, BTK, TNFRSF17, IGJ, RBPJ, and MZB1
SLAMF7, BTK, TNFRSF17, IGLL5, RBPJ, and MZB1
SLAMF7, BTK, IGJ, IGLL5, RBPJ, and MZB1
SLAMF7, TNFRSF17, IGJ, IGLL5, RBPJ, and MZB1
BTK, TNFRSF17, IGJ, IGLL5, RBPJ, and MZB1
CD19, SLAMF7, BTK, TNFRSF17, IGJ, and IGLL5
CD19, SLAMF7, BTK, TNFRSF17, IGJ, and RBPJ
CD19, SLAMF7, BTK, TNFRSF17, IGJ, and MZB1
CD19, SLAMF7, BTK, TNFRSF17, IGLL5, and RBPJ
CD19, SLAMF7, BTK, TNFRSF17, IGLL5, and MZB1
CD19, SLAMF7, BTK, TNFRSF17, RBPJ, and MZB1
BANK1, SLAMF7, BTK, TNFRSF17, IGJ, and IGLL5
BANK1, SLAMF7, BTK, TNFRSF17, IGJ, and RBPJ
BANK1, SLAMF7, BTK, TNFRSF17, IGJ, and MZB1
BANK1, SLAMF7, BTK, TNFRSF17, IGLL5, and RBPJ
BANK1, SLAMF7, BTK, TNFRSF17, IGLL5, and MZB1
BANK1, SLAMF7, BTK, TNFRSF17, RBPJ, and MZB1
JCHAIN, SLAMF7, BTK, TNFRSF17, IGJ, and IGLL5
JCHAIN, SLAMF7, BTK, TNFRSF17, IGJ, and RBPJ
JCHAIN, SLAMF7, BTK, TNFRSF17, IGJ, and MZB1
JCHAIN, SLAMF7, BTK, TNFRSF17, IGLL5, and RBPJ
JCHAIN, SLAMF7, BTK, TNFRSF17, IGLL5, and MZB1
JCHAIN, SLAMF7, BTK, TNFRSF17, RBPJ, and MZB1

TABLE 8
Exemplary Seven-Gene Combinations of B Cell Signature Genes
CD79A, SLAMF7, BTK, TNFRSF17, IGJ, IGLL5, and RBPJ
CD79A, SLAMF7, BTK, TNFRSF17, IGJ, IGLL5, and MZB1
CD79A, SLAMF7, BTK, TNFRSF17, IGJ, RBPJ, and MZB1
CD79A, SLAMF7, BTK, TNFRSF17, IGLL5, RBPJ, and MZB1
CD79A, SLAMF7, BTK, IGJ, IGLL5, RBPJ, and MZB1
CD79A, SLAMF7, TNFRSF17, IGJ, IGLL5, RBPJ, and MZB1
CD79A, BTK, TNFRSF17, IGJ, IGLL5, RBPJ, and MZB1
SLAMF7, BTK, TNFRSF17, IGJ, IGLL5, RBPJ, and MZB1
CD19, SLAMF7, BTK, TNFRSF17, IGJ, IGLL5, and RBPJ
CD19, SLAMF7, BTK, TNFRSF17, IGJ, IGLL5, and MZB1
CD19, SLAMF7, BTK, TNFRSF17, IGJ, RBPJ, and MZB1
CD19, SLAMF7, BTK, TNFRSF17, IGLL5, RBPJ, and MZB1
CD19, SLAMF7, BTK, IGJ, IGLL5, RBPJ, and MZB1
CD19, SLAMF7, TNFRSF17, IGJ, IGLL5, RBPJ, and MZB1
CD19, BTK, TNFRSF17, IGJ, IGLL5, RBPJ, and MZB1
BANK1, SLAMF7, BTK, TNFRSF17, IGJ, IGLL5, and RBPJ
BANK1, SLAMF7, BTK, TNFRSF17, IGJ, IGLL5, and MZB1
BANK1, SLAMF7, BTK, TNFRSF17, IGJ, RBPJ, and MZB1
BANK1, SLAMF7, BTK, TNFRSF17, IGLL5, RBPJ, and MZB1
BANK1, SLAMF7, BTK, IGJ, IGLL5, RBPJ, and MZB1
BANK1, SLAMF7, TNFRSF17, IGJ, IGLL5, RBPJ, and MZB1
BANK1, BTK, TNFRSF17, IGJ, IGLL5, RBPJ, and MZB1
JCHAIN, SLAMF7, BTK, TNFRSF17, IGJ, IGLL5, and RBPJ
JCHAIN, SLAMF7, BTK, TNFRSF17, IGJ, IGLL5, and MZB1
JCHAIN, SLAMF7, BTK, TNFRSF17, IGJ, RBPJ, and MZB1
JCHAIN, SLAMF7, BTK, TNFRSF17, IGLL5, RBPJ, and MZB1
JCHAIN, SLAMF7, BTK, IGJ, IGLL5, RBPJ, and MZB1
JCHAIN, SLAMF7, TNFRSF17, IGJ, IGLL5, RBPJ, and MZB1
JCHAIN, BTK, TNFRSF17, IGJ, IGLL5, RBPJ, and MZB1

In some instances, the genes comprise CD79A, CD19, BANK1, JCHAIN, SLAMF7, BTK, 5 TNFRSF17, IGJ, IGLL5, RBPJ, and MZB1.

In some instances, the expression level is a nucleic acid expression level. For example, in some instances, the nucleic acid expression level is an mRNA expression level. The mRNA expression level may be determined using any suitable approach, e.g., any approach disclosed herein. In some instances, the mRNA expression level is determined by RNA-seq, RT-qPCR, qPCR, multiplex qPCR or RT-qPCR, microarray analysis, SAGE, MassARRAY technique, FISH, or a combination thereof. In some instances, the mRNA expression level is detected using RNA-seq.

In other instances, the expression level is a protein expression level. The protein expression level may be determined using any suitable approach, e.g., any approach disclosed herein. In some instances, the protein expression level is determined by IHC, immunofluorescence, mass spectrometry, flow cytometry, and Western blot, or a combination thereof.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is a FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

(ii) Gene Signatures Associated with Plasma B Cells

In some aspects, the B cell signature used in conjunction with the compositions and methods of the invention is a plasma B cell signature. Any suitable plasma B cell signature may be used. For example, the plasma B cell signature may include one or more (e.g., 1, 2, 3, 4, 5, 6, 7, 8, 9, 10, 11, 12, 13, or 14) genes set forth in Table 9.

TABLE 9
Exemplary Plasma B Cell Signature Genes
MZB1
DERL3
JSRP1
TNFRSF17
SLAMF7
IGHG2
IGHGP
IGLV3-1
IGLV6-57
IGHA2
IGKV4-1
IGKV1-12
IGLC7
IGLL5

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the method comprises determining the expression level of one of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some instances, the method comprises determining the expression level of MZB1.

In some instances, the method comprises determining the expression level of DERL3.

In some instances, the method comprises determining the expression level of JSRP1.

In some instances, the method comprises determining the expression level of TNFRSF17.

In some instances, the method comprises determining the expression level of SLAMF7.

In some instances, the method comprises determining the expression level of IGHG2.

In some instances, the method comprises determining the expression level of IGHGP.

In some instances, the method comprises determining the expression level of IGLV3-1.

In some instances, the method comprises determining the expression level of IGLV6-57.

In some instances, the method comprises determining the expression level of IGHA2.

In some instances, the method comprises determining the expression level of IGKV4-1.

In some instances, the method comprises determining the expression level of IGKV1-12.

In some instances, the method comprises determining the expression level of IGLC7.

In some instances, the method comprises determining the expression level of IGLL5.

In some instances, the immune-score expression level of the one or more genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), wherein the benefit comprises an extension in the individual's OS as compared to treatment without the PD-L1 axis binding antagonist.

In some instances, the immune-score expression level of the one or more genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

In some instances, the genes comprise two or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

For example, provided herein is a method of identifying an individual having a cancer who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of two or more (e.g., 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the two or more genes that is above a reference immune-score expression level of the two or more genes identifies the individual as one who may benefit from a treatment comprising a PD-L1 axis binding antagonist.

In another example, provided herein is a method of selecting a therapy for an individual having a cancer, the method comprising determining the expression level of two or more (e.g., 2, 3, 4, 5, 6, 7, 8, 9, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the two or more genes that is above a reference immune-score expression level of the two or more genes identifies the individual as one who may benefit from a treatment comprising a PD-L1 axis binding antagonist.

In a further example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of two or more (e.g., 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the two or more genes that is above a reference immune-score expression level of the two or more genes identifies the individual as one who may benefit from a treatment comprising a PD-L1 axis binding antagonist, wherein the benefit comprises an extension in the individual's OS as compared to treatment without the PD-L1 axis binding antagonist.

In yet another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of two or more (e.g., 2, 3, 4, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the two or more genes that is above a reference immune-score expression level of the two or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), wherein the benefit comprises an extension in the individual's OS as compared to treatment without the PD-L1 axis binding antagonist.

In some instances, the immune-score expression level of the two or more genes in the sample is above the reference immune-score expression level and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

Any combination of plasma B cell signature genes may be determined. For example, the combination may include two genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 10. In another example, the combination may include three genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 11. In another example, the combination may include four genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 12. In another example, the combination may include five genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 13. In another example, the combination may include six genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 14. In another example, the combination may include seven genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 15.

TABLE 10
Exemplary Two-Gene Combinations of Plasma B Cell Signature Genes
MZB1 and DERL3
MZB1 and JSRP1
MZB1 and TNFRSF17
MZB1 and SLAMF7
MZB1 and IGHG2
MZB1 and IGHGP
MZB1 and IGLV3-1
MZB1 and IGLV6-57
MZB1 and IGHA2
MZB1 and IGKV4-1
MZB1 and IGKV1-12
MZB1 and IGLC7
MZB1 and IGLL5
DERL3 and JSRP1
DERL3 and TNFRSF17
DERL3 and SLAMF7
DERL3 and IGHG2
DERL3 and IGHGP
DERL3 and IGLV3-1
DERL3 and IGLV6-57
DERL3 and IGHA2
DERL3 and IGKV4-1
DERL3 and IGKV1-12
DERL3 and IGLC7
DERL3 and IGLL5
JSRP1 and TNFRSF17
JSRP1 and SLAMF7
JSRP1 and IGHG2
JSRP1 and IGHGP
JSRP1 and IGLV3-1
JSRP1 and IGLV6-57
JSRP1 and IGHA2
JSRP1 and IGKV4-1
JSRP1 and IGKV1-12
JSRP1 and IGLC7
JSRP1 and IGLL5
TNFRSF17 and SLAMF7
TNFRSF17 and IGHG2
TNFRSF17 and IGHGP
TNFRSF17 and IGLV3-1
TNFRSF17 and IGLV6-57
TNFRSF17 and IGHA2
TNFRSF17 and IGKV4-1
TNFRSF17 and IGKV1-12
TNFRSF17 and IGLC7
TNFRSF17 and IGLL5
SLAMF7 and IGHG2
SLAMF7 and IGHGP
SLAMF7 and IGLV3-1
SLAMF7 and IGLV6-57
SLAMF7 and IGHA2
SLAMF7 and IGKV4-1
SLAMF7 and IGKV1-12
SLAMF7 and IGLC7
SLAMF7 and IGLL5
IGHG2 and IGHGP
IGHG2 and IGLV3-1
IGHG2 and IGLV6-57
IGHG2 and IGHA2
IGHG2 and IGKV4-1
IGHG2 and IGKV1-12
IGHG2 and IGLC7
IGHG2 and IGLL5

TABLE 11
Exemplary Three-Gene Combinations of Plasma
B Cell Signature Genes

MZB1, DERL3, and JSRP1
MZB1, DERL3, and TNFRSF17
MZB1, DERL3, and SLAMF7
MZB1, DERL3, and IGHG2
MZB1, DERL3, and IGHGP
MZB1, DERL3, and IGLV3-1
MZB1, DERL3, and IGLV6-57
MZB1, DERL3, and IGHA2
MZB1, DERL3, and IGKV4-1
MZB1, DERL3, and IGKV1-12
MZB1, DERL3, and IGLC7
MZB1, DERL3, and IGLL5
MZB1, JSRP1, and TNFRSF17
MZB1, JSRP1, and SLAMF7
MZB1, JSRP1, and IGHG2
MZB1, JSRP1, and IGHGP
MZB1, JSRP1, and IGLV3-1
MZB1, JSRP1, and IGLV6-57
MZB1, JSRP1, and IGHA2
MZB1, JSRP1, and IGKV4-1
MZB1, JSRP1, and IGKV1-12
MZB1, JSRP1, and IGLC7
MZB1, JSRP1, and IGLL5
MZB1, TNFRSF17, and SLAMF7
MZB1, TNFRSF17, and IGHG2
MZB1, TNFRSF17, and IGHGP
MZB1, TNFRSF17, and IGLV3-1
MZB1, TNFRSF17, and IGLV6-57
MZB1, TNFRSF17, and IGHA2
MZB1, TNFRSF17, and IGKV4-1
MZB1, TNFRSF17, and IGKV1-12
MZB1, TNFRSF17, and IGLC7
MZB1, TNFRSF17, and IGLL5

TABLE 12
Exemplary Four-Gene Combinations of Plasma B Cell Signature Genes
MZB1, DERL3, JSRP1, and TNFRSF17
MZB1, DERL3, JSRP1, and SLAMF7
MZB1, DERL3, JSRP1, and IGHG2
MZB1, DERL3, JSRP1, and IGHGP
MZB1, DERL3, JSRP1, and IGLV3-1
MZB1, DERL3, JSRP1, and IGLV6-57
MZB1, DERL3, JSRP1, and IGHA2
MZB1, DERL3, JSRP1, and IGKV4-1
MZB1, DERL3, JSRP1, and IGKV1-12
MZB1, DERL3, JSRP1, and IGLC7
MZB1, DERL3, JSRP1, and IGLL5
MZB1, DERL3, TNFRSF17, and SLAMF7
MZB1, DERL3, TNFRSF17, and IGHG2
MZB1, DERL3, TNFRSF17, and IGHGP
MZB1, DERL3, TNFRSF17, and IGLV3-1
MZB1, DERL3, TNFRSF17, and IGLV6-57
MZB1, DERL3, TNFRSF17, and IGHA2
MZB1, DERL3, TNFRSF17, and IGKV4-1
MZB1, DERL3, TNFRSF17, and IGKV1-12
MZB1, DERL3, TNFRSF17, and IGLC7
MZB1, DERL3, TNFRSF17, and IGLL5
MZB1, DERL3, SLAMF7, and IGHG2
MZB1, DERL3, SLAMF7, and IGHGP
MZB1, DERL3, SLAMF7, and IGLV3-1
MZB1, DERL3, SLAMF7, and IGLV6-57
MZB1, DERL3, SLAMF7, and IGHA2
MZB1, DERL3, SLAMF7, and IGKV4-1
MZB1, DERL3, SLAMF7, and IGKV1-12
MZB1, DERL3, SLAMF7, and IGLC7
MZB1, DERL3, SLAMF7, and IGLL5
MZB1, DERL3, IGHG2, and IGHGP
MZB1, DERL3, IGHG2, and IGLV3-1
MZB1, DERL3, IGHG2, and IGLV6-57
MZB1, DERL3, IGHG2, and IGHA2
MZB1, DERL3, IGHG2, and IGKV4-1
MZB1, DERL3, IGHG2, and IGKV1-12
MZB1, DERL3, IGHG2, and IGLC7
MZB1, DERL3, IGHG2, and IGLL5

TABLE 13
Exemplary Five-Gene Combinations of Plasma B Cell Signature Genes
MZB1, DERL3, JSRP1, TNFRSF17, and SLAMF7
MZB1, DERL3, JSRP1, TNFRSF17, and IGHG2
MZB1, DERL3, JSRP1, TNFRSF17, and IGHGP
MZB1, DERL3, JSRP1, TNFRSF17, and IGLV3-1
MZB1, DERL3, JSRP1, TNFRSF17, and IGLV6-57
MZB1, DERL3, JSRP1, TNFRSF17, and IGHA2
MZB1, DERL3, JSRP1, TNFRSF17, and IGKV4-1
MZB1, DERL3, JSRP1, TNFRSF17, and IGKV1-12
MZB1, DERL3, JSRP1, TNFRSF17, and IGLC7
MZB1, DERL3, JSRP1, TNFRSF17, and IGLL5
MZB1, DERL3, JSRP1, SLAMF7, and IGHG2
MZB1, DERL3, JSRP1, SLAMF7, and IGHGP
MZB1, DERL3, JSRP1, SLAMF7, and IGLV3-1
MZB1, DERL3, JSRP1, SLAMF7, and IGLV6-57
MZB1, DERL3, JSRP1, SLAMF7, and IGHA2
MZB1, DERL3, JSRP1, SLAMF7, and IGKV4-1
MZB1, DERL3, JSRP1, SLAMF7, and IGKV1-12
MZB1, DERL3, JSRP1, SLAMF7, and IGLC7
MZB1, DERL3, JSRP1, SLAMF7, and IGLL5
MZB1, DERL3, JSRP1, IGHG2, and IGHGP
MZB1, DERL3, JSRP1, IGHG2, and IGLV3-1
MZB1, DERL3, JSRP1, IGHG2, and IGLV6-57
MZB1, DERL3, JSRP1, IGHG2, and IGHA2
MZB1, DERL3, JSRP1, IGHG2, and IGKV4-1
MZB1, DERL3, JSRP1, IGHG2, and IGKV1-12
MZB1, DERL3, JSRP1, IGHG2, and IGLC7
MZB1, DERL3, JSRP1, IGHG2, and IGLL5

TABLE 14
Exemplary Six-Gene Combinations of Plasma B Cell Signature Genes
MZB1, DERL3, JSRP1, TNFRSF17, SLAMF7, and IGHG2
MZB1, DERL3, JSRP1, TNFRSF17, SLAMF7, and IGHGP
MZB1, DERL3, JSRP1, TNFRSF17, SLAMF7, and IGLV3-1
MZB1, DERL3, JSRP1, TNFRSF17, SLAMF7, and IGLV6-57
MZB1, DERL3, JSRP1, TNFRSF17, SLAMF7, and IGKV4-1
MZB1, DERL3, JSRP1, TNFRSF17, SLAMF7, and IGKV1-12
MZB1, DERL3, JSRP1, TNFRSF17, SLAMF7, and IGLC7
MZB1, DERL3, JSRP1, TNFRSF17, SLAMF7, and IGLL5
MZB1, DERL3, JSRP1, TNFRSF17, IGHG2, and IGHGP
MZB1, DERL3, JSRP1, TNFRSF17, IGHG2, and IGLV3-1
MZB1, DERL3, JSRP1, TNFRSF17, IGHG2, and IGLV6-57
MZB1, DERL3, JSRP1, TNFRSF17, IGHG2, and IGKV4-1
MZB1, DERL3, JSRP1, TNFRSF17, IGHG2, and IGKV1-12
MZB1, DERL3, JSRP1, TNFRSF17, IGHG2, and IGLC7
MZB1, DERL3, JSRP1, TNFRSF17, IGHG2, and IGLL5
MZB1, DERL3, JSRP1, TNFRSF17, IGHGP, and IGLV3-1
MZB1, DERL3, JSRP1, TNFRSF17, IGHGP, and IGLV6-57
MZB1, DERL3, JSRP1, TNFRSF17, IGHGP, and IGKV4-1
MZB1, DERL3, JSRP1, TNFRSF17, IGHGP, and IGKV1-12
MZB1, DERL3, JSRP1, TNFRSF17, IGHGP, and IGLC7
MZB1, DERL3, JSRP1, TNFRSF17, IGHGP, and IGLL5
MZB1, DERL3, JSRP1, TNFRSF17, IGLV3-1, and IGLV6-57
MZB1, DERL3, JSRP1, TNFRSF17, IGLV3-1, and IGKV4-1
MZB1, DERL3, JSRP1, TNFRSF17, IGLV3-1, and IGKV1-12
MZB1, DERL3, JSRP1, TNFRSF17, IGLV3-1, and IGLC7
MZB1, DERL3, JSRP1, TNFRSF17, IGLV3-1, and IGLL5

TABLE 15
Exemplary Seven-Gene Combinations of Plasma B Cell Signature Genes
MZB1, DERL3, JSRP1, TNFRSF17, SLAMF7, IGHG2, and IGHGP
MZB1, DERL3, JSRP1, TNFRSF17, SLAMF7, IGHG2, and IGLV3-1
MZB1, DERL3, JSRP1, TNFRSF17, SLAMF7, IGHG2, and IGLV6-57
MZB1, DERL3, JSRP1, TNFRSF17, SLAMF7, IGHG2, and IGHA2
MZB1, DERL3, JSRP1, TNFRSF17, SLAMF7, IGHG2, and IGKV4-1
MZB1, DERL3, JSRP1, TNFRSF17, SLAMF7, IGHG2, and IGKV1-12
MZB1, DERL3, JSRP1, TNFRSF17, SLAMF7, IGHG2, and IGLC7
MZB1, DERL3, JSRP1, TNFRSF17, SLAMF7, IGHG2, and IGLL5
MZB1, DERL3, JSRP1, TNFRSF17, SLAMF7, IGHGP, and IGLV3-1
MZB1, DERL3, JSRP1, TNFRSF17, SLAMF7, IGHGP, and IGLV6-57
MZB1, DERL3, JSRP1, TNFRSF17, SLAMF7, IGHGP, and IGHA2
MZB1, DERL3, JSRP1, TNFRSF17, SLAMF7, IGHGP, and IGKV4-1
MZB1, DERL3, JSRP1, TNFRSF17, SLAMF7, IGHGP, and IGKV1-12
MZB1, DERL3, JSRP1, TNFRSF17, SLAMF7, IGHGP, and IGLC7
MZB1, DERL3, JSRP1, TNFRSF17, SLAMF7, IGHGP, and IGLL5
MZB1, DERL3, JSRP1, TNFRSF17, SLAMF7, IGLV3-1, and IGLV6-57
MZB1, DERL3, JSRP1, TNFRSF17, SLAMF7, IGLV3-1, and IGHA2
MZB1, DERL3, JSRP1, TNFRSF17, SLAMF7, IGLV3-1, and IGKV4-1
MZB1, DERL3, JSRP1, TNFRSF17, SLAMF7, IGLV3-1, and IGKV1-12
MZB1, DERL3, JSRP1, TNFRSF17, SLAMF7, IGLV3-1, and IGLC7
MZB1, DERL3, JSRP1, TNFRSF17, SLAMF7, IGLV3-1, and IGLL5

In some instances, the genes comprise MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some instances, the expression level is a nucleic acid expression level. For example, in some instances, the nucleic acid expression level is an mRNA expression level. The mRNA expression level may be determined using any suitable approach, e.g., any approach disclosed herein. In some instances, the mRNA expression level is determined by RNA-seq, RT-qPCR, qPCR, multiplex qPCR or RT-qPCR, microarray analysis, SAGE, MassARRAY technique, FISH, or a combination thereof. In some instances, the mRNA expression level is detected using RNA-seq.

In other instances, the expression level is a protein expression level. The protein expression level may be determined using any suitable approach, e.g., any approach disclosed herein. In some instances, the protein expression level is determined by IHC, immunofluorescence, mass spectrometry, flow cytometry, and Western blot, or a combination thereof.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is a FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### B. Tertiary Lymphoid Structure (TLS)

In some aspects, the methods provided herein may involve determining the presence of a TLS in a sample (e.g., a tumor sample) from an individual having a cancer (e.g., lung cancer (e.g., NSCLC), bladder cancer (e.g., UC), kidney cancer (e.g., RCC), or breast cancer (e.g., TNBC)).

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the presence of a tertiary lymphoid structure (TLS) in a sample (e.g., a tumor sample) from the individual, wherein the presence of a TLS in the sample identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the presence of a TLS in a sample (e.g., a tumor sample) from the individual, wherein the presence of a TLS in the sample identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the sample from the individual is determined to have the presence of a TLS and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

Any suitable approach can be used to determine the presence of a TLS in a sample. For example, in some instances, the presence of a TLS is determined by histological staining, IHC, immunofluorescence, or gene expression analysis.

Any suitable histological staining approach can be used. For example, in some instances, the histological staining comprises hematoxylin and eosin (H&E) staining.

Any suitable IHC or immunofluorescence approach can be used. In some instances, the IHC or immunofluorescence comprises detecting CD62L, L-selectin, CD40, or CD8, e.g., using an antibody (e.g., an anti-CD62L antibody, an anti-L-selectin antibody, an anti-CD40 antibody, and/or an anti-CD8 antibody). In some instances, CD62L or L-selectin is detected using a MECA-79 antibody.

In some instances, the gene expression analysis comprises determining the expression level of a TLS gene signature in the sample. For example, the gene expression analysis may involve determining the expression level of any TLS signature disclosed herein (see, e.g., Section II, Subsection C below). In some instances, the TLS gene signature comprises one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13. In some instances, the genes comprise two or more of CCL2, CCL3, CCL4, CCL5, CCL8, CCL18, CCL19, CCL21, CXCL9, CXCL10, CXCL11, and CXCL13.

In some instances, the sample may include one TLS. In other instances, the sample may include more than one TLS, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, or more TLS.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is an FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### C. TLS Signatures

In some aspects, the methods provided herein may involve determining an expression level of one or more genes in a TLS signature. Any suitable TLS signature may be used. For example, the TLS signature may include one or more (e.g., 1, 2, 3, 4, 5, 6, 7, 8, 9, 10, 11, or 12) genes set forth in Table 16.

TABLE 16
Exemplary TLS Signature Genes
CCL2
CCL3
CCL4
CCL5
CCL8
CCL18
CCL19
CCL21
CXCL9
CXCL10
CXCL11
CXCL13

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the one or more genes that is above a reference immune-score expression level of the one or more genes identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the one or more genes that is above a reference immune-score expression level of the one or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the immune-score expression level of the one or more (e.g., 1, 2, 3, 4, 5, 6, 7, 8, 9, 10, 11, or 12) genes in the sample is above the reference immune-score expression level of the one or more genes and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

In some instances, the genes comprise two or more of CCL2, CCL3, CCL4, CCL5, CCL8, CCL18, CCL19, CCL21, CXCL9, CXCL10, CXCL11, and CXCL13.

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of two or more (e.g., 2, 3, 4, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the immune-score expression level of the two or more genes in the sample is above the reference immune-score expression level and the method further comprises administering to the individual an effective amount of a PD-L1 axis binding antagonist. Any suitable PD-L1 axis binding antagonist may be administered, e.g., any PD-L1 axis binding antagonist provided herein (e.g., as described below in Section IV).

In some instances, the reference immune-score expression level is an immune-score expression level of the two or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of the two or more genes in the reference population.

In some instances, the genes comprise three or more of CCL2, CCL3, CCL4, CCL5, CCL8, CCL18, CCL19, CCL21, CXCL9, CXCL10, CXCL11, and CXCL13. In some instances, the genes comprise four or more of CCL2, CCL3, CCL4, CCL5, CCL8, CCL18, CCL19, CCL21, CXCL9, CXCL10, CXCL11, and CXCL13. In some instances, the genes comprise five or more of CCL2, CCL3, CCL4, CCL5, CCL8, CCL18, CCL19, CCL21, CXCL9, CXCL10, CXCL11, and CXCL13. In some instances, the genes comprise six or more of CCL2, CCL3, CCL4, CCL5, CCL8, CCL18, CCL19, CCL21, CXCL9, CXCL10, CXCL11, and CXCL13. In some instances, the genes comprise seven or more of CCL2, CCL3, CCL4, CCL5, CCL8, CCL18, CCL19, CCL21, CXCL9, CXCL10, CXCL11, and CXCL13. In some instances, the genes comprise eight or more of CCL2, CCL3, CCL4, CCL5, CCL8, CCL18, CCL19, CCL21, CXCL9, CXCL10, CXCL11, and CXCL13. In some instances, the genes comprise nine or more of CCL2, CCL3, CCL4, CCL5, CCL8, CCL18, CCL19, CCL21, CXCL9, CXCL10, CXCL11, and CXCL13. In some instances, the genes comprise ten or more of CCL2, CCL3, CCL4, CCL5, CCL8, CCL18, CCL19, CCL21, CXCL9, CXCL10, CXCL11, and CXCL13. In some instances, the genes comprise eleven or more of CCL2, CCL3, CCL4, CCL5, CCL8, CCL18, CCL19, CCL21, CXCL9, CXCL10, CXCL11, and CXCL13.

Any combination of TLS signature genes may be determined, e.g., any combination of two genes selected from CCL2, CCL3, CCL4, CCL5, CCL8, CCL18, CCL19, CCL21, CXCL9, CXCL10, CXCL11, and CXCL13; any combination of three genes selected from CCL2, CCL3, CCL4, CCL5, CCL8, CCL18, CCL19, CCL21, CXCL9, CXCL10, CXCL11, and CXCL13; any combination of four genes selected from CCL2, CCL3, CCL4, CCL5, CCL8, CCL18, CCL19, CCL21, CXCL9, CXCL10, CXCL11, and CXCL13; any combination of five genes selected from CCL2, CCL3, CCL4, CCL5, CCL8, CCL18, CCL19, CCL21, CXCL9, CXCL10, CXCL11, and CXCL13; any combination of six genes selected from CCL2, CCL3, CCL4, CCL5, CCL8, CCL18, CCL19, CCL21, CXCL9, CXCL10, CXCL11, and CXCL13; any combination of seven genes selected from CCL2, CCL3, CCL4, CCL5, CCL8, CCL18, CCL19, CCL21, CXCL9, CXCL10, CXCL11, and CXCL13; any combination of eight genes selected from CCL2, CCL3, CCL4, CCL5, CCL8, CCL18, CCL19, CCL21, CXCL9, CXCL10, CXCL11, and CXCL13; any combination of nine genes selected from CCL2, CCL3, CCL4, CCL5, CCL8, CCL18, CCL19, CCL21, CXCL9, CXCL10, CXCL11, and CXCL13; any combination of ten genes selected from CCL2, CCL3, CCL4, CCL5, CCL8, CCL18, CCL19, CCL21, CXCL9, CXCL10, CXCL11, and CXCL13; any combination of eleven genes selected from CCL2, CCL3, CCL4, CCL5, CCL8, CCL18, CCL19, CCL21, CXCL9, CXCL10, CXCL11, and CXCL13; or any combination of twelve genes selected from CCL2, CCL3, CCL4, CCL5, CCL8, CCL18, CCL19, CCL21, CXCL9, CXCL10, CXCL11, and CXCL13.

In some instances, the genes comprise CCL2, CCL3, CCL4, CCL5, CCL8, CCL18, CCL19, CCL21, CXCL9, CXCL10, CXCL11, and CXCL13.

In some instances, the expression level is a nucleic acid expression level. For example, in some instances, the nucleic acid expression level is an mRNA expression level. The mRNA expression level may be determined using any suitable approach, e.g., any approach disclosed herein. In some instances, the mRNA expression level is determined by RNA-seq, RT-qPCR, qPCR, multiplex qPCR or RT-qPCR, microarray analysis, SAGE, MassARRAY technique, FISH, or a combination thereof. In some instances, the mRNA expression level is detected using RNA-seq.

In other instances, the expression level is a protein expression level. The protein expression level may be determined using any suitable approach, e.g., any approach disclosed herein. In some instances, the protein expression level is determined by IHC, immunofluorescence, mass spectrometry, flow cytometry, and Western blot, or a combination thereof.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is a, FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### D. B Cell Number and Clonally Expanded B Cells

In some aspects, the methods provided herein may involve determining the presence and/or number of B cells in a sample from an individual. In some aspects, the methods provided herein may involve determining the presence and/or number of clonally expanded B cells in a sample from an individual.

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the number of B cells in a sample (e.g., a tumor sample) from the individual, wherein a number of B cells in the sample that is above a reference number of B cells identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the number of B cells in a sample (e.g., a tumor sample) from the individual, wherein a number of B cells in the tumor sample that is above a reference number of B cells identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the number of B cells in the sample is above the reference number and the method further comprises administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Any suitable PD-L1 axis binding antagonist may be administered, e.g., any PD-L1 axis binding antagonist provided herein (e.g., as described below in Section IV).

The presence and/or number of any suitable type of B cell may be determined. For example, in some instances, the B cells comprise CD79+ B cells, IgG+ B cells, and/or plasma cells.

In some embodiments, the presence and/or number of clonally expanded B cells may be determined.

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., an anti-PD-L1 antibody, e.g., atezolizumab) or a PD-1 binding antagonist (e.g., an anti-PD-1 antibody)), the method comprising determining whether the individual has clonally expanded B cells in a sample (e.g., a tumor sample) from the individual, wherein clonally expanded B cells in the sample identify the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining whether the individual has clonally expanded B cells in a sample (e.g., a tumor sample) from the individual, wherein clonally expanded B cells in the sample identify the individual as one who may benefit from a treatment comprising a PD-L1 axis binding antagonist (e.g., an anti-PD-L1 antibody, e.g., atezolizumab) or a PD-1 binding antagonist (e.g., an anti-PD-1 antibody)).

In some instances, the sample (e.g., the tumor sample) comprises clonally expanded B cells and the method further comprises administering to the individual an effective amount of a PD-L1 axis binding antagonist (e.g., an anti-PD-L1 antibody, e.g., atezolizumab) or a PD-1 binding antagonist (e.g., an anti-PD-1 antibody)).

The clonally expanded B cells may be any type of B cell. For example, in some instances, the clonally expanded B cells are clonally expanded plasma cells.

Clonally expanded B cells may be detected by any suitable approach. For example, in some instances, clonally expanded B cells are detected by measuring the diversity of the B cell receptor (BCR) gene repertoire in the tumor sample. In some instances, a Shannon Diversity Index (SDI) of the BCR gene repertoire in the tumor sample from the individual that is below a reference SDI identifies the individual as one who may benefit from a treatment comprising a PD-L1 axis binding antagonist.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is an FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### E. T Effector Signatures

Any of the methods described herein may further include determining the presence and/or expression level of one or more T effector signature genes. Any suitable T effector signature may be used. For example, the T effector signature may include one or more (e.g., 1, 2, 3, 4, 5, 6, 7, or 8) genes set forth in Table 17. In some instances, CD274 is further detected.

TABLE 17
Exemplary T effector Signature Genes
CD8A
EOMES
GZMA
TBX21
IFNG
GZMB
CXCL9
CXCL10

For example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual, wherein an immune-score expression level of (i) the one or more genes set forth in Table 1 and (ii) the one or more of genes CD8A, EOMES, GZMA, TBX21, IFNG, GZMB, CXCL9, and CXCL10 that is above a reference immune-score expression level of the one or more genes set forth in Table 1 and the one or more of genes CD8A, EOMES, GZMA, TBX21, IFNG, GZMB, CXCL9, and CXCL10 identifies the individual as one who may benefit from a treatment comprising a PD-L1 axis binding antagonist.

In another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual, wherein an immune-score expression level of (i) the one or more genes set forth in Table 1 and (ii) the one or more of genes CD8A, EOMES, GZMA, TBX21, IFNG, GZMB, CXCL9, and CXCL10 that is above a reference immune-score expression level of the one or more genes set forth in Table 1 and the one or more of genes CD8A, EOMES, GZMA, TBX21, IFNG, GZMB, CXCL9, and CXCL10 identifies the individual as one who may benefit from a treatment comprising a PD-L1 axis binding antagonist (e.g., an anti-PD-L1 antibody, e.g., atezolizumab) or a PD-1 binding antagonist (e.g., an anti-PD-1 antibody)).

In some instances, the immune-score expression level of (i) the one or more genes set forth in Table 1 and (ii) the one or more of genes CD8A, EOMES, GZMA, TBX21, IFNG, GZMB, CXCL9, and CXCL10 in the sample is above the reference immune-score expression level and the method further comprises administering to the individual an effective amount of a PD-L1 axis binding antagonist. Any suitable PD-L1 axis binding antagonist may be administered, e.g., any PD-L1 axis binding antagonist provided herein (e.g., as described below in Section IV).

In some instances, the reference immune-score expression level is an immune-score expression level of (i) the one or more genes set forth in Table 1 and (ii) the one or more of genes CD8A, EOMES, GZMA, TBX21, IFNG, GZMB, CXCL9, and CXCL10 in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of the (i) the one or more genes set forth in Table 1 and (ii) the one or more of genes CD8A, EOMES, GZMA, TBX21, IFNG, GZMB, CXCL9, and CXCL10 in the reference population.

In another example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more B cell signature genes and (ii) the one or more T effector signature genes that is above a reference immune-score expression level of the one or more B cell signature genes and the one or more T effector signature genes identifies the individual as one who may benefit from a treatment comprising a PD-L1 axis binding antagonist.

In yet another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more B cell signature genes and (ii) the one or more T effector signature genes that is above a reference immune-score expression level of the one or more B cell signature genes and the one or more T effector signature genes identifies the individual as one who may benefit from a treatment comprising a PD-L1 axis binding antagonist (e.g., an anti-PD-L1 antibody, e.g., atezolizumab) or a PD-1 binding antagonist (e.g., an anti-PD-1 antibody)).

In some instances, the immune-score expression level of (i) the one or more B cell signature genes and (ii) the one or more T effector signature genes in the sample is above the reference immune-score expression level and the method further comprises administering to the individual an effective amount of a PD-L1 axis binding antagonist. Any suitable PD-L1 axis binding antagonist may be administered, e.g., any PD-L1 axis binding antagonist provided herein (e.g., as described below in Section IV).

In some instances, the reference immune-score expression level is an immune-score expression level of (i) the one or more B cell signature genes and (ii) the one or more T effector signature genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of (i) the one or more B cell signature genes and (ii) the one or more T effector signature genes in the reference population.

In another example, provided herein is a method of identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the method comprising determining the expression level of (i) one or more (e.g., 1, 2, 3, 4, 5, 6, 7, 8, 9, 10, 11, or 12) of TLS signature genes CCL2, CCL3, CCL4, CCL5, CCL8, CCL18, CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more TLS signature genes and (ii) the one or more T effector signature genes that is above a reference immune-score expression level of the one or more B cell signature genes and the one or more T effector signature genes identifies the individual as one who may benefit from a treatment comprising a PD-L1 axis binding antagonist.

In yet another example, provided herein is a method of selecting a therapy for an individual having a cancer (e.g., lung cancer (e.g., NSCLC), bladder cancer (e.g., UC), kidney cancer (e.g., RCC), or breast cancer (e.g., TNBC)), the method comprising determining the expression level of (i) one or more (e.g., 1, 2, 3, 4, 5, 6, 7, 8, 9, 10, 11, or 12) of TLS signature genes CCL2, CCL3, CCL4, CCL5, CCL8, CCL18,

CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more TLS signature genes and (ii) the one or more T effector signature genes that is above a reference immune-score expression level of the one or more B cell signature genes and the one or more T effector signature genes identifies the individual as one who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the immune-score expression level of (i) the one or more TLS signature genes and (ii) the one or more T effector signature genes in the sample is above the reference immune-score expression level and the method further comprises administering to the individual an effective amount of a PD-L1 axis binding antagonist. Any suitable PD-L1 axis binding antagonist may be administered, e.g., any PD-L1 axis binding antagonist provided herein (e.g., as described below in Section IV).

In some instances, the reference immune-score expression level is an immune-score expression level of (i) the one or more TLS signature genes and (ii) the one or more T effector signature genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of (i) the one or more TLS signature genes and (ii) the one or more T effector signature genes in the reference population.

In some instances of any of the preceding methods, the presence and/or expression level of CD274 is further determined.

In any of the methods described herein, the method may include determining the expression level of CD79A, CD274, and one or more (e.g., 1, 2, 3, 4, 5, 6, 7, or 8) of T effector signature genes CD8A, EOMES, GZMA, TBX21, IFNG, GZMB, CXCL9, and CXCL10.

### F. Determination of Expression Levels

(i) Detection Methods

The immune-score expression level of the genes described herein (e.g., one or more genes set forth in any one of Tables 1-17) may be based on a nucleic acid expression level, and preferably, an mRNA expression level. Presence and/or expression levels/amount of the genes described herein can be determined qualitatively and/or quantitatively based on any suitable criterion known in the art, including but not limited to DNA, mRNA, cDNA, proteins, protein fragments, and/or gene copy number.

In some instances, nucleic acid expression levels of the genes described herein (e.g., one or more genes set forth in any one of Tables 1-17) may be measured by polymerase chain reaction (PCR)-based assays, e.g., quantitative PCR, real-time PCR, quantitative real-time PCR (qRT-PCR), reverse transcriptase PCR (RT-PCR), and reverse transcriptase quantitative PCR (RT-qPCR). Platforms for performing quantitative PCR assays include Fluidigm (e.g., BIOMARK™ HD System). Other amplification-based methods include, for example, transcript-mediated amplification (TMA), strand displacement amplification (SDA), nucleic acid sequence-based amplification (NASBA), and signal amplification methods such as bDNA.

In some instances, nucleic acid expression levels of the genes described herein (e.g., one or more genes set forth in any one of Tables 1-17) also may be measured by sequencing-based techniques, such as, for example, RNA-seq, serial analysis of gene expression (SAGE), high-throughput sequencing technologies (e.g., massively parallel sequencing), and Sequenom MassARRAY® technology. Nucleic acid expression levels (e.g., expression levels of one or more of genes set forth in any one of Tables 1-17)) also may be measured by, for example, NanoString nCounter, and high-coverage expression profiling (HiCEP). Additional protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Other methods for detecting nucleic acid levels of the genes described herein (e.g., one or more genes set forth in any one of Tables 1-17) include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene.

Primers and probes may be labeled with a detectable marker, such as, for example, a radioisotope, fluorescent compound, bioluminescent compound, a chemiluminescent compound, metal chelator, or enzyme. Such probes and primers can be used to detect the presence of expressed genes, such as one or more of genes set forth in any one of Tables 1-17 in a sample. As will be understood by the skilled artisan, many different primers and probes may be prepared based on the sequences provided herein (or, in the case of genomic DNA, their adjacent sequences) and used effectively to amplify, clone, and/or determine the presence and/or expression levels of the genes described herein.

Other methods to detect nucleic acid expression levels of the genes described herein (e.g., one or more genes set forth in any one of Tables 1-17) include electrophoresis, Northern and Southern blot analyses, in situ hybridization (e.g., single or multiplex nucleic acid in situ hybridization), RNAse protection assays, and microarrays (e.g., Illumina BEADARRAY™ technology; Beads Array for Detection of Gene Expression (BADGE)).

In some instances, the immune-score expression level of the genes described herein (e.g., one or more genes set forth in any one of Tables 1-17) can be analyzed by a number of methodologies, including, but not limited to, RNA-seq, PCR, RT-qPCR, qPCR, multiplex qPCR, multiplex RT-qPCR, NANOSTRING® nCOUNTER® Gene Expression Assay, microarray analysis, serial analysis of gene expression (SAGE), Northern blot analysis, MassARRAY, ISH, and whole genome sequencing, or combinations thereof.

In further instances, the immune-score expression level of the genes described herein (e.g., one or more genes set forth in any one of Tables 1-17) may be detected in the sample using a method selected from the group consisting of RNA-seq, RT-qPCR, qPCR, multiplex qPCR, multiplex RT-qPCR, microarray analysis, SAGE, MassARRAY technique, FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, and ISH, or combinations thereof.

(ii) RT-qPCR

In some instances, nucleic acid expression levels of the genes described herein (e.g., one or more genes set forth in any one of Tables 1-17) can be detected using reverse transcription quantitative polymerase chain reaction (RT-qPCR). The technique of RT-qPCR is a form of PCR wherein the nucleic acid to be amplified is RNA that is first reverse transcribed into cDNA and the amount of PCR product is measured at each step in a PCR reaction. As RNA cannot serve as a template for PCR, the first step in gene expression profiling by PCR is the reverse transcription of the RNA template into cDNA, followed by its amplification in a PCR reaction. For example, reverse transcriptases may include avilo myeloblastosis virus reverse transcriptase (AMY-RT) or Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif, USA), following the manufacturer's instructions. The derived cDNA can then be used as a template in the subsequent PCR reaction.

A variation of the PCR technique is quantitative real time PCR (qRT-PCR), which measures PCR product accumulation through a dual-labeled fluorogenic probe (i.e., TAQMAN® probe). The technique of quantitative real time polymerase chain reaction refers to a form of PCR wherein the amount of PCR product is measured at each step in a PCR reaction. This technique has been described in various publications including Cronin et al., Am. J. Pathol. 164(I):35-42 (2004); and Ma et al., Cancer Cell 5:607-616 (2004). Real time PCR is compatible both with quantitative competitive PCR, where an internal competitor for each target sequence is used for normalization, and/or with quantitative comparative PCR using a normalization gene contained within the sample, or a housekeeping gene for PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: Godfrey et al., Malec. Diagnostics 2: 84-91 (2000); Specht et al., Am. J. Pathol. 158: 419-29 (2001)). Briefly, a representative process starts with cutting a section (e.g., a 10 microgram section) of a paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

The nucleic acid expression level determined by an amplification-based method (e.g., RT-qPCR) may be expressed as a cycle threshold value (Ct). From this value, a normalized expression level for each gene can be determined, e.g., using the delta Ct (dCt) method as follows: Ct(Control/Reference Gene)−Ct(Gene of Interest/Target Gene)=dCt (Gene of Interest/Target Gene). One of skill in the art will appreciate that the dCt value obtained may be a negative dCt value or a positive dCt value. As defined herein, a higher dCt value indicates a higher expression level of the gene of interest relative to the control gene. Conversely, a lower dCt value indicates a lower expression level of the gene of interest relative to the control gene. In cases where the expression levels of a plurality of genes has been determined, the expression level for each gene, e.g., expressed as a dCt value, may then be used to determine a single value that represents an aggregate or composite expression level for the plurality of genes (e.g., an immune-score expression level). The immune-score expression level may be the mean or median of dCt values determined for each target gene/gene of interest. Thus, in some instances, the immune-score expression level described herein may be the mean or median of dCt values determined for one or more of genes set forth in any one of Tables 1-17. As defined herein, a higher averaged dCt or median dCt value indicates a higher aggregative expression level of the plurality of target genes relative to the control gene (or plurality of control genes). A lower averaged dCt or median dCt value indicates a lower aggregative expression level of the plurality of target genes relative to the control gene (or plurality of control genes). As described herein, an immune-score expression level may in turn be compared to a reference immune-score expression level as further defined herein.

In some embodiments, the nucleic acid expression levels described herein may be determined using a method including: (a) obtaining or providing a sample from the individual, wherein the sample includes a tumor tissue sample (e.g., a paraffin-embedded, formalin-fixed NSCLC, UC, RCC, or TNBC tumor tissue sample); (b) isolating mRNA from the sample; (c) performing reverse transcription of the mRNA into cDNA (e.g., for one or more genes set forth in any one of Tables 1-17); (d) amplifying the cDNA (e.g., for one or more genes set forth in any one of Tables 1-17) using PCR; and (e) quantifying the nucleic acid expression levels (e.g., for one or more genes set forth in any one of Tables 1-17).

One or more genes (e.g., one or more genes set forth in any one of Tables 1-17) may be detected in a single assay depending on the primers or probes used. Further, the assay may be performed across one or more tubes (e.g., one, two, three, four, five, or six or more tubes).

In some instances, the method further comprises (f) normalizing the nucleic acid expression level of the gene(s) (e.g., one or more genes set forth in any one of Tables 1-17) in the sample to the expression level of one or more reference genes (e.g., one, two, three, four, five, six, seven, eight, nine, or more reference genes, e.g., a housekeeping gene). For example, RT-qPCR may be used to analyze the immune-score expression level of the genes described herein (e.g., one or more genes set forth in any one of Tables 1-17) to generate an immune-score expression level that reflects a normalized, averaged dCT value for the analyzed genes.

(iii) RNA-Seq

In some instances, nucleic acid expression levels of the genes described herein (e.g., one or more genes set forth in any one of Tables 1-17) can be detected using RNA-seq. RNA-seq, also called Whole Transcriptome Shotgun Sequencing (WTSS), refers to the use of high-throughput sequencing technologies to sequence and/or quantify cDNA in order to obtain information about a sample's RNA content. Publications describing RNA-Seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer” Nature 458 (7234): 97-101 (January 2009).

(iv) Samples

The samples described herein may be taken, for example, from an individual who is suspected of having, or is diagnosed as having a cancer (e.g., lung cancer (e.g., NSCLC), bladder cancer (e.g., UC), kidney cancer (e.g., RCC), or breast cancer (e.g., TNBC)), and hence is likely in need of treatment, or from a healthy individual who is not suspected of having a cancer or who does not have cancer but has a family history of a cancer. For assessment of gene expression, samples, such as those containing cells, or proteins or nucleic acids produced by these cells, may be used in the methods of the present invention. The expression level of a gene can be determined by assessing the amount (e.g., the absolute amount or concentration) of the markers in a sample (e.g., a tissue sample, e.g., a tumor tissue sample, such as a biopsy). In addition, the level of a gene can be assessed in bodily fluids or excretions containing detectable levels of genes. Bodily fluids or secretions useful as samples in the present invention include, e.g., blood, urine, saliva, stool, pleural fluid, lymphatic fluid, sputum, ascites, prostatic fluid, cerebrospinal fluid (CSF), or any other bodily secretion or derivative thereof. The word “blood” is meant to include whole blood, plasma, serum, or any derivative of blood. Assessment of a gene in such bodily fluids or excretions can sometimes be preferred in circumstances where an invasive sampling method is inappropriate or inconvenient. In other embodiments, a tumor tissue sample is preferred.

The sample may be frozen, fresh, fixed (e.g., formalin fixed), centrifuged, and/or embedded (e.g., paraffin embedded), etc. The cell sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation, such as formalin fixation.

In some embodiments, the sample is a clinical sample. In another instance, the sample is used in a diagnostic assay, such as a diagnostic assay or diagnostic method of the invention. In some instances, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For example, samples of lung cancer lesions may be obtained by resection, bronchoscopy, fine needle aspiration, bronchial brushings, or from sputum, pleural fluid, or blood. Genes or gene products can be detected from cancer or tumor tissue or from other body samples such as urine, sputum, serum, or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In some instances, the sample from the individual is a tissue sample, a whole blood sample, a plasma sample, a serum sample, or a combination thereof. In some instances, the sample is a tissue sample. In some instances, the sample is a tumor tissue sample. In some instances, the sample is obtained prior to treatment with a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). In some instances, the tissue sample is formalin-fixed and paraffin-embedded (FFPE) sample, an archival sample, a fresh sample, or a frozen sample.

In some instances, the sample from the individual is a tissue sample. In some instances, the tissue sample is a tumor tissue sample (e.g., biopsy tissue). In some instances, the tumor tissue sample includes tumor cells, tumor-infiltrating immune cells, stromal cells, or a combination thereof. In some instances, the tissue sample is lung tissue. In some instances, the tissue sample is bladder tissue. In some instances, the tissue sample is renal tissue. In some instances, the tissue sample is breast tissue. In some instances, the tissue sample is skin tissue. In some instances, the tissue sample is pancreatic tissue. In some instances, the tissue sample is gastric tissue. In some instances, the tissue sample is esophageal tissue. In some instances, the tissue sample is mesothelial tissue. In some instances, the tissue sample is thyroid tissue. In some instances, the tissue sample is colorectal tissue. In some instances, the tissue sample is head or neck tissue. In some instances, the tissue sample is osteosarcoma tissue. In some instances, the tissue sample is prostate tissue. In some instances, the tissue sample is ovarian tissue, HCC (liver), blood cells, lymph nodes, or bone/bone marrow.

In some instances, the tumor tissue sample is extracted from a malignant cancerous tumor (i.e., cancer). In some instances, the cancer is a solid tumor, or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors. In some instances, the cancer NSCLC. In some instances, the cancer is second-line or third-line locally advanced or metastatic non-small cell lung cancer. In some instances, the cancer is adenocarcinoma. In some instances, the cancer is squamous cell carcinoma.

(v) RNA Extraction

Prior to detecting the level of a nucleic acid, mRNA may be isolated from a target sample. In some instances, the mRNA is total RNA isolated from tumors or tumor cell lines or, alternatively, normal tissues or cell lines. RNA can be isolated from a variety of tumor tissues, including breast, lung, colon, prostate, brain, liver, kidney, pancreas, stomach, gall bladder, spleen, thymus, testis, ovary, uterus, etc., the corresponding normal tissues, or tumor cell lines. If the source of mRNA is a primary tumor, mRNA can be extracted, for example, from frozen or archived paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., Bio Techniques 18:42044 (1995). In particular, RNA isolation can be performed using a purification kit, buffer set, and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated, for example, by using RNA Stat-60 (TelTest). RNA prepared from tumor tissue samples can also be isolated, for example, by cesium chloride density gradient centrifugation.

(vi) Immune-Score Expression Level

The immune-score expression level may reflect the expression levels of one or more genes described herein (e.g., one or more genes set forth in any one of Tables 1-17). In certain instances, to determine an immune-score expression level, the detected expression level of each gene is normalized using any one of the standard normalization methods known in the art. One of skill in the art will appreciate that the normalization method used may depend on the gene expression methodology used (e.g., one or more housekeeping genes may be used for normalization in the context of an RT-qPCR methodology, but a whole genome or substantially whole genome may be used as a normalization baseline in the context of an RNA-seq methodology). For example, the detected expression level of each gene assayed can be normalized for both differences in the amount of the gene(s) assayed, variability in the quality of the samples used, and/or variability between assay runs.

In some instances, normalization may be accomplished by detecting expression of certain one or more normalizing gene(s), including reference gene(s) (e.g., a housekeeping). For example, in some instances, the nucleic acid expression levels detected using the methods described herein (e.g., for one or more genes set forth in any one of Tables 1-17) may be normalized to the expression level of one or more reference genes (e.g., one, two, three, four, five, six, seven, eight, nine, or more reference genes, e.g., a housekeeping gene). Alternatively, normalization can be based on the average signal or median signal of all of the assayed genes. On a gene-by-gene basis, a measured normalized amount of a subject tumor mRNA can be compared to the amount found in a reference immune-score expression level. The presence and/or expression level/amount measured in a particular subject sample to be analyzed will fall at some percentile within this range, which can be determined by methods well known in the art.

In some instances, to determine an immune-score expression level, the detected expression level of each assayed gene is not normalized.

The immune-score expression level may reflect the aggregate or composite expression level of a single gene or a plurality of genes described herein (e.g., for one or more genes set forth in any one of Tables 1-17). Any statistical approaches known in the art may be used to determine the immune-score expression level.

For example, the immune-score expression level may reflect the median expression level, mean expression level, or a numerical value that reflects the aggregated Z-score expression level for the combination of genes assayed (e.g., for one or more genes set forth in any one of Tables 1-17).

In some instances, the immune-score expression level reflects the median normalized expression level, mean normalized expression level, or a numerical value that reflects the aggregated Z-score normalized expression level for the combinations of genes assayed (e.g., for one or more genes set forth in any one of Tables 1-17).

For example, the immune-score expression level may reflect an average (mean) of the expression levels of each gene in a combination of two or more genes set forth in any one of Tables 1-17). In some instances, the immune-score expression level reflects an average (mean) of the normalized expression levels of each gene in a combination of two or more genes set forth in any one of Tables 1-17 (e.g., normalized to a reference gene, e.g., a housekeeping gene). In some instances, the immune-score expression level reflects a median of the expression levels of each gene in a combination of two or more genes set forth in any one of Tables 1-17. In some instances, the immune-score expression level reflects a median of the normalized expression levels of each gene in a combination of two or more genes set forth in any one of Tables 1-17 (e.g., normalized to a reference gene, e.g., a housekeeping gene). In some instances, the immune-score expression level reflects the Z-score for each gene in a combination of two or more genes set forth in any one of Tables 1-17.

(vii) Reference Immune-Score Expression Level

The reference immune-score expression level may be a value derived from analysis of any of the reference populations described herein. In some instances, the reference immune-score expression level may be a “cut-off” value selected based on a reference immune-score expression level that divides a reference population into subsets, e.g., subsets that exhibit significant differences (e.g., statistically significant differences) in treatment response to a PD-L1 axis binding antagonist therapy and a non-PD-L1 axis binding antagonist therapy. In such instances, relative treatment response may be evaluated based on progression-free survival (PFS) or overall survival (OS), expressed, for example, as a hazard ratio (HR) (e.g., progression-free survival HR (PFS HR) or overall survival HR (OS HR)).

In certain instances, the reference immune-score expression level is an immune-score expression level of one or more genes set forth in any one of Tables 1-17 in a reference population that significantly (e.g., statistically significantly) separates a first subset of individuals who have been treated with a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above the reference immune-score expression level (i.e., above the cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is an immune-score expression level of one or more genes set forth in any one of Tables 1-17 in a reference population that substantially optimally separates a first subset of individuals who have been treated with a PD-L1 axis binding antagonist (e.g., a PD-L1 binding antagonist (e.g., anti-PD-L1 antibody, e.g., atezolizumab) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the cut-off), wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy.

In certain particular instances, the reference immune-score expression level is an immune-score expression level of one or more genes set forth in any one of Tables 1-17 in a reference population that optimally separates a first subset of individuals who have been treated with a PD-L1 axis binding antagonist (e.g., a PD-L1 binding antagonist (e.g., anti-PD-L1 antibody, e.g., atezolizumab) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the cut-off), wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy.

In certain instances, the reference immune-score expression level is an immune-score expression level of one or more genes set forth in any one of Tables 1-17 in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy below the reference immune-score expression level (i.e., below the cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is an immune-score expression level of one or more genes set forth in any one of Tables 1-17 in a reference population that substantially optimally separates a first subset of individuals who have been treated with a PD-L1 axis binding antagonist (e.g., a PD-L1 binding antagonist (e.g., anti-PD-L1 antibody, e.g., atezolizumab) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In certain particular instances, the reference immune-score expression level is an immune-score expression level of one or more genes set forth in any one of Tables 1-17 in a reference population that optimally separates a first subset of individuals who have been treated with a PD-L1 axis binding antagonist (e.g., a PD-L1 binding antagonist (e.g., anti-PD-L1 antibody, e.g., atezolizumab) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

The reference immune-score expression level may be determined from any number of individuals in a reference population and/or any number of reference samples (e.g., reference cell, reference tissue, control sample, control cell, or control tissue). The reference sample may be a single sample or a combination of multiple samples. A reference immune-score expression level based on a reference sample may be based on any number of reference samples (e.g., 2 or more, 5 or more, 10 or more, 50 or more, 100 or more, 500 or more, or 1000 or more reference samples). In certain instances, a reference sample includes pooled mRNA samples derived from samples obtained from multiple individuals. Further, a reference immune-score expression level based on a reference population, or samples therefrom, may be based on any number of individuals in the reference population (e.g., 2 or more, 5 or more, 10 or more, 50 or more, 100 or more, 500 or more, or 1000 or more individuals in a reference population). Any statistical methods known in the art may be used to determine a reference immune-score expression level from measurements based on multiple samples or multiple individuals in a reference population. See e.g., Sokal R. R. and Rholf, F. J. (1995) “Biometry: the principles and practice of statistics in biological research,” W.H. Freeman and Co. New York, N.Y.

(viii) Reference Populations

The reference immune-score expression level may reflect the expression level(s) of one or more genes described herein (e.g., one or more genes set forth in any one of Tables 1-17) in one or more reference populations (or reference samples), or as a pre-assigned reference value.

In some instances, the reference immune-score expression level is an immune-score expression level for one or more genes set forth in any one of Tables 1-17 in a reference population.

In some instances, the reference population is a population of individuals having a cancer. In some instances, the reference population is a population of individuals having lung cancer (e.g., NSCLC). In some instances, the reference population is a population of individuals having kidney cancer (e.g., RCC). In some instances, the reference population is a population of individuals having bladder cancer (e.g., UC). In some instances, the reference population is a population of individuals having breast cancer (e.g., TNBC). In some instances, the reference population is a population of individuals who do not have a cancer.

Further, the reference population may include one or more subsets of individuals (e.g., one or more, two or more, three or more, four or more, five or more, six or more, seven or more, eight or more, nine or more, or ten or more subsets).

In some instances, the reference population is a population of individuals having the cancer, wherein the population of individuals includes a subset of individuals who have been treated with at least one dose (e.g., at least one, at least two, at least three, at least four, at least five, at least six, at least seven, at least eight, at least nine, at least ten, or more than ten doses) of a therapy including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). In some instances, the therapy including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) is a monotherapy. In other instances, the therapy including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) is a combination treatment that includes, in addition to the PD-L1 axis binding antagonist, at least one additional therapeutic agent (e.g., an anti-cancer therapy (e.g., an anti-neoplastic agent, a chemotherapeutic agent, a growth inhibitory agent, a cytotoxic agent, a radiotherapy, or combinations thereof)).

In some instances, the reference population is a population of individuals having the cancer, wherein the population of individuals includes a subset of individuals who have been treated with a non-PD-L1 axis binding antagonist therapy (e.g., an anti-cancer therapy, (e.g., an anti-neoplastic agent, a chemotherapeutic agent, a growth inhibitory agent, a cytotoxic agent, a radiotherapy, or combinations thereof)) that does not include a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In some instances, the reference population includes a combination of individuals from different subsets. For example, in some instances, the reference population may be a population of individuals having the cancer, the population of individuals consisting of (i) a first subset of individuals who have been treated with a PD-L1 axis binding antagonist therapy (e.g., a PD-L1 binding antagonist therapy) that includes a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) and (ii) a second subset of individuals who have been treated with a non-PD-L1 axis binding antagonist therapy (e.g., a non-PD-L1 binding antagonist therapy) that does not include a PD-L1 axis binding antagonist (e.g., an anti-cancer therapy (e.g., an anti-neoplastic agent, a chemotherapeutic agent, a growth inhibitory agent, a cytotoxic agent, a radiotherapy, or combinations thereof). The PD-L1 axis binding antagonist therapy (e.g., a PD-L1 binding antagonist therapy) in the first subset may have been administered as either a monotherapy or a combination therapy.

### III. METHODS OF TREATMENT AND THERAPEUTIC USES

Also provided herein are methods, medicaments, and uses thereof, for treating an individual having a cancer (e.g., lung cancer (e.g., NSCLC), bladder cancer (e.g., UC), kidney cancer (e.g., RCC), or breast cancer (e.g., TNBC)), the methods including administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) based on the presence and/or expression level of one or more of any of the biomarkers disclosed herein that have been determined in a sample from the individual.

Any of the methods provided herein may include determining the presence and/or expression level of any biomarker disclosed herein. In some instances, any of the methods provided herein may include administering a PD-L1 binding antagonist to an individual based on a prior determination of the presence and/or expression level of any biomarker disclosed herein. In other instances, any of the methods provided herein may include administering a PD-L1 binding antagonist to an individual prior to determination of the presence and/or expression level of any biomarker disclosed herein.

Any of the methods, medicaments, and uses may include or involve any of the PD-L1 axis binding antagonists disclosed herein (e.g., in Section IV).

For example, the biomarker may include the presence and/or expression level of a biomarker set forth in any one of Tables 1-17 in a sample obtained from an individual; the presence of a TLS in a sample obtained from an individual; the number of B cells in a sample obtained from an individual; the presence of clonally expanded B cells in a sample from the individual; and/or a combination thereof. Any suitable sample may be used, e.g., any sample type disclosed herein, including a tumor sample.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, 14, 15, 16, 17, 18, 19, or 20) genes set forth in Table 1 in a sample from the individual, wherein an immune-score expression level of the one or more genes in the sample is determined to be above a reference immune-score expression level of the two or more genes; and (b) administering an effective amount of a PD-L1 axis binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, 14, 15, 16, 17, 18, 19, or 20) genes set forth in Table 1 in a sample from the individual that is above a reference immune-score expression level of the one or more genes, the method comprising administering to the individual an effective amount of a PD-L1 axis binding antagonist (e.g., an anti-PD-L1 antibody, e.g., atezolizumab) or a PD-1 binding antagonist (e.g., an anti-PD-1 antibody)).

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in progression-free survival (PFS), or an increase in best confirmed overall response (BOOR). In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is FFPE sample, an archival sample, a fresh sample, or a frozen sample. In some instances, the tumor tissue sample is an FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is a non-small cell lung cancer (NSCLC). In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### A. B Cell Signatures

(i) Gene Signatures Associated with B Cells

In some aspects, the methods provided herein may involve administering a PD-L1 axis binding antagonist to an individual based on an expression level of one or more genes in a B cell signature. Any suitable B cell signature may be used. For example, the B cell signature may include one or more (e.g., 1, 2, 3, 4, 5, 6, 7, 8, 9, 10, or 11) genes set forth in Table 2.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes.

In some instances, the method comprises determining the expression level of one of genes CD79A, CD19, BANK1, JCHAIN, SLAMF7, BTK, TNFRSF17, IGJ, IGLL5, RBPJ, and MZB1.

In some instances, the method comprises determining the expression level of CD79A.

In some instances, the method comprises determining the expression level of CD19.

In some instances, the method comprises determining the expression level of BANK1.

In some instances, the method comprises determining the expression level of JCHAIN.

In some instances, the method comprises determining the expression level of SLAMF7.

In some instances, the method comprises determining the expression level of BTK.

In some instances, the method comprises determining the expression level of TNFRSF17.

In some instances, the method comprises determining the expression level of IGJ.

In some instances, the method comprises determining the expression level of IGLL5.

In some instances, the method comprises determining the expression level of RBPJ.

In some instances, the method comprises determining the expression level of MZB1.

In some instances, the expression level of one of genes CD79A, CD19, BANK1, JCHAIN, SLAMF7, BTK, TNFRSF17, IGJ, IGLL5, RBPJ, and MZB1 has been determined.

In some instances, the expression level of CD79A has been determined.

In some instances, the expression level of CD19 has been determined.

In some instances, the expression level of BANK1 has been determined.

In some instances, the expression level of JCHAIN has been determined.

In some instances, the expression level of SLAMF7 has been determined.

In some instances, the expression level of BTK has been determined.

In some instances, the expression level of TNFRSF17 has been determined.

In some instances, the expression level of IGJ has been determined.

In some instances, the expression level of IGLL5 has been determined.

In some instances, the expression level of RBPJ has been determined.

In some instances, the expression level of MZB1 has been determined.

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In yet another example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist to the individual.

In a further example still, provided herein is the use a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In some instances, the genes comprise two or more of CD79A, CD19, BANK1, JCHAIN, SLAMF7, BTK, TNFRSF17, IGJ, IGLL5, RBPJ, and MZB1.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of two or more (e.g., 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes.

Any combination of B cell signature genes may be determined. For example, the combination may include two genes selected from CD79A, CD19, BANK1, JCHAIN, SLAMF7, BTK, TNFRSF17, IGJ, IGLL5, RBPJ, and MZB1, e.g., any one of the combinations set forth in Table 3. In another example, the combination may include three genes selected from CD79A, CD19, BANK1, JCHAIN, SLAMF7, BTK, TNFRSF17, IGJ, IGLL5, RBPJ, and MZB1, e.g., any one of the combinations set forth in Table 4. In another example, the combination may include four genes selected from CD79A, CD19, BANK1, JCHAIN, SLAMF7, BTK, TNFRSF17, IGJ, IGLL5, RBPJ, and MZB1, e.g., any one of the combinations set forth in Table 5. In another example, the combination may include five genes selected from CD79A, CD19, BANK1, JCHAIN, SLAMF7, BTK, TNFRSF17, IGJ, IGLL5, RBPJ, and MZB1, e.g., any one of the combinations set forth in Table 6. In another example, the combination may include six genes selected from CD79A, CD19, BANK1, JCHAIN, SLAMF7, BTK, TNFRSF17, IGJ, IGLL5, RBPJ, and MZB1, e.g., any one of the combinations set forth in Table 7. In another example, the combination may include seven genes selected from CD79A, CD19, BANK1, JCHAIN, SLAMF7, BTK, TNFRSF17, IGJ, IGLL5, RBPJ, and MZB1, e.g., any one of the combinations set forth in Table 8.

In some instances, the genes comprise CD79A, CD19, BANK1, JCHAIN, SLAMF7, BTK, TNFRSF17, IGJ, IGLL5, RBPJ, and MZB1.

In some instances, the expression level is a nucleic acid expression level. For example, in some instances, the nucleic acid expression level is an mRNA expression level. The mRNA expression level may be determined using any suitable approach, e.g., any approach disclosed herein. In some instances, the mRNA expression level is determined by RNA-seq, RT-qPCR, qPCR, multiplex qPCR or RT-qPCR, microarray analysis, SAGE, MassARRAY technique, FISH, or a combination thereof. In some instances, the mRNA expression level is detected using RNA-seq.

In other instances, the expression level is a protein expression level. The protein expression level may be determined using any suitable approach, e.g., any approach disclosed herein. In some instances, the protein expression level is determined by IHC, immunofluorescence, mass spectrometry, flow cytometry, and Western blot, or a combination thereof.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is a FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

(ii) Gene Signatures Associated with Plasma B Cells

In some aspects, the methods provided herein may involve administering a PD-L1 axis binding antagonist to an individual based on an expression level of one or more genes in a plasma B cell signature. Any suitable plasma B cell signature may be used. For example, the plasma B cell signature may include one or more (e.g., 1, 2, 3, 4, 5, 6, 7, 8, 9, 10, 11, 12, 13, or 14) genes set forth in Table 9.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes.

In some instances, the method comprises determining the expression level of one of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

In some instances, the method comprises determining the expression level of MZB1.

In some instances, the method comprises determining the expression level of DERL3.

In some instances, the method comprises determining the expression level of JSRP1.

In some instances, the method comprises determining the expression level of TNFRSF17.

In some instances, the method comprises determining the expression level of SLAMF7.

In some instances, the method comprises determining the expression level of IGHG2.

In some instances, the method comprises determining the expression level of IGHGP.

In some instances, the method comprises determining the expression level of IGLV3-1.

In some instances, the method comprises determining the expression level of IGLV6-57.

In some instances, the method comprises determining the expression level of IGHA2.

In some instances, the method comprises determining the expression level of IGKV4-1.

In some instances, the method comprises determining the expression level of IGKV1-12.

In some instances, the method comprises determining the expression level of IGLC7.

In some instances, the method comprises determining the expression level of IGLL5.

In some instances, the expression level of one of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 has been determined.

In some instances, the expression level of MZB1 has been determined.

In some instances, the expression level of DERL3 has been determined.

In some instances, the expression level of JSRP1 has been determined.

In some instances, the expression level of TNFRSF17 has been determined.

In some instances, the expression level of SLAMF7 has been determined.

In some instances, the expression level of IGHG2 has been determined.

In some instances, the expression level of IGHGP has been determined.

In some instances, the expression level of IGLV3-1 has been determined.

In some instances, the expression level of IGLV6-57 has been determined.

In some instances, the expression level of IGHA2 has been determined.

In some instances, the expression level of IGKV4-1 has been determined.

In some instances, the expression level of IGKV1-12 has been determined.

In some instances, the expression level of IGLC7 has been determined.

In some instances, the expression level of IGLL5 has been determined.

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In yet another example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and (b) administering an effective amount of a PD-L1 axis binding antagonist to the individual.

In a further example still, provided herein is the use a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

### B. TLS

In some aspects, the methods provided herein may involve administering a PD-L1 axis binding antagonist to an individual based on the presence of a TLS in a sample (e.g., a tumor sample) from an individual having a cancer (e.g., lung cancer (e.g., NSCLC), bladder cancer (e.g., UC), kidney cancer (e.g., RCC), or breast cancer (e.g., TNBC)).

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the presence of a TLS in a sample (e.g., a tumor sample) from the individual; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have the presence of a TLS in a sample (e.g., a tumor sample) from the individual, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the presence of a TLS in a sample (e.g., a tumor sample) from the individual; and (b) administering an effective amount of a PD-L1 axis binding antagonist to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have the presence of a TLS in a sample (e.g., a tumor sample) from the individual.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the presence of a TLS in a sample (e.g., a tumor sample) from the individual; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have the presence of a TLS in a sample (e.g., a tumor sample) from the individual.

Any suitable approach can be used to determine the presence of a TLS in a sample. For example, in some instances, the presence of a TLS is determined by histological staining, IHC, immunofluorescence, or gene expression analysis.

Any suitable histological staining approach can be used. For example, in some instances, the histological staining comprises H&E staining.

Any suitable IHC or immunofluorescence approach can be used. In some instances, the IHC or immunofluorescence comprises detecting CD62L, L-selectin, CD40, or CD8, e.g., using an antibody (e.g., an anti-CD62L antibody, an anti-L-selectin antibody, an anti-CD40 antibody, and/or an anti-CD8 antibody). In some instances, CD62L or L-selectin is detected using a MECA-79 antibody.

In some instances, the gene expression analysis comprises determining the expression level of a TLS gene signature in the sample. For example, the gene expression analysis may involve determining the expression level of any TLS signature disclosed herein (see, e.g., Section II, Subsection C below). In some instances, the TLS gene signature comprises one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13. In some instances, the genes comprise two or more of CCL2, CCL3, CCL4, CCL5, CCL8, CCL18, CCL19, CCL21, CXCL9, CXCL10, CXCL11, and CXCL13.

In some instances, the sample may include one TLS. In other instances, the sample may include more than one TLS, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, or more TLS.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is an FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### C. TLS Signatures

In some aspects, the methods provided herein may involve administering a PD-L1 axis binding antagonist to an individual based on an expression level of one or more genes in a TLS signature. Any suitable TLS signature may be used. For example, the TLS signature may include one or more (e.g., 1, 2, 3, 4, 5, 6, 7, 8, 9, 10, 11, or 12) genes set forth in Table 16.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the one or more genes in the sample is determined to be above a reference immune-score expression level of the one or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the one or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the one or more genes in the sample is determined to be above a reference immune-score expression level of the one or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the one or more genes.

In a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the one or more genes in the sample is determined to be above a reference immune-score expression level of the one or more genes; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the one or more genes.

Any suitable immune-score reference expression level may be used. In some instances, the immune-score reference expression level is an immune-score expression level of the one or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the one or more genes in the reference population. In some instances, the median expression level is the median of a mean Z score of the expression level of each of the one or more genes in the reference population.

In some instances, the genes comprise two or more of CCL2, CCL3, CCL4, CCL5, CCL8, CCL18, CCL19, CCL21, CXCL9, CXCL10, CXCL11, and CXCL13.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes.

In a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of two or more (e.g., 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes.

In some instances, the reference immune-score expression level is an immune-score expression level of the two or more genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of the two or more genes in the reference population.

In some instances, the genes comprise three or more of CCL2, CCL3, CCL4, CCL5, CCL8, CCL18, CCL19, CCL21, CXCL9, CXCL10, CXCL11, and CXCL13. In some instances, the genes comprise four or more of CCL2, CCL3, CCL4, CCL5, CCL8, CCL18, CCL19, CCL21, CXCL9, CXCL10, CXCL11, and CXCL13. In some instances, the genes comprise five or more of CCL2, CCL3, CCL4, CCL5, CCL8, CCL18, CCL19, CCL21, CXCL9, CXCL10, CXCL11, and CXCL13. In some instances, the genes comprise six or more of CCL2, CCL3, CCL4, CCL5, CCL8, CCL18, CCL19, CCL21, CXCL9, CXCL10, CXCL11, and CXCL13. In some instances, the genes comprise seven or more of CCL2, CCL3, CCL4, CCL5, CCL8, CCL18, CCL19, CCL21, CXCL9, CXCL10, CXCL11, and CXCL13. In some instances, the genes comprise eight or more of CCL2, CCL3, CCL4, CCL5, CCL8, CCL18, CCL19, CCL21, CXCL9, CXCL10, CXCL11, and CXCL13. In some instances, the genes comprise nine or more of CCL2, CCL3, CCL4, CCL5, CCL8, CCL18, CCL19, CCL21, CXCL9, CXCL10, CXCL11, and CXCL13. In some instances, the genes comprise ten or more of CCL2, CCL3, CCL4, CCL5, CCL8, CCL18, CCL19, CCL21, CXCL9, CXCL10, CXCL11, and CXCL13. In some instances, the genes comprise eleven or more of CCL2, CCL3, CCL4, CCL5, CCL8, CCL18, CCL19, CCL21, CXCL9, CXCL10, CXCL11, and CXCL13.

Any combination of TLS signature genes may be determined, e.g., any combination of two genes selected from CCL2, CCL3, CCL4, CCL5, CCL8, CCL18, CCL19, CCL21, CXCL9, CXCL10, CXCL11, and CXCL13; any combination of three genes selected from CCL2, CCL3, CCL4, CCL5, CCL8, CCL18, CCL19, CCL21, CXCL9, CXCL10, CXCL11, and CXCL13; any combination of four genes selected from CCL2, CCL3, CCL4, CCL5, CCL8, CCL18, CCL19, CCL21, CXCL9, CXCL10, CXCL11, and CXCL13; any combination of five genes selected from CCL2, CCL3, CCL4, CCL5, CCL8, CCL18, CCL19, CCL21, CXCL9, CXCL10, CXCL11, and CXCL13; any combination of six genes selected from CCL2, CCL3, CCL4, CCL5, CCL8, CCL18, CCL19, CCL21, CXCL9, CXCL10, CXCL11, and CXCL13; any combination of seven genes selected from CCL2, CCL3, CCL4, CCL5, CCL8, CCL18, CCL19, CCL21, CXCL9, CXCL10, CXCL11, and CXCL13; any combination of eight genes selected from CCL2, CCL3, CCL4, CCL5, CCL8, CCL18, CCL19, CCL21, CXCL9, CXCL10, CXCL11, and CXCL13; any combination of nine genes selected from CCL2, CCL3, CCL4, CCL5, CCL8, CCL18, CCL19, CCL21, CXCL9, CXCL10, CXCL11, and CXCL13; any combination of ten genes selected from CCL2, CCL3, CCL4, CCL5, CCL8, CCL18, CCL19, CCL21, CXCL9, CXCL10, CXCL11, and CXCL13; any combination of eleven genes selected from CCL2, CCL3, CCL4, CCL5, CCL8, CCL18, CCL19, CCL21, CXCL9, CXCL10, CXCL11, and CXCL13; or any combination of twelve genes selected from CCL2, CCL3, CCL4, CCL5, CCL8, CCL18, CCL19, CCL21, CXCL9, CXCL10, CXCL11, and CXCL13.

In some instances, the genes comprise CCL2, CCL3, CCL4, CCL5, CCL8, CCL18, CCL19, CCL21, CXCL9, CXCL10, CXCL11, and CXCL13.

In some instances, the expression level is a nucleic acid expression level. For example, in some instances, the nucleic acid expression level is an mRNA expression level. The mRNA expression level may be determined using any suitable approach, e.g., any approach disclosed herein. In some instances, the mRNA expression level is determined by RNA-seq, RT-qPCR, qPCR, multiplex qPCR or RT-qPCR, microarray analysis, SAGE, MassARRAY technique, FISH, or a combination thereof. In some instances, the mRNA expression level is detected using RNA-seq.

In other instances, the expression level is a protein expression level. The protein expression level may be determined using any suitable approach, e.g., any approach disclosed herein. In some instances, the protein expression level is determined by IHC, immunofluorescence, mass spectrometry, flow cytometry, and Western blot, or a combination thereof.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is a, FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### D. B Cell Number and Clonally Expanded B Cells

In some aspects, the methods provided herein may involve administering a PD-L1 axis binding antagonist to an individual based on the presence and/or number of B cells in a sample from an individual. In some aspects, the methods provided herein may involve determining the presence and/or number of clonally expanded B cells in a sample from an individual.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the number of B cells in a tumor sample from the individual; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have a number of B cells in a tumor sample from the individual that is above a reference number of B cells, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the number of B cells in a tumor sample from the individual; and (b) administering an effective amount of a PD-L1 axis binding antagonist to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have a number of B cells in a tumor sample from the individual that is above a reference number of B cells.

In a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the number of B cells in a tumor sample from the individual; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have a number of B cells in a tumor sample from the individual that is above a reference number of B cells.

The presence and/or number of any suitable type of B cell may be determined. For example, in some instances, the B cells comprise CD79+ B cells, IgG+ B cells, and/or plasma cells.

In some embodiments, the presence and/or number of clonally expanded B cells may be determined.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining that the individual has clonally expanded B cells in a tumor sample from the individual; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have clonally expanded B cells in a tumor sample from the individual, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining that the individual has clonally expanded B cells in a tumor sample from the individual; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for use in treating cancer (e.g., lung cancer (e.g., NSCLC), bladder cancer (e.g., UC), kidney cancer (e.g., RCC), or breast cancer (e.g., TNBC)) in an individual that has been determined to have clonally expanded B cells in a tumor sample from the individual.

In a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining that the individual has clonally expanded B cells in a tumor sample from the individual; and (b) administering an effective amount of a PD-L1 axis binding antagonist to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament treating cancer (e.g., lung cancer (e.g., NSCLC), bladder cancer (e.g., UC), kidney cancer (e.g., RCC), or breast cancer (e.g., TNBC)) in an individual that has been determined to have clonally expanded B cells in a tumor sample from the individual.

The clonally expanded B cells may be any type of B cell. For example, in some instances, the clonally expanded B cells are clonally expanded plasma cells.

Clonally expanded B cells may be detected by any suitable approach. For example, in some instances, clonally expanded B cells are detected by measuring the diversity of the B cell receptor (BCR) gene repertoire in the tumor sample. In some instances, a Shannon Diversity Index (SDI) of the BCR gene repertoire in the tumor sample from the individual that is below a reference SDI identifies the individual as one who may benefit from a treatment comprising a PD-L1 axis binding antagonist.

Any suitable sample may be used. In some instances, the sample is a tissue sample, a cell sample, a whole blood sample, a plasma sample, a serum sample, or a combination thereof. In some instances, the tissue sample is a tumor tissue sample. In some instances, the tumor sample is a tumor tissue sample. In some instances, the tumor tissue sample comprises tumor cells, tumor-infiltrating immune cells, stromal cells, NAT cells, or a combination thereof. In some instances, the tumor tissue sample is an FFPE sample, an archival sample, a fresh sample, or a frozen sample. In some instances, the tumor tissue sample is an FFPE sample.

The cancer may be any suitable cancer type. In some instances, the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy. In some instances, the cancer is a lung cancer, a kidney cancer, a bladder cancer, or a breast cancer. In some instances, the lung cancer is an NSCLC. In some instances, the NSCLC is non-squamous NSCLC. In some instances, the NSCLC is squamous NSCLC.

In some instances, the benefit comprises an extension in the individual's OS, an extension in the individual's PFS, and/or an improvement in the individual's BOOR, as compared to treatment without the PD-L1 axis binding antagonist. In some instances, the benefit comprises an extension in the individual's OS as compared to treatment without the PD-L1 axis binding antagonist.

### E. T Effector Signatures

In some aspects, the methods provided herein may involve administering a PD-L1 axis binding antagonist to an individual based on the presence and/or expression level of one or more T effector signature genes. Any suitable T effector signature may be used. For example, the T effector signature may include one or more (e.g., 1, 2, 3, 4, 5, 6, 7, or 8) genes set forth in Table 17. In some instances, CD274 is further detected.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual, wherein an immune-score expression level of (i) the one or more genes set forth in Table 1 and (ii) the one or more of genes CD8A, EOMES, GZMA, TBX21, IFNG, GZMB, CXCL9, and CXCL10 in the sample is determined to be above a reference immune-score expression level of (i) the one or more genes set forth in Table 1 and (ii) the one or more of genes CD8A, EOMES, GZMA, TBX21, IFNG, GZMB, CXCL9, and CXCL10; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual, wherein an immune-score expression level of (i) the one or more genes set forth in Table 1 and (ii) the one or more of genes CD8A, EOMES, GZMA, TBX21, IFNG, GZMB, CXCL9, and CXCL10 in the sample is determined to be above a reference immune-score expression level of (i) the one or more genes set forth in Table 1 and (ii) the one or more of genes CD8A, EOMES, GZMA, TBX21, IFNG, GZMB, CXCL9, and CXCL10; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual, wherein an immune-score expression level of (i) the one or more genes set forth in Table 1 and (ii) the one or more of genes CD8A, EOMES, GZMA, TBX21, IFNG, GZMB, CXCL9, and CXCL10 in the sample is determined to be above a reference immune-score expression level of (i) the one or more genes set forth in Table 1 and (ii) the one or more of genes CD8A, EOMES, GZMA, TBX21, IFNG, GZMB, CXCL9, and CXCL10; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual that is above a reference immune-score expression level of (i) the one or more genes set forth in Table 1 and (ii) the one or more of genes CD8A, EOMES, GZMA, TBX21, IFNG, GZMB, CXCL9, and CXCL10, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual that is above a reference immune-score expression level of (i) the one or more genes set forth in Table 1 and (ii) the one or more of genes CD8A, EOMES, GZMA, TBX21, IFNG, GZMB, CXCL9, and CXCL10.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, 11, 12, 13, 14, 15, 16, 17, 18, 19, or 20) of genes set forth in Table 1 and (ii) one or more (e.g., 1, 2, 3, 4, 5, 6, 7, or 8) of genes CD8A, EOMES, GZMA, TBX21, IFNG, GZMB, CXCL9, and CXCL10 in a sample from the individual that is above a reference immune-score expression level of (i) the one or more genes set forth in Table 1 and (ii) the one or more of genes CD8A, EOMES, GZMA, TBX21, IFNG, GZMB, CXCL9, and CXCL10.

In some instances, the reference immune-score expression level is an immune-score expression level of (i) the one or more genes set forth in Table 1 and (ii) the one or more of genes CD8A, EOMES, GZMA, TBX21, IFNG, GZMB, CXCL9, and CXCL10 in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of the (i) the one or more genes set forth in Table 1 and (ii) the one or more of genes CD8A, EOMES, GZMA, TBX21, IFNG, GZMB, CXCL9, and CXCL10 in the reference population.

For example, provided herein is a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more B cell signature genes and (ii) the one or more T effector signature genes in the sample is determined to be above a reference immune-score expression level of (i) the one or more B cell signature genes and (ii) the one or more T effector signature genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual that is above a reference immune-score expression level of (i) the one or more B cell signature genes and (ii) the one or more T effector signature genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more B cell signature genes and (ii) the one or more T effector signature genes in the sample is determined to be above a reference immune-score expression level of (i) the one or more B cell signature genes and (ii) the one or more T effector signature genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual that is above a reference immune-score expression level of (i) the one or more B cell signature genes and (ii) the one or more T effector signature genes.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more B cell signature genes and (ii) the one or more T effector signature genes in the sample is determined to be above a reference immune-score expression level of (i) the one or more B cell signature genes and (ii) the one or more T effector signature genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, or 11) of B cell signature genes CD79A, CD19, BANK1, JCHAIN, SLAMF7, BTK, TNFRSF17, IGJ, IGLL5, RBPJ, and MZB1 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual that is above a reference immune-score expression level of (i) the one or more B cell signature genes and (ii) the one or more T effector signature genes.

In some instances, the reference immune-score expression level is an immune-score expression level of (i) the one or more B cell signature genes and (ii) the one or more T effector signature genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of (i) the one or more B cell signature genes and (ii) the one or more T effector signature genes in the reference population.

In another example, provided herein is a method of treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, 11, or 12) of TLS signature genes CCL2, CCL3, CCL4, CCL5, CCL8, CCL18, CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual that is above a reference immune-score expression level of (i) the one or more TLS signature genes and (ii) the one or more T effector signature genes, the method comprising administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In yet another example, provided herein is a PD-L1 axis binding antagonist for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, 11, or 12) of TLS signature genes CCL2, CCL3, CCL4, CCL5, CCL8, CCL18, CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more TLS signature genes and (ii) the one or more T effector signature genes in the sample is determined to be above a reference immune-score expression level of (i) the one or more TLS signature genes and (ii) the one or more T effector signature genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example, provided herein is a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, 11, or 12) of TLS signature genes CCL2, CCL3, CCL4, CCL5, CCL8, CCL18, CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual that is above a reference immune-score expression level of (i) the one or more TLS signature genes and (ii) the one or more T effector signature genes.

In yet a further example, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for use in a method of treating an individual having a cancer (e.g., lung cancer (e.g., NSCLC), bladder cancer (e.g., UC), kidney cancer (e.g., RCC), or breast cancer (e.g., TNBC)), the method comprising: (a) determining the expression level of (i) one or more (e.g., 1, 2, 3, 4, 5, 6, 7, 8, 9, 10, 11, or 12) of TLS signature genes CCL2, CCL3, CCL4, CCL5, CCL8, CCL18, CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual, wherein an immune-score expression level of (i) the one or more TLS signature genes and (ii) the one or more T effector signature genes in the sample is determined to be above a reference immune-score expression level of (i) the one or more TLS signature genes and (ii) the one or more T effector signature genes; and (b) administering an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) to the individual.

In a further example still, provided herein is the use of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) in the manufacture of a medicament for treating cancer (e.g., lung cancer (e.g., NSCLC), bladder cancer (e.g., UC), kidney cancer (e.g., RCC), or breast cancer (e.g., TNBC)) in an individual that has been determined to have an immune-score expression level of (i) one or more (e.g., 1, 2, 3, 4, 5, 6, 7, 8, 9, 10, 11, or 12) of TLS signature genes CCL2, CCL3, CCL4, CCL5, CCL8, CCL18, CCL19, CCL21, CXCL9, CXCL10, CXCL11, and CXCL13 and (ii) one or more (e.g., 1, 2, 3, 4, 5, 6, 7, or 8) of T effector signature genes CD8A, EOMES, GZMA, TBX21, IFNG, GZMB, CXCL9, and CXCL10 in a sample from the individual that is above a reference immune-score expression level of (i) the one or more TLS signature genes and (ii) the one or more T effector signature genes.

In some instances, the reference immune-score expression level is an immune-score expression level of (i) the one or more TLS signature genes and (ii) the one or more T effector signature genes in a reference population. In some instances, the reference population is a population of individuals having the cancer. In some instances, the population of individuals comprises a first subset of individuals who have been treated with a PD-L1 axis binding antagonist and a second subset of individuals who have been treated with therapy that does not comprise a PD-L1 axis binding antagonist. In some instances, the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist. In some instances, the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof. In some instances, the therapy that does not comprise a PD-L1 axis binding antagonist comprises a chemotherapeutic agent. In some instances, the chemotherapeutic agent is docetaxel. In some instances, responsiveness to treatment comprises an extension in OS, an extension in PFS, or an increase in BOOR. In some instances, responsiveness to treatment comprises an extension in OS. In some instances, the reference immune-score expression level is a median of the expression level of each of the two or more genes in the reference population. In some instances, the median expression level is the median of a mean Z score of the expression level of each of (i) the one or more TLS signature genes and (ii) the one or more T effector signature genes in the reference population.

In some instances of any of the preceding methods, the presence and/or expression level of CD274 is further determined.

In any of the methods described herein, the method may include determining the expression level of CD79A, CD274, and one or more (e.g., 1, 2, 3, 4, 5, 6, 7, or 8) of T effector signature genes CD8A, EOMES, GZMA, TBX21, IFNG, GZMB, CXCL9, and CXCL10.

### F. Administration

The PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), or compositions thereof, and/or any additional therapeutic agent(s) utilized in the methods, uses, assays, and kits described herein can be formulated for administration or administered by any suitable approach, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in creams, or in lipid compositions. The compositions utilized in the methods described herein can also be administered systemically or locally. The method of administration can vary depending on various factors (e.g., the compound or composition being administered, and the severity of the condition, disease, or disorder being treated). In some instances, the PD-L1 axis binding antagonist (e.g., PD-L1 binding antagonist, e.g., an anti-PD-L1 antibody, e.g., atezolizumab) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The PD-L1 axis binding antagonist (e.g., PD-L1 binding antagonist, e.g., an anti-PD-L1 antibody, e.g., atezolizumab) and any additional therapeutic agent(s) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The PD-L1 axis binding antagonist (e.g., PD-L1 binding antagonist, e.g., an anti-PD-L1 antibody, e.g., atezolizumab) need not be, but is optionally formulated with and/or administered concurrently with, one or more agents currently used to prevent or treat the disorder in question. The effective amount of such other agents depends on the amount of the PD-L1 axis binding antagonist (e.g., PD-L1 binding antagonist, e.g., an anti-PD-L1 antibody, e.g., atezolizumab) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a cancer (e.g., a lung cancer (NSCLC), a bladder cancer, (UC), a kidney cancer (RCC), or a breast cancer (TNBC)), the appropriate dosage of a PD-L1 axis binding antagonist (e.g., PD-L1 binding antagonist, e.g., an anti-PD-L1 antibody, e.g., atezolizumab) described herein (when used alone or in combination with one or more other additional therapeutic agent(s)) will depend on the type of disease to be treated, the severity and course of the disease, whether the PD-L1 axis binding antagonist (e.g., PD-L1 binding antagonist, e.g., an anti-PD-L1 antibody, e.g., atezolizumab) is administered for preventive or therapeutic purposes, previous therapy, the patient's clinical history and response to the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), and the discretion of the attending physician. The PD-L1 axis binding antagonist (e.g., PD-L1 binding antagonist, e.g., an anti-PD-L1 antibody, e.g., atezolizumab) is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody))). An initial higher loading dose followed by one or more lower doses may be administered. However, other dosage regimens may be useful. The progress of this therapy is easily monitored by conventional techniques and assays.

In some instances, an effective amount of the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be between about 60 mg to about 5000 mg (e.g., between about 60 mg to about 4500 mg, between about 60 mg to about 4000 mg, between about 60 mg to about 3500 mg, between about 60 mg to about 3000 mg, between about 60 mg to about 2500 mg, between about 650 mg to about 2000 mg, between about 60 mg to about 1500 mg, between about 100 mg to about 1500 mg, between about 300 mg to about 1500 mg, between about 500 mg to about 1500 mg, between about 700 mg to about 1500 mg, between about 1000 mg to about 1500 mg, between about 1000 mg to about 1400 mg, between about 1100 mg to about 1300 mg, between about 1150 mg to about 1250 mg, between about 1175 mg to about 1225 mg, or between about 1190 mg to about 1210 mg, e.g., about 1200 mg±5 mg, about 1200±2.5 mg, about 1200±1.0 mg, about 1200±0.5 mg, about 1200±0.2 mg, or about 1200±0.1 mg). In some instances, the methods include administering to the individual the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) at about 1200 mg (e.g., a fixed dose of about 1200 mg or about 15 mg/kg).

In some instances, the amount of the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) administered to individual (e.g., human) may be in the range of about 0.01 to about 50 mg/kg of the individual's body weight (e.g., between about 0.01 to about 45 mg/kg, between about 0.01 mg/kg to about 40 mg/kg, between about 0.01 mg/kg to about 35 mg/kg, between about 0.01 mg/kg to about 30 mg/kg, between about 0.1 mg/kg to about 30 mg/kg, between about 1 mg/kg to about 30 mg/kg, between about 2 mg/kg to about 30 mg/kg, between about 5 mg/kg to about 30 mg/kg, between about 5 mg/kg to about 25 mg/kg, between about 5 mg/kg to about 20 mg/kg, between about 10 mg/kg to about mg/kg, or between about 12 mg/kg to about 18 mg/kg, e.g., about 15±2 mg/kg, about 15±1 mg/kg, about 15±0.5 mg/kg, about 15±0.2 mg/kg, or about 15±0.1 mg/kg). In some instances, the methods include administering to the individual the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) at about 15 mg/kg.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) is administered to the individual (e.g., a human) at 1200 mg intravenously every three weeks (q3w). The dose may be administered as a single dose or as multiple doses (e.g., 2, 3, 4, 5, 6, 7, or more than 7 doses), such as infusions.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered at a dose of about 840 mg every two weeks, e.g., intravenously.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered at a dose of about 1200 mg every three weeks, e.g., intravenously.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered at a dose of about 1680 mg every four weeks, e.g., intravenously.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered intravenously (e.g., by infusion) over 60 minutes. In some instances, for example, if the first dose is tolerated, subsequent doses may be administered intravenously (e.g., by infusion) over 30 minutes.

In some instances, atezolizumab may be administered at a dose of about 840 mg every two weeks intravenously.

In some instances, atezolizumab may be administered at a dose of about 1200 mg every three weeks intravenously.

In some instances, atezolizumab may be administered at a dose of about 1680 mg every four weeks e.g., intravenously.

In some instances, atezolizumab may be administered at a dose of 840 mg every two weeks intravenously.

In some instances, atezolizumab may be administered at a dose of 1200 mg every three weeks intravenously.

In some instances, atezolizumab may be administered at a dose of 1680 mg every four weeks e.g., intravenously.

Atezolizumab may be administered intravenously (e.g., by infusion) over 60 minutes. In some instances, for example, if the first dose is tolerated, subsequent doses of atezolizumab may be administered intravenously (e.g., by infusion) over 30 minutes.

The dose of the antibody administered in a combination treatment may be reduced as compared to a single treatment. The progress of this therapy is easily monitored by conventional techniques. In one instance, the PD-L1 axis binding antagonist (e.g., PD-L1 binding antagonist, e.g., anti-PD-L1 antibody, e.g., atezolizumab) is administered as a monotherapy to the individual to treat a cancer. In other instances, the PD-L1 axis binding antagonist (e.g., PD-L1 binding antagonist, e.g., anti-PD-L1 antibody, e.g., atezolizumab) is administered as a combination therapy, as described herein, to the individual to treat a cancer.

### G. Indications

The methods and medicaments described herein are useful for treating a patient having a cancer (e.g., lung cancer (e.g., NSCLC), bladder cancer (e.g., UC), kidney cancer (e.g., RCC), or breast cancer (e.g., TNBC)) by administering to the individual an effective amount of a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). For example, the cancer may be a lung cancer, a kidney cancer, a bladder cancer, a breast cancer, a colorectal cancer, an ovarian cancer, a pancreatic cancer, a gastric carcinoma, an esophageal cancer, mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy.

In some instances, the cancer is a lung cancer. For example, the lung cancer may be a non-small cell lung cancer (NSCLC), including but not limited to a locally advanced or metastatic (e.g., stage IIIB, stage IV, or recurrent) NSCLC. In some instances, the lung cancer (e.g., NSCLC) is unresectable/inoperable lung cancer (e.g., NSCLC). In some instances, the lung cancer is a chemotherapy-naïve lung cancer (e.g., a chemotherapy-naïve metastatic NSCLC (mNSCLC)). In some instances, the lung cancer is a non-squamous lung cancer (e.g., a non-squamous mNSCLC). In some instances, the lung cancer is a stage IV lung cancer (e.g., a stage IV mNSCLC). In some instances, the lung cancer is a recurrent lung cancer (e.g., a recurrent mNSCLC). In some instances, the patient having the lung cancer (e.g., NSCLC) has an EGFR or ALKgenomic alteration. In some instances, the patient having lung cancer with a EGFR or ALKgenomic alteration has disease progression/treatment intolerance with one or more approved tyrosine kinase inhibitors (TKI).

In some instances, the cancer may be a bladder cancer. For example, the bladder cancer may be a urothelial carcinoma (UC), including but not limited to a non-muscle invasive urothelial carcinoma, a muscle-invasive urothelial carcinoma, or a metastatic urothelial carcinoma. In some instances, the urothelial carcinoma is a metastatic urothelial carcinoma.

In some instances, the cancer may be a kidney cancer. For example, the kidney cancer may be a renal cell carcinoma (RCC), including stage I RCC, stage II RCC, stage III RCC, stage IV RCC, or recurrent RCC.

In some instances, the cancer may be a breast cancer. In some instances, the breast cancer may be a triple-negative breast cancer. For example, the breast cancer may be triple-negative breast cancer, estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer.

In some instances, the individual having a cancer, e.g., cancers described herein, has not been previously treated for the cancer. For example, the individual having a cancer has not previously received a PD-L1 axis binding antagonist therapy (e.g., a PD-L1 binding antagonist (e.g., an anti-PD-L1 antibody, e.g., atezolizumab) or a PD-1 binding antagonist (e.g., an anti-PD-1 antibody)).

In some instances, the individual having a cancer has previously received treatment for the cancer. In some instances, the individual having a cancer has previously received treatment including a non-PD-L1 axis binding antagonist therapy (e.g., an anti-cancer therapy (e.g., a cytotoxic agent, a growth-inhibitory agent, a radiation therapy, an anti-angiogenic agent, or a combination thereof)).

H. Combination therapies

In any of the methods herein, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in combination with an effective amount of one or more additional therapeutic agents. Suitable additional therapeutic agents include, for example, an anti-neoplastic agent, a chemotherapeutic agent, a growth inhibitory agent, a cytotoxic agent, a radiotherapy, or combinations thereof.

In some instances, the methods further involve administering to the patient an effective amount of one or more additional therapeutic agents. In some instances, the additional therapeutic agent is selected from the group consisting of a cytotoxic agent, a chemotherapeutic agent, a growth-inhibitory agent, a radiation therapy agent, an anti-angiogenic agent, and combinations thereof. In some instances, a PD-L1 axis binding antagonist (e.g., a PD-L1 binding antagonist, e.g., an anti-PD-L1 antibody, e.g., atezolizumab) may be administered in conjunction with a chemotherapy or chemotherapeutic agent. In some instances, a PD-L1 axis binding antagonist (e.g., a PD-L1 binding antagonist, e.g., an anti-PD-L1 antibody, e.g., atezolizumab) may be administered in conjunction with a radiation therapy agent. In some instances, a PD-L1 axis binding antagonist (e.g., a PD-L1 binding antagonist, e.g., an anti-PD-L1 antibody, e.g., atezolizumab) may be administered in conjunction with a targeted therapy or targeted therapeutic agent. In some instances, a PD-L1 axis binding antagonist (e.g., a PD-L1 binding antagonist, e.g., an anti-PD-L1 antibody, e.g., atezolizumab) may be administered in conjunction with an immunotherapy or immunotherapeutic agent, for example a monoclonal antibody. In some instances, the additional therapeutic agent is an agonist directed against an activating co-stimulatory molecule. In some instances, the additional therapeutic agent is an antagonist directed against an inhibitory co-stimulatory molecule.

Such combination therapies noted above encompass combined administration (where two or more therapeutic agents are included in the same or separate formulations), and separate administration, in which case, administration of a PD-L1 axis binding antagonist (e.g., a PD-L1 binding antagonist, e.g., an anti-PD-L1 antibody, e.g., atezolizumab) can occur prior to, simultaneously, and/or following, administration of the additional therapeutic agent or agents. In one instance, administration of PD-L1 axis binding antagonist (e.g., PD-L1 binding antagonist, e.g., an anti-PD-L1 antibody, e.g., atezolizumab) and administration of an additional therapeutic agent occur within about one month, or within about one, two or three weeks, or within about one, two, three, four, five, or six days, of each other.

Without wishing to be bound to theory, it is thought that enhancing T-cell stimulation, by promoting an activating co-stimulatory molecule or by inhibiting a negative co-stimulatory molecule, may promote tumor cell death thereby treating or delaying progression of cancer. In some instances, a PD-L1 axis binding antagonist (e.g., a PD-L1 binding antagonist, e.g., an anti-PD-L1 antibody, e.g., atezolizumab) may be administered in conjunction with an agonist directed against an activating co-stimulatory molecule. In some instances, an activating co-stimulatory molecule may include CD40, CD226, CD28, OX40, GITR, CD137, CD27, HVEM, or CD127. In some instances, the agonist directed against an activating co-stimulatory molecule is an agonist antibody that binds to CD40, CD226, CD28, OX40, GITR, CD137, CD27, HVEM, or CD127. In some instances, a PD-L1 axis binding antagonist (e.g., a PD-L1 binding antagonist, e.g., an anti-PD-L1 antibody, e.g., atezolizumab) may be administered in conjunction with an antagonist directed against an inhibitory co-stimulatory molecule. In some instances, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some instances, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some instances, a PD-L1 axis binding antagonist (e.g., a PD-L1 binding antagonist, e.g., an anti-PD-L1 antibody, e.g., atezolizumab) may be administered in conjunction with an antagonist directed against CTLA-4 (also known as CD152), e.g., a blocking antibody. In some instances, a PD-L1 axis binding antagonist (e.g., a PD-L1 binding antagonist, e.g., an anti-PD-L1 antibody, e.g., atezolizumab) may be administered in conjunction with ipilimumab (also known as MDX-010, MDX-101, or YERVOY®). In some instances, a PD-L1 axis binding antagonist (e.g., a PD-L1 binding antagonist, e.g., an anti-PD-L1 antibody, e.g., atezolizumab) may be administered in conjunction with tremelimumab (also known as ticilimumab or CP-675,206). In some instances, a PD-L1 axis binding antagonist (e.g., a PD-L1 binding antagonist, e.g., an anti-PD-L1 antibody, e.g., atezolizumab) may be administered in conjunction with an antagonist directed against B7-H3 (also known as CD276), e.g., a blocking antibody. In some instances, a PD-L1 axis binding antagonist (e.g., a PD-L1 binding antagonist, e.g., an anti-PD-L1 antibody, e.g., atezolizumab) may be administered in conjunction with MGA271. In some instances, a PD-L1 axis binding antagonist (e.g., a PD-L1 binding antagonist, e.g., an anti-PD-L1 antibody, e.g., atezolizumab) may be administered in conjunction with an antagonist directed against a TGF-beta, e.g., metelimumab (also known as CAT-192), fresolimumab (also known as GC1008), or LY2157299.

In some instances, a PD-L1 axis binding antagonist (e.g., a PD-L1 binding antagonist, e.g., an anti-PD-L1 antibody, e.g., atezolizumab) may be administered in conjunction with a treatment comprising adoptive transfer of a T-cell (e.g., a cytotoxic T-cell or CTL) expressing a chimeric antigen receptor (CAR). In some instances, a PD-L1 axis binding antagonist (e.g., a PD-L1 binding antagonist, e.g., an anti-PD-L1 antibody, e.g., atezolizumab) may be administered in conjunction with a treatment comprising adoptive transfer of a T-cell comprising a dominant-negative TGF beta receptor, e.g., a dominant-negative TGF beta type II receptor. In some instances, a PD-L1 axis binding antagonist (e.g., a PD-L1 binding antagonist, e.g., an anti-PD-L1 antibody, e.g., atezolizumab) may be administered in conjunction with a treatment comprising a HERCREEM protocol (see, e.g., ClinicalTrials.gov Identifier NCT00889954).

In some instances, a PD-L1 axis binding antagonist (e.g., a PD-L1 binding antagonist, e.g., an anti-PD-L1 antibody, e.g., atezolizumab) may be administered in conjunction with an agonist directed against CD137 (also known as TNFRSF9, 4-1 BB, or ILA), e.g., an activating antibody. In some instances, a PD-L1 axis binding antagonist (e.g., a PD-L1 binding antagonist, e.g., an anti-PD-L1 antibody, e.g., atezolizumab) may be administered in conjunction with urelumab (also known as BMS-663513). In some instances, a PD-L1 axis binding antagonist (e.g., a PD-L1 binding antagonist, e.g., an anti-PD-L1 antibody, e.g., atezolizumab) may be administered in conjunction with an agonist directed against CD40, e.g., an activating antibody. In some instances, a PD-L1 axis binding antagonist (e.g., a PD-L1 binding antagonist, e.g., an anti-PD-L1 antibody, e.g., atezolizumab) may be administered in conjunction with CP-870893. In some instances, a PD-L1 axis binding antagonist (e.g., a PD-L1 binding antagonist, e.g., an anti-PD-L1 antibody, e.g., atezolizumab) may be administered in conjunction with an agonist directed against OX40 (also known as CD134), e.g., an activating antibody. In some instances, a PD-L1 axis binding antagonist (e.g., a PD-L1 binding antagonist, e.g., an anti-PD-L1 antibody, e.g., atezolizumab) may be administered in conjunction with an anti-OX40 antibody (e.g., AgonOX). In some instances, a PD-L1 axis binding antagonist (e.g., a PD-L1 binding antagonist, e.g., an anti-PD-L1 antibody, e.g., atezolizumab) may be administered in conjunction with an agonist directed against CD27, e.g., an activating antibody. In some instances, a PD-L1 axis binding antagonist (e.g., a PD-L1 binding antagonist, e.g., an anti-PD-L1 antibody, e.g., atezolizumab) may be administered in conjunction with CDX-1127. In some instances, a PD-L1 axis binding antagonist (e.g., a PD-L1 binding antagonist, e.g., an anti-PD-L1 antibody, e.g., atezolizumab) may be administered in conjunction with an antagonist directed against indoleamine-2,3-dioxygenase (IDO). In some instances, with the IDO antagonist is 1-methyl-D-tryptophan (also known as 1-D-MT).

In some instances, a PD-L1 axis binding antagonist (e.g., a PD-L1 binding antagonist, e.g., an anti-PD-L1 antibody, e.g., atezolizumab) may be administered in conjunction with an antibody-drug conjugate. In some instances, the antibody-drug conjugate comprises mertansine or monomethyl auristatin E (MMAE). In some instances, a PD-L1 axis binding antagonist (e.g., a PD-L1 binding antagonist, e.g., an anti-PD-L1 antibody, e.g., atezolizumab) may be administered in conjunction with an anti-NaPi2b antibody-MMAE conjugate (also known as DNIB0600A or RG7599). In some instances, a PD-L1 axis binding antagonist (e.g., a PD-L1 binding antagonist, e.g., an anti-PD-L1 antibody, e.g., atezolizumab) may be administered in conjunction with trastuzumab emtansine (also known as T-DM1, ado-trastuzumab emtansine, or KADCYLA®, Genentech). In some instances, a PD-L1 axis binding antagonist (e.g., a PD-L1 binding antagonist, e.g., an anti-PD-L1 antibody, e.g., atezolizumab) may be administered in conjunction with DMUC5754A. In some instances, a PD-L1 axis binding antagonist (e.g., a PD-L1 binding antagonist, e.g., an anti-PD-L1 antibody, e.g., atezolizumab) may be administered in conjunction with an antibody-drug conjugate targeting the endothelin B receptor (EDNBR), e.g., an antibody directed against EDNBR conjugated with MMAE.

In some instances, a PD-L1 axis binding antagonist (e.g., a PD-L1 binding antagonist, e.g., an anti-PD-L1 antibody, e.g., atezolizumab) may be administered in conjunction with an anti-angiogenesis agent. In some instances, a PD-L1 axis binding antagonist (e.g., a PD-L1 binding antagonist, e.g., an anti-PD-L1 antibody, e.g., atezolizumab) may be administered in conjunction with an antibody directed against a VEGF, e.g., VEGF-A. In some instances, a PD-L1 axis binding antagonist (e.g., a PD-L1 binding antagonist, e.g., an anti-PD-L1 antibody, e.g., atezolizumab) may be administered in conjunction with bevacizumab (also known as AVASTIN®, Genentech). For example, atezolizumab may be administered in combination with bevacizumab. In further instances, atezolizumab may be administered in combination with bevacizumab and one or more chemotherapeutic agents (e.g., carboplatin and/or paclitaxel). In certain instances, atezolizumab may be administered in combination with bevacizumab, carboplatin, and paclitaxel. In some instances, a PD-L1 axis binding antagonist (e.g., a PD-L1 binding antagonist, e.g., an anti-PD-L1 antibody, e.g., atezolizumab) may be administered in conjunction with an antibody directed against angiopoietin 2 (also known as Ang2). In some instances, a PD-L1 axis binding antagonist (e.g., a PD-L1 binding antagonist, e.g., an anti-PD-L1 antibody, e.g., atezolizumab) may be administered in conjunction with MEDI3617.

The VEGF antagonist (e.g., bevacizumab) administered to the individual (e.g., human) in conjunction with a PD-L1 axis binding antagonist (e.g., a PD-L1 binding antagonist, e.g., an anti-PD-L1 antibody, e.g., atezolizumab) may be in the range of about 0.01 to about 50 mg/kg of the individual's body weight (e.g., between about 0.01 to about 45 mg/kg, between about 0.01 mg/kg to about 40 mg/kg, between about 0.01 mg/kg to about 35 mg/kg, between about 0.01 mg/kg to about 30 mg/kg, between about 0.1 mg/kg to about 30 mg/kg, between about 1 mg/kg to about 30 mg/kg, between about 2 mg/kg to about 30 mg/kg, between about 5 mg/kg to about 30 mg/kg, between about 5 mg/kg to about 25 mg/kg, between about 5 mg/kg to about 20 mg/kg, between about 10 mg/kg to about 20 mg/kg, or between about 12 mg/kg to about 18 mg/kg, e.g., about 15±2 mg/kg, about 15±1 mg/kg, about 15±0.5 mg/kg, about ±0.2 mg/kg, or about 15±0.1 mg/kg). For example, in some instances, the methods include administering to the individual a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) at about 1200 mg in conjunction with a VEGF antagonist (e.g., bevacizumab) at about 15 mg/kg of the individual's body weight. The method may further include administration of one or more chemotherapeutic agents, such as carboplatin and/or paclitaxel.

In some instances, a PD-L1 axis binding antagonist (e.g., a PD-L1 binding antagonist, e.g., an anti-PD-L1 antibody, e.g., atezolizumab) may be administered in conjunction with an antineoplastic agent. In some instances, a PD-L1 axis binding antagonist (e.g., a PD-L1 binding antagonist, e.g., an anti-PD-L1 antibody, e.g., atezolizumab) may be administered in conjunction with an agent targeting CSF-1R (also known as M-CSFR or CD115). In some instances, a PD-L1 axis binding antagonist (e.g., a PD-L1 binding antagonist, e.g., an anti-PD-L1 antibody, e.g., atezolizumab) may be administered in conjunction with anti-CSF-1 R (also known as IMC—CS4). In some instances, a PD-L1 axis binding antagonist (e.g., a PD-L1 binding antagonist, e.g., an anti-PD-L1 antibody, e.g., atezolizumab) may be administered in conjunction with an interferon, for example interferon alpha or interferon gamma. In some instances, a PD-L1 axis binding antagonist (e.g., a PD-L1 binding antagonist, e.g., an anti-PD-L1 antibody, e.g., atezolizumab) may be administered in conjunction with Roferon-A (also known as recombinant Interferon alpha-2a). In some instances, a PD-L1 axis binding antagonist (e.g., a PD-L1 binding antagonist, e.g., an anti-PD-L1 antibody, e.g., atezolizumab) may be administered in conjunction with GM-CSF (also known as recombinant human granulocyte macrophage colony stimulating factor, rhu GM-CSF, sargramostim, or LEUKINE®). In some instances, a PD-L1 axis binding antagonist (e.g., a PD-L1 binding antagonist, e.g., an anti-PD-L1 antibody, e.g., atezolizumab) may be administered in conjunction with IL-2 (also known as aldesleukin or PROLEUKIN®). In some instances, a PD-L1 axis binding antagonist (e.g., a PD-L1 binding antagonist, e.g., an anti-PD-L1 antibody, e.g., atezolizumab) may be administered in conjunction with IL-12. In some instances, a PD-L1 axis binding antagonist (e.g., a PD-L1 binding antagonist, e.g., an anti-PD-L1 antibody, e.g., atezolizumab) may be administered in conjunction with an antibody targeting CD20. In some instances, the antibody targeting CD20 is obinutuzumab (also known as GA101 or GAZYVA®) or rituximab. In some instances, a PD-L1 axis binding antagonist (e.g., a PD-L1 binding antagonist, e.g., an anti-PD-L1 antibody, e.g., atezolizumab) may be administered in conjunction with an antibody targeting GITR. In some instances, the antibody targeting GITR is TRX518.

In some instances, a PD-L1 axis binding antagonist (e.g., a PD-L1 binding antagonist, e.g., an anti-PD-L1 antibody, e.g., atezolizumab) may be administered in conjunction with a cancer vaccine. In some instances, the cancer vaccine is a peptide cancer vaccine, which in some instances is a personalized peptide vaccine. In some instances, the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a PD-L1 axis binding antagonist (e.g., a PD-L1 binding antagonist, e.g., an anti-PD-L1 antibody, e.g., atezolizumab) may be administered in conjunction with an adjuvant. In some instances, a PD-L1 axis binding antagonist (e.g., a PD-L1 binding antagonist, e.g., an anti-PD-L1 antibody, e.g., atezolizumab) may be administered in conjunction with a treatment comprising a TLR agonist, e.g., Poly-ICLC (also known as HILTONOL®), LPS, MPL, or CpG ODN. In some instances, a PD-L1 axis binding antagonist (e.g., a PD-L1 binding antagonist, e.g., an anti-PD-L1 antibody, e.g., atezolizumab) may be administered in conjunction with tumor necrosis factor (TNF) alpha (TNF-α). In some instances, a PD-L1 axis binding antagonist (e.g., a PD-L1 binding antagonist, e.g., an anti-PD-L1 antibody, e.g., atezolizumab) may be administered in conjunction with IL-1. In some instances, a PD-L1 axis binding antagonist (e.g., a PD-L1 binding antagonist, e.g., an anti-PD-L1 antibody, e.g., atezolizumab) may be administered in conjunction with HMGB1. In some instances, a PD-L1 axis binding antagonist (e.g., a PD-L1 binding antagonist, e.g., an anti-PD-L1 antibody, e.g., atezolizumab) may be administered in conjunction with an IL-10 antagonist. In some instances, a PD-L1 axis binding antagonist (e.g., a PD-L1 binding antagonist, e.g., an anti-PD-L1 antibody, e.g., atezolizumab) may be administered in conjunction with an IL-4 antagonist. In some instances, a PD-L1 axis binding antagonist (e.g., a PD-L1 binding antagonist, e.g., an anti-PD-L1 antibody, e.g., atezolizumab) may be administered in conjunction with an IL-13 antagonist. In some instances, a PD-L1 axis binding antagonist (e.g., a PD-L1 binding antagonist, e.g., an anti-PD-L1 antibody, e.g., atezolizumab) may be administered in conjunction with an HVEM antagonist. In some instances, a PD-L1 axis binding antagonist (e.g., a PD-L1 binding antagonist, e.g., an anti-PD-L1 antibody, e.g., atezolizumab) may be administered in conjunction with an ICOS agonist, e.g., by administration of ICOS-L, or an agonistic antibody directed against ICOS. In some instances, a PD-L1 axis binding antagonist (e.g., a PD-L1 binding antagonist, e.g., an anti-PD-L1 antibody, e.g., atezolizumab) may be administered in conjunction with a treatment targeting CX3CL1. In some instances, a PD-L1 axis binding antagonist (e.g., a PD-L1 binding antagonist, e.g., an anti-PD-L1 antibody, e.g., atezolizumab) may be administered in conjunction with a treatment targeting CXCL9. In some instances, a PD-L1 axis binding antagonist (e.g., a PD-L1 binding antagonist, e.g., an anti-PD-L1 antibody, e.g., atezolizumab) may be administered in conjunction with a treatment targeting CXCL10. In some instances, a PD-L1 axis binding antagonist (e.g., a PD-L1 binding antagonist, e.g., an anti-PD-L1 antibody, e.g., atezolizumab) may be administered in conjunction with a treatment targeting CCL5. In some instances, a PD-L1 axis binding antagonist (e.g., a PD-L1 binding antagonist, e.g., an anti-PD-L1 antibody, e.g., atezolizumab) may be administered in conjunction with an LFA-1 or ICAM1 agonist. In some instances, a PD-L1 axis binding antagonist (e.g., a PD-L1 binding antagonist, e.g., an anti-PD-L1 antibody, e.g., atezolizumab) may be administered in conjunction with a Selectin agonist.

In some instances, a PD-L1 axis binding antagonist (e.g., a PD-L1 binding antagonist, e.g., an anti-PD-L1 antibody, e.g., atezolizumab) may be administered in conjunction with a targeted therapy. In some instances, a PD-L1 axis binding antagonist (e.g., a PD-L1 binding antagonist, e.g., an anti-PD-L1 antibody, e.g., atezolizumab) may be administered in conjunction with an inhibitor of B-Raf. In some instances, a PD-L1 axis binding antagonist (e.g., a PD-L1 binding antagonist, e.g., an anti-PD-L1 antibody, e.g., atezolizumab) may be administered in conjunction with vemurafenib (also known as ZELBORAF®). In some instances, a PD-L1 axis binding antagonist (e.g., a PD-L1 binding antagonist, e.g., an anti-PD-L1 antibody, e.g., atezolizumab) may be administered in conjunction with dabrafenib (also known as TAFINLAR®). In some instances, a PD-L1 axis binding antagonist (e.g., a PD-L1 binding antagonist, e.g., an anti-PD-L1 antibody, e.g., atezolizumab) may be administered in conjunction with erlotinib (also known as TARCEVA®). In some instances, a PD-L1 axis binding antagonist (e.g., a PD-L1 binding antagonist, e.g., an anti-PD-L1 antibody, e.g., atezolizumab) may be administered in conjunction with an inhibitor of a MEK, such as MEK1 (also known as MAP2K1) or MEK2 (also known as MAP2K2). In some instances, a PD-L1 axis binding antagonist (e.g., a PD-L1 binding antagonist, e.g., an anti-PD-L1 antibody, e.g., atezolizumab) may be administered in conjunction with cobimetinib (also known as GDC-0973 or XL-518). In some instances, a PD-L1 axis binding antagonist (e.g., a PD-L1 binding antagonist, e.g., an anti-PD-L1 antibody, e.g., atezolizumab) may be administered in conjunction with trametinib (also known as MEKINIST®). In some instances, a PD-L1 axis binding antagonist (e.g., a PD-L1 binding antagonist, e.g., an anti-PD-L1 antibody, e.g., atezolizumab) may be administered in conjunction with an inhibitor of K-Ras. In some instances, a PD-L1 axis binding antagonist (e.g., a PD-L1 binding antagonist, e.g., an anti-PD-L1 antibody, e.g., atezolizumab) may be administered in conjunction with an inhibitor of c-Met. In some instances, a PD-L1 axis binding antagonist (e.g., a PD-L1 binding antagonist, e.g., an anti-PD-L1 antibody, e.g., atezolizumab) may be administered in conjunction with onartuzumab (also known as MetMAb). In some instances, a PD-L1 axis binding antagonist (e.g., a PD-L1 binding antagonist, e.g., an anti-PD-L1 antibody, e.g., atezolizumab) may be administered in conjunction with an inhibitor of Alk. In some instances, a PD-L1 axis binding antagonist (e.g., a PD-L1 binding antagonist, e.g., an anti-PD-L1 antibody, e.g., atezolizumab) may be administered in conjunction with AF802 (also known as CH5424802 or alectinib). In some instances, a PD-L1 axis binding antagonist (e.g., a PD-L1 binding antagonist, e.g., an anti-PD-L1 antibody, e.g., atezolizumab) may be administered in conjunction with an inhibitor of a phosphatidylinositol 3-kinase (P¹³K). In some instances, a PD-L1 axis binding antagonist (e.g., a PD-L1 binding antagonist, e.g., an anti-PD-L1 antibody, e.g., atezolizumab) may be administered in conjunction with BKM120. In some instances, a PD-L1 axis binding antagonist (e.g., a PD-L1 binding antagonist, e.g., an anti-PD-L1 antibody, e.g., atezolizumab) may be administered in conjunction with idelalisib (also known as GS-1101 or CAL-101). In some embodiments, a PD-L1 axis binding antagonist (e.g., a PD-L1 binding antagonist, e.g., an anti-PD-L1 antibody, e.g., atezolizumab) may be administered in conjunction with perifosine (also known as KRX-0401). In some embodiments, a PD-L1 axis binding antagonist (e.g., a PD-L1 binding antagonist, e.g., an anti-PD-L1 antibody, e.g., atezolizumab) may be administered in conjunction with an inhibitor of an Akt. In some embodiments, a PD-L1 axis binding antagonist (e.g., a PD-L1 binding antagonist, e.g., an anti-PD-L1 antibody, e.g., atezolizumab) may be administered in conjunction with MK2206. In some instances, a PD-L1 axis binding antagonist (e.g., a PD-L1 binding antagonist, e.g., an anti-PD-L1 antibody, e.g., atezolizumab) may be administered in conjunction with GSK690693. In some instances, a PD-L1 axis binding antagonist (e.g., a PD-L1 binding antagonist, e.g., an anti-PD-L1 antibody, e.g., atezolizumab) may be administered in conjunction with GDC-0941. In some instances, a PD-L1 axis binding antagonist (e.g., a PD-L1 binding antagonist, e.g., an anti-PD-L1 antibody, e.g., atezolizumab) may be administered in conjunction with an inhibitor of mTOR. In some instances, a PD-L1 axis binding antagonist (e.g., a PD-L1 binding antagonist, e.g., an anti-PD-L1 antibody, e.g., atezolizumab) may be administered in conjunction with sirolimus (also known as rapamycin). In some instances, a PD-L1 axis binding antagonist (e.g., a PD-L1 binding antagonist, e.g., an anti-PD-L1 antibody, e.g., atezolizumab) may be administered in conjunction with temsirolimus (also known as CCI-779 or TORISEL®). In some instances, a PD-L1 axis binding antagonist (e.g., a PD-L1 binding antagonist, e.g., an anti-PD-L1 antibody, e.g., atezolizumab) may be administered in conjunction with everolimus (also known as RAD001). In some instances, a PD-L1 axis binding antagonist (e.g., a PD-L1 binding antagonist, e.g., an anti-PD-L1 antibody, e.g., atezolizumab) may be administered in conjunction with ridaforolimus (also known as AP-23573, MK-8669, or deforolimus). In some instances, a PD-L1 axis binding antagonist (e.g., a PD-L1 binding antagonist, e.g., an anti-PD-L1 antibody, e.g., atezolizumab) may be administered in conjunction with OSI-027. In some instances, a PD-L1 axis binding antagonist (e.g., a PD-L1 binding antagonist, e.g., an anti-PD-L1 antibody, e.g., atezolizumab) may be administered in conjunction with AZD8055. In some instances, a PD-L1 axis binding antagonist (e.g., a PD-L1 binding antagonist, e.g., an anti-PD-L1 antibody, e.g., atezolizumab) may be administered in conjunction with INK128. In some instances, a PD-L1 axis binding antagonist (e.g., a PD-L1 binding antagonist, e.g., an anti-PD-L1 antibody, e.g., atezolizumab) may be administered in conjunction with a dual PI3K/mTOR inhibitor. In some instances, a PD-L1 axis binding antagonist (e.g., a PD-L1 binding antagonist, e.g., an anti-PD-L1 antibody, e.g., atezolizumab) may be administered in conjunction with XL765. In some instances, a PD-L1 axis binding antagonist (e.g., a PD-L1 binding antagonist, e.g., an anti-PD-L1 antibody, e.g., atezolizumab) may be administered in conjunction with GDC-0980. In some instances, a PD-L1 axis binding antagonist (e.g., a PD-L1 binding antagonist, e.g., an anti-PD-L1 antibody, e.g., atezolizumab) may be administered in conjunction with BEZ235 (also known as NVP-BEZ235). In some instances, a PD-L1 axis binding antagonist (e.g., a PD-L1 binding antagonist, e.g., an anti-PD-L1 antibody, e.g., atezolizumab) may be administered in conjunction with BGT226. In some instances, a PD-L1 axis binding antagonist (e.g., a PD-L1 binding antagonist, e.g., an anti-PD-L1 antibody, e.g., atezolizumab) may be administered in conjunction with GSK2126458. In some instances, a PD-L1 axis binding antagonist (e.g., a PD-L1 binding antagonist, e.g., an anti-PD-L1 antibody, e.g., atezolizumab) may be administered in conjunction with PF-04691502. In some instances, a PD-L1 axis binding antagonist (e.g., a PD-L1 binding antagonist, e.g., an anti-PD-L1 antibody, e.g., atezolizumab) may be administered in conjunction with PF-05212384 (also known as PKI-587).

(i) Combination Therapies in Clinical Trials

PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) can be administered to an individual in conjunction with one or more additional therapeutic agents, wherein, prior or subsequent to treatment, the individual has undergone diagnostic testing according to any one of the diagnostic methods described herein and has been identified as one who is likely to benefit from treatment with a

PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). As described further below, the additional therapeutic agents may be one that has been tested or is undergoing testing in a clinical trial for cancer therapies that include atezolizumab.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with obinutuzumab and polatuzumab vedotin (e.g., in the treatment of lymphoma (e.g., relapsed or refractory follicular lymphoma or diffuse large B-cell lymphoma)), as in the clinical trial NCT02729896.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel (e.g., albumin-bound paclitaxel (nab-paclitaxel (ABRAXANE®), e.g., in the treatment of breast cancer (e.g., TNBC)), as in the clinical trial NCT02530489.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®) (e.g., in the treatment of locally advanced or metastatic tumors (e.g., in breast cancer, cervical cancer, kidney cancer, gastric cancer, ovarian cancer, or bladder cancer), as in the clinical trial NCT01633970.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®) and leucovorin/oxaliplatin/5-fluorouracil (FOLFOX) (e.g., in the treatment of locally advanced or metastatic tumors, e.g., in breast cancer, cervical cancer, kidney cancer, gastric cancer, ovarian cancer, or bladder cancer), as in the clinical trial NCT01633970.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel (e.g., albumin-bound paclitaxel (nab-paclitaxel (ABRAXANE®)) and carboplatin (e.g., PARAPLATIN®) (e.g., in the treatment of locally advanced or metastatic tumors, e.g., in the treatment of lung cancer (NSCLC), breast cancer, cervical cancer, kidney cancer, gastric cancer, ovarian cancer, or bladder cancer), as in the clinical trial NCT01633970.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel (e.g., albumin-bound paclitaxel (nab-paclitaxel (ABRAXANE®)), e.g., in the treatment of locally advanced or metastatic tumors (e.g., in the treatment of lung cancer (NSCLC), breast cancer, cervical cancer, kidney cancer, gastric cancer, ovarian cancer, or bladder cancer), as in the clinical trial NCT01633970.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with pemetrexed (e.g., ALIMTA®) and carboplatin (e.g., PARAPLATIN®) (e.g., in the treatment of locally advanced or metastatic tumors, e.g., in the treatment of breast cancer, cervical cancer, kidney cancer, gastric cancer, ovarian cancer, or bladder cancer), as in the clinical trial NCT01633970.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with etoposide (e.g., ETOPOPHOS®, TOPOSAR®) and carboplatin (e.g., PARAPLATIN®) (e.g., in the treatment of lung cancer (e.g., small cell lung cancer (SCLC))), as in the clinical trial NCT02748889.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel (e.g., albumin-bound paclitaxel (nab-paclitaxel (ABRAXANE®)) and carboplatin (e.g., PARAPLATIN®) (e.g., in the treatment of locally advanced or metastatic tumors, e.g., in the treatment of lung cancer (NSCLC)), as in the clinical trial NCT02716038.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with epacadostat (e.g., INCB024360) (e.g., in the treatment of lung cancer (e.g., NSCLC) or bladder cancer (e.g., urothelial carcinoma)), as in the clinical trial NCT02298153.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with radiation therapy and a chemotherapy (e.g., carboplatin and/or paclitaxel), e.g., in the treatment of lung cancer (e.g., NSCLC), as in the clinical trial NCT02525757.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with veliparib (e.g., in the treatment of breast cancer, e.g., TNBC, BRCA1 gene mutation, BRCA2 gene mutation, estrogen receptor negative breast cancer, Her2/Neu negative breast cancer, stage IIIA breast cancer, stage IIIB breast cancer, stage IIIC breast cancer, or stage IV breast cancer), as in the clinical trial NCT02849496.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with alectinib (also known as ALECENSA®) (e.g., in the treatment of lung cancer (e.g., NSCLC), as in the clinical trial NCT02013219.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with erlotinib (also known as TARCEVA®) (e.g., in the treatment of lung cancer (e.g., NSCLC), as in the clinical trial NCT02013219.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with MTIG7192A (e.g., in the treatment of advanced metastatic tumors), as in the clinical trial NCT02794571.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with vemurafenib (also known as ZELBORAF®) (e.g., in the treatment of skin cancer (e.g., a malignant melanoma), as in the clinical trial NCT01656642.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with vemurafenib (also known as ZELBORAF®) and cobimetinib (also known as (COTELLIC®) (e.g., in the treatment of skin cancer (e.g., a malignant melanoma)), as in the clinical trial NCT01656642.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®, Genentech) (e.g., in the treatment of ovarian, fallopian tube, or peritoneal cancer), as in the clinical trial NCT02839707.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with obinutuzumab (e.g., in the treatment of lymphoma (e.g., lymphocytic lymphoma or relapsed refractory or chronic lymphocytic leukemia (CLL))), as in the clinical trial NCT02846623.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with carboplatin and pemetrexed (e.g., in the treatment of lung cancer (e.g., NSCLC)), as in clinical trial NCT02657434.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cisplatin and pemetrexed (e.g., in the treatment of lung cancer (e.g., NSCLC)), as in the clinical trial NCT02657434.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with tazemetostat (e.g., in the treatment of lymphoma (e.g., follicular lymphoma or diffuse large b-cell lymphoma)), as in the clinical trial NCT02220842.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with obinutuzumab (e.g., in the treatment of lymphoma (e.g., follicular lymphoma or diffuse large b-cell lymphoma)), as in the clinical trial NCT02220842.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with lenalidomide (e.g., in the treatment of multiple myeloma), as in the clinical trial NCT02431208.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with daratumumab (e.g., in the treatment of multiple myeloma), as in the clinical trial NCT02431208.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with daratumumab and lenalidomide (e.g., in the treatment of multiple myeloma), as in the clinical trial NCT02431208.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with daratumumab and pomalidomide (e.g., in the treatment of multiple myeloma), as in the clinical trial NCT02431208.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®, Genentech) (e.g., in the treatment of kidney cancer (e.g., renal cell carcinoma)), as in the clinical trial NCT02420821.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with stereotactic body radiation (e.g., in the treatment of lung cancer (e.g., NSCLC)), as in the clinical trial NCT02400814.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with rociletinib (e.g., in the treatment of lung cancer (e.g., NSCLC)), as in the clinical trial NCT02630186.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with GDC-0919 (e.g., in the treatment of a solid tumor (e.g., renal cell cancer (RCC), urothelial carcinoma (UC), triple-negative breast cancer (TNBC), non-small cell lung cancer (NSCLC), melanoma, head and neck squamous cell carcinoma (HNSCC), gastric cancer, ovarian cancer, cervical cancer, endometrial cancer, or Merkel cell cancer)), as in the clinical trial NCT02471846.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with radium-223 dichloride (e.g., in the treatment of lung prostate cancer (e.g., castrate-resistant prostate cancer)), as in the clinical trial NCT02814669.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with MOXR0916 (e.g., in the treatment of a solid tumor (e.g., locally advanced or metastatic solid tumors)), as in the clinical trial NCT02410512.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®, Genentech) and MOXR0916 (e.g., in the treatment of a solid tumor (e.g., locally advanced or metastatic solid tumors)), as in the clinical trial NCT02410512.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with azacitidine (e.g., in the treatment of a solid tumor (e.g., myelodysplastic syndromes)), as in the clinical trial NCT02508870.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel (e.g., albumin-bound paclitaxel (nab-paclitaxel (ABRAXANE®)) (e.g., in the treatment of breast cancer (e.g., TNBC))) as in the clinical trial NCT02425891.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with lenalidomide and obinutuzumab (e.g., in the treatment of lymphoma), as in the clinical trial NCT02631577.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with etoposide (e.g., ETOPOPHOS®, TOPOSAR®) and carboplatin (e.g., PARAPLATIN®) (e.g., in the treatment of lung cancer (e.g., small cell lung cancer (SCLC))), as in the clinical trial NCT02763579.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with ipilimumab (e.g., in the treatment of locally advanced or metastatic solid tumors), as in the clinical trial NCT02174172.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with interferon alfa-2b (e.g., in the treatment of locally advanced or metastatic solid tumors (e.g., NSCLC, melanoma, or RCC)), as in the clinical trial NCT02174172.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with hypofractionated image-guided radiotherapy (e.g., in the treatment of lung cancer (e.g., NSCLC)), as in the clinical trial NCT02463994.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with CDX-1401 (e.g., in the treatment of lung cancer (e.g., NSCLC)), as in the clinical trial NCT02495636.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with CDX-1401 (e.g., in the treatment of lung cancer (e.g., NSCLC)), as in the clinical trial NCT02495636.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with trastuzumab and pertuzumab (e.g., in the treatment of breast cancer (e.g., Her2-positive breast cancer)), as in the clinical trial NCT02605915.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with trastuzumab emtansine (e.g., in the treatment of breast cancer (e.g., Her2-positive breast cancer)), as in the clinical trial NCT02605915.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with doxorubicin and cyclophosphamide (e.g., in the treatment of breast cancer (e.g., Her2-positive breast cancer)), as in the clinical trial NCT02605915.

In some instances, the PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with trastuzumab, pertuzumab, and docetaxel (e.g., in the treatment of breast cancer (e.g., Her2-positive breast cancer)), as in the clinical trial NCT02605915.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®) (e.g., in the treatment of kidney cancer (e.g., advanced non-clear cell kidney cancer)), as in the clinical trial NCT02724878.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with CMB305 (e.g., in the treatment of sarcoma (e.g., myxoid/round cell liposarcoma, synovial sarcoma, metastatic sarcoma, recurrent adult soft tissue sarcoma, locally advanced sarcoma, or liposarcoma)), as in the clinical trial NCT02609984.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with R07009789 (e.g., in the treatment of solid cancers (e.g., locally advanced and metastatic solid tumors)), as in the clinical trial NCT02304393.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with Bacille Calmette-Guérin (also known as ONCOTICE®) (e.g., in the treatment of bladder cancer (e.g., non-muscle invasive bladder cancer)), as in the clinical trial NCT02792192.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with stereotactic body radiation therapy (e.g., in the treatment of lung cancer (e.g., NSCLC)), as in the clinical trial NCT02599454.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with carboplatin, and nab-paclitaxel (also known as ABRAXANE®) (e.g., in the treatment of breast cancer (e.g., invasive ductal breast carcinoma)), as in the clinical trial NCT02620280.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with carboplatin, nab-paclitaxel (also known as ABRAXANE®), and an adjuvant chemotherapy including AC or EC (adriamycin or epirubicin and cyclophosphamide) or FEC (fluorouracil, epirubicin, and cyclophosphamide) (e.g., in the treatment of breast cancer (e.g., invasive ductal breast carcinoma)), as in the clinical trial NCT02620280.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with gemcitabine and carboplatin or cisplatin (e.g., in the treatment of urothelial carcinoma), as in the clinical trial NCT02807636.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel and carboplatin (e.g., in the treatment of lung cancer (e.g., NSCLC, e.g., non-squamous NSCLC)), as in the clinical trial NCT02366143.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab, paclitaxel, and carboplatin (e.g., in the treatment of lung cancer (e.g., NSCLC, e.g., non-squamous NSCLC)), as in the clinical trial NCT02366143.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cergutuzumab (also known as R06895882) (e.g., in the treatment of locally advanced and/or metastatic solid tumors), as in the clinical trial NCT02350673.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bendamustine and obinutuzumab (e.g., in the treatment of lymphoma (e.g., diffuse large B-cell lymphoma or follicular lymphoma)), as in the clinical trial NCT02596971.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bendamustine, cyclophosphamide, obinutuzumab, prednisone, and vincristine (e.g., in the treatment of lymphoma (e.g., diffuse large B-cell lymphoma or follicular lymphoma)), as in the clinical trial NCT02596971.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cyclophosphamide, doxorubicin, obinutuzumab, prednisone, and vincristine (e.g., in the treatment of lymphoma (e.g., diffuse large B-cell lymphoma or follicular lymphoma)), as in the clinical trial NCT02596971.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cyclophosphamide, doxorubicin, prednisone, vincristine, and rituximab (e.g., in the treatment of lymphoma (e.g., diffuse large B-cell lymphoma or follicular lymphoma)), as in the clinical trial NCT02596971.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with R06958688 (e.g., in the treatment of locally advanced and/or metastatic solid tumors (e.g., carcinoembryonic antigen (CEA)-positive solid tumors)), as in the clinical trial NCT02650713.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with acetylsalicylic acid (e.g., in the treatment of ovarian cancer (e.g., ovarian neoplasms)), as in the clinical trial NCT02659384.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (e.g., in the treatment of ovarian cancer (e.g., ovarian neoplasms)), as in the clinical trial NCT02659384.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with vanucizumab (also known as R05520985) (e.g., in the treatment of locally advanced and/or metastatic solid tumors), as in the clinical trial NCT01688206.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with carboplatin and nab-paclitaxel (e.g., in the treatment of lung cancer (e.g., non-squamous NSCLC)), as in the clinical trial NCT02367781.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®) (e.g., in the treatment of kidney cancer (e.g., renal cell carcinoma)), as in the clinical trial NCT01984242.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cobimetinib (also known as GDC-0973) (e.g., in the treatment of locally advanced or metastatic solid tumors), as in the clinical trial NCT01988896.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with R05509554 (e.g., in the treatment of locally advanced solid tumors (e.g., locally advanced and/or metastatic triple negative breast cancer, ovarian cancer, bladder cancer, gastric cancer, or soft tissue sarcoma)), as in the clinical trial NCT02323191.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with varlilumab (e.g., in the treatment of advanced cancer (e.g., melanoma, RCC, triple negative breast cancer, bladder cancer, head and neck cancer, or non-small cell lung cancer)), as in the clinical trial NCT02543645.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cobimetinib (e.g., in the treatment of colorectal cancer), as in the clinical trial NCT02788279.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with cobimetinib (e.g., in the treatment of colorectal cancer), as in the clinical trial NCT02788279.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®) (e.g., in the treatment of solid tumors), as in the clinical trial NCT02715531.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with bevacizumab (also known as AVASTIN®), leucovorin, oxaliplatin, and optionally, capecitabine (e.g., in the treatment of solid tumors), as in the clinical trial NCT02715531.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with nab-paclitaxel and gemcitabine (e.g., in the treatment of solid tumors), as in the clinical trial NCT02715531.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with oxaliplatin, leucovorin, 5-fluorouracil (5-FU), oxaliplatin, and cisplatin (e.g., in the treatment of solid tumors), as in the clinical trial NCT02715531.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with nab-paclitaxel and carboplatin (e.g., in the treatment of lung cancer (e.g., squamous NSCLC)), as in the clinical trial NCT02367794.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with paclitaxel and carboplatin (e.g., in the treatment of lung cancer (e.g., squamous NSCLC)), as in the clinical trial NCT02367794.

In some instances, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) may be administered in conjunction with CPI-444 (e.g., in the treatment of advanced cancers (e.g., non-small cell lung cancer, malignant melanoma, renal cell cancer, triple negative breast cancer, colorectal cancer with microsatellite instability (MSI), and bladder cancer)), as in the clinical trial NCT02655822.

### IV. PD-L1 AXIS BINDING ANTAGONISTS

PD-L1 axis binding antagonists include PD-L1 binding antagonists, PD-1 binding antagonists, and PD-L2 binding antagonists. PD-1 (programmed death 1) is also referred to in the art as “programmed cell death 1,” “PDCD1,” “CD279,” and “SLEB2.” An exemplary human PD-L1 is shown in UniProtKB/Swiss-Prot Accession No.Q9NZQ7.1. An exemplary human PD-1 is shown in UniProtKB/Swiss-Prot Accession No. Q15116. PD-L1 (programmed death ligand 1) is also referred to in the art as “programmed cell death 1 ligand 1,” “PDCD1LG1,” “CD274,” “B7-H,” and “PDL1.” PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some embodiments, PD-L1, PD-1, and PD-L2 are human PD-L1, PD-1, and PD-L2. The PD-L1 axis binding antagonist may, in some instances, be a PD-L1 binding antagonist, a PD-1 binding antagonist, or a PD-L2 binding antagonist.

Any of the methods, compositions for use, uses, kits, or articles of manufacture described herein may include or involve any of the PDL-L1 axis binding antagonists described below.

In one aspect, the disclosure provides a PD-L1 axis binding antagonist for use, e.g., in any one of the methods disclosed herein. In one embodiment, the PD-L1 axis binding antagonist is used for treatment of a cancer. In one such embodiment, the method further comprises administering to the individual an effective amount of at least one additional therapeutic agent, e.g., any additional therapeutic agent disclosed herein.

In another aspect, the disclosure provides for the use of a PD-L1 axis binding antagonist in the manufacture or preparation of a medicament, e.g., for use in any one of the methods disclosed herein. In one embodiment, the medicament is for treatment of a cancer. In one such embodiment, the method further comprises administering to the individual an effective amount of at least one additional therapeutic agent, e.g., any additional therapeutic agent disclosed herein.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. In some instances, the PD-L1 binding antagonist is an antibody. In some instances, the antibody is selected from the group consisting of: atezolizumab, YW243.55.570, MDX-1105, MED14736 (durvalumab), and MSB0010718C (avelumab).

In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. In some instances, the anti-PD-L1 antibody described herein binds to human PD-L1. In some particular instances, the anti-PD-L1 antibody is atezolizumab (CAS Registry Number: 1422185-06-5). Atezolizumab (Genentech) is also known as MPDL3280A.

In some instances, the anti-PD-L1 antibody comprises a heavy chain variable region (HVR-H) comprising an HVR-H1, HVR-H2, and HVR-H3 sequence, wherein:
(a) the HVR-H1 sequence is GFTFSDSWIH (SEQ ID NO: 1);
(b) the HVR-H2 sequence is AWISPYGGSTYYADSVKG (SEQ ID NO: 2); and
(c) the HVR-H3 sequence is RHWPGGFDY (SEQ ID NO: 3).

In some instances, the anti-PD-L1 antibody further comprises a light chain variable region (HVR-L) comprising an HVR-L1, HVR-L2, and HVR-L3 sequence, wherein:
(a) the HVR-L1 sequence is RASQDVSTAVA (SEQ ID NO: 4);
(b) the HVR-L2 sequence is SASFLYS (SEQ ID NO: 5); and
(c) the HVR-L3 sequence is QQYLYHPAT (SEQ ID NO: 6).

In some instances, the anti-PD-L1 antibody comprises a heavy chain and a light chain sequence, wherein:
(a) the heavy chain variable (VH) region sequence comprises the amino acid sequence:

(SEQ ID NO: 7)
EVQLVESGGGLVQPGGSLRLSCAASGFTFSDSWI
HWVRQAPGKGLEWVAWISPYGGSTYYADSVKGRF
TISADTSKNTAYLQMNSLRAEDTAVYYCARRHWP
GGFDYWGQGTLVTVSS;

and
(b) the light chain variable (VL) region sequence comprises the amino acid sequence:

(SEQ ID NO: 8)
DIQMTQSPSSLSASVGDRVTITCRASQDVSTAVAWYQQKP
GKAPKLLIYSASFLYSGVPSRFSGSGSGTDFTLTISSLOP
EDFATYYCQQYLYHPATFGQGTKVEIKR.

In some instances, the anti-PD-L1 antibody comprises a heavy chain and a light chain sequence, wherein:
(a) the heavy chain comprises the amino acid sequence:

(SEQ ID NO: 9)
EVQLVESGGGLVQPGGSLRLSCAASGFTFSDSWIHWVRQA
PGKGLEWVAWISPYGGSTYYADSVKGRFTISADTSKNTAY
LQMNSLRAEDTAVYYCARRHWPGGFDYWGQGTLVTVSSAS
TKGPSVFPLAPSSKSTSGGTAALGCLVKDYFPEPVTVSWN
SGALTSGVHTFPAVLQSSGLYSLSSVVTVPSSSLGTQTYI
CNVNHKPSNTKVDKKVEPKSCDKTHTCPPCPAPELLGGPS
VFLFPPKPKDTLMISRTPEVTCVVVDVSHEDPEVKFNWYV
DGVEVHNAKTKPREEQYASTYRVVSVLTVLHQDWLNGKEY
KCKVSNKALPAPIEKTISKAKGQPREPQVYTLPPSREEMT
KNQVSLTCLVKGFYPSDIAVEWESNGQPENNYKTTPPVLD
SDGSFFLYSKLTVDKSRWQQGNVFSCSVMHEALHNHYTQK
SLSLSPG;

and
(b) the light chain comprises the amino acid sequence:

(SEQ ID NO: 10)
DIQMTQSPSSLSASVGDRVTITCRASQDVSTAVAWYQQKP
GKAPKLLIYSASFLYSGVPSRFSGSGSGTDFTLTISSLOP
EDFATYYCQQYLYHPATFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 7); (b) a VL domain comprising an amino acid sequence comprising having at least 95% sequence identity (e.g., at least 95%, 96%, 97%, 98%, or 99% sequence identity) to, or the sequence of (SEQ ID NO: 8); or (c) a VH domain as in (a) and a VL domain as in (b). In other instances, the anti-PD-L1 antibody is selected from the group consisting of YW243.55.570, MDX-1105, MED14736 (durvalumab), and MSB0010718C (avelumab). Antibody YW243.55.570 is an anti-PD-L1 described in PCT Pub. No. WO 2010/077634. MDX-1105, also known as BMS-936559, is an anti-PD-L1 antibody described in PCT Pub. No. WO 2007/005874. MED14736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference.

It is expressly contemplated that such PD-L1 binding antagonist antibodies for use in any of the instances enumerated above may have any of the features, singly or in combination, described in any one of Subsections 1-7 of Section C below.

### B. PD-1 Binding Antagonists

In some instances, the PD-L1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. In some instances, the PD-1 binding antagonist is an antibody. In some instances, the antibody is selected from the group consisting of: MDX 1106 (nivolumab), MK-3475 (pembrolizumab), MEDI-0680 (AMP-514), PDR001, REGN2810, and BGB-108. In some instances, the PD-1 binding antagonist is an Fc-fusion protein. For example, in some instances, the Fc-fusion protein is AMP-224.

In some embodiments, the PD-1 binding antagonist is a molecule that inhibits the binding of PD-1 to its ligand binding partners. In a specific aspect the PD-1 ligand binding partners are PD-L1 and/or PD-L2. In another embodiment, a PD-L1 binding antagonist is a molecule that inhibits the binding of PD-L1 to its binding ligands. In a specific aspect, PD-L1 binding partners are PD-1 and/or B7-1. In another embodiment, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody), for example, as described below. In some embodiments, the anti-PD-1 antibody is selected from the group consisting of MDX-1106 (nivolumab), MK-3475 (pembrolizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in WO2006/121168. MK-3475, also known as pembrolizumab or lambrolizumab, is an anti-PD-1 antibody described in WO 2009/114335. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. AMP-224, also known as B7-DCIg, is a PD-L2-Fc fusion soluble receptor described in WO 2010/027827 and WO 2011/066342.

In some embodiments, the anti-PD-1 antibody is MDX-1106. Alternative names for “MDX-1106” include MDX-1106-04, ONO-4538, BMS-936558, and nivolumab. In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO: 11 and/or a light chain variable region comprising the light chain variable region amino acid sequence from SEQ ID NO: 12.

In a still further embodiment, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 11)
QVQLVESGGGVVQPGRSLRLDCKASGITFSNSGMHWVRQA
PGKGLEWVAVIWYDGSKRYYADSVKGRFTISRDNSKNTLF
LQMNSLRAEDTAVYYCATNDDYWGQGTLVTVSSASTKGPS
VFPLAPCSRSTSESTAALGCLVKDYFPEPVTVSWNSGALT
SGVHTFPAVLQSSGLYSLSSVVTVPSSSLGTKTYTCNVDH
KPSNTKVDKRVESKYGPPCPPCPAPEFLGGPSVFLFPPKP
KDTLMISRTPEVTCVVVDVSQEDPEVQFNWYVDGVEVHNA
KTKPREEQFNSTYRVVSVLTVLHQDWLNGKEYKCKVSNKG
LPSSIEKTISKAKGQPREPQVYTLPPSQEEMTKNQVSLTC
LVKGFYPSDIAVEWESNGQPENNYKTTPPVLDSDGSFFLY
SRLTVDKSRWQEGNVFSCSVMHEALHNHYTQKSLSLSLGK,

and
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 12)
EIVLTQSPATLSLSPGERATLSCRASQSVSSYLAWYQQKP
GQAPRLLIYDASNRATGIPARFSGSGSGTDFTLTISSLEP
EDFAVYYCQQSSNWPRTFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC.

It is expressly contemplated that such PD-1 binding antagonist antibodies for use in any of the instances enumerated above may have any of the features, singly or in combination, described in any one of Subsections 1-7 of Section C below.

### C. Antibodies

1. Antibody Affinity

In certain instances, an antibody provided herein (e.g., an anti-PD-L1 antibody or an anti-PD-1 antibody) has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, Kd is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20TH; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, Kd is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20TH) surfactant (PBST) at 25° C. at a flow rate of approximately μl/min. Association rates (kw) and dissociation rates (k_{off}) are calculated using a simple one-to-one

Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an antibody (e.g., an anti-PD-L1 antibody or an anti-PD-1 antibody) provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). Fora review of scFv fragments, see, e.g., PluckthOn, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain instances, an antibody (e.g., an anti-PD-L1 antibody or an anti-PD-1 antibody) provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl. Acad. Sci. USA 86:10029-10033 (1989); US Pat. Nos. 5, 821, 337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol, 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol Chem. 272:10678-10684 (1997) and Rosok et al., J. Biol Chem. 271:22611-22618 (1996)).

4. Human Antibodies

In certain instances, an antibody (e.g., an anti-PD-L1 antibody or an anti-PD-1 antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies (e.g., anti-PD-L1 antibodies and anti-PD-1 antibodies) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: US Patent No. and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In any one of the above aspects, an antibody (e.g., an anti-PD-L1 antibody or an anti-PD-1 antibody) provided herein may be a multispecific antibody, for example, a bispecific antibody. Multispecific antibodies are monoclonal antibodies that have binding specificities for at least two different sites. In certain instances, an antibody provided herein is a multispecific antibody, e.g., a bispecific antibody. In certain instances, one of the binding specificities is for PD-L1 and the other is for any other antigen. In certain instances, bispecific antibodies may bind to two different epitopes of PD-L1. Bispecific antibodies may also be used to localize cytotoxic agents to cells which express PD-L1. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (see, e.g., WO 2009/089004A1); cross-linking two or more antibodies or fragments (see, e.g., U.S. Pat. No. 4,676,980, and Brennan et al., Science 229: 81 (1985)); using leucine zippers to produce bi-specific antibodies (see, e.g., Kostelny et al., J. Immunol. 148(5): 1547-1553 (1992)); using “diabody” technology for making bispecific antibody fragments (see, e.g., Hollinger et al., Proc. Natl. Acad. Sci. USA 90:6444-6448 (1993)); using single-chain Fv (sFv) dimers (see, e.g., Gruber et al., J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to PD-L1 as well as another, different antigen.

7. Antibody Variants

In certain instances, amino acid sequence variants of the antibodies provided herein (e.g., anti-PD-L1 antibodies and anti-PD-1 antibodies) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

### I. Substitution, Insertion, and Deletion Variants

In certain instances, antibody variants having one or more amino acid substitutions are provided. Sites of interest for substitutional mutagenesis include the HVRs and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved Antibody-Dependent Cell-Mediated Cytotoxicity (ADCC) or Complement Dependent Cytotoxicity (CDC).

TABLE A
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen-contacting residues in the HVRs. In certain instances of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

II. Glycosylation Variants

In certain instances, antibodies of the invention can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention may be made in order to create antibody variants with certain improved properties.

In one instance, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, for example, U.S. Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl. No. US 2003/0157108 A1; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

III. Fc Region Variants

In certain instances, one or more amino acid modifications may be introduced into the Fc region of an antibody of the invention, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g., Hellstrom, I. et al. Proc. Natl. Acad. Sci. USA 83:7059-7063 (1986)) and Hellstrom, I et al., Proc. Natl. Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI))). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. USA 95:652-656 (1998). C1g binding assays may also be carried out to confirm that the antibody is unable to bind C1g and hence lacks CDC activity. See, e.g., C1g and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg et al., Blood. 101:1045-1052 (2003); and Cragg et al., Blood. 103:2738-2743 (2004)). FcRn binding and in vivo clearance/half-life determinations can also be performed using methods known in the art (see, e.g., Petkova et al. Intl. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001))

In certain instances, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1g binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

IV. Cysteine Engineered Antibody Variants

In certain instances, it may be desirable to create cysteine engineered antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

V. Antibody Derivatives

In certain instances, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

VI. Immunoconjugates

The invention also provides immunoconjugates comprising an antibody provided herein (e.g., an anti-PD-L1 antibody or an anti-PD-1 antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one instance, an immunoconjugate is an antibody-drug conjugate (ADC) in which an antibody is conjugated to one or more drugs, including but not limited to a maytansinoid (see U.S. Pat. Nos. 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron. Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate) which are commercially available (e.g., from Pierce Biotechnology, Inc., Rockford, IL., U.S.A).

### V. PHARMACEUTICAL COMPOSITIONS AND FORMULATIONS

Pharmaceutical compositions and formulations as described herein can be prepared by mixing the active ingredient(s) (e.g., an anti-PD-L1 antibody, e.g., atezolizumab) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®; Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. It is understood that any of the above pharmaceutical compositions or formulations may include an immunoconjugate described herein in place of, or in addition to, a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The compositions and formulations herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, for example, by filtration through sterile filtration membranes.

### VI. ARTICLES OF MANUFACTURE AND KITS

In another aspect of the disclosure, an article of manufacture or kit containing materials useful for the treatment, prevention, and/or diagnosis of individuals is provided.

In some instances, such articles of manufacture or kits can be used to identify an individual having a cancer (e.g., lung cancer (e.g., NSCLC), bladder cancer (e.g., UC), kidney cancer (e.g., RCC), or breast cancer (e.g., TNBC)) who may benefit from a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). Such articles of manufacture or kits may include (a) reagents for determining the immune-score expression level of one or more of genes in a sample from the individual and (b) instructions for using the reagents to identify an individual having a cancer (e.g., lung cancer (e.g., NSCLC), bladder cancer (e.g., UC), kidney cancer (e.g., RCC), or breast cancer (e.g., TNBC)) who may benefit from a treatment including a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

For example, in some instances, the article of manufacture or kit includes (a) reagents for determining the immune-score expression level of one or more genes set forth in any one of Tables 1-17 in a sample from the individual and (b) instructions for using the reagents to identify an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)).

In another example, provided herein is a kit comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) and instructions to administer the PD-L1 axis binding antagonist to an individual having a cancer (e.g., lung cancer (e.g., NSCLC), bladder cancer (e.g., UC), kidney cancer (e.g., RCC), or breast cancer (e.g., TNBC)) who has been identified as one who may benefit from a treatment comprising the PD-L1 binding antagonist in accordance with any one of the methods disclosed herein.

In another example, provided herein is a kit comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)) and instructions to administer the PD-L1 axis binding antagonist to an individual having a cancer (e.g., lung cancer (e.g., NSCLC), bladder cancer (e.g., UC), kidney cancer (e.g., RCC), or breast cancer (e.g., TNBC)) who has been selected for a treatment comprising the PD-L1 binding antagonist in accordance with the method of any one of the methods disclosed herein.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising means for determining the expression level of one or more (e.g., 1, 2, 3, 4, 5, 6, 7, 8, 9, 10, or 11) of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising reagents for determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's overall survival (OS) as compared to treatment without the PD-L1 axis binding antagonist.

In some instances, the kit comprises reagents for determining the expression level of two or more of genes CD79A, CD19, BANK1, JCHAIN, SLAMF7, BTK, TNFRSF17, IGJ, IGLL5, RBPJ, and MZB1 in a sample from the individual. In some instances, the kit comprises reagents for determining the expression level of three or more of genes CD79A, CD19, BANK1, JCHAIN, SLAMF7, BTK, TNFRSF17, IGJ, IGLL5, RBPJ, and MZB1 in a sample from the individual. In some instances, the kit comprises reagents for determining the expression level of four or more of genes CD79A, CD19, BANK1, JCHAIN, SLAMF7, BTK, TNFRSF17, IGJ, IGLL5, RBPJ, and MZB1 in a sample from the individual. In some instances, the kit comprises reagents for determining the expression level of five or more of genes CD79A, CD19, BANK1, JCHAIN, SLAMF7, BTK, TNFRSF17, IGJ, IGLL5, RBPJ, and MZB1 in a sample from the individual. In some instances, the kit comprises reagents for determining the expression level of six or more of genes CD79A, CD19, BANK1, JCHAIN, SLAMF7, BTK, TNFRSF17, IGJ, IGLL5, RBPJ, and MZB1 in a sample from the individual. In some instances, the kit comprises reagents for determining the expression level of seven or more of genes CD79A, CD19, BANK1, JCHAIN, SLAMF7, BTK, TNFRSF17, IGJ, IGLL5, RBPJ, and MZB1 in a sample from the individual. In some instances, the kit comprises reagents for determining the expression level of CD79A, CD19, BANK1, JCHAIN, SLAMF7, BTK, TNFRSF17, IGJ, IGLL5, RBPJ, and MZB1 in a sample from the individual.

The kit may include reagents for determining the expression level of any combination of B cell signature genes. For example, the combination may include two genes selected from CD79A, CD19, BANK1, JCHAIN, SLAMF7, BTK, TNFRSF17, IGJ, IGLL5, RBPJ, and MZB1, e.g., any one of the combinations set forth in Table 3. In another example, the combination may include three genes selected from CD79A, CD19, BANK1, JCHAIN, SLAMF7, BTK, TNFRSF17, IGJ, IGLL5, RBPJ, and MZB1, e.g., any one of the combinations set forth in Table 4. In another example, the combination may include four genes selected from CD79A, CD19, BANK1, JCHAIN, SLAMF7, BTK, TNFRSF17, IGJ, IGLL5, RBPJ, and MZB1, e.g., any one of the combinations set forth in Table 5. In another example, the combination may include five genes selected from CD79A, CD19, BANK1, JCHAIN, SLAMF7, BTK, TNFRSF17, IGJ, IGLL5, RBPJ, and MZB1, e.g., any one of the combinations set forth in Table 6. In another example, the combination may include six genes selected from CD79A, CD19, BANK1, JCHAIN, SLAMF7, BTK, TNFRSF17, IGJ, IGLL5, RBPJ, and MZB1, e.g., any one of the combinations set forth in Table 7. In another example, the combination may include seven genes selected from CD79A, CD19, BANK1, JCHAIN, SLAMF7, BTK, TNFRSF17, IGJ, IGLL5, RBPJ, and MZB1, e.g., any one of the combinations set forth in Table 8.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising means for determining the expression level of one or more (e.g., 1, 2, 3, 4, 5, 6, 7, 8, 9, 10, 11, 12, 13, or 14) of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising reagents for determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

In some instances, the kit comprises reagents for determining the expression level of two or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual. In some instances, the kit comprises reagents for determining the expression level of three or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual. In some instances, the kit comprises reagents for determining the expression level of four or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual. In some instances, the kit comprises reagents for determining the expression level of five or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual. In some instances, the kit comprises reagents for determining the expression level of six or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual. In some instances, the kit comprises reagents for determining the expression level of seven or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual. In some instances, the kit comprises reagents for determining the expression level of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual.

The kit may include reagents for determining the expression level of any combination of plasma B cell signature genes. For example, the combination may include two genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 10. In another example, the combination may include three genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 11. In another example, the combination may include four genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 12. In another example, the combination may include five genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 13. In another example, the combination may include six genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 14. In another example, the combination may include seven genes selected from MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5, e.g., any one of the combinations set forth in Table 15.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising reagents for determining the presence of a TLS in a tumor sample from the individual, wherein the presence of a TLS in the tumor sample identifies the individual as one who may benefit from a treatment comprising a PD-L1 axis binding antagonist.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising reagents for determining the expression level of one or more (e.g., 1, 2, 3, 4, 5, 6, 7, 8, 9, 10, 11, or 12) of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the one or more genes that is above a reference immune-score expression level of the one or more genes identifies the individual as one who may benefit from a treatment comprising a PD-L1 axis binding antagonist.

In some instances, the kit comprises reagents for determining the expression level of two or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of three or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of four or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of five or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of six or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of seven or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of eight or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of nine or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of ten or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of eleven or more of genes CCL2, CCL3, CCL4, CCL5, CCL8, CCL18, CCL19, CCL21, CXCL9, CXCL10, CXCL11, and CXCL13 in a sample from the individual. In some instances, the kit comprises reagents for determining the expression level of CCL2, CCL3, CCL4, CCL5, CCL8, CCL18, CCL19, CCL21, CXCL9, CXCL10, CXCL11, and CXCL13 in a sample from the individual.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising reagents for determining the number of B cells in a tumor sample from the individual, wherein a number of B cells in the tumor sample that is above a reference number of B cells identifies the individual as one who may benefit from a treatment comprising a PD-L1 axis binding antagonist.

In another example, provided herein is a kit for identifying an individual having a cancer (e.g., lung cancer (e.g., NSCLC), bladder cancer (e.g., UC), kidney cancer (e.g., RCC), or breast cancer (e.g., TNBC)) who may benefit from a treatment comprising a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)), the kit comprising reagents for determining whether the individual has clonally expanded B cells in a tumor sample from the individual, wherein clonally expanded B cells in the sample identify the individual as one who may benefit from a treatment comprising a PD-L1 axis binding antagonist.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, florescent, or radioisotope label.

In some instances, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of embodiments. In one instance, the article of manufacture or kit includes a container, a label on the container, and a composition contained within the container, wherein the composition includes one or more polynucleotides that hybridize to a complement of a gene listed herein (e.g., one or more genes set forth in any one of Tables 1-17) under stringent conditions, and the label on the container indicates that the composition can be used to evaluate the presence of a gene listed herein (e.g., one or more of genes one or more genes set forth in any one of Tables 1-17) in a sample, and wherein the kit includes instructions for using the polynucleotide(s) for evaluating the presence of the gene RNA or DNA in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

In one instance, the article of manufacture or kit includes a container, a label on the container, and a composition contained within the container, wherein the composition includes one or more antibodies that specifically bind to any biomarker disclosed herein (e.g., one or more genes set forth in any one of Tables 1-17, a TLS, or a B cell (including a clonally expanded B cell)) under stringent conditions, and the label on the container indicates that the composition can be used to evaluate the presence of a biomarker listed herein in a sample, and wherein the kit includes instructions for using the antibody or antibodies or evaluating the presence of the biomarker in a particular sample type.

### VII. EXAMPLE

The following is an example of the methods of the invention. It is understood that various other embodiments may be practiced, given the general description provided above.

### Example 1. Tumor Infiltrating B Cells, Including Plasma B Cells, and Tertiary Lymphoid Structures are Associated with Tumor Response to PD-L1 Blockade in NSCLC

An unsupervised differential analysis of gene expression was conducted in pre-treatment biopsies from the Phase 2 POPLAR trial (NCT01903993). The study confirmed the findings of the Phase 3 OAK (NCT02008227) and BIRCH (NCT02031458) clinical trials. This analysis, which is described in further detail in this Example, revealed a strong association of the presence of B cells, particularly plasma B cells, and tertiary lymphoid structure (TLS) in the tumors of patients who went on to show a sustained survival benefit with atezolizumab.

Unsupervised Differential Gene Expression Analysis Shows B Cell Genes Associate with Overall Survival (OS) Post Atezolizumab Treatment

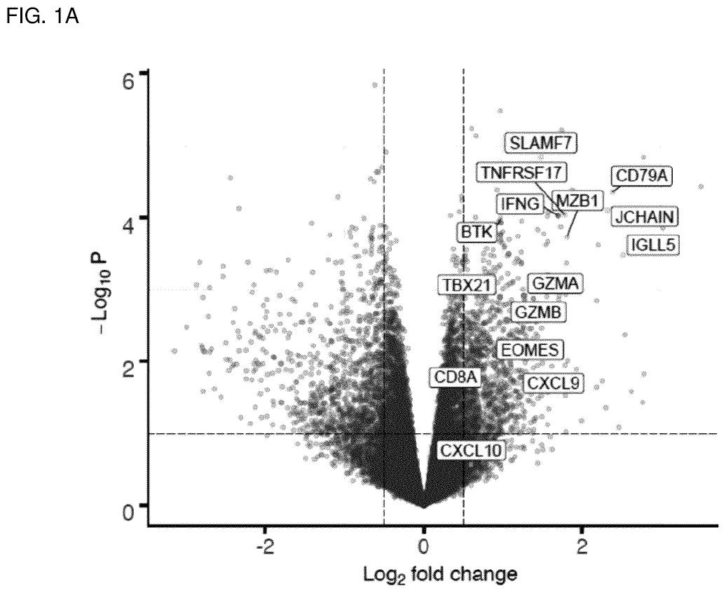

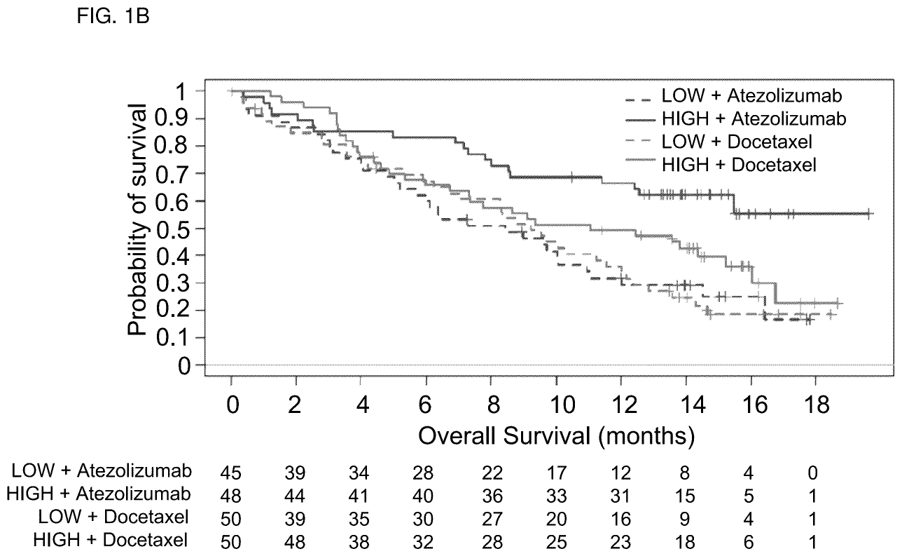

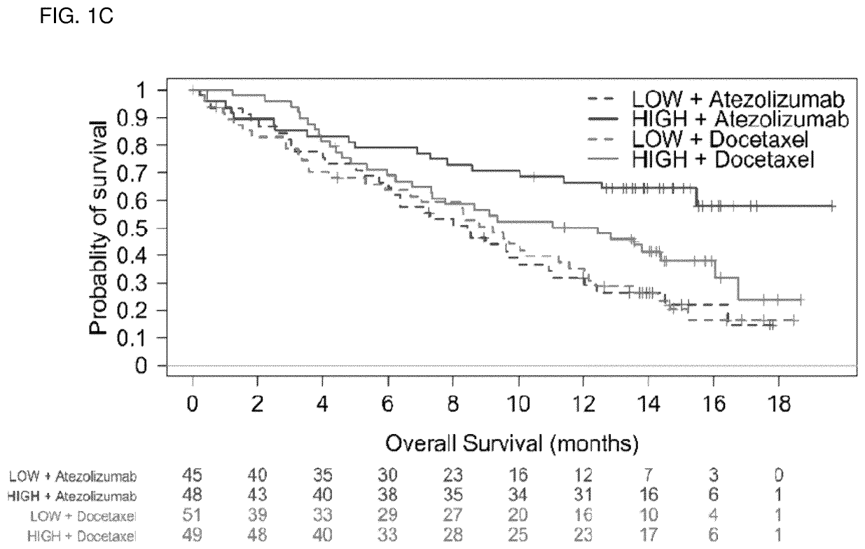

We analyzed genome-wide RNA-sequence data derived from archival tumor samples of patients from the POPLAR trial. We compared the gene expression data from atezolizumab treated patients with survival of <6 months (n=24) to those with survival ≥12 months (n=43) (Lee, J., et al. Reinvigorating Exhausted T Cells by Blockade of the PD-1 Pathway. For lmmunopathol. Dis. Therap. 6, 7-17 (2015)). Unsupervised differential gene expression analysis revealed that apart from the expected T cell effector genes, several B cell genes were significantly associated with OS after correcting for multiple hypothesis testing (FIGS. 1A and 7).

To evaluate whether these B cell gene signatures (CD79A, SLAMF7, BTK, TNFRSF17, IGJ or JCHAIN, IGLL5, RBPJ, and MZB1) were associated with OS for both treatment arms, the mean z-score of these genes was associated with benefit in the atezolizumab arm (>median, HR=0.39, CI=0.22-0.68, p<0.001) and less associated with chemotherapy or docetaxel treatment (relative to docetaxel, HR=0.74, CI=0.52-1.07, p=0.1) (FIG. 1B). Of all these genes, the B cell receptor gene (CD79A) was one of the most significant differentially expressed gene, and high expression of CD79A was associated with OS benefit in the atezolizumab treatment arm (>median, HR=0.36, CI=0.2-0.64, p<0.001) (FIG. 1C).

A B Cell Transcriptional Signature Associates with Prolonged OS in Patients Treated with Atezolizumab

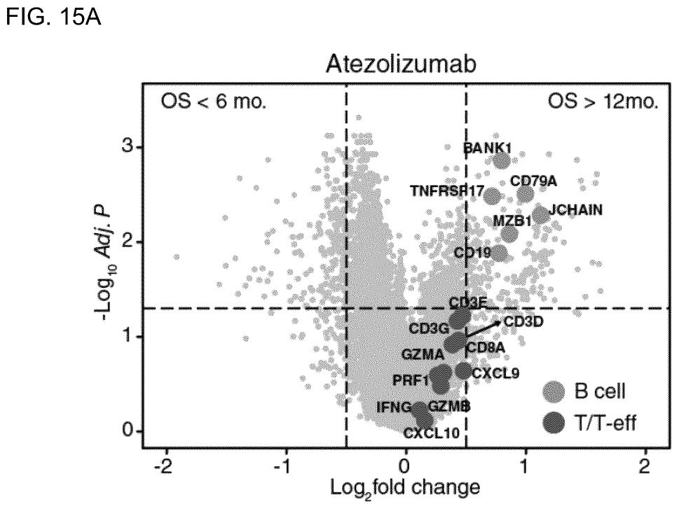

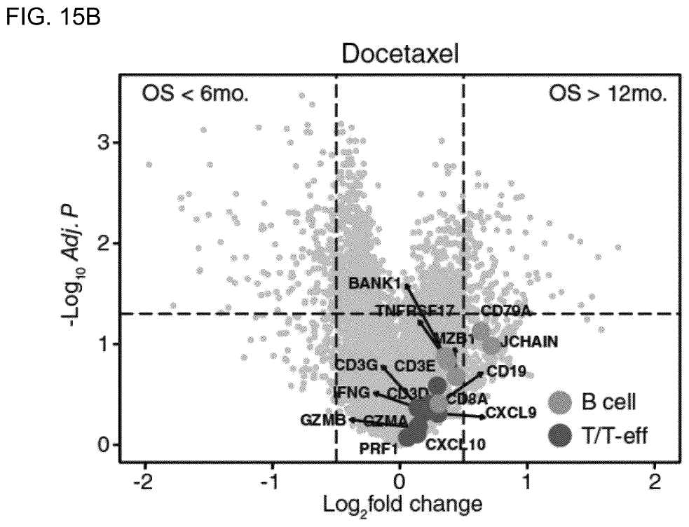

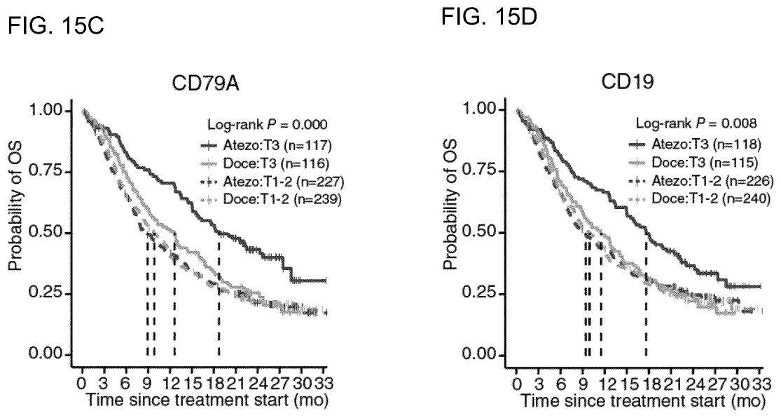

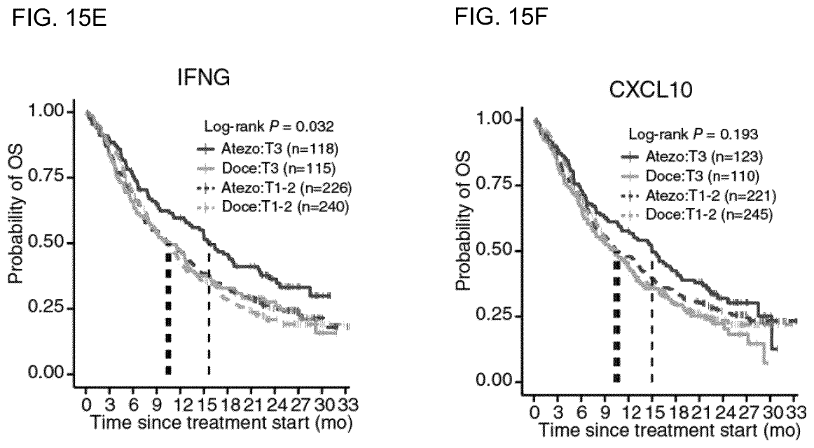

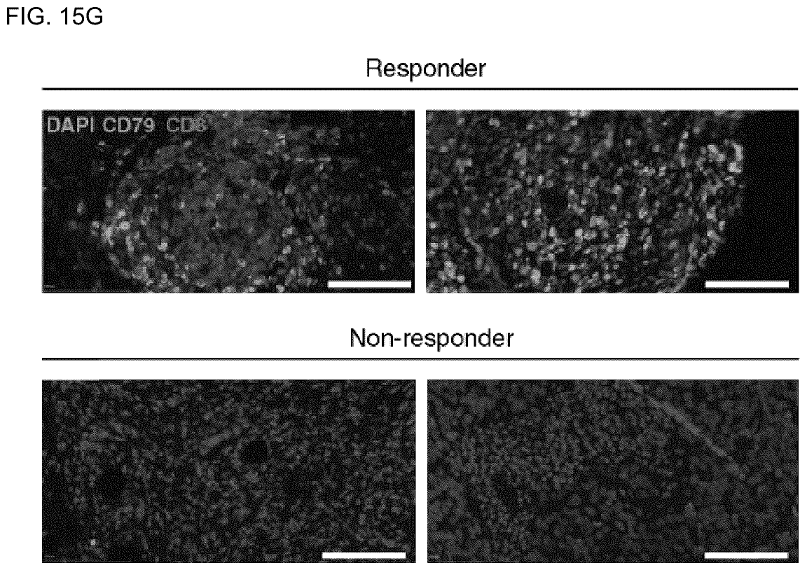

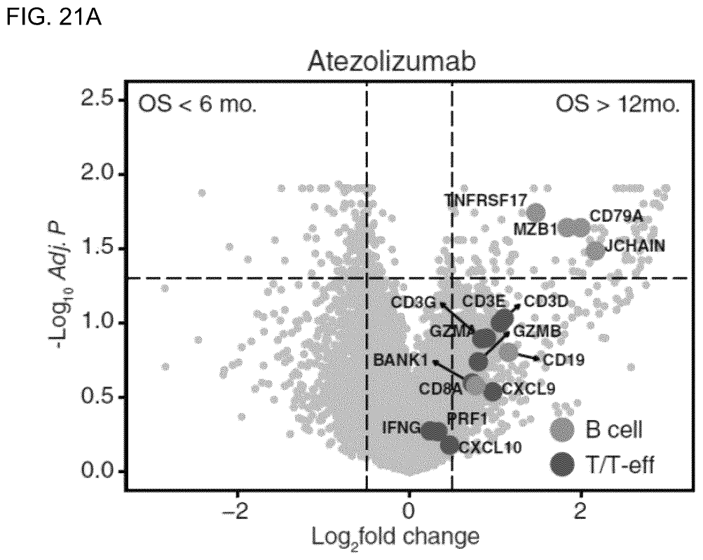

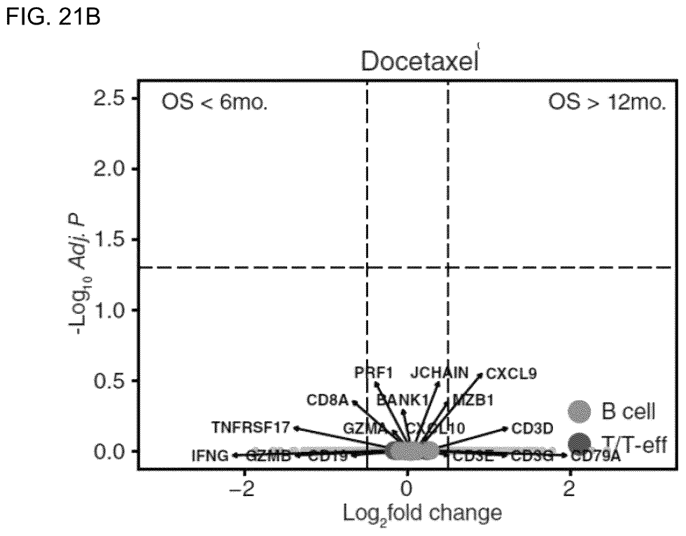

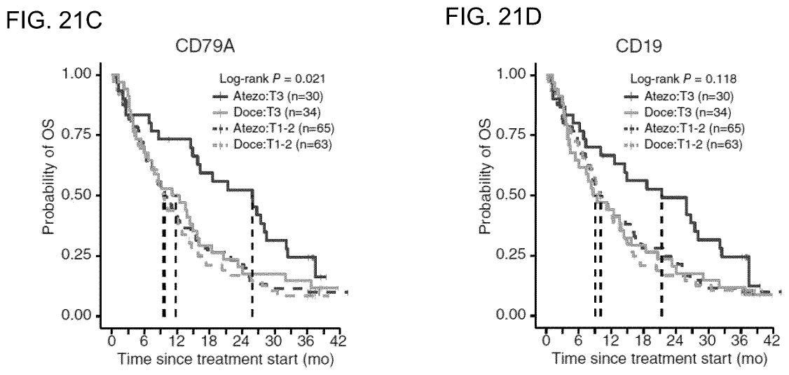

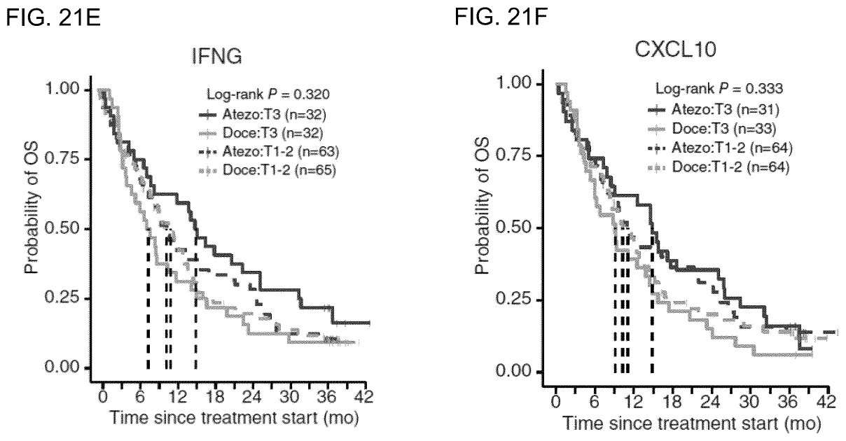

We additionally sought to identify differentially expressed genes (DEGs) between tumors from responders and non-responders to atezolizumab in OAK, comparing patients with long survival (>12 months, n=313) and those with short survival (<6 months, n=205) within each treatment arm. 817 genes were differentially expressed between patients with long or short OS with atezolizumab (FDR-corrected P<0.01, absolute logFC>=0.5). The top over-expressed genes were enriched for genes associated with B and plasma cell biology, including CD19, CD79A, BANK1, JCHAIN, MZB1, and TNFRSF17 (BCMA) (FIG. 15A). Genes associated with cytotoxic T-cell and IFN signals (CD3E,D,G, CD8A, GZMA-B, IFNG, CXCL9-10) were also detected, albeit not significantly. None of these genes were significant in the docetaxel arm (FIG. 15B), suggesting a predictive value of these B/plasma cell genes specifically for response to atezolizumab. These observations were validated in the phase II randomized clinical trial POPLAR (FIGS. 21A and 21B).

We then validated results from linear modeling by deriving Kaplan-Meier curves in OAK, categorizing gene expression by tertiles and comparing high expression (T3) vs. low/intermediate (T1-T2) combined. High expression of the B cell markers CD79A (HR atezolizumab: 0.54 [0.41-0.72]; HR chemo: 0.88 [0.67-1.13]) and CD19 (HR atezolizumab: 0.65 [0.50-0.86]; HR chemo: 0.94 [0.72-1.2]) provided increased OS benefit over IFNG (HR atezolizumab: 0.73 [0.55-0.96]; HR chemo: 0.95 [0.73-1.23]) or the IFN-inducible chemokine CXCL10 (HR atezolizumab: 0.85 [0.65-1.11]; HR chemo: 1.11 [0.85-1.44]) (FIGS. 15C-15F). Again, similar results were observed for POPLAR (FIGS. 21C-21F).

To confirm these transcriptional findings at the protein level, we evaluated baseline tumor samples from two responders and two non-responders to atezolizumab by immunofluorescence (IF) staining for CD8, CD79 and Ki67 using multiplex IF. Abundant CD79+ B cells were detected in tumors from patients with objective responses compared to those without (FIG. 15G). In addition, multiplex immunohistochemistry (INC) detected regions where CD8+ T cells were in close proximity (9.9±1.69 μm apart) with CD79+ B cells suggestive of B cell-T cell interactions.

Identification of B Cell Subsets by Single Cell RNA-Seq

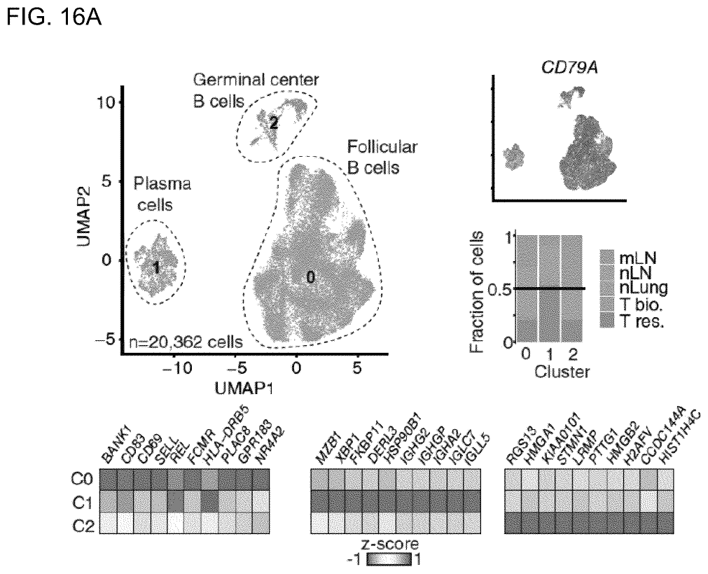

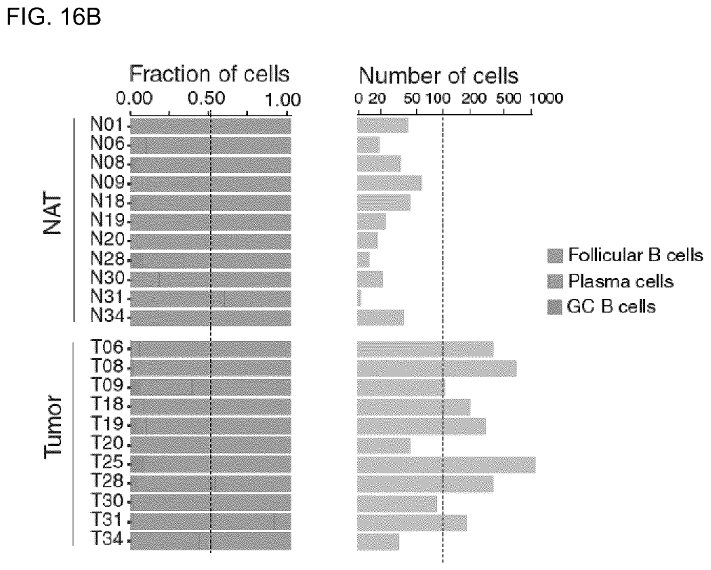

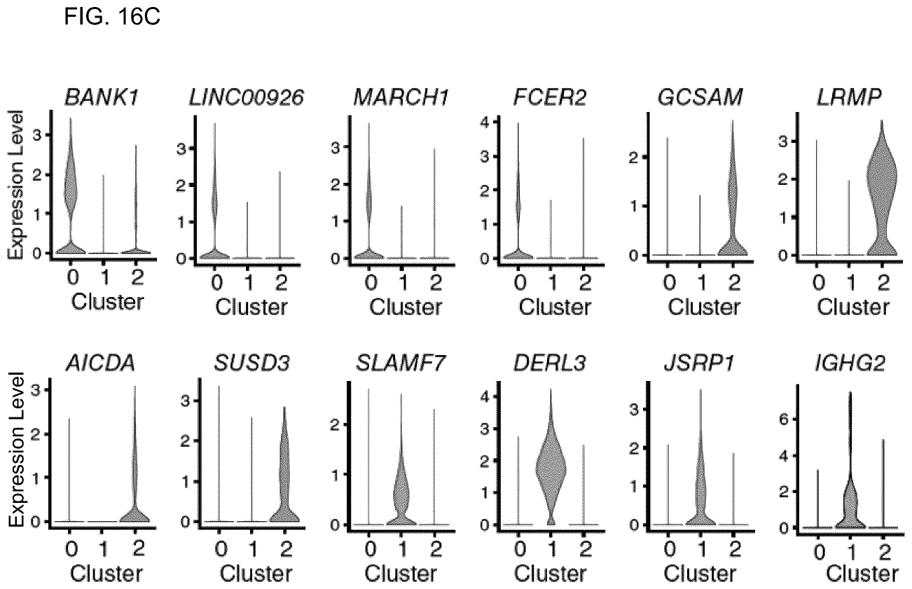

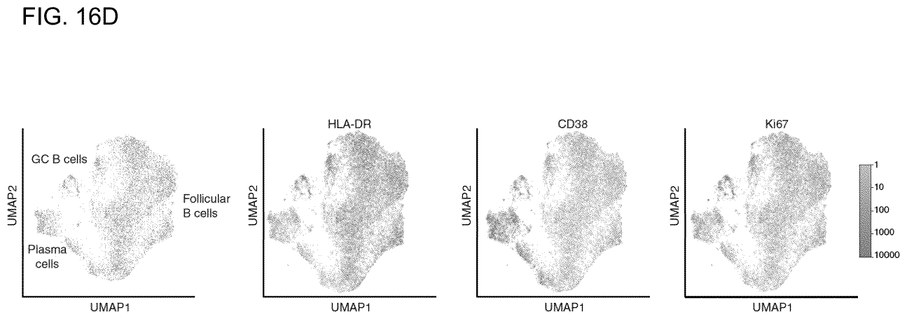

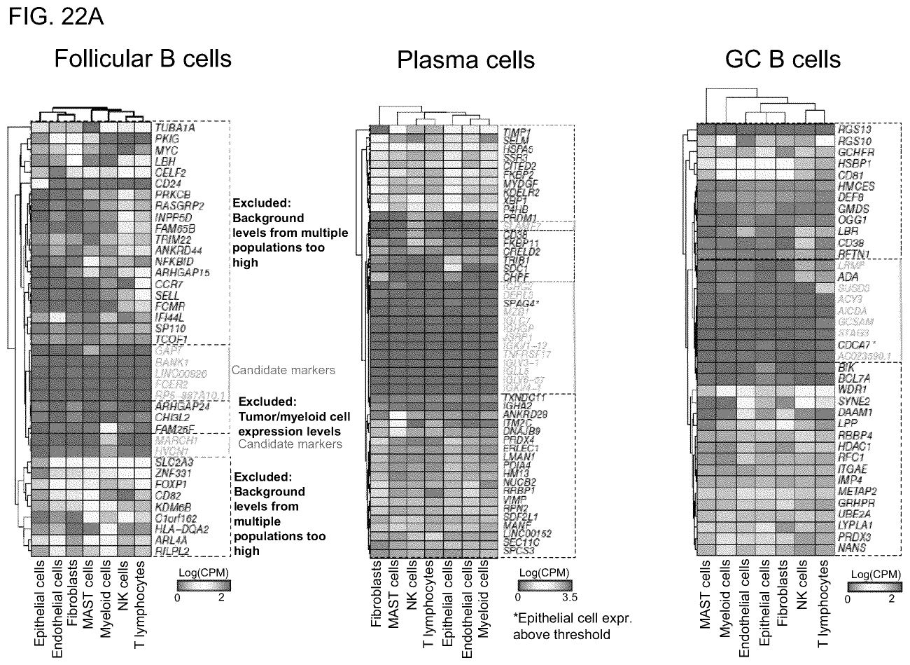

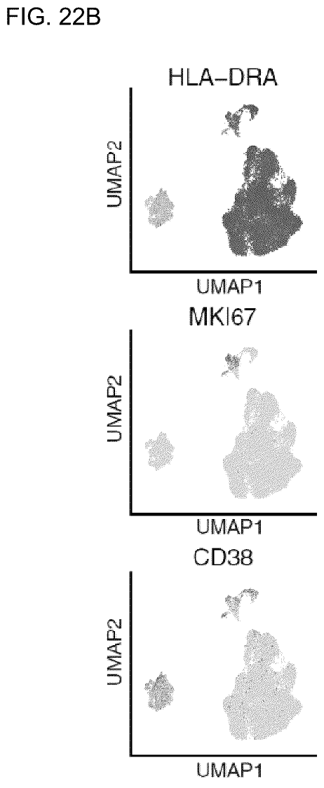

To further characterize the B cell compartment involved in response to atezolizumab, we analyzed a large scRNA-seq dataset of 208,506 cells collected from lung and peripheral tissue from 44 NSCLC patients, focusing on the B/plasma cell populations. Aggregating cells from tumor and draining lymph node compartments, dimensionality reduction with uniform manifold approximation and projection (UMAP) and graph-based clustering identified three major CD79A+B cell subsets across tumors, including follicular B cells, germinal center (GC) B cells and plasma cells (FIG. 16A). While we found an increased number of B/plasma cells in tumor tissue compared to normal adjacent tissue (NAT), the composition of GC, follicular B and plasma cells within these B cell populations was similar between tumor and NAT from the same patient (FIG. 16B). Transcriptional signatures specific to each subset were identified. Follicular B cells were enriched for CD83, CD69, SELL and BANK1 (Aiba et al., 2006). Plasma cells were enriched for several immunoglobulin transcripts (IGHG2, 137 IGHGP, IGHA2), MZB1, DERL3 and XBP1. Germinal center B cells were enriched for HMGA1, HMGB2, and RGS13. We refined these signatures by probing the expression of these transcripts across multiple immune (myeloid, NK and T cells), stromal (fibroblasts, endothelial cells) and epithelial/tumor cell populations (FIG. 22A) from the same patients, only retaining genes specific for B cell compartments. We further focused only on markers with a high degree of specificity for the individual B cell populations (FIGS. 16C and 22A)

We complemented this transcriptional approach with mass cytometry, using a 38-marker CyTOF panel on single cell suspensions from six procured NSCLC tumors. This analysis confirmed the presence of three intratumoral B cell populations, based on CD19, HLA-DR, CD38 and Ki67 staining (plasma cells: CD38++, HLA-DR-, Ki67-; GC B cells: CD38+, HLA-DR+, Ki67+; Follicular B cells: CD38-, HLA-DR+, Ki67-) (FIG. 16D). These specific markers were also validated for similar expression patterns in the scRNA-seq dataset (FIG. 22B). In total, single-cell analysis identified three main intratumoral B cell subpopulations with highly specific markers.

High Plasma Cell Signature Predicts OS Benefit from Atezolizumab

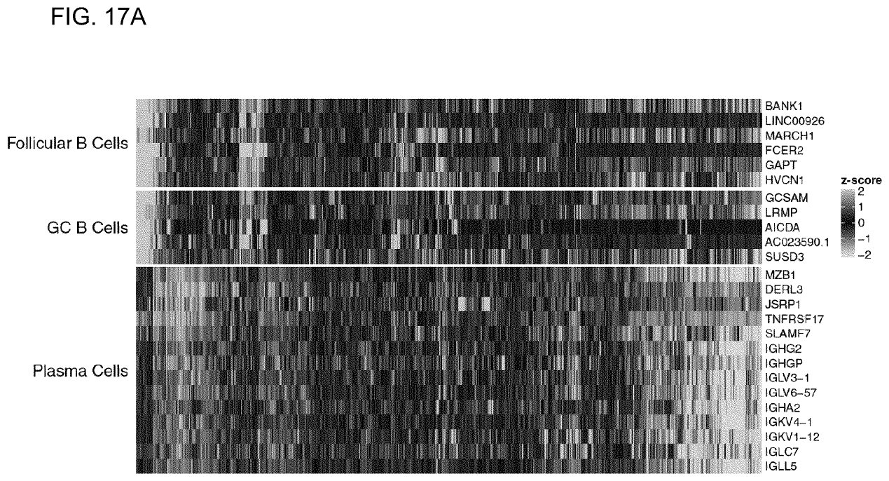

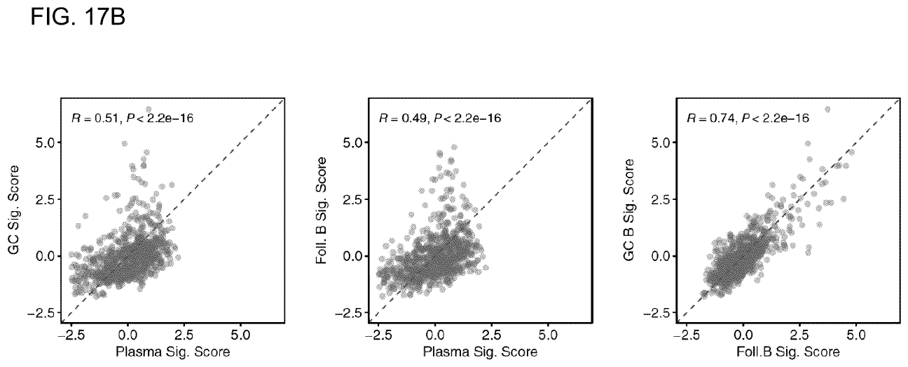

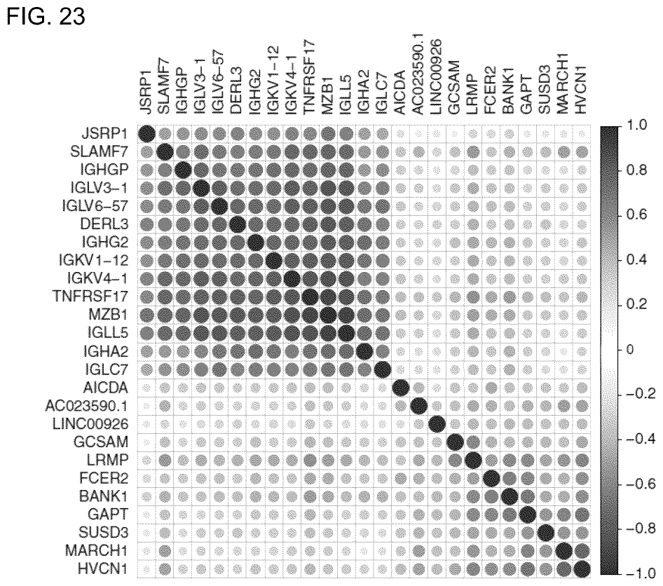

We then asked whether these B cell signatures identified from scRNA-seq analyses could predict patient survival following treatment with atezolizumab or docetaxel. We first validated that signatures derived from scRNA-seq could be applied to bulk RNA-seq data by ensuring gene co-expression through hierarchical clustering (FIG. 17A) and gene-to-gene correlation (FIG. 23). These analyses revealed high correlation between germinal center and follicular B cells in bulk tumors (FIG. 17B, R=0.74), while plasma cell-high tumors appeared more distinct. The plasma cell, GC B cell and follicular B cell signatures are summarized in Table 18.

TABLE 18
Follicular B cell, GC B cell, and plasma B cell gene signatures

| Follicular B cells | GC B cells | Plasma cells |
|---|---|---|
| BANK1 | GCSAM | MZB1 |
| LINC00926 | LRMP | DERL3 |
| FCER2 | AICDA | JSRP1 |
| GAPT | AC023590.1 | TNFRSF17 |
| HVCN1 | SUSD3 | SLAMF7 |
| | | IGHG2 |
| | | IGHGP |
| | | IGLV3-1 |
| | | IGLV6-57 |
| | | IGHA2 |
| | | IGKV4-1 |
| | | IGKV1-12 |
| | | IGLC7 |
| | | IGLL5 |

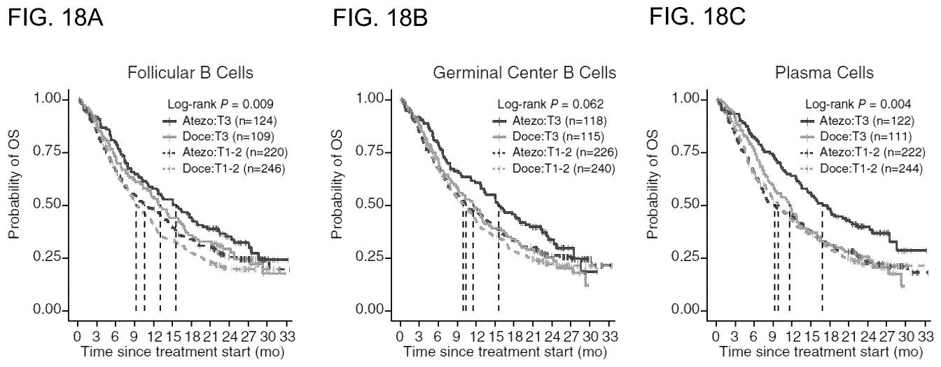

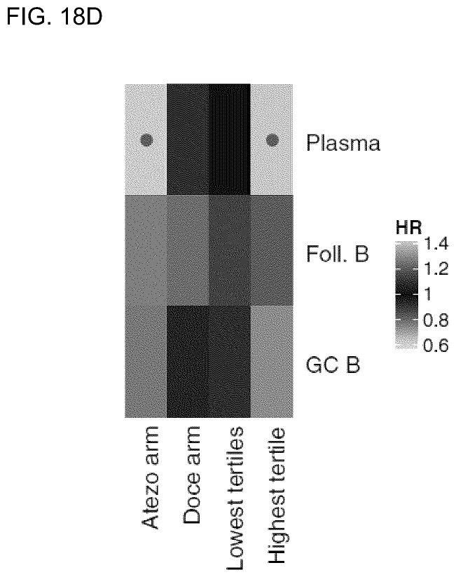

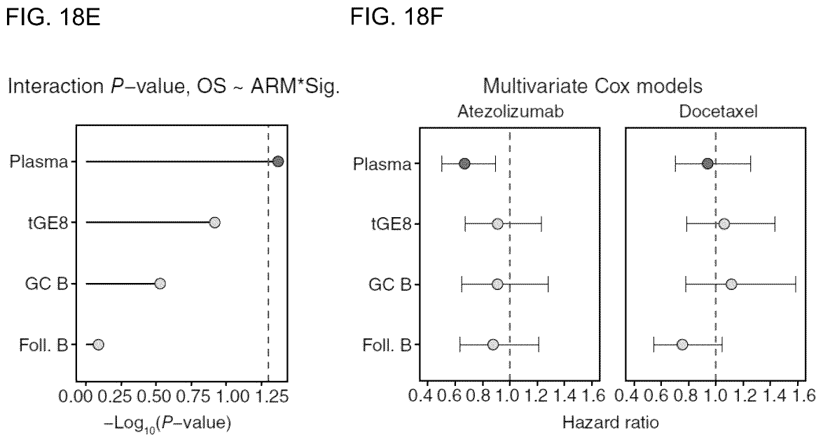

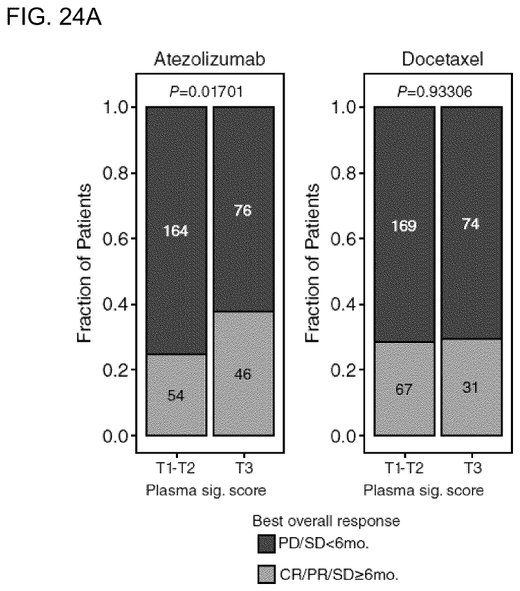

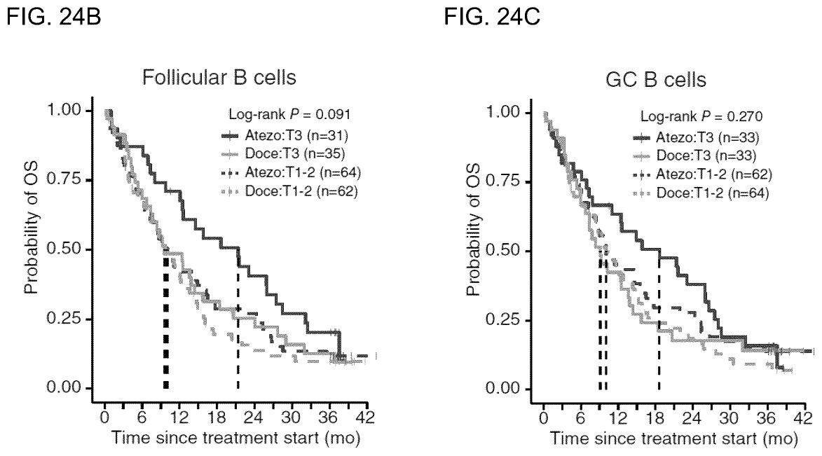

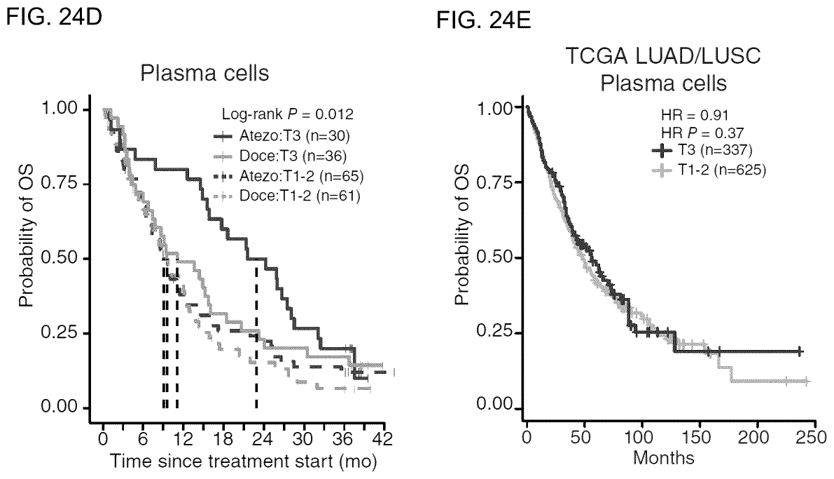

We then dichotomized these signatures using tertile split and comparing high (T3) vs. low/intermediate (T1/T2) expression groups. Kaplan-Meier analysis revealed that, while a trend towards OS benefit was observed with atezolizumab for the three signatures, only the plasma cell signature was significant (atezolizumab high vs. low HR=0.63 [0.48-0.83]; chemo high vs. low HR=0.92 [0.71-1.2]; atezolizumab high vs. chemo high HR=0.64 [0.47-0.88]) (FIGS. 18A-18D). Moreover, plasma cell high tumors showed significantly more patients who experienced a best overall response of complete response, partial response, or durable stable disease (SD 6 months) (FIG. 24A). There was a similar strong stratification of overall survival by the plasma cell signature in POPLAR (FIGS. 24B-24D). The predictive value of the plasma cell signature was confirmed in models testing the interaction of each signature with treatment arms (FIG. 18E). Importantly, neither the GC B cell, follicular B cell nor our 8-gene T-effector signature tGE8 (composed of IFNG, CXCL9, CD8A, GZMA, GZMB, CXCL10, PRF1, TBX21) were significant in these models (FIG. 18E). The predictive value of the plasma cell signature also held in multivariate models including all four signatures (atezolizumab HR=0.67 [0.50-0.89]; chemo HR=0.94 [0.70-1.26]), confirming that the effect observed with the plasma cell signature is specific and independent of CD8 T cell presence (FIG. 18F). In other indications, increased B cells have been described to have prognostic implications in TOGA; however, our data indicate a strong predictive signal for plasma cell-rich NSCLC tumors specifically with immune checkpoint inhibition. Indeed, the plasma cell signature dichotomized by tertile split in TOGA NSCLC tumors had no impact on stratification of overall survival (FIG. 24E). In total, these data suggest that plasma-cell rich tumors portend overall survival benefit in specifically immune checkpoint inhibitor treated patients.

The Plasma Cell Signature is Enriched in Tumors with Tertiary Lymphoid Structures and/or Lymphoid Aggregates

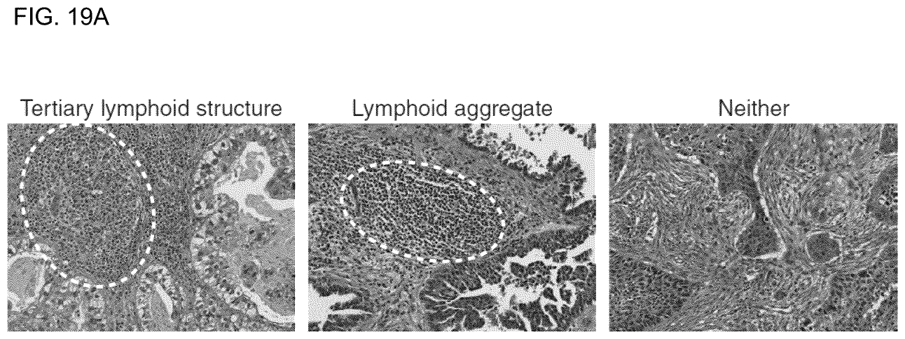

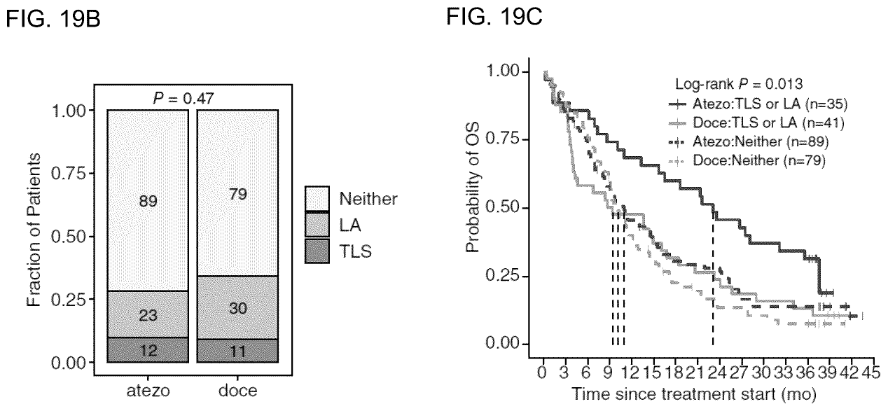

We evaluated tumor hematoxylin & eosin (H&E) slides for the presence of TLS or lymphoid aggregates only (LA, no observed germinal centers) in POPLAR (FIG. 19A). Of the 254 patient samples analyzed, 9% had TLS-like structures with germinal centers, 21% had lymphoid aggregates (with no observed germinal centers), and the remaining 70% had no detectable TLS-like or lymphoid aggregates. Lymph node metastases samples (4% of all samples) were excluded from this analysis (FIG. 19B). Patients with tumors containing TLS and/or LA exhibited significantly increased 191 OS when treated with atezolizumab (HR=0.60 [0.38-0.94]) but not with docetaxel (HR=0.93 [0.62-0.1.41]) (FIG. 19C).

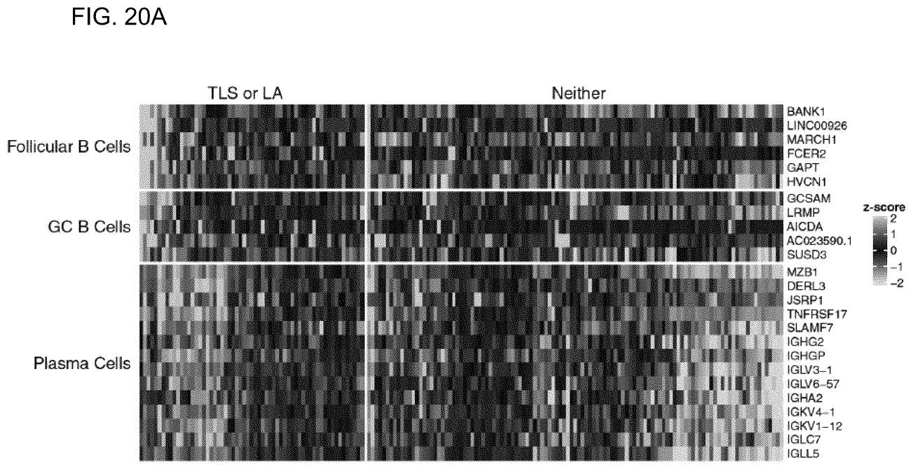

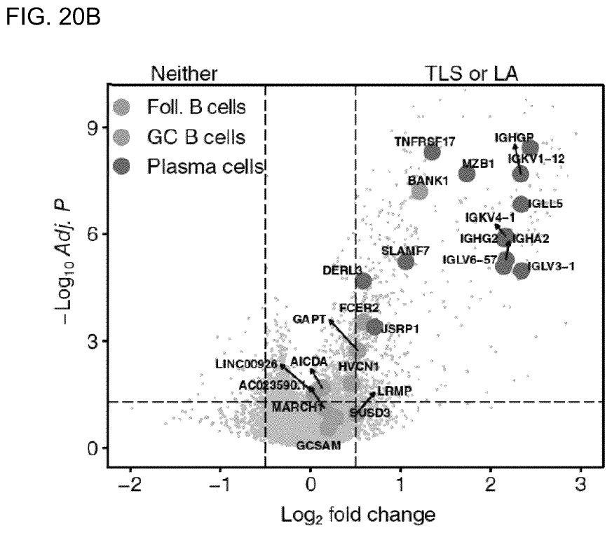

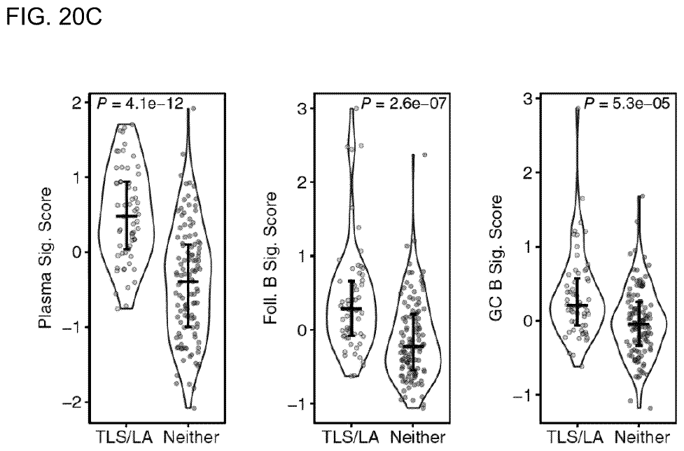

A linear model was then applied to identify DEGs between TLS/LA+ and TLS/LA-tumors. 928 genes were differentially expressed between the two groups (FDR-corrected P<0.01, absolute logFC>=TLS/LA+ tumors were highly enriched for genes from each of the three intratumoral B cell subsets, but especially, plasma cell genes including MZB1, TNFRSF17(BCMA) and immunoglobulins (FIGS. 20A and 20B). Quantitative analysis of the three B cell signatures confirmed the increase in plasma cells in TLS/LA+ tumors (P=4.1e-12). GC B cell (P=2.6e-07) and follicular B cell signatures (P=5.3e-05) were also increased in TLS/LA+ tumors (FIG. 20C).

Overall, our study integrating bulk transcriptomes from two large randomized clinical trials of atezolizumab vs. docetaxel, as well as single-cell RNA and protein measurement, demonstrated that baseline plasma cell enrichment in NSCLC tumors is a strong predictor of OS benefit following immune checkpoint blockade.

B Cell Association with Clinical Benefit Post Atezolizumab Treatment Confirmed in Independent Clinical Trial Datasets

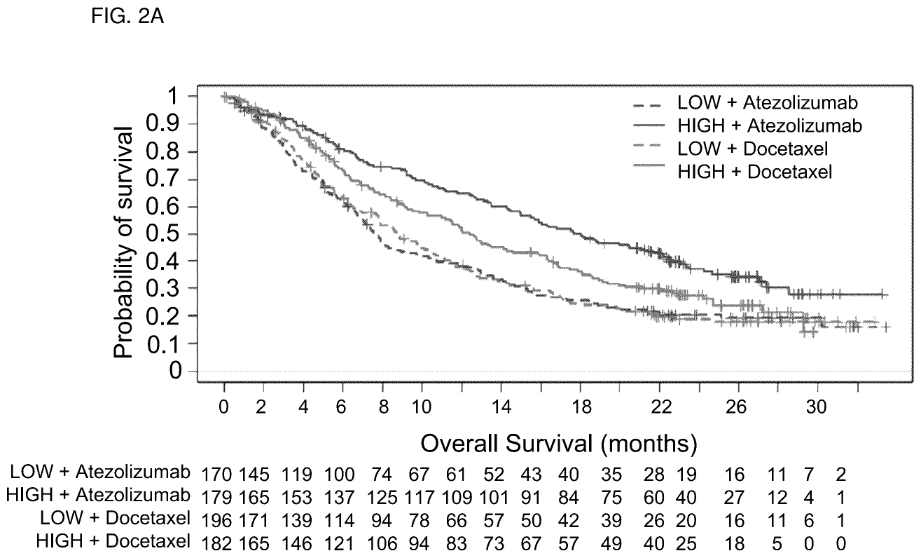

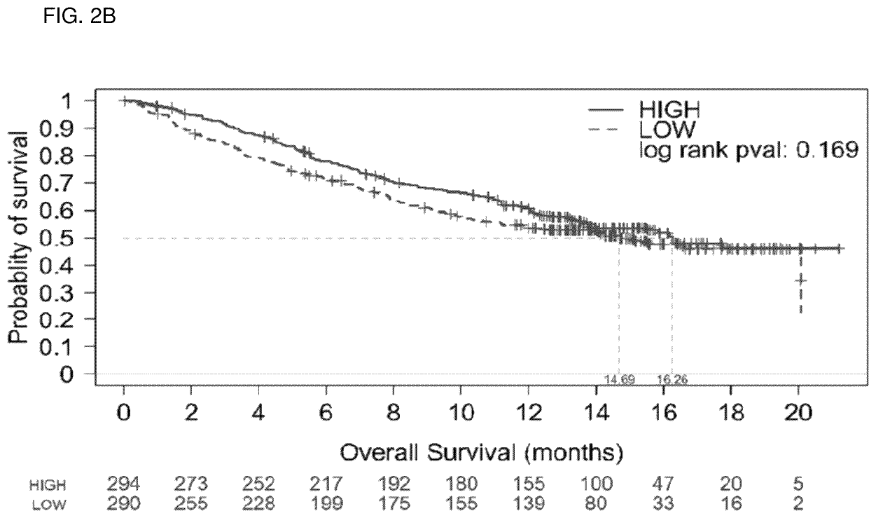

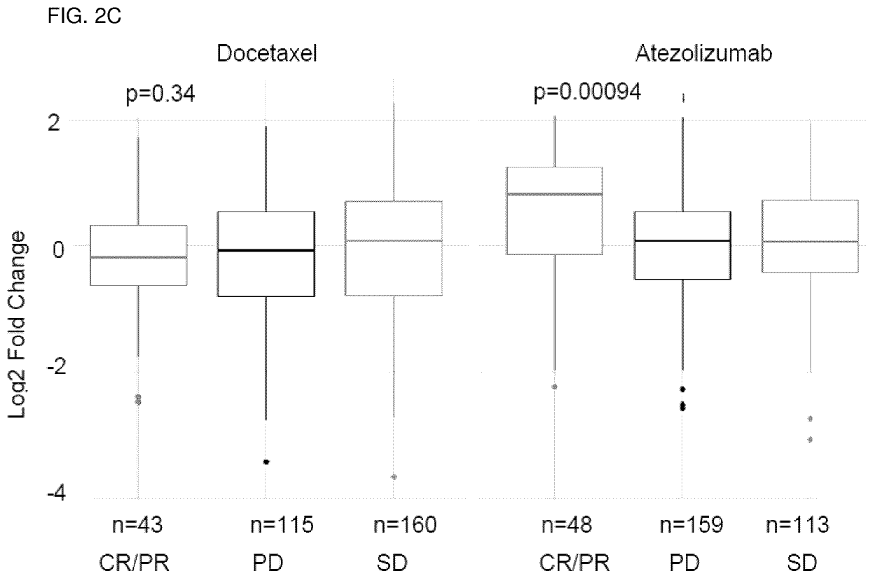

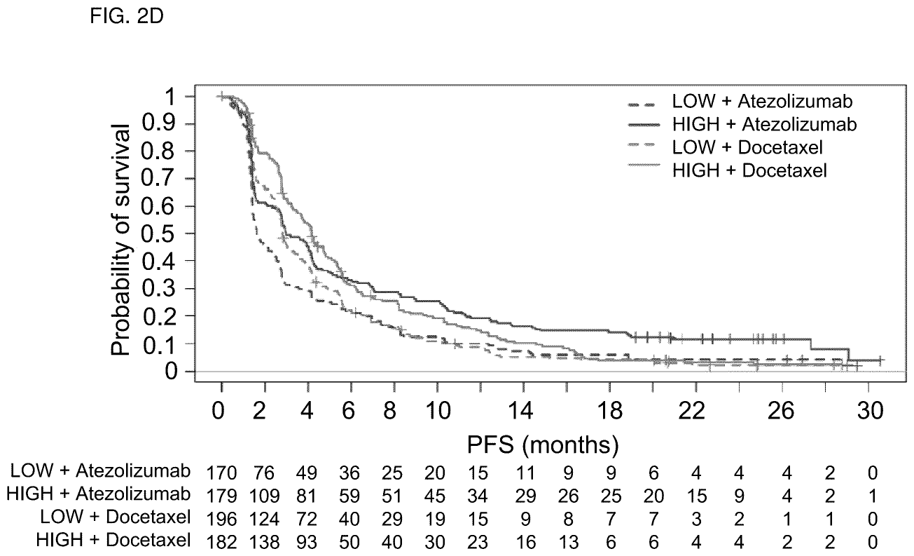

To confirm the above findings, we analyzed two independent clinical datasets, namely BIRCH (n=591) and OAK (n=727). Interestingly, in the randomized Phase 3 OAK study, high expression of the B-cell signature was associated with OS benefit in both chemotherapy and atezolizumab treatment arms, with greater benefit observed in the atezolizumab treated arm (FIG. 2A). These data suggest that high expression of the B-cell signature may have both prognostic and predictive components for OS (>median, HR relative to docetaxel=0.84, CI=0.71-1, p=0.056, and HR relative to <median B cell gene set within the atezolizumab arm=0.54, CI=0.42-0.7, p<0.001). Similarly, in the monotherapy single arm PD-L1 selected (>5% positivity on immune cells or tumor cells) Phase 3 BIRCH study, high expression of the B cell signature was found to be associated with OS benefit in patients treated with atezolizumab (HR=0.78, CI=0.62-0.99, p=0.044) (FIG. 2B).

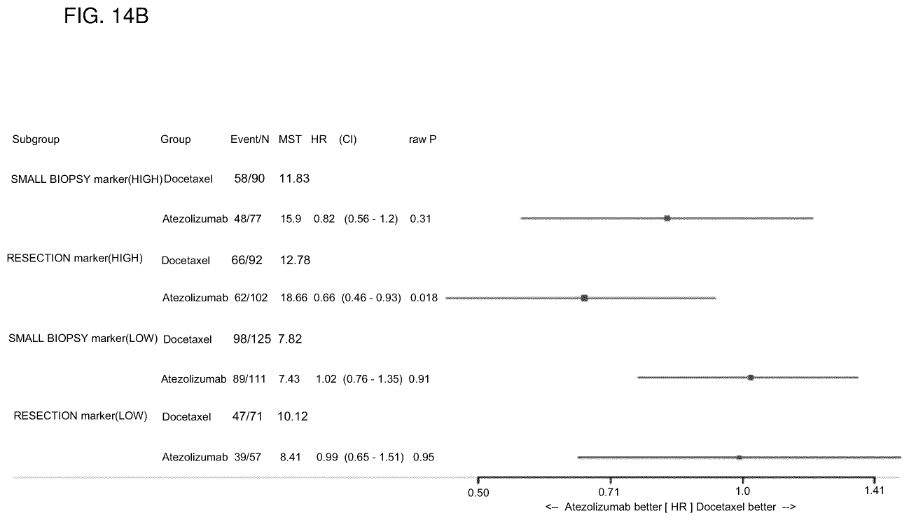

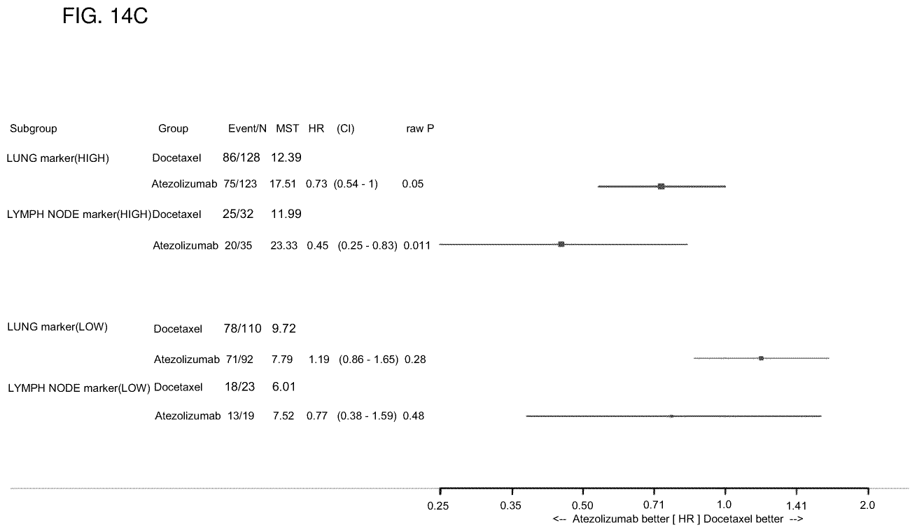

We also evaluated best-confirmed overall response (BOOR) and progression free survival (PFS) in the OAK study. Responders to atezolizumab treatment showed significantly higher expression of B cell signature genes than non-responders and this difference was not observed in patients treated with docetaxel (FIG. 2C). Similar to the association with OS, high expression of the B cell gene signature was associated with greater PFS benefit in both treatment arms with greater benefit observed with atezolizumab (>median, HR=0.54, CI=0.42-0.7, p<0.001) or relative to docetaxel (HR=0.84, CI=0.71-1, p=0.056) (FIG. 2D). High B cell signature was associated with greater benefit in lung tumors (lymph node metastases excluded), irrespective of histology, or sample type (biopsies or resections) (FIGS. 14A-14C).

Presence of B Cells was Confirmed in Patient NSCLC Samples

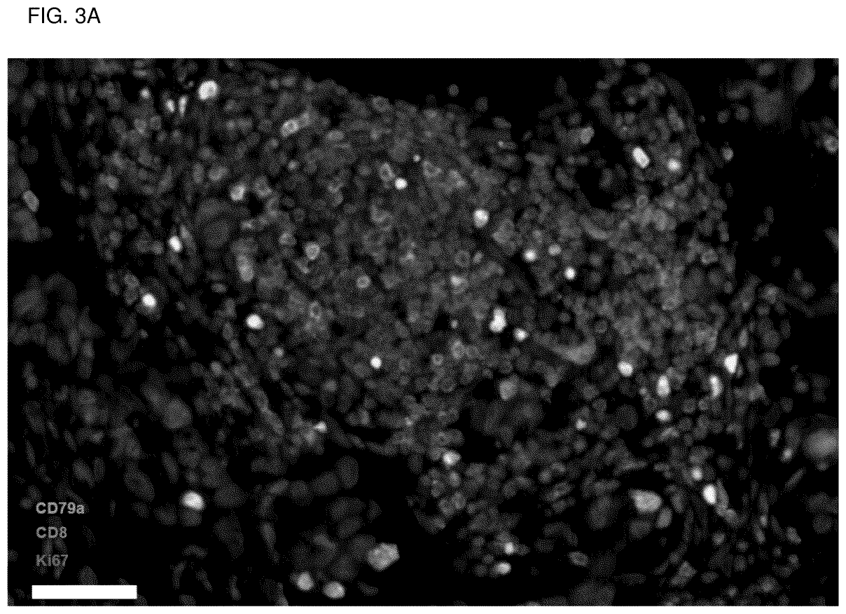

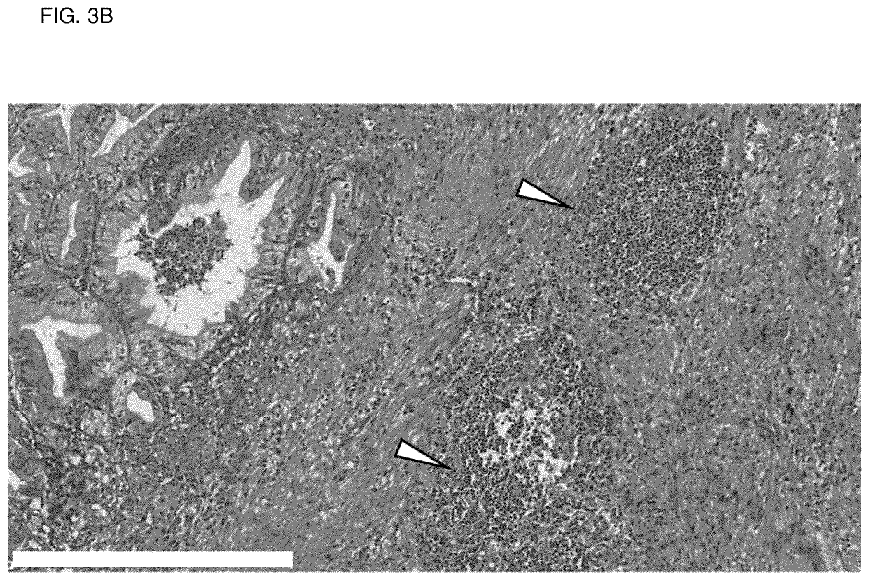

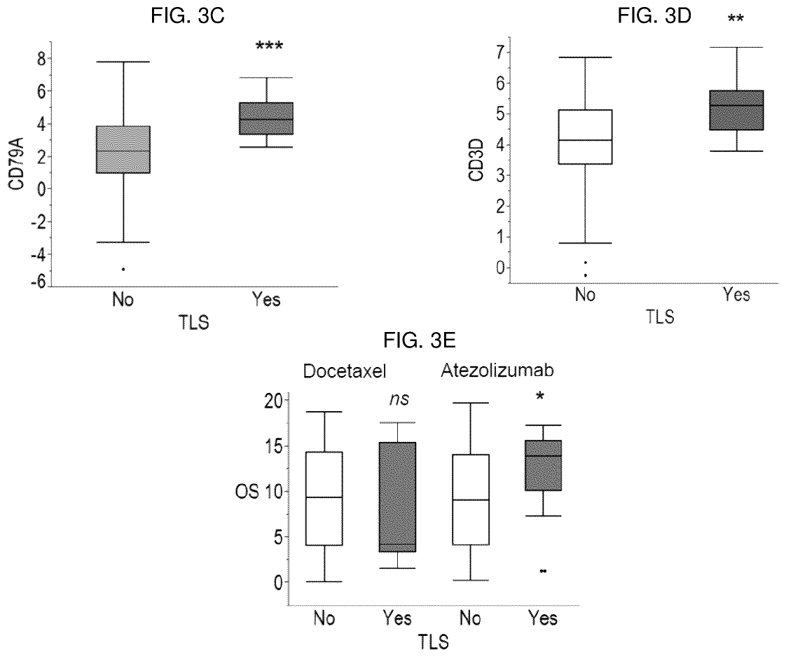

To confirm whether these gene expression findings translated to the presence of CD79+ B cells in the NSCLC tumors, we evaluated samples from baseline tumor samples of 20 patients by immunofluorescence (IF) staining for CD8, CD79 and Ki67 using a multiplex IF. The patient samples had either high or low CD79A gene expression (FIG. 3A). Abundant CD79+ B cells were detected in NSCLC samples with high CD79A RNA. In addition, the multiplex immunohistochemistry (INC) detected regions where CD8+ T cells were in close proximity (9.9±1.69 μm apart) with CD79+ B cells suggestive of B cell-T cell interactions.

Association of B Cell Gene CD79A with OS is not Dependent on T Cell Genes

The presence of T cells in the tumor microenvironment could upregulate cytokines and chemokines that recruit other immune cells non-specifically. To evaluate whether B cells present in the tumor may have been bystander cells recruited into the tumor as a result of infiltrating T cells, and given that immune cell genes are typically highly correlated (for e.g. CD79A and CD8A r 2=0.58, CD3D and CD79A r 2=0.61 in our datasets), we evaluated whether the association of CD79A (B cell receptor gene) is dependent on the presence of CD3D, CD3E, CD8A genes and To gene signatures using Cox proportional hazards multivariate regression analysis. This approach has been used to evaluate tumor-infiltrating lymphocyte (TIL) anatomy in colorectal cancer (CRC) and novel immune signatures in hepatocellular carcinoma (HCC) (Berntsson, J., et al. The clinical impact of tumour-infiltrating lymphocytes in colorectal cancer differs by anatomical subsite: A cohort study. Int. J. Cancer 141, 1654-1666 (2017); Tian, M. X., et al. Tissue-infiltrating lymphocytes signature predicts survival in patients with early/intermediate stage hepatocellular carcinoma. BMC. Med. 17, 106 (2019), Using Cox proportional hazards multivariate regression analysis, we determined that the association of CD79A with OS remained statistically significant after controlling for CD3D, CD3E, CD8A gene or To gene expression as co-variates (Table 19). This demonstrates that CD79A gene expression at baseline has a significant impact on OS hazard ratio in patients receiving atezolizumab treatment independent of T cell genes in the tumor microenvironment.

TABLE 19
Statistical analysis associated with Cox proportional hazard survival time
analysis comparing CD79a to CD3 genes from POPLAR
Correlation with OS compared to CD3D

| Gene | coef | exp(coef) | se(coef) | z | p |
|---|---|---|---|---|---|
| CD79A | −0.2554 | 0.7746 | 0.0789 | −3.24 | 0.0012 |
| CD3D | −0.1329 | 0.8755 | 0.1426 | −0.93 | 0.3512 |

Likelihood ratio test = 23.9 on 2 df, p = 6.44e−06, n = 92, number of events = 51
Correlation with OS compared to CD3E

| Gene | coef | exp(coef) | se(coef) | z | p |
|---|---|---|---|---|---|
| CD79A | −0.2595 | 0.7714 | − 0.0759 | −3.42 | 0.00063 |
| CD3E | −0.1272 | 0.8806 | 0.1359 | −0.94 | 0.34943 |

Likelihood ratio test = 23.9 on 2 df, p = 6.44e−06, n = 92, number of events = 51
Correlation with OS compared to CD8A

| | | | | | |
|---|---|---|---|---|---|
| Gene | coef | exp(coef) | se(coef) | z | p |
| CD79A | −0.28891 | 0.74908 | 0.06485 | −4.455 | 8.38e−06 |
| CD8A | −0.07526 | 0.92750 | 0.11792 | −0.638 | 0.523 |

Likelihood ratio test = 23.45 on 2 df, p = 8.106e−06, n = 92, number of events = 51
Correlation with OS compared to T_{eff} signature

| Gene | coef | exp(coef) | se(coef) | z | p |
|---|---|---|---|---|---|
| CD79A | −0.27119 | 0.76248 | 0.07028 | −3.859 | 0.000114 |
| Teff | −0.17754 | 0.83732 | 0.20371 | −0.872 | 0.383456 |

Likelihood ratio test = 23.95 on 2 df, p = 6.298e−06, n = 93, number of events = 52

CD79A+ High Tumors Show the Presence of Tertiary Lymphoid Structures (TLS)

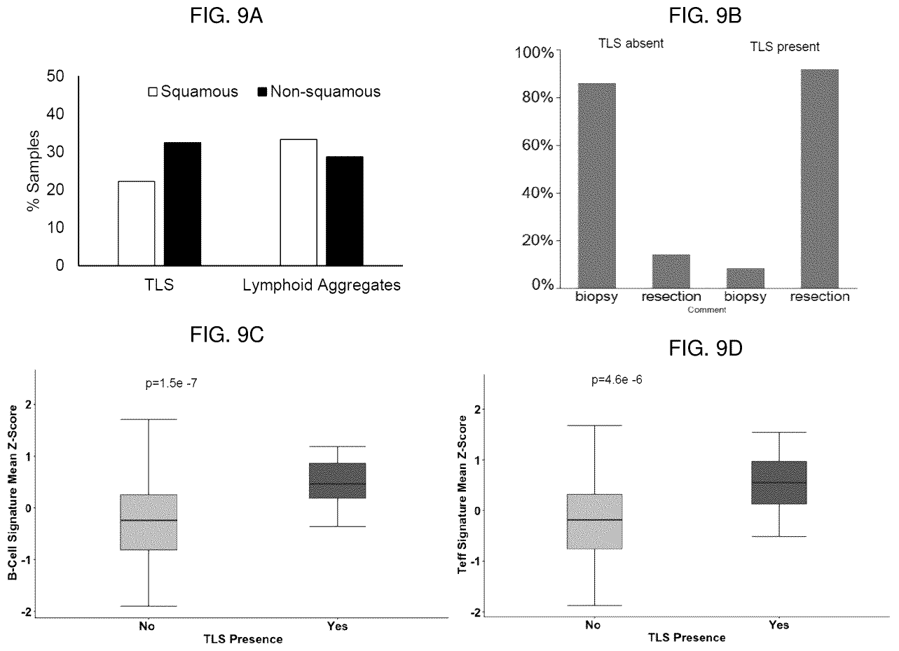

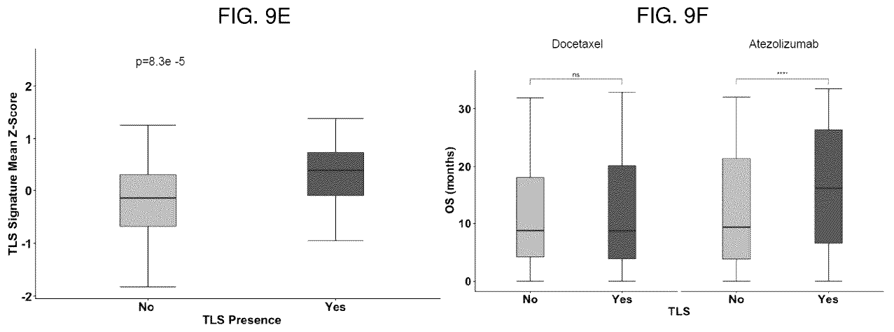

To better understand the role of B cells in these tumors, we evaluated tumor hematoxylin & eosin (H&E) slides for the presence of TLS (FIG. 3B). TLS are frequently observed in tissues affected by chronic inflammation as a result of infection, autoimmunity, cancer, and allograft rejection. These highly ordered structures, resembling the cellular composition of lymphoid follicles, are thought to mimic the activities of germinal centers and contribute to the local control of adaptive immune responses. Of the 290 NSCLC patient samples analyzed, 31% had TLS with germinal centers, 30% had lymphoid aggregates (no observed germinal centers), and the remaining 39% had no detectable TLS or lymphoid aggregates. Lymph node metastases samples (4% of all samples) were excluded from this analysis. In this cohort, both squamous and non-squamous NSCLC types had similar distribution of TLS and of lymphoid aggregates, and were predominantly detected in resected samples (FIGS. 9A and 9B). Importantly, tumors with high CD79A gene expression were highly correlated with presence of TLS (FIG. 9C).

While TLS were detected in roughly equal numbers of patient tumors for both treatment arms, TLS positive patients treated with atezolizumab showed association with OS benefit that was not observed in TLS positive patients treated with docetaxel in the POPLAR (FIG. 3E) and OAK studies (FIG. 9F).

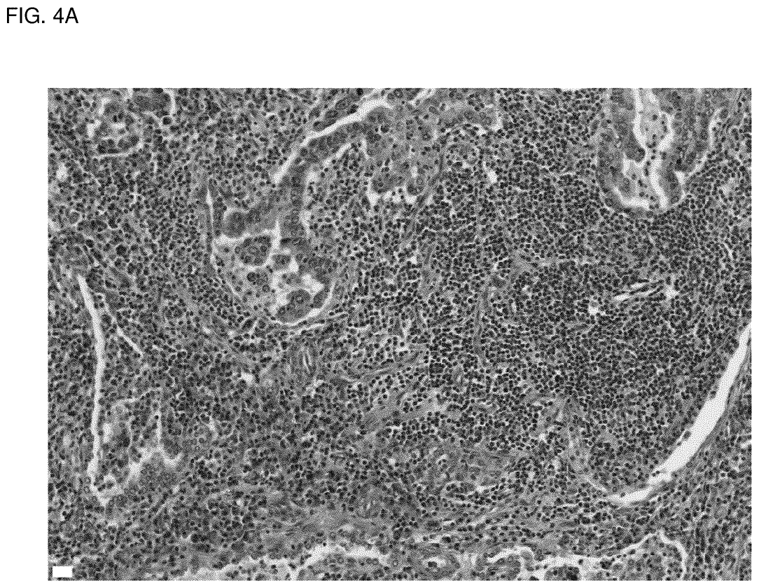

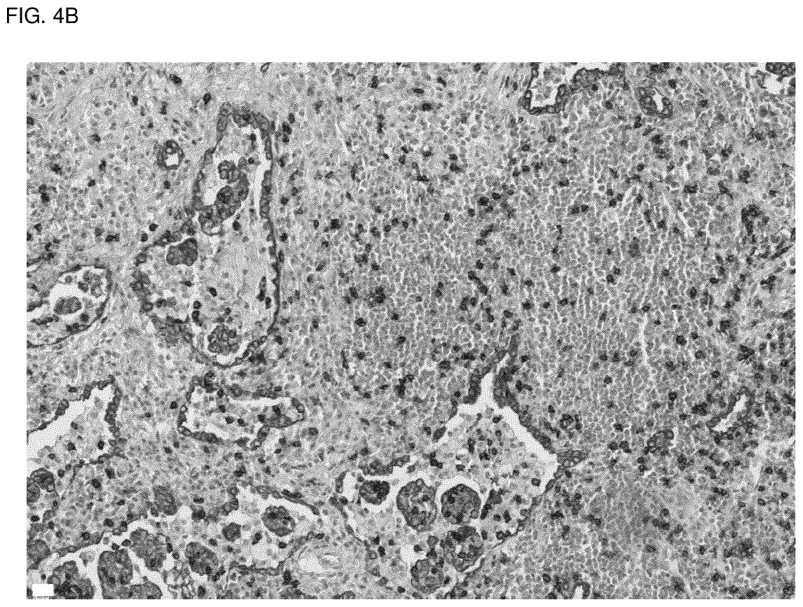

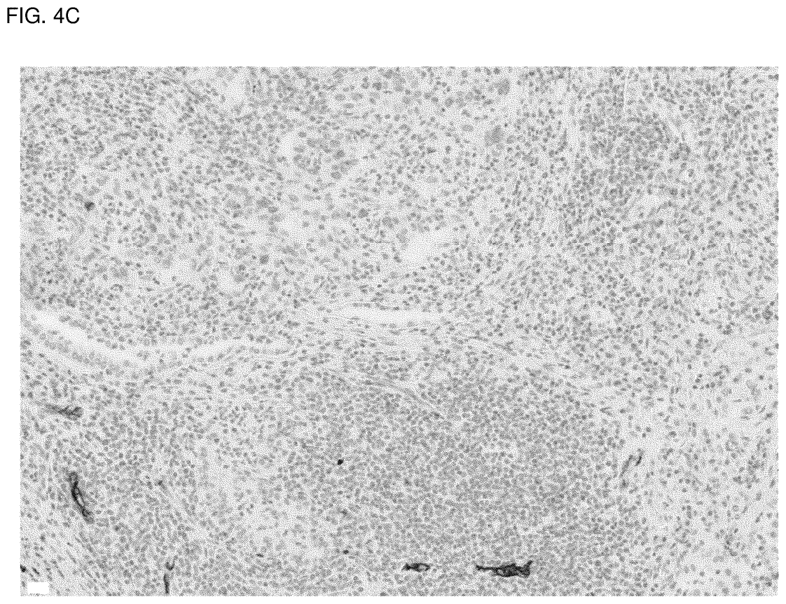

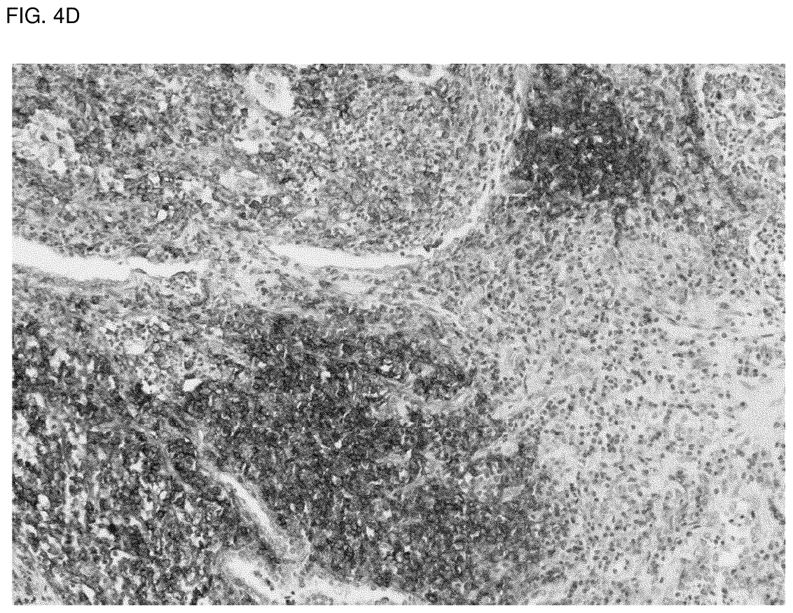

To further confirm that these structures identified by H&E staining were indeed TLS, IHC was performed on a subset of samples to detect specialized vasculatures or high endothelial venules (HEVs) that are known to be associated with TLS and mediate lymphocyte trafficking to secondary lymphoid organs (Colbeck, E. J., et al. Tertiary Lymphoid Structures in Cancer: Drivers of Antitumor Immunity, Immunosuppression, or Bystander Sentinels in Disease? Front. Immunol. 8, 1830 (2017); Engelhard, V. H., et al. Immune Cell Infiltration and Tertiary Lymphoid Structures as Determinants of Antitumor Immunity. J. Immunol. 200, 432-442 (2018)). The MECA-79 monoclonal antibody recognizes peripheral lymph node addressins (PNAd) by targeting CD62L or L-selectin that is exclusively expressed by HEVs. TLS positive patient tumor samples stained positive for MECA-79, CD40, and CD8 by IHC staining (FIGS. 4A-4D).

TLS Gene Signatures are Associated with Atezolizumab OS Benefit

The TLS analysis by H&E was performed on a limited number (n=290) of samples as described above. To extend this analysis to the larger gene expression dataset available to us, a previously reported TLS gene signature was used. We verified the H&E based TLS samples were associated with the published TLS gene expression signature (FIG. 9E), and were also associated with the B cell gene signature and the To gene signatures as expected (FIGS. 9C and 9D) (Zhu, G., et al. Tumor-Associated Tertiary Lymphoid Structures: Gene-Expression Profiling and Their Bioengineering. Front. Immunol. 8, 767 (2017); Colbeck, E. J., et al. Tertiary Lymphoid Structures in Cancer: Drivers of Antitumor Immunity, Immunosuppression, or Bystander Sentinels in Disease? Front. Immunol. 8, 1830 (2017); Bremnes, R. M., et al. The Role of Tumor-Infiltrating Lymphocytes in Development, Progression, and Prognosis of Non-Small Cell Lung Cancer. J. Thorac. Oncol. 11, 789-800 (2016); Germain, C., et al. Presence of B cells in tertiary lymphoid structures is associated with a protective immunity in patients with lung cancer. Am. J. Respir. Crit. Care. Med. 189, 832-844 (2014)).

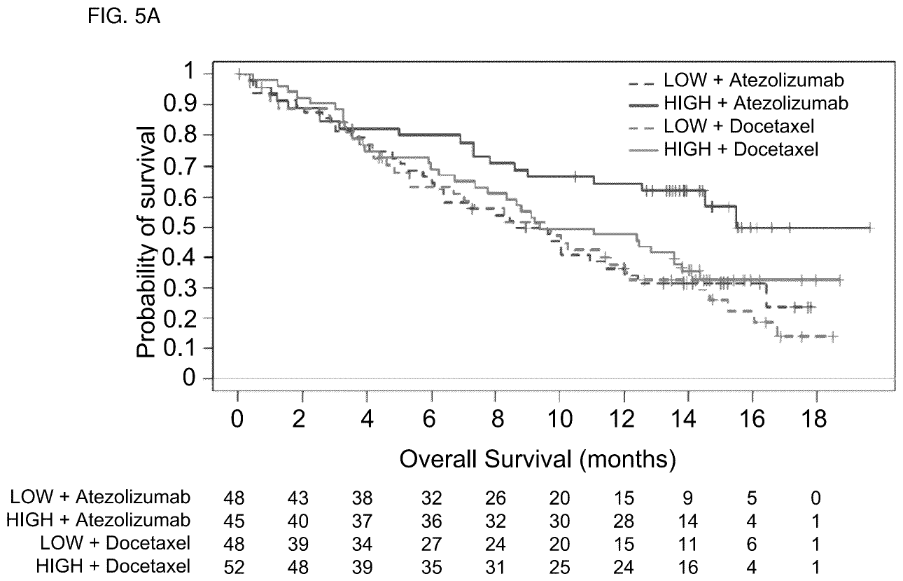

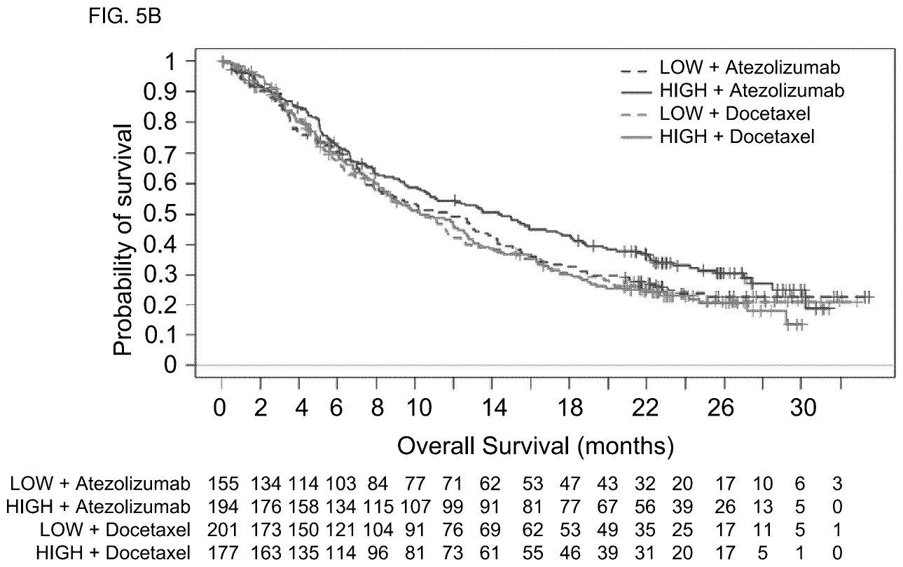

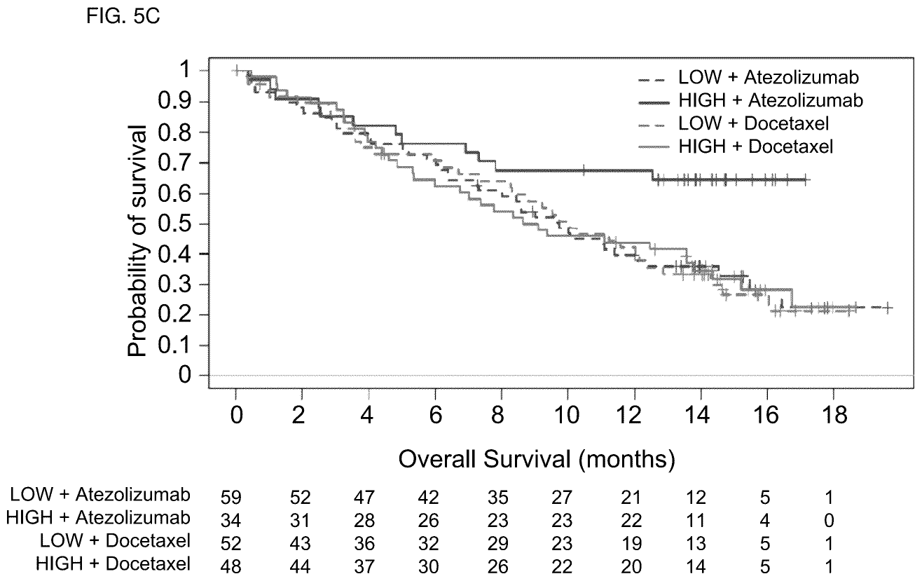

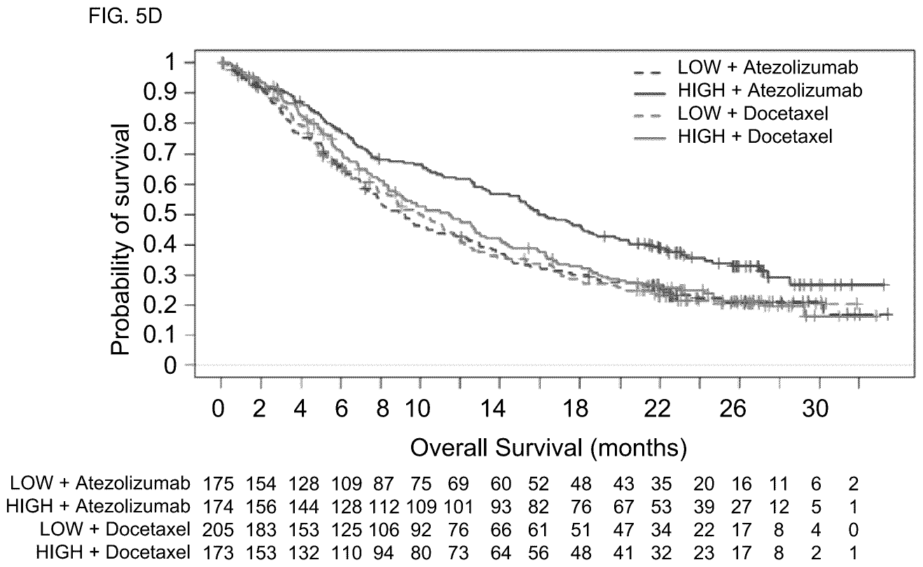

Patient tumors with high TLS gene signature were significantly associated with OS benefit in the atezolizumab treatment arm (>median, HR=0.48, CI=0.27-0.86, p=0.013) in POPLAR and (>median, HR=0.82, CI=0.64-1.06, p=0.14) in OAK (FIGS. 5A and 5B). Similarly, using another TLS related gene signature identified by Cabrita et al., the germinal center initiation signature showed significant survival benefit with atezolizumab treatment (>median, HR=0.44, CI=0.23-0.84, p=0.013) in POPLAR and (>median, HR=0.66, CI=0.51-0.86, p<0.01) in OAK (FIGS. 5C and 5D) (Cabrita, R., et al. Tertiary lymphoid structures improve immunotherapy and survival in melanoma. Nature 577, 561-565 (2020)).

Plasma Cell Gene Signatures are Associated with Atezolizumab OS Benefit

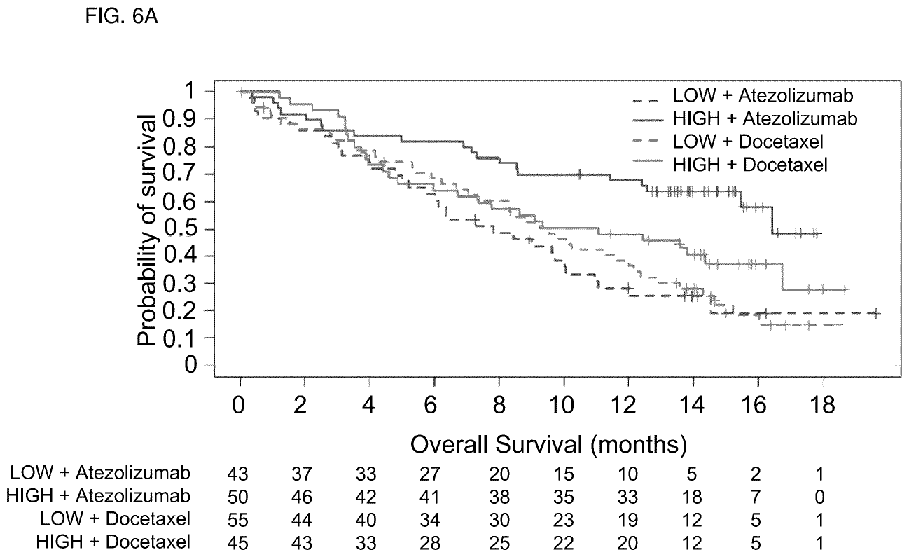

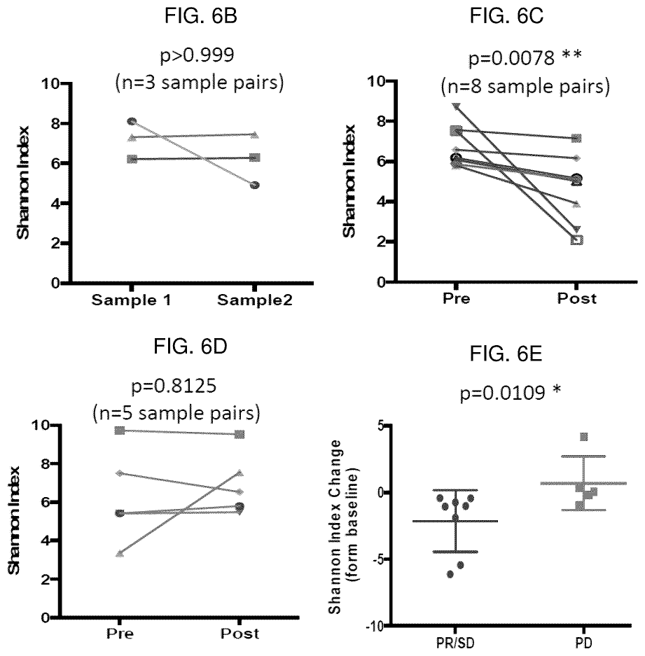

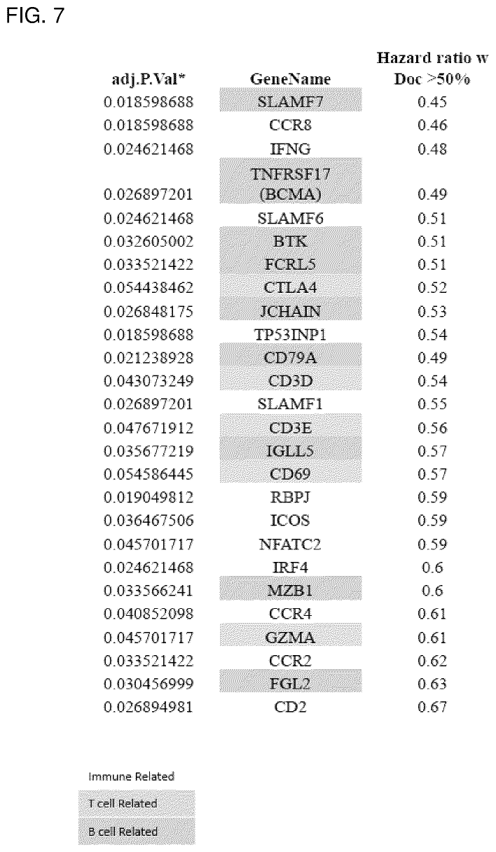

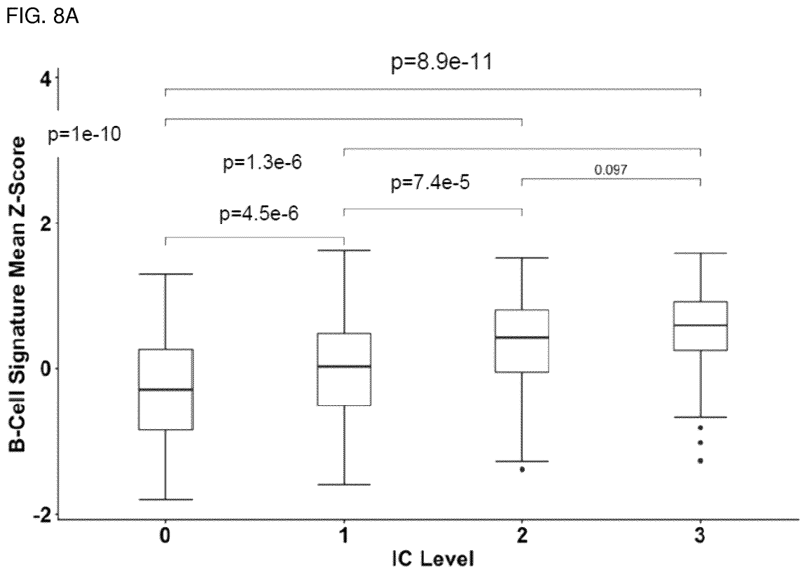

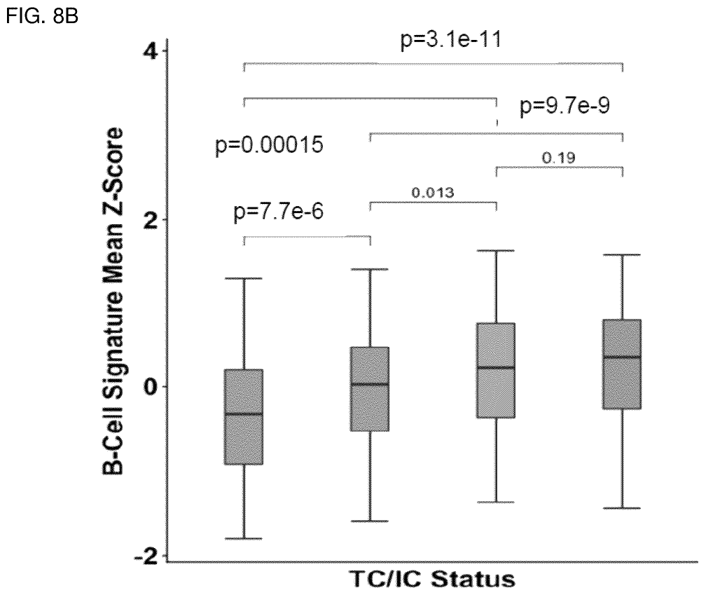

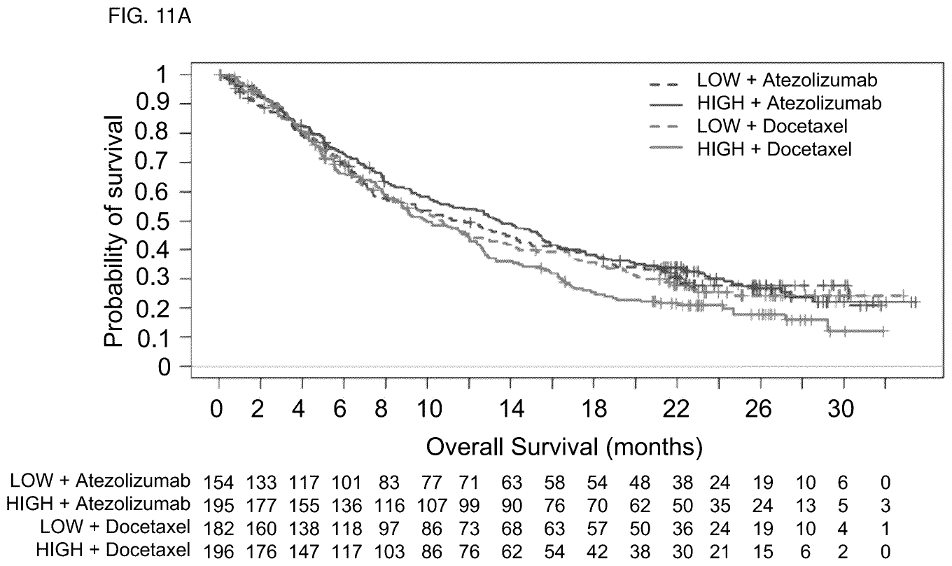

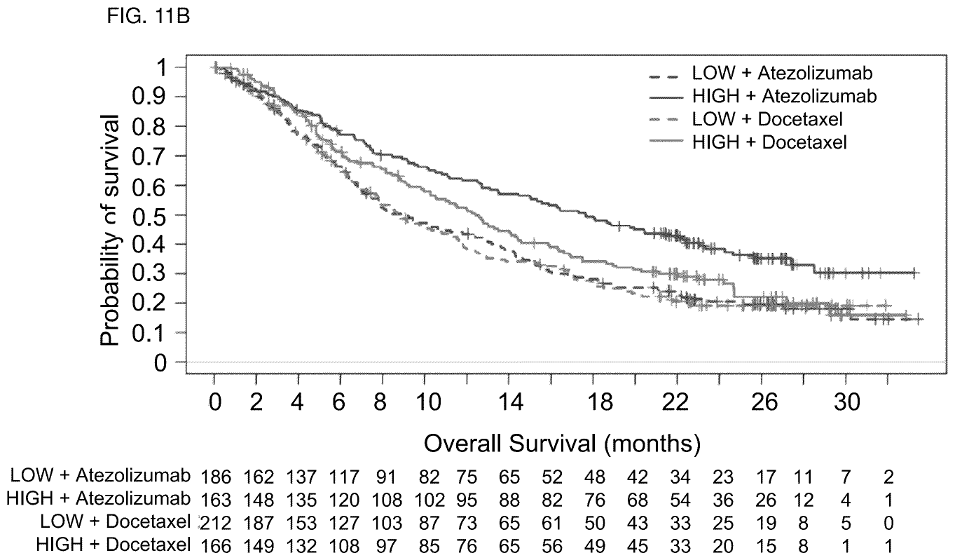

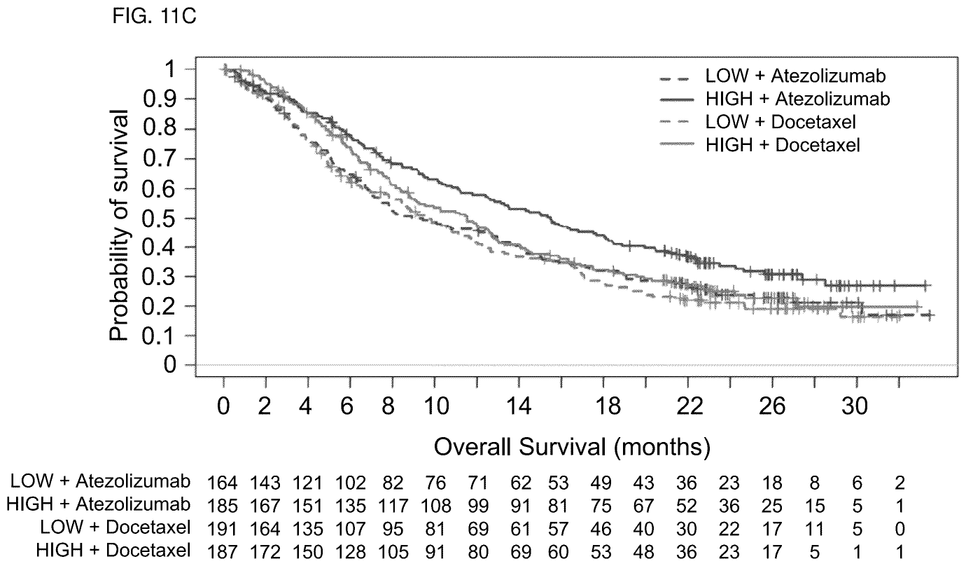

To further elucidate the B cell subtypes that were present in the tumors, we analyzed several known B cell signatures for their ability to predict survival (FIGS. 11A-11C) (Newman, A. M., et al. Robust enumeration of cell subsets from tissue expression profiles. Nat. Methods 12, 453-457 (2015)). The OS benefit with atezolizumab treatment showed one of the strongest associations with a published plasma cell signature consisting of 2-gene set, TNFRSF17 (or BCMA), which is expressed when B cells differentiate into plasma cells, and immunoglobulin joining region (IGJ or JCHAIN) that is common to all antibodies (Kroeger, D. R., et al. Tumor-Infiltrating Plasma Cells Are Associated with Tertiary Lymphoid Structures, Cytolytic T-Cell Responses, and Superior Prognosis in Ovarian Cancer. Clin. Cancer Res. 22, 3005-3015 (2016)). High expression of this two-gene plasma cell signature was strongly associated with OS benefit with atezolizumab treatment (>median, HR=0.58, CI=0.44-0.74, p<0.001) in OAK) (FIG. 6A).

Reduction of Shannon Diversity Index (SDI) of BCR (B Cell Receptor) Repertoire is Associated with Disease Control

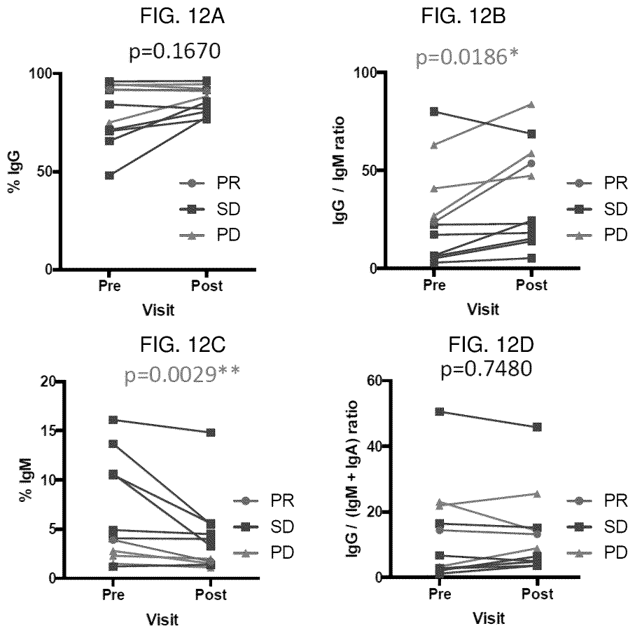

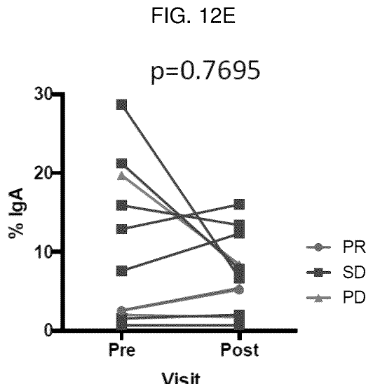

Given that plasma cell signatures were highly associated with clinical benefit, we wanted to investigate if there were any changes in the BCR repertoire or clonality post atezolizumab treatment in patients showing clinical benefit. We analyzed 13 pairs of matched pre- and post-treatment biopsies from the atezolizumab monotherapy FIR (NCT01846416) and BIRCH trials as shown in FIGS. 6A-6E. All post-treatment samples were collected at the time of disease progression. The median time of collection was 184.5 days after initial treatment for patients with partial response (PR) or patients with stable disease (SD), and 51 days for patients with progressive disease (PD). The only PR patient analyzed here had a PFS of 12.9 months. The 7 SD patients and 5 PD patients had median PFS of 5.52 and 1.35 months, respectively. Due to censoring, the median OS was not reached in either group. Tumor RNA was isolated and sequenced for BCR genes. RNA based clonotype repertoire sequencing allowed us to determine isotype and exclude signal dilution from non-expressing B cells (Schalper, K. A., et al. Neoadjuvant nivolumab modifies the tumor immune microenvironment in resectable glioblastoma. Nat. Med. 25, 470-476 (2019); Smith, C. C., et al. Using RNA Sequencing to Characterize the Tumor Microenvironment. Methods Mol. Biol. 2055, 245-272 (2020)). A majority of the BCRs sequenced were of the IgG isotypes and not IgA or IgM, suggesting that the tumor infiltrating B cells were mostly IgG+ B cells (FIGS. 12A-12F).

Once BCR cDNA libraries were generated and sequenced, the BCR repertoires were evaluated by the SDI, which is a measure of B-cell clonality (Chaudhary, N. & Wesemann, D. R. Analyzing Immunoglobulin Repertoires. Front. Immunol. 9, 462 (2018); Greiff, V., et al. A bioinformatic framework for immune repertoire diversity profiling enables detection of immunological status. Genome Med. 7, 49 (2015); Kaplinsky, J. et al. Robust estimates of overall immune-repertoire diversity from high-throughput measurements on samples. Nat. Commun. 7, 11881 (2016)). The SDI for BCR sequences from pre-post atezolizumab treatment pairs (n=11) of NSCLC patient tumor samples were compared with pre-treatment tumor sample pairs (n=3), where both samples were taken before treatment initiation, as an experimental control. The paired samples taken pre-atezolizumab treatment from 3 patients showed no change in the SDI (FIG. 6B). Among the patients whose pre- and post-atezolizumab treatment samples were analyzed, 7 patients with SD and 1 with PR, showed a significant decrease in Shannon Index in post-treatment tumors compared to pre-treatment samples (p=0.0078, FIG. 6C). In comparison, the 5 patients with PD had no significant change of the index following the treatment (p=0.8125) (FIG. 6D). These observations suggest that within this limited subset of patients who showed clinical benefit with atezolizumab (PR and SD), there was a concomitant decrease in BCR diversity. This is suggestive of clonal expansion or enrichment of specific Ig+ B cells in the tumors with atezolizumab treatment. The 5 patients who progressed on atezolizumab treatment showed no evidence of clonal enrichment on treatment (FIG. 6E).

### DISCUSSION

Our data show that infiltration of B cells, specifically surface Ig+ B cells and plasma cells, into the tumor microenvironment, is a key factor in determining OS benefit for NSCLC patients treated with atezolizumab. Our data demonstrates that the presence of B cells and existence of TLS at baseline determine atezolizumab mediated OS benefit, with findings validated from three large independent clinical trial cohorts (one Phase 2 [n-1931 and two Phase 3 studies [n=591 and 727], respectively).

In our study, gene expression analyses revealed a strong association between B cell markers (memory & plasma) and survival benefit with atezolizumab treatment (FIGS. 1A-1C, 2A-2D, and 11A-11C) and includes a plasma gene signature of BCMA and the IGJ genes (Kroeger, D. R., et al. Tumor-Infiltrating Plasma Cells Are Associated with Tertiary Lymphoid Structures, Cytolytic T-Cell Responses, and Superior Prognosis in Ovarian Cancer. Clin. Cancer Res. 22, 3005-3015 (2016)). Our data from sequencing the BCR shows that intra-tumoral B cell diversity is decreased in patients showing clinical benefit to atezolizumab using the SDI, and suggest B cell clonal expansion.

Our analysis shows CD79+ B cells and CD8⁺ T cells present in close proximity when visualized by immunofluorescence imaging, supporting a role of these cells as APC.

In this study, we observed a strong correlation of CD79A gene expression with presence of TLS, as determined by H&E staining. The prevalence of TLS in NSCLC in predominantly early stage squamous NSCLC have been reported to be present in >95% of patients, of which 50% are present without a germinal center (Silina, K., et al. Germinal Centers Determine the Prognostic Relevance of Tertiary Lymphoid Structures and Are Impaired by Corticosteroids in Lung Squamous Cell Carcinoma. Cancer Res. 78, 1308-1320 (2018)). Our analysis from metastatic NSCLC suggests that TLS prevalence is ˜30% and another 30% have lymphoid aggregates without a germinal center. Our analysis also suggests the presence of TLS by histology and gene expression signatures shows a significant association with OS post-atezolizumab treatment, suggesting they may play an important role in a sustained immune response to tumors with PD1/PD-L1 blockade.

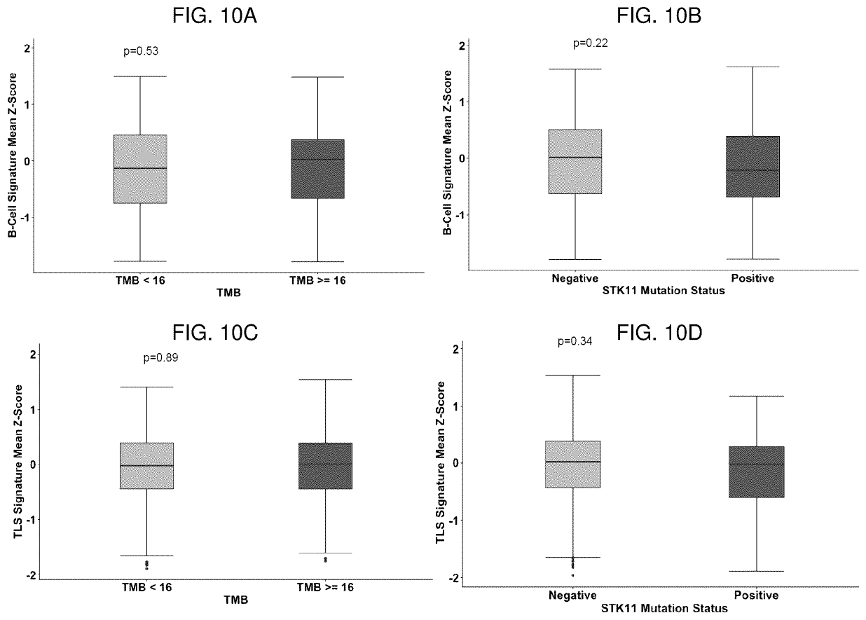

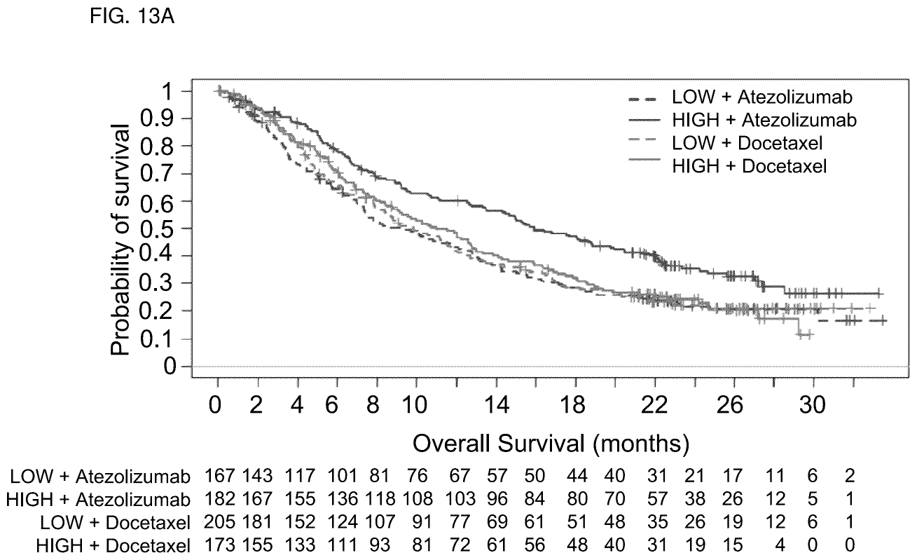

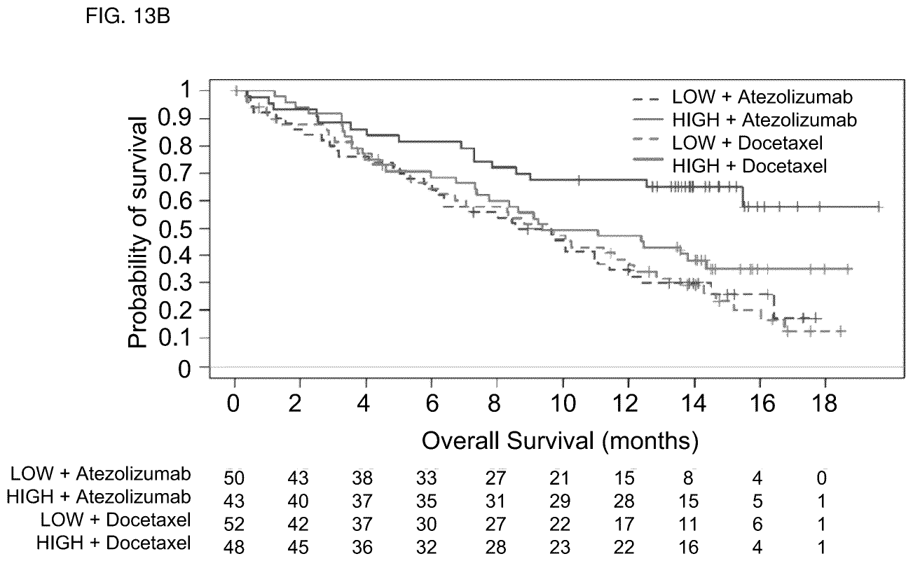

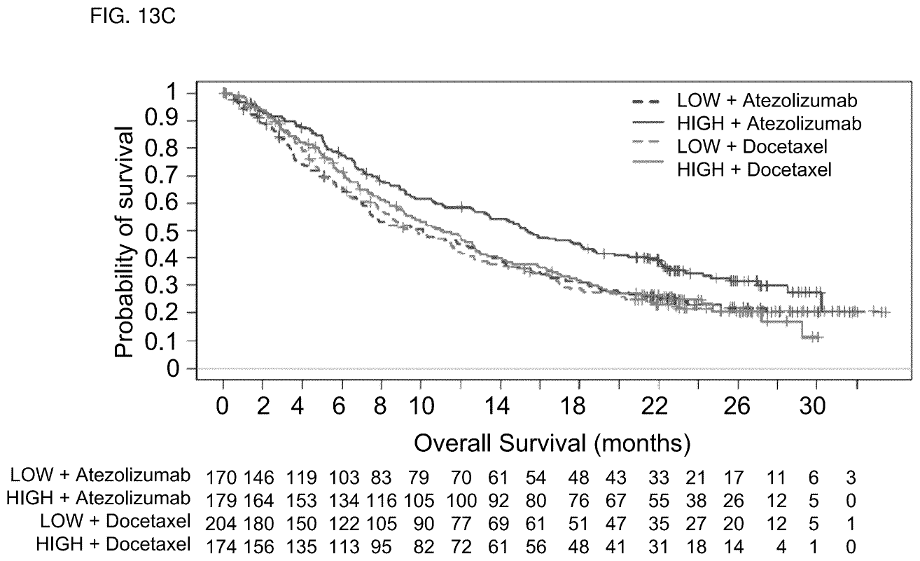

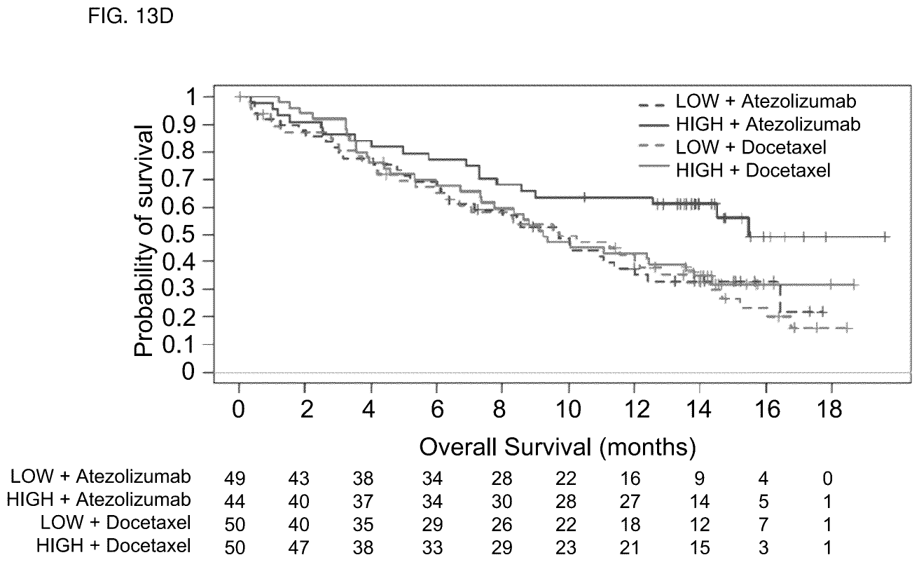

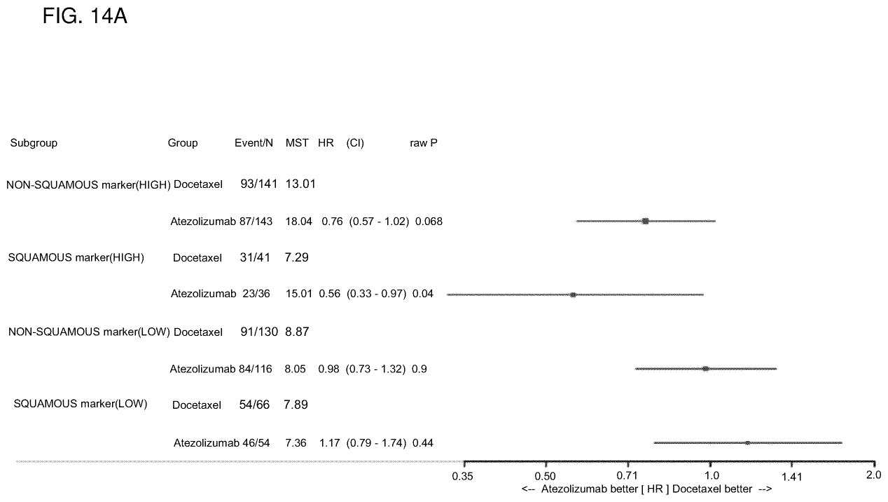

Our data suggest that treatment with atezolizumab involves both the cytolytic and antibody-mediated humoral effector mechanisms, mediated by To and B cells, respectively. This is shown using a composite biomarker composed of T effector 8-gene signature (tGE) and CD79A (OS HR relative to <median in atezolizumab arm for tGE HR=0.7, CI=0.55-0.91, p<0.01; and for tGE+CD79A HR=0.65, CI=0.50-0.84, p<0.01) in the OAK study (FIGS. 13A-13D) (Kowanetz, M., et al. Differential regulation of PD-L1 expression by immune and tumor cells in NSCLC and the response to treatment with atezolizumab (anti-PD-L1). Proc. Natl. Acad. Sci. USA 115, E10119-E10126 (2018)). With regard to other atezolizumab biomarkers, the B cell signature correlates with increasing PD-L1 on immune cells, likely reflecting an inflamed tumor microenvironment (FIGS. 10A-10D). However neither the B cell or the TLS gene signatures are correlated with tumor mutation burden (TMB) and STK11 mutation (FIGS. 10A—(Gandara, D. R., et al. Blood-based tumor mutational burden as a predictor of clinical benefit in non-small-cell lung cancer patients treated with atezolizumab. Nat. Med. 24, 1441-1448 (2018); Hellmann, M. D., et al. Genomic Features of Response to Combination Immunotherapy in Patients with Advanced Non-Small-Cell Lung Cancer. Cancer Cell. 33, 843-852 e844 (2018)).

Additionally, our data show that the presence of intratumoral B cells, especially plasma cells, at baseline is a key factor in determining OS benefit in NSCLC patients treated with atezolizumab. To our knowledge, our data provide the first substantial evidence that the presence of plasma cells and existence of organized lymphoid structures such as TLS at baseline are associated with atezolizumab but not with chemotherapy-mediated OS benefit. To do so, transcriptomic data were analyzed from two large independent randomized clinical trial cohorts (OAK, n=699 and POPLAR, n=192), comprising the largest publicly available transcriptomic NSCLC database 217 treated with CPI or chemotherapy and associated clinical outcome.

In our study, gene expression analyses revealed a strong association between B cell markers and OS benefit with atezolizumab treatment. We utilized single-cell analyses to derive and validate specific and robust gene signatures to reliably deconvolve follicular B cells, GC B cells, and plasma cells from bulk tumor transcriptomes. While each B cell subset had some association with outcomes, plasma cells seem to be the most important. This is perhaps indicative of a productive local germinal center reaction whereby mature plasma cells are the end product. These data are bolstered by similarly significant clinical benefit in patients with histologically identified TLS or TLS-like structures. Taken together, these data suggest that tumors with high plasma cell infiltration identify tumors with TLS or TLS like structures that can provide sustained tumor control once stimulated by CPI.

Interestingly, tumors with TLS or lymphoid aggregates comprised ˜30% of all NSCLC tumors analyzed and were enriched for B/plasma cell signatures. In our cohorts, the presence of TLS, identified by histology, associated significantly with improved OS following treatment with atezolizumab, suggesting they may play an important role in sustained intratumoral immune responses with PD-(L)1 blockade. When we applied the plasma cell signature to TOGA NSCLC transcriptomes that were not primarily treated with CPI, we found no association with overall survival, suggesting that infiltration of plasma cells is not simply prognostic in NSCLC. Overall, our data in the context of two large randomized clinical trials of atezolizumab vs. chemotherapy show a strong predictive association between B/plasma cells and overall survival that is specific to the mechanism of immunotherapy.

The mechanisms by which B/plasma cells contribute to anti-tumoral immunity remain unclear. Our analysis shows CD79⁺B cells and CD8⁺ T cells present in close proximity when visualized by immunofluorescence imaging, supporting a role of these cells as APC. Class-switched and memory B cells were enriched in tumors from CIT responders in melanoma and RCC, where they may promote antigen presentation to prime cytotoxic T cells.

Our analysis shows a significant correlation between TLS defined by histology and B cell subset gene signatures, which clearly points to a common underlying B cell biology likely including a humoral immune response that contributes to the overall response to atezolizumab. Our findings also clearly show that increased frequency of B and plasma cells at baseline associates with improved clinical outcome in NSCLC patients treated with atezolizumab, independently of cytotoxic T cell signatures.

In sum, our analysis shows significant correlation between TLS defined by histology, B cells IHC, TLS, and germinal center related gene signatures, which points to a common underlying B cell biology including a humoral immune response that contributes to an atezolizumab mediated response. Our findings also show that more B cells and plasma cells at baseline independent of T cells improve the clinical outcome for NSCLC patients treated with atezolizumab.

Materials and Methods

Patient Population

This study was performed using tissue samples from the open-label, randomized Phase 2 POPLAR (NCT01903993) and Phase 3 OAK trials (NCT02008227) that evaluated atezolizumab versus docetaxel in patients with NSCLC who progressed on post-platinum chemotherapy (Rittmeyer, A., et al. Atezolizumab versus docetaxel in patients with previously treated non-small-cell lung cancer (OAK): a phase 3, open-label, multicentre randomised controlled trial. Lancet 389,255-265 (2017); Fehrenbacher, L., et al. Atezolizumab versus docetaxel for patients with previously treated non-small-cell lung cancer (POPLAR): a multicentre, open-label, phase 2 randomised controlled trial. Lancet 387, 1837-1846 (2016)). Where referenced, tissue-derived RNA was used from the Phase 2 FIR (NCT01846416) and BIRCH (NCT02031458) single arm trials that evaluated atezolizumab in patients with locally advanced or metastatic NSCLC (Peters, S., et al. Phase II Trial of Atezolizumab As First-Line or Subsequent Therapy for Patients With Programmed Death-Ligand 1-Selected Advanced Non-Small-Cell Lung Cancer (BIRCH). J. Clin. Oncol. 35, 2781-2789 (2017); Spigel, D. R., et al. FIR: Efficacy, Safety, and Biomarker Analysis of a Phase II Open-Label Study of Atezolizumab in PD-L1-Selected Patients With NSCLC. J. Thorac. Oncol. 13, 1733-1742 (2018)). Patients in all trials received either 1200 mg atezolizumab IV every 3 weeks (q3w) until disease progression or loss of clinical benefit, or 75 mg/m 2 docetaxel IV q3w until PD. Both FIR and BIRCH reported improved response rates relative to historical averages; POPLAR and OAK studies demonstrated significant improvement in OS with atezolizumab versus docetaxel, regardless of PD-L1 expression (Rittmeyer, A., et al. Atezolizumab versus docetaxel in patients with previously treated non-small-cell lung cancer (OAK): a phase 3, open-label, multicentre randomised controlled trial. Lancet 389, 255-265 (2017); Fehrenbacher, L., et al. Atezolizumab versus docetaxel for patients with previously treated non-small-cell lung cancer (POPLAR): a multicentre, open-label, phase 2 randomised controlled trial. Lancet 387, 1837-1846 (2016)). Protocol approval was obtained from independent ethics committees for each participating site for all studies and an independent data monitoring committee reviewed the safety data. No crossover was allowed, and OS was a primary endpoint.

Gene Expression Analysis

All transcriptome profiles were generated using TruSeq RNA Access technology (Illumina®) for 192 patients from the POPLAR, 699 patients from OAK, 137 patients from FIR, and 591 patients from BIRCH trials. Alignment of RNAseq reads to ribosomal RNA sequences was performed to remove ribosomal reads. NCI Build 38 human reference genome was then used to align the remaining reads using GSNAP version 2013-10⁻¹⁰ wherein a maximum of two mismatches per 75 base sequence (parameters: ‘-M 2 -n 10 -B 2 -i 1 -N 1 -w 200000 -E 1 -pairmax-rna=200000 --clip-overlap) was allowed. Transcript annotation was based on the Ensembl genes database (release 77). To quantify gene expression levels, the number of reads mapped to the exons of each RefSeq gene was calculated in a strand-specific manner using the functionality provided by the R package Genomic Alignments (Bioconductor) (Mariathasan, S., et al. TGFbeta attenuates tumour response to PD-L1 blockade by contributing to exclusion of T cells. Nature 554, 544-548 (2018); Lawrence, M., et al. Software for computing and annotating genomic ranges. PLoS. Comput. Biol. 9, e1003118 (2013)).

To identify biology associated with OS benefit with atezolizumab, we grouped patients with either survival of <6 months (n=24) or >12 months (n=43) (Fehrenbacher, L., et al. Atezolizumab versus docetaxel for patients with previously treated non-small-cell lung cancer (POPLAR): a multicentre, open-label, phase 2 randomised controlled trial. Lancet 387, 1837-1846 (2016)). Differentially expressed genes between these two groups were determined using the R package limma (Bioconductor), which implements an empirical Bayesian approach to estimate gene expression changes using moderated t-tests (Mariathasan, S., et al. TGFbeta attenuates tumour response to PD-L1 blockade by contributing to exclusion of T cells. Nature 554, 544-548 (2018); Ritchie, M. E., et al. limma powers differential expression analyses for RNA-sequencing and microarray studies. Nucleic Acids Res. 43, e47 (2015)).

Elsewhere, a Cox proportional hazards regression analysis was also used to assess the interdependence of the contributions of key B cell and T cells genes on overall survival benefit. Also, paired t-test was used as indicated.

Gene Signatures

B cells signatures tested here include a large number of markers and their various associated genes for naïve B cells, memory B cells and plasma cells (Newman, A. M., et al. Robust enumeration of cell subsets from tissue expression profiles. Nat. Methods 12, 453-457 (2015); Bindea, G., et al. Spatiotemporal dynamics of intratumoral immune cells reveal the immune landscape in human cancer. Immunity 39, 782-795 (2013); Iglesia, M. D., et al. Prognostic B-cell signatures using mRNA-seq in patients with subtype-specific breast and ovarian cancer. Clin. Cancer Res. 20, 3818-3829 (2014); Palmer, C., Diehn, M., Alizadeh, A. A. & Brown, P. O. Cell-type specific gene expression profiles of leukocytes in human peripheral blood. BMC. Genomics 7, 115 (2006); Suzuki, A., et al. Investigation of molecular biomarker candidates for diagnosis and prognosis of chronic periodontitis by bioinformatics analysis of pooled microarray gene expression datasets in Gene Expression Omnibus (GEO). BMC. Oral Health 19, 52 (2019)). Naïve B cells gene signature: ABCB4, BCL7A, BENDS, BRAF, IL4R, LINC00921, MEP1A, MICAL3, NIPSNAP3B, PSG2, SELL, TCL1A, UGT1A8, ZNF286A; memory B cells gene signature: AIM2, ALOX5, CLCA3P, FAM65B, IFNA10, IL7, NPIPB15, SP140, TNFRSF13B, TRAF4, ZBTB32; plasma cells gene signature: ABCB9, AMPD1, ANGPT4, ATXN80S, C11, CCr10, HIST1H2AE, HIST1H2BG, IGHE, KCNA3, KCNG2, LOC100130100, MAN1A1, MANEA, MAST1, MROH7, MZB1, PAX7, PDK1, RASGRP3, REN, SPAG4, ST6GALNAC4, TGM5, UGT2B17, ZBP1, ZNF165; TLS gene signature as described in the review by Zhu et al. (Zhu, G., et al. Tumor-Associated Tertiary Lymphoid Structures: Gene-Expression Profiling and Their Bioengineering. Front. Immunol. 8, 767 (2017)): CCL2, CCL3, CCL4, CCL5, CCL8, CCL18, CCL19, CCL21, CXCL9, CXCL10, CXCL11, CXCL13; B cell gene signature derived from this study: CD79A, SLAMF7, BTK, TNFRSF17 or BCMA, IGJ or JCHAIN, IGLL5, RBPJ, MZB1; and T cell effector gene signature: CD8A, EOMES, GZMA, TBX21, IFNG, GZMB, CXCL9, CXCL10 (Fehrenbacher, L., et al. Atezolizumab versus docetaxel for patients with previously treated non-small-cell lung cancer (POPLAR): a multicentre, open-label, phase 2 randomised controlled trial. Lancet 387, 1837-1846 (2016)).

scRNAseq Processing and Population-Specific Signatures

Samples were obtained from Gene Expression Omnibus (GEO) under accession GSE131907 as raw UMI counts per cell. Additionally, annotations of cell type and sample origin for each cell were retrieved. These cell type annotations from the authors were used to isolate B cells from tumor and normal adjacent tissue as well as draining lymph nodes from the expression matrix. The Seurat package (v3.1.4) was used for downstream analysis of B cells in R (3.6.2). We filtered cells with less than 500 measured genes or more than 10 percent mitochondrial reads and normalized the expression counts of the remaining cells to log(CPM/100+1). Principal component analysis was performed on the 2000 most variable genes, and the first 30 principle components were used for UMAP dimensionality reduction and graph-based clustering. Clusters were determined using a resolution of 0.3. Markers for each cluster were detected by comparing all cells in a particular cluster to the rest of the cells in the dataset using Wilcoxon's rank sum test adjusted for multiple testing with Benjamini Hochberg. From the initial dataset we removed non-B cell contaminant clusters (CD3⁺ T cells, GZMB+ LILRA4⁺ pDCs, and HBA2⁺ red blood cells). After removal of these contaminant cells, variable gene detection, PCA, and UMAP dimensionality reduction were re-run with the parameters described above. Markers for each B cell subset were identified as genes with an adjusted p-value<0.001 and logFC>0.5 comparing B cells in a cluster to all other cells in the dataset. To guarantee B-cell specific expression of markers, only marker genes that were not expressed by non-B cells in the full dataset including stromal, tumor, and non-B immune cells (average log(CPM/100+1)<1 across non B cell populations) were further retained. Heatmaps for visual comparisons were created using the pheatmap R package (1.0.12).

CyTOF Sample Acquisition, Staining and Data Processing

Six fresh NSCLC tumor samples were procured from a commercial vendor (Discovery Life Sciences) as part of adult patients undergoing surgical resection. After overnight fixation, cells were washed with 3 mL of MaxPar® cell staining buffer and centrifuged at 800×g for 5 minutes. After aspiration of the wash buffer and resuspension of the cell pellet, another round of wash was performed with 4 mL of MaxPar® Water (Fluidigm). Cells were resuspended in 1 mL of MaxPar® Water and counted. After obtaining cell counts, 3 mL of MaxPar® Water was added and cells were pelleted one final time prior to instrument acquisition. Before introduction into the Helios™, a CyTOF® System (Fluidigm), pelleted cells were resuspended with 1× MaxPar® Water containing EQ™ Four Element Calibration Beads (Fluidigm) then filtered using a 12×75 mm tube with a 35 μm nylon mesh cell-strainer cap (Corning). All FCS files were normalized together using the MATLAB® (MathWorks) normalizer and analyzed using FlowJo® software (FlowJo, LLC, Ashland, Oregon). Protein marker expression intensities from mass cytometry analysis were aggregated from multiple samples and transformed using the inverse hyperbolic sine function. Dimensionality reduction was applied to the transformed expression matrix using the uniform manifold approximation and projection (UMAP) package with the following default parameters: min dist:0.1, n neighbors: 15, n components: 2, and metric: euclidean. Individual samples were downsampled to 8,000 cells per sample in CD45⁺ and CD8⁺ populations. UMAP coordinates were appended to “.fcs” files as additional channels for integration with manual gating analysis in FlowJo (FlowJo, LLC).

Immunofluorescence and Tertiary Lymphoid Analysis

Triple immunofluorescence (CD8/CD79/Ki-67) was performed on 4 μm sections of formalin fixed paraffin embedded (FFPE) tumor samples, following deparaffinization, rehydration, and epitope retrieval with Target Retrieval Solution pH 6 for 20 minutes at 99° C. Each staining round began with quenching in 3% hydrogen peroxide. Sections were incubated in anti-CD79a rabbit monoclonal (SP18) (Thermo Fischer Scientific, MA5-14556) diluted 1:300, detected with PowerVision Poly-horse radish peroxidase (HRP) anti-rabbit, and amplified with Alexa-Fluor 488 Tyramide. Elution was performed in Target Retrieval Solution pre-warmed to 99° C. for 20 minutes. Sections were incubated in anti-CD8 mouse monoclonal C8/144B (Dako, M7103) diluted to 1.5 μg/ml, detected with PowerVision Poly-HRP anti-mouse, and amplified with Alexa-Fluor 647 Tyramide. Following an elution step, sections were incubated in anti-Ki67 mouse monoclonal (MIB-1) (Dako, M7240) diluted to 1 μg/ml, detected with PowerVision Poly-HRP anti-mouse, and amplified with Alexa-Fluor 555 Tyramide. Slides were counterstained with 4′,6-diamidino-2-phenylindole (DAPI). Images of fluorescent slides were acquired on a NanoZoomer XR.

TLS were identified using H&E stained FFPE sections and examined by a pathologist (HK) to identify lymphoid aggregates resembling TLS. We defined TLS as those that morphologically resembled lymphoid aggregates areas with well-defined zones (Dieu-Nosjean, M. C., et al. Tertiary lymphoid structures in cancer and beyond. Trends Immunol. 35, 571-580 (2014)). Representative slides were selected for IHC using MECA-79 that binds to peripheral lymph node addressins.

BCR Repertoire Analysis

Of 32 tumor samples, 16 pairs (9 from BIRCH, 7 from FIR) were collected from NSCLC patients treated with atezolizumab (Tan, P., et al. Regulative role of the CXCL13-CXCR5 axis in the tumor microenvironment. Precis. Clin. Med. 1, 49-56 (2018); Bruno, T.C. Evaluating the antitumor role of B cells in patients with non-small cell lung cancer. J. Clin. Oncol. 35, 75 (2017)). RNA was extracted from FFPE tumors using High Pure FFPE RNA isolation Kit (Roche). BOR cDNA libraries were generated using Immunoverse IgH Kit (ArcherDX, Inc., Boulder, CO). Libraries were quantified by KAPA Universal Library Quantification Kit (KAPA Biosystems, Wilmington, MA). Libraries were pooled at equimolar concentrations and sequenced using a MiSeq Reagent Kit v3 (600-cycle) and Illumina MiSeq instrument (Illumina, Inc. San Diego, CA). Sequence data was analyzed by Archer® analysis software to calculate the SDI of each library (Greiff, V., et al. A bioinformatic framework for immune repertoire diversity profiling enables detection of immunological status. Genome Med. 7, 49 (2015); Kaplinsky, J. et al. Robust estimates of overall immune-repertoire diversity from high-throughput measurements on samples. Nat. Commun. 7, 11881 (2016)). Statistical analysis of SDI between sample pairs of the same patients was done using Wilcoxon matched-pairs signed rank test, and comparison of changes of SDI pre/post treatment between different groups of patients was analyzed by Mann Whitney test.

### VIII. OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Exemplary embodiments of the invention include those enumerated below.

1. A method of identifying an individual having a cancer who may benefit from a treatment comprising a PD-L1 axis binding antagonist, the method comprising determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

2. A method of selecting a therapy for an individual having a cancer, the method comprising determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

3. The method of embodiment 1 or 2, wherein the immune-score expression level of the two or more genes in the sample is above the reference immune-score expression level and the method further comprises administering to the individual an effective amount of a PD-L1 axis binding antagonist.

4. A method of treating an individual having a cancer, the method comprising:
(a) determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and
(b) administering an effective amount of a PD-L1 axis binding antagonist to the individual.

5. A method of treating cancer in an individual that has been determined to have an immune-score expression level of two or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes, the method comprising administering to the individual an effective amount of a PD-L1 axis binding antagonist.

6. The method of any one of embodiments 1-5, wherein the immune-score reference expression level is an immune-score expression level of the two or more genes in a reference population.

7. The method of embodiment 6, wherein the reference population is a population of individuals having the cancer.

8. The method of embodiment 7, wherein the population of individuals comprises a first subset of individuals who have been treated with a PD-L1 axis binding antagonist and a second subset of individuals who have been treated with therapy that does not comprise a PD-L1 axis binding antagonist.

9. The method of embodiment 8, wherein the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

10. The method of embodiment 8 or 9, wherein the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

11. The method of embodiment 10, wherein the therapy that does not comprise a PD-L1 axis binding antagonist comprises a chemotherapeutic agent.

12. The method of embodiment 11, wherein the chemotherapeutic agent is docetaxel.

13. The method of any one of embodiments 9-12, wherein responsiveness to treatment comprises an extension in OS, an extension in progression-free survival (PFS), or an increase in best confirmed overall response (BOOR).

14. The method of embodiment 13, wherein responsiveness to treatment comprises an extension in OS.

15. The method of any one of embodiments 6-14, wherein the reference immune-score expression level is a median of the expression level of each of the two or more genes in the reference population.

16. The method of any one of embodiments 1-15, wherein the genes comprise three or more of CD79A, CD19, BANK1, JCHAIN, SLAMF7, BTK, TNFRSF17, IGJ, IGLL5, RBPJ, and MZB1.

17. The method of any one of embodiments 1-16, wherein the genes comprise four or more of CD79A, CD19, BANK1, JCHAIN, SLAMF7, BTK, TNFRSF17, IGJ, IGLL5, RBPJ, and MZB1.

18. The method of any one of embodiments 1-17, wherein the genes comprise five or more of CD79A, CD19, BANK1, JCHAIN, SLAMF7, BTK, TNFRSF17, IGJ, IGLL5, RBPJ, and MZB1.

19. The method of any one of embodiments 1-18, wherein the genes comprise six or more of CD79A, CD19, BANK1, JCHAIN, SLAMF7, BTK, TNFRSF17, IGJ, IGLL5, RBPJ, and MZB1.

20. The method of any one of embodiments 1-19, wherein the genes comprise seven, eight, nine, ten, or more of CD79A, CD19, BANK1, JCHAIN, SLAMF7, BTK, TNFRSF17, IGJ, IGLL5, RBPJ, and MZB1.

21. The method of any one of embodiments 1-20, wherein the genes comprise CD79A, CD19, BANK1, JCHAIN, SLAMF7, BTK, TNFRSF17, IGJ, IGLL5, RBPJ, and MZB1.

22. A method of identifying an individual having a cancer who may benefit from a treatment comprising a PD-L1 axis binding antagonist, the method comprising determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's overall survival (OS) as compared to treatment without the PD-L1 axis binding antagonist.

23. A method of selecting a therapy for an individual having a cancer, the method comprising determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

24. The method of embodiment 22 or 23, wherein the immune-score expression level of the one or more genes in the sample is above the reference immune-score expression level and the method further comprises administering to the individual an effective amount of a PD-L1 axis binding antagonist.

25. A method of treating an individual having a cancer, the method comprising:
(a) determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and
(b) administering an effective amount of a PD-L1 axis binding antagonist to the individual.

26. A method of treating cancer in an individual that has been determined to have an immune-score expression level of one or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, the method comprising administering to the individual an effective amount of a PD-L1 axis binding antagonist, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

27. The method of any one of embodiments 22-26, wherein the immune-score expression level of one of genes CD79A, CD19, BANK1, JCHAIN, SLAMF7, BTK, TNFRSF17, IGJ, IGLL5, RBPJ, and MZB1 is determined.

28. The method of embodiment 27, wherein the immune-score expression level of CD79A is determined.

29. The method of any one of embodiments 22-28, wherein the immune-score reference expression level is an immune-score expression level of the one or more genes in a reference population.

30 The method of embodiment 29, wherein the reference population is a population of individuals having the cancer.

31. The method of embodiment 30, wherein the population of individuals comprises a first subset of individuals who have been treated with a PD-L1 axis binding antagonist and a second subset of individuals who have been treated with therapy that does not comprise a PD-L1 axis binding antagonist.

32. The method of embodiment 31, wherein the immune-score reference expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

33. The method of embodiment 31 or 32, wherein the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

34. The method of embodiment 33, wherein the therapy that does not comprise a PD-L1 axis binding antagonist comprises a chemotherapeutic agent.

35 The method of embodiment 34, wherein the chemotherapeutic agent is docetaxel.

36. The method of any one of embodiments 32-35, wherein responsiveness to treatment comprises an extension in OS, an extension in PFS, or an increase in BOOR.

37. The method of embodiment 36, wherein responsiveness to treatment comprises an extension in OS.

38. The method of any one of embodiments 29-37, wherein the immune-score reference expression level is a median of the expression level of each of the one or more genes in the reference population.

39. The method of embodiment 38, wherein the median expression level is the median of a mean Z score of the expression level of each of the two or more genes in the reference population.

40 The method of any one of embodiments 22-39, wherein the genes comprise two or more of CD79A, CD19, BANK1, JCHAIN, SLAMF7, BTK, TNFRSF17, IGJ, IGLL5, RBPJ, and MZB1.

41. The method of any one of embodiments 1-5 and 40, wherein the two or more genes comprise TNFRSF17 and IGJ.

42. The method of any one of embodiments 1-5 and 41, wherein the two genes consist of TNFRSF17 and IGJ.

43. The method of any one of embodiments 22-41, wherein the genes comprise three or more of CD79A, CD19, BANK1, JCHAIN, SLAMF7, BTK, TNFRSF17, IGJ, IGLL5, RBPJ, and MZB1.

44. The method of any one of embodiments 22-41 and 43, wherein the genes comprise four or more of CD79A, CD19, BANK1, JCHAIN, SLAMF7, BTK, TNFRSF17, IGJ, IGLL5, RBPJ, and MZB1.

45 The method of any one of embodiments 22-41, 43, and 44, wherein the genes comprise five or more of CD79A, CD19, BANK1, JCHAIN, SLAMF7, BTK, TNFRSF17, IGJ, IGLL5, RBPJ, and MZB1.

46. The method of any one of embodiments 22-41 and 43-45, wherein the genes comprise six or more of CD79A, CD19, BANK1, JCHAIN, SLAMF7, BTK, TNFRSF17, IGJ, IGLL5, RBPJ, and MZB1.

47. The method of any one of embodiments 22-41 and 43-46, wherein the genes comprise seven, eight, nine, ten, or more of CD79A, CD19, BANK1, JCHAIN, SLAMF7, BTK, TNFRSF17, IGJ, IGLL5, RBPJ, and MZB1.

48. The method of any one of embodiments 22-41 and 43-47, wherein the genes comprise CD79A, CD19, BANK1, JCHAIN, SLAMF7, BTK, TNFRSF17, IGJ, IGLL5, RBPJ, and MZB1.

49. The method of embodiment 48, wherein the genes consist of CD79A, CD19, BANK1, JCHAIN, SLAMF7, BTK, TNFRSF17, IGJ, IGLL5, RBPJ, and MZB1.

50. A method of identifying an individual having a cancer who may benefit from a treatment comprising a PD-L1 axis binding antagonist, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist.

51. A method of selecting a therapy for an individual having a cancer, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist.

52. The method of embodiment 50 or 51, wherein the immune-score expression level of the one or more genes in the sample is above the reference immune-score expression level and the method further comprises administering to the individual an effective amount of a PD-L1 axis binding antagonist.

53. A method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and
(b) administering an effective amount of a PD-L1 axis binding antagonist to the individual.

54. A method of treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, the method comprising administering to the individual an effective amount of a PD-L1 axis binding antagonist.

55 The method of any one of embodiments 50-54, wherein the immune-score reference expression level is an immune-score expression level of the one or more genes in a reference population.

56. The method of embodiment 55, wherein the reference population is a population of individuals having the cancer.

57. The method of embodiment 56, wherein the population of individuals comprises a first subset of individuals who have been treated with a PD-L1 axis binding antagonist and a second subset of individuals who have been treated with therapy that does not comprise a PD-L1 axis binding antagonist.

58. The method of embodiment 57, wherein the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

59. The method of embodiment 57 or 58, wherein the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

60. The method of embodiment 59, wherein the therapy that does not comprise a PD-L1 axis binding antagonist comprises a chemotherapeutic agent.

61. The method of embodiment 60, wherein the chemotherapeutic agent is docetaxel.

62. The method of any one of embodiments 58-61, wherein responsiveness to treatment comprises an extension in OS, an extension in PFS, or an increase in BOOR.

63. The method of embodiment 62, wherein responsiveness to treatment comprises an extension in OS.

64. The method of any one of embodiments 55-63, wherein the reference immune-score expression level is a median of the expression level of each of the one or more genes in the reference population.

65. The method of any one of embodiments 50-64, wherein the genes comprise two or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

66. The method of any one of embodiments 50-65, wherein the genes comprise three or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

67. The method of any one of embodiments 50-66, wherein the genes comprise four or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

68. The method of any one of embodiments 50-67, wherein the genes comprise five or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

69. The method of any one of embodiments 50-68, wherein the genes comprise six or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

70. The method of any one of embodiments 50-69, wherein the genes comprise seven or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

71. The method of any one of embodiments 50-70, wherein the genes comprise eight or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

72. The method of any one of embodiments 50-71, wherein the genes comprise nine or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

73. The method of any one of embodiments 50-72, wherein the genes comprise 10 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

74. The method of any one of embodiments 50-73, wherein the genes comprise 11 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

75. The method of any one of embodiments 50-74, wherein the genes comprise 12 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

76. The method of any one of embodiments 50-75, wherein the genes comprise 13 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

77. The method of any one of embodiments 50-76, wherein the genes comprise MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

78. A method of identifying an individual having a cancer who may benefit from a treatment comprising a PD-L1 axis binding antagonist, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

79. A method of selecting a therapy for an individual having a cancer, the method comprising determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

80. The method of embodiment 78 or 79, wherein the immune-score expression level of the one or more genes in the sample is above the reference immune-score expression level and the method further comprises administering to the individual an effective amount of a PD-L1 axis binding antagonist.

81. A method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and
(b) administering an effective amount of a PD-L1 axis binding antagonist to the individual.

82. A method of treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, the method comprising administering to the individual an effective amount of a PD-L1 axis binding antagonist, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

83. The method of any one of embodiments 78-82, wherein the immune-score reference expression level is an immune-score expression level of the one or more genes in a reference population.

84. The method of embodiment 83, wherein the reference population is a population of individuals having the cancer.

85. The method of embodiment 84, wherein the population of individuals comprises a first subset of individuals who have been treated with a PD-L1 axis binding antagonist and a second subset of individuals who have been treated with therapy that does not comprise a PD-L1 axis binding antagonist.

86. The method of embodiment 85, wherein the immune-score reference expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference immune-score expression level, wherein the individual's responsiveness to treatment with the PD-L1 axis binding antagonist is significantly improved relative to the individual's responsiveness to treatment with the therapy that does not comprise a PD-L1 axis binding antagonist.

87. The method of embodiment 85 or 86, wherein the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

88. The method of embodiment 87, wherein the therapy that does not comprise a PD-L1 axis binding antagonist comprises a chemotherapeutic agent.

89. The method of embodiment 88, wherein the chemotherapeutic agent is docetaxel.

90. The method of any one of embodiments 86-89, wherein responsiveness to treatment comprises an extension in OS, an extension in PFS, or an increase in BOOR. in OS.

91. The method of embodiment 90, wherein responsiveness to treatment comprises an extension in OS.

92. The method of any one of embodiments 83-91, wherein the immune-score reference expression level is a median of the expression level of each of the one or more genes in the reference population.

93. The method of embodiment 92, wherein the median expression level is the median of a mean Z score of the expression level of each of the one or more genes in the reference population.

94. The method of any one of embodiments 78-93, wherein the genes comprise two or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

95. The method of any one of embodiments 78-94, wherein the genes comprise three or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

96. The method of any one of embodiments 78-95, wherein the genes comprise four or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

97. The method of any one of embodiments 78-96, wherein the genes comprise five or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

98. The method of any one of embodiments 78-97, wherein the genes comprise six or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

99. The method of any one of embodiments 78-98, wherein the genes comprise seven or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

100. The method of any one of embodiments 78-99, wherein the genes comprise eight or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

101. The method of any one of embodiments 78-100, wherein the genes comprise nine or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

102. The method of any one of embodiments 78-101, wherein the genes comprise 10 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

103. The method of any one of embodiments 78-102, wherein the genes comprise 11 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

104. The method of any one of embodiments 78-103, wherein the genes comprise 12 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

105. The method of any one of embodiments 78-104, wherein the genes comprise 13 or more of MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

106. The method of any one of embodiments 78-105, wherein the genes comprise MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5.

107. A method of identifying an individual having a cancer who may benefit from a treatment comprising a PD-L1 axis binding antagonist, the method comprising determining the presence of a tertiary lymphoid structure (TLS) in a tumor sample from the individual, wherein the presence of a TLS in the tumor sample identifies the individual as one who may benefit from a treatment comprising a PD-L1 axis binding antagonist.

108. A method of selecting a therapy for an individual having a cancer, the method comprising determining the presence of a TLS in a tumor sample from the individual, wherein the presence of a TLS in the tumor sample identifies the individual as one who may benefit from a treatment comprising a PD-L1 axis binding antagonist.

109. The method of embodiment 107 or 108, wherein the sample from the individual is determined to have the presence of a TLS and the method further comprises administering to the individual an effective amount of a PD-L1 axis binding antagonist.

110. A method of treating an individual having a cancer, the method comprising:
(a) determining the presence of a TLS in a tumor sample from the individual; and
(b) administering an effective amount of a PD-L1 axis binding antagonist to the individual.

111. A method of treating cancer in an individual that has been determined to have the presence of a TLS in a tumor sample from the individual, the method comprising administering to the individual an effective amount of a PD-L1 axis binding antagonist.

112. The method of any one of embodiments 107-111, wherein the presence of a TLS is determined by histological staining, immunohistochemistry (INC), immunofluorescence, or gene expression analysis.

113. The method of embodiment 112, wherein the histological staining comprises hematoxylin and eosin (H&E) staining.

114. The method of embodiment 112, wherein the IHC or immunofluorescence comprises detecting CD62L, L-selectin, CD40, or CD8.

115. The method of embodiment 114, wherein CD62L or L-selectin is detected using a MECA-79 antibody.

116. The method of embodiment 112, wherein the gene expression analysis comprises determining the expression level of a TLS gene signature in the sample.

117. The method of embodiment 116, wherein the TLS gene signature comprises one or more of genes CCL2, CCL3, CCL4, CCL5, CCL8, CCL18, CCL19, CCL21, CXCL9, CXCL10, CXCL11, and CXCL13.

118. The method of embodiment 117, wherein the genes comprise two or more of CCL2, CCL3, CCL4, CCL5, CCL8, CCL18, CCL19, CCL21, CXCL9, CXCL10, CXCL11, and CXCL13.

119. A method of identifying an individual having a cancer who may benefit from a treatment comprising a PD-L1 axis binding antagonist, the method comprising determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

120. A method of selecting a therapy for an individual having a cancer, the method comprising determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

121. The method of embodiment 119 or 120, wherein the immune-score expression level of the two or more genes in the sample is above the reference immune-score expression level and the method further comprises administering to the individual an effective amount of a PD-L1 axis binding antagonist.

122. A method of treating an individual having a cancer, the method comprising:
(a) determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and
(b) administering an effective amount of a PD-L1 axis binding antagonist to the individual.

123. A method of treating cancer in an individual that has been determined to have an immune-score expression level of two or more of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes, the method comprising administering to the individual an effective amount of a PD-L1 axis binding antagonist.

124. The method of any one of embodiments 119-123, wherein the reference immune-score expression level is an immune-score expression level of the two or more genes in a reference population.

125. The method of embodiment 124, wherein the reference population is a population of individuals having the cancer.

126. The method of embodiment 125, wherein the population of individuals comprises a first subset of individuals who have been treated with a PD-L1 axis binding antagonist and a second subset of individuals who have been treated with therapy that does not comprise a PD-L1 axis binding antagonist.

127. The method of embodiment 126, wherein the reference immune-score expression level significantly separates each of the first and second subsets of individuals based on a significant difference between an individual's responsiveness to treatment with the PD-L1 axis binding antagonist and an individual's responsiveness to treatment with the therapy that does not comprise a PD-L1 axis binding antagonist above the reference expression level, wherein the individual's responsiveness to treatment with the PD-L1 axis binding antagonist is significantly improved relative to the individual's responsiveness to treatment with the therapy that does not comprise a PD-L1 axis binding antagonist.

128. The method of embodiment 126 or 127, wherein the therapy that does not comprise a PD-L1 axis binding antagonist comprises an anti-neoplastic agent, a chemotherapeutic agent, a growth inhibitory agent, an anti-angiogenic agent, a radiation therapy, a cytotoxic agent, or a combination thereof.

129. The method of embodiment 128, wherein the therapy that does not comprise a PD-L1 axis binding antagonist comprises a chemotherapeutic agent.

130. The method of embodiment 129, wherein the chemotherapeutic agent is docetaxel.

131. The method of any one of embodiments 127-130, wherein responsiveness to treatment comprises an extension in OS, an extension in PFS, or an increase in BOOR.

132. The method of embodiment 131, wherein responsiveness to treatment comprises an extension in OS.

133. The method of any one of embodiments 125-132, wherein the reference immune-score expression level is a median of the expression level of each of the two or more genes in the reference population.

134. The method of embodiment 133, wherein the median expression level is the median of a mean Z score of the expression level of each of the two or more genes in the reference population.

135. The method of any one of embodiments 110-134, wherein the genes comprise three or more of CCL2, CCL3, CCL4, CCL5, CCL8, CCL18, CCL19, CCL21, CXCL9, CXCL10, CXCL11, and CXCL13.

136. The method of any one of embodiments 119-135, wherein the genes comprise four or more of CCL2, CCL3, CCL4, CCL5, CCL8, CCL18, CCL19, CCL21, CXCL9, CXCL10, CXCL11, and CXCL13.

137. The method of any one of embodiments 119-136, wherein the genes comprise five or more of CCL2, CCL3, CCL4, CCL5, CCL8, CCL18, CCL19, CCL21, CXCL9, CXCL10, CXCL11, and CXCL13.

138. The method of any one of embodiments 119-137, wherein the genes comprise six or more of CCL2, CCL3, CCL4, CCL5, CCL8, CCL18, CCL19, CCL21, CXCL9, CXCL10, CXCL11, and CXCL13.

139. The method of any one of embodiments 119-138, wherein the genes comprise seven or more of CCL2, CCL3, CCL4, CCL5, CCL8, CCL18, CCL19, CCL21, CXCL9, CXCL10, CXCL11, and CXCL13.

140. The method of any one of embodiments 119-139, wherein the genes comprise eight or more of CCL2, CCL3, CCL4, CCL5, CCL8, CCL18, CCL19, CCL21, CXCL9, CXCL10, CXCL11, and CXCL13.

141. The method of any one of embodiments 119-140, wherein the genes comprise nine or more of CCL2, CCL3, CCL4, CCL5, CCL8, CCL18, CCL19, CCL21, CXCL9, CXCL10, CXCL11, and CXCL13.

142. The method of any one of embodiments 119-141, wherein the genes comprise ten or more of CCL2, CCL3, CCL4, CCL5, CCL8, CCL18, CCL19, CCL21, CXCL9, CXCL10, CXCL11, and CXCL13.

143. The method of any one of embodiments 119-142, wherein the genes comprise eleven or more of CCL2, CCL3, CCL4, CCL5, CCL8, CCL18, CCL19, CCL21, CXCL9, CXCL10, CXCL11, and CXCL13.

144. The method of any one of embodiments 119-143, wherein the genes comprise CCL2, CCL3, CCL4, CCL5, CCL8, CCL18, CCL19, CCL21, CXCL9, CXCL10, CXCL11, and CXCL13.

145. The method of any one of embodiments 1-106 and 119-144, wherein the expression level is a nucleic acid expression level.

146. The method of embodiment 145, wherein the nucleic acid expression level is an mRNA expression level.

147. The method of embodiment 146, wherein the mRNA expression level is determined by RNA-seq, RT-qPCR, qPCR, multiplex qPCR or RT-qPCR, microarray analysis, SAGE, MassARRAY technique, FISH, or a combination thereof.

148. The method of embodiment 147, wherein the mRNA expression level is detected using RNA-seq.

149. The method of any one of embodiments 1-106 and 119-144, wherein the expression level is a protein expression level.

150. The method of embodiment 149, wherein the protein expression level is determined by IHC, immunofluorescence, mass spectrometry, flow cytometry, and Western blot, or a combination thereof.

151. The method of any one of embodiments 1-106 and 119-150, wherein the expression level is detected in tumor cells, tumor-infiltrating immune cells, stromal cells, normal adjacent tissue (NAT) cells, or a combination thereof.

152. A method of identifying an individual having a cancer who may benefit from a treatment comprising a PD-L1 axis binding antagonist, the method comprising determining the number of B cells in a tumor sample from the individual, wherein a number of B cells in the tumor sample that is above a reference number of B cells identifies the individual as one who may benefit from a treatment comprising a PD-L1 axis binding antagonist.

153. A method of selecting a therapy for an individual having a cancer, the method comprising determining the number of B cells in a tumor sample from the individual, wherein a number of B cells in the tumor sample that is above a reference number of B cells identifies the individual as one who may benefit from a treatment comprising a PD-L1 axis binding antagonist.

154. The method of embodiment 152 or 153, wherein the number of B cells in the sample is above the reference number and the method further comprises administering to the individual an effective amount of a PD-L1 axis binding antagonist.

155. A method of treating an individual having a cancer, the method comprising:
(a) determining the number of B cells in a tumor sample from the individual; and
(b) administering an effective amount of a PD-L1 axis binding antagonist to the individual.

156. A method of treating cancer in an individual that has been determined to have a number of B cells in a tumor sample from the individual that is above a reference number of B cells, the method comprising administering to the individual an effective amount of a PD-L1 axis binding antagonist.

157. The method of any one of embodiments 152-156, wherein the B cells comprise CD79⁺B cells, IgG+ B cells, and/or plasma cells.

158. The method of embodiment 157, wherein the B cells comprise plasma cells.

159. A method of identifying an individual having a cancer who may benefit from a treatment comprising a PD-L1 axis binding antagonist, the method comprising determining whether the individual has clonally expanded B cells in a tumor sample from the individual, wherein clonally expanded B cells in the sample identify the individual as one who may benefit from a treatment comprising a PD-L1 axis binding antagonist.

160. A method of selecting a therapy for an individual having a cancer, the method comprising determining whether the individual has clonally expanded B cells in a tumor sample from the individual, wherein clonally expanded B cells in the sample identify the individual as one who may benefit from a treatment comprising a PD-L1 axis binding antagonist.

161. The method of embodiment 159 or 160, wherein the tumor sample comprises clonally expanded B cells and the method further comprises administering to the individual an effective amount of a PD-L1 axis binding antagonist.

162. A method of treating an individual having a cancer, the method comprising:
(a) determining that the individual has clonally expanded B cells in a tumor sample from the individual; and
(b) administering an effective amount of a PD-L1 axis binding antagonist to the individual.

163. A method of treating cancer in an individual that has been determined to have clonally expanded B cells in a tumor sample from the individual, the method comprising administering to the individual an effective amount of a PD-L1 axis binding antagonist.

164. The method of any one of embodiments 159-163, wherein the clonally expanded B cells are clonally expanded plasma cells.

165. The method of any one of embodiments 159-164, wherein clonally expanded B cells are detected by measuring the diversity of the B cell receptor (BCR) gene repertoire in the tumor sample.

166. The method of embodiment 165, wherein a Shannon Diversity Index (SDI) of the BCR gene repertoire in the tumor sample from the individual that is below a reference SDI identifies the individual as one who may benefit from a treatment comprising a PD-L1 axis binding antagonist.

167. The method of any one of embodiments 1-106 and 119-151, wherein the sample is a tissue sample, a cell sample, a whole blood sample, a plasma sample, a serum sample, or a combination thereof.

168. The method of embodiment 167, wherein the tissue sample is a tumor tissue sample.

169. The method of any one of embodiments 107-118 and 152-166, wherein the tumor sample is a tumor tissue sample.

170. The method of embodiment 168 or 169, wherein the tumor tissue sample comprises tumor cells, tumor-infiltrating immune cells, stromal cells, NAT cells, or a combination thereof.

171. The method of any one of embodiments 168-170, wherein the tumor tissue sample is a formalin-fixed and paraffin-embedded (FFPE) sample, an archival sample, a fresh sample, or a frozen sample.

172. The method of embodiment 171, wherein the tumor tissue sample is an FFPE sample.

173. The method of any one of embodiments 1-172, wherein the cancer is a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a Merkel cell cancer, or a hematologic malignancy.

174. The method of embodiment 173, wherein the cancer is a lung cancer, a kidney cancer, a bladder cancer, or a breast cancer.

175. The method of embodiment 174, wherein the lung cancer is a non-small cell lung cancer (NSCLC).

176. The method of embodiment 175, wherein the NSCLC is non-squamous NSCLC.

177. The method of embodiment 175, wherein the NSCLC is squamous NSCLC.

178. The method of any one of embodiments 1-3, 6-52, 55-109, 112-121, 124-154, 157-161, and 164-177, wherein the benefit comprises an extension in the individual's OS, an extension in the individual's PFS, and/or an improvement in the individual's BOOR, as compared to treatment without the PD-L1 axis binding antagonist.

179. The method of embodiment 178, wherein the benefit comprises an extension in the individual's OS as compared to treatment without the PD-L1 axis binding antagonist.

180. The method of any one of embodiments 1-179, wherein the PD-L1 axis binding antagonist is selected from the group consisting of a PD-L1 binding antagonist, a PD-1 binding antagonist, and a PD-L2 binding antagonist.

181. The method of embodiment 180, wherein the PD-L1 axis binding antagonist is a PD-L1 binding antagonist.

182. The method of embodiment 181, wherein the PD-L1 binding antagonist inhibits the binding of PD-L1 to one or more of its ligand binding partners.

183. The method of embodiment 182, wherein the PD-L1 binding antagonist inhibits the binding of PD-L1 to PD-1.

184. The method of embodiment 182, wherein the PD-L1 binding antagonist inhibits the binding of PD-L1 to B7-1.

185. The method of any one of embodiments 182-184, wherein the PD-L1 binding antagonist inhibits the binding of PD-L1 to both PD-1 and B7-1.

186. The method of any one of embodiments 182-185, wherein the PD-L1 binding antagonist is an antibody or antigen-binding fragment thereof.

187. The method of embodiment 186, wherein the antibody is selected from the group consisting of atezolizumab, MDX-1105, MED14736 (durvalumab), and MSB0010718C (avelumab).

188. The method of embodiment 186, wherein the antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

189. The method of embodiment 186, wherein the antibody comprises a heavy chain variable region comprising the amino acid sequence of SEQ ID NO: 7 and a light chain variable region comprising the amino acid sequence of SEQ ID NO: 8.

190. The method of embodiment 180, wherein the PD-L1 axis binding antagonist is a PD-1 binding antagonist.

191. The method of embodiment 190, wherein the PD-1 binding antagonist inhibits the binding of PD-1 to one or more of its ligand binding partners.

192. The method of embodiment 191, wherein the PD-1 binding antagonist inhibits the binding of PD-1 to PD-L1.

193. The method of embodiment 191, wherein the PD-1 binding antagonist inhibits the binding of PD-1 to PD-L2.

194. The method of any one of embodiments 191-193, wherein the PD-1 binding antagonist inhibits the binding of PD-1 to both PD-L1 and PD-L2.

195. The method of any one of embodiments 191-194, wherein the PD-1 binding antagonist is an antibody or antigen-binding fragment thereof.

196. The method of embodiment 195, wherein the antibody is selected from the group consisting of: MDX-1106 (nivolumab), MK-3475 (pembrolizumab), MEDI-0680 (AMP-514), PDR001, REGN2810, and BGB-108.

197. The method of any one of embodiments 190-194, wherein the PD-1 binding antagonist is an Fc-fusion protein.

198. The method of embodiment 197, wherein the Fc-fusion protein is AMP-224.

199. The method of any one of embodiments 1-198, wherein the individual has not been previously treated for the cancer.

200. The method of embodiment 199, wherein the individual has not been previously administered a PD-L1 axis binding antagonist.

201. The method of embodiment 199 or 200, wherein the cancer is NSCLC, and wherein the individual has no EGFR or ALK genomic tumor aberrations.

202. The method of any one of embodiments 1-198, wherein the individual has previously been treated for the cancer.

203. The method of embodiment 202, wherein the individual has previously been treated for the cancer by administration of a platinum-containing chemotherapeutic agent to the individual, and wherein the individual has failed to respond to the chemotherapeutic agent.

204. The method of any one of embodiments 4-21, 25-48, 53-77, 81-106, 110-118, 122-151, 155-158, and 162-203, wherein the PD-L1 axis binding antagonist is administered as a monotherapy.

205. The method of any one of embodiments 4-21, 25-48, 53-77, 81-106, 110-118, 122-151, 155-158, and 162-203, wherein the method further comprises administering an effective amount of one or more additional therapeutic agents.

206. The method of embodiment 205, wherein the one or more additional therapeutic agents comprise an anti-neoplastic agent, a chemotherapeutic agent, a growth inhibitory agent, an anti-angiogenic agent, a radiation therapy, a cytotoxic agent, an immunomodulatory agent, or a combination thereof.

207. The method of any one of embodiments 1-206, wherein the individual is a human. 208. A kit comprising a PD-L1 axis binding antagonist and instructions to administer the PD-L1 axis binding antagonist to an individual who has been identified as one who may benefit from a treatment comprising the PD-L1 binding antagonist in accordance with the method of any one of embodiments 1-3, 6-52, 55-109, 111-121, 124-154, 157-161, and 164-203.

209. A kit comprising a PD-L1 axis binding antagonist and instructions to administer the PD-L1 axis binding antagonist to an individual who has been selected for a treatment comprising the PD-L1 binding antagonist in accordance with the method of any one of embodiments 2, 3, 6-21, 23, 27-49, 51, 79, 83-106, 108, 112-118, 120, 124-151, 153, 154, 157, 158, 160, 161, and 164-203 210. A kit for identifying an individual having a cancer who may benefit from a treatment comprising a PD-L1 axis binding antagonist, the kit comprising reagents for determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes that is above a reference immune-score expression level of the two or more genes identifies the individual as one who may benefit from a treatment comprising a PD-L1 axis binding antagonist.

211. A kit for identifying an individual having a cancer who may benefit from a treatment comprising a PD-L1 axis binding antagonist, the kit comprising reagents for determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's overall survival (OS) as compared to treatment without the PD-L1 axis binding antagonist.

212. A kit for identifying an individual having a cancer who may benefit from a treatment comprising a PD-L1 axis binding antagonist, the kit comprising reagents for determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's overall survival (OS) as compared to treatment without the PD-L1 axis binding antagonist.

213. A kit for identifying an individual having a cancer who may benefit from a treatment comprising a PD-L1 axis binding antagonist, the kit comprising reagents for determining the presence of a tertiary lymphoid structure (TLS) in a tumor sample from the individual, wherein the presence of a TLS in the tumor sample identifies the individual as one who may benefit from a treatment comprising a PD-L1 axis binding antagonist.

214. A kit for identifying an individual having a cancer who may benefit from a treatment comprising a PD-L1 axis binding antagonist, the kit comprising reagents for determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes that is above a reference immune-score expression level of the two or more genes identifies the individual as one who may benefit from a treatment comprising a PD-L1 axis binding antagonist.

215. A kit for identifying an individual having a cancer who may benefit from a treatment comprising a PD-L1 axis binding antagonist, the kit comprising reagents for determining the number of B cells in a tumor sample from the individual, wherein a number of B cells in the tumor sample that is above a reference number of B cells identifies the individual as one who may benefit from a treatment comprising a PD-L1 axis binding antagonist, optionally wherein the B cells comprise plasma cells.

216. A kit for identifying an individual having a cancer who may benefit from a treatment comprising a PD-L1 axis binding antagonist, the kit comprising reagents for determining whether the individual has clonally expanded B cells in a tumor sample from the individual, wherein clonally expanded B cells in the sample identify the individual as one who may benefit from a treatment comprising a PD-L1 axis binding antagonist.

217. A PD-L1 axis binding antagonist for use in a method of treating an individual having a cancer, the method comprising:
(a) determining the expression level of two or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the two or more genes in the sample is determined to be above a reference immune-score expression level of the two or more genes; and
(b) administering an effective amount of a PD-L1 axis binding antagonist to the individual.

218. A PD-L1 axis binding antagonist for treating cancer in an individual that has been determined to have an immune-score expression level of two or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the two or more genes.

219. A PD-L1 axis binding antagonist for use in a method of treating an individual having a cancer, the method comprising:
(a) determining the expression level of one or more of genes CD79A, CD19, BANK1, JCHAIN, SLAMF7, BTK, TNFRSF17, IGJ, IGLL5, RBPJ, and MZB1 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and
(b) administering an effective amount of a PD-L1 axis binding antagonist to the individual.

220. A PD-L1 axis binding antagonist for use in treating cancer in an individual that has been determined to have an immune-score expression level of one or more of genes CD79A, CD19, BANK1, JCHAIN, SLAMF7, BTK, TNFRSF17, IGJ, IGLL5, RBPJ, and MZB1 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

221. A PD-L1 axis binding antagonist for use in a method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes; and
(b) administering an effective amount of a PD-L1 axis binding antagonist to the individual.

222. A PD-L1 axis binding antagonist for treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes.

223. A PD-L1 axis binding antagonist for use in a method of treating an individual having a cancer, the method comprising:
(a) determining the expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual, wherein an immune-score expression level of the one or more genes in the sample is determined to be above a reference immune-score expression level of the one or more genes, thereby identifying the individual as one who may benefit from a treatment comprising a PD-L1 axis binding antagonist, wherein the benefit comprises an extension in the individual's OS as compared to treatment without the PD-L1 axis binding antagonist; and
(b) administering an effective amount of a PD-L1 axis binding antagonist to the individual.

224. A PD-L1 axis binding antagonist for use in treating cancer in an individual that has been determined to have an immune-score expression level of one or more of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a sample from the individual that is above a reference immune-score expression level of the one or more genes, wherein an immune-score expression level of the one or more genes that is above a reference immune-score expression level of the one or more genes identifies the individual as one who may benefit from a treatment comprising a PD-L1 axis binding antagonist, wherein the benefit comprises an extension in the individual's OS as compared to treatment without the PD-L1 axis binding antagonist.

225. A PD-L1 axis binding antagonist for use in a method of treating an individual having a cancer, the method comprising:
(a) determining the presence of a TLS in a tumor sample from the individual; and
(b) administering an effective amount of a PD-L1 axis binding antagonist to the individual.

226. A PD-L1 axis binding antagonist for use in treating cancer in an individual that has been determined to have the presence of a TLS in a tumor sample from the individual.

227. A PD-L1 axis binding antagonist for use in a method of treating an individual having a cancer, the method comprising:
(a) determining the expression level of two or more of genes CCL2, CCL3, CCL4, CCL5, CCL8, CCL18, CCL19, CCL21, CXCL9, CXCL10, CXCL11, and CXCL13 in a sample from the individual, wherein an immune-score expression level of the two or more genes in the sample is determined to be above a reference immune-score expression level of the two or more genes; and
(b) administering an effective amount of a PD-L1 axis binding antagonist to the individual.

228. A PD-L1 axis binding antagonist for use in treating cancer in an individual that has been determined to have an immune-score expression level of two or more of genes CCL2, CCL3, CCL4, CCL5, CCL8, CCL18, CCL19, CCL21, CXCL9, CXCL10, CXCL11, and CXCL13 in a sample from the individual that is above a reference immune-score expression level of the two or more genes.

229. A PD-L1 axis binding antagonist for use in a method of treating an individual having a cancer, the method comprising:
(a) determining the number of B cells in a tumor sample from the individual, optionally wherein the B cells comprise plasma cells; and
(b) administering an effective amount of a PD-L1 axis binding antagonist to the individual.

230. A PD-L1 axis binding antagonist for use in treating cancer in an individual that has been determined to have a number of B cells in a tumor sample from the individual that is above a reference number of B cells, optionally wherein the B cells comprise plasma cells.

231. A PD-L1 axis binding antagonist for use in a method of treating an individual having a cancer, the method comprising:
(a) determining that the individual has clonally expanded B cells in a tumor sample from the individual; and
(b) administering an effective amount of a PD-L1 axis binding antagonist to the individual.

232. A PD-L1 axis binding antagonist for use in treating cancer in an individual that has been determined to have clonally expanded B cells in a tumor sample from the individual.

233. The kit of any one of embodiments 208-216 or the PD-L1 axis binding antagonist for use in accordance with any one of embodiments 217-232, wherein the PD-L1 axis binding antagonist is an anti-PD-L1 antagonist antibody, optionally wherein the anti-PD-L1 antagonist antibody comprises a heavy chain variable region comprising the amino acid sequence of SEQ ID NO: 7 and a light chain variable region comprising the amino acid sequence of SEQ ID NO: 8, optionally wherein the anti-PD-L1 antagonist antibody comprises a heavy chain comprising the amino acid sequence of SEQ ID NO: 9 and a light chain comprising the amino acid sequence of SEQ ID NO: 10, optionally wherein the anti-PD-L1 antagonist antibody is atezolizumab.

1-77. (canceled)

78. A method of treating non-small cell lung cancer (NSCLC) in a human individual that has been determined to have an immune-score expression level of genes CD79A, CD19, BANK1, JCHAIN, SLAMF7, BTK, TNFRSF17, IGJ, IGLL5, RBPJ, and MZB1 in a tumor tissue sample from the individual that is above a reference immune-score expression level of the genes, and has thereby been identified as one who may benefit from a treatment comprising an anti-PD-L1 antagonist antibody, the method comprising administering to the individual an effective amount of an anti-PD-L1 antagonist antibody, wherein the anti-PD-L1 antagonist antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

79. A method of treating NSCLC in a human individual that has been determined to have an immune-score expression level of genes MZB1, DERL3, JSRP1, TNFRSF17, SLAMF7, IGHG2, IGHGP, IGLV3-1, IGLV6-57, IGHA2, IGKV4-1, IGKV1-12, IGLC7, and IGLL5 in a tumor tissue sample from the individual that is above a reference immune-score expression level of the genes, and has thereby been identified as one who may benefit from a treatment comprising an anti-PD-L1 antagonist antibody, the method comprising administering to the individual an effective amount of an anti-PD-L1 antagonist antibody, wherein the anti-PD-L1 antagonist antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

80. A method of treating NSCLC in a human individual that has been determined to have an immune-score expression level of genes CCL2, CCL3, CCL4, CCL5, CCL8, CCL18, CCL19, CCL21, CXCL9, CXCL10, CXCL11, and CXCL13 in a tumor tissue sample from the individual that is above a reference immune-score expression level of the genes, and has thereby been identified as one who may benefit from a treatment comprising an anti-PD-L1 antagonist antibody, the method comprising administering to the individual an effective amount of an anti-PD-L1 antagonist antibody, wherein the anti-PD-L1 antagonist antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

81. The method of claim 78, wherein the reference immune-score expression level is an immune-score expression level of the genes in a reference population, wherein the reference population is a population of individuals having the NSCLC.

82. The method of claim 81, wherein the population of individuals comprises a first subset of individuals who have been treated with the anti-PD-L1 antagonist antibody and a second subset of individuals who have been treated with therapy that does not comprise the anti-PD-L1 antagonist antibody.

83. The method of claim 82, wherein:
(i) the reference immune-score expression level significantly separates each of the first and second subsets of individuals based on a significant difference between an individual's responsiveness to treatment with the anti-PD-L1 antagonist antibody and an individual's responsiveness to treatment with the therapy that does not comprise the anti-PD-L1 antagonist antibody above the reference immune-score expression level, wherein the individual's responsiveness to treatment with the anti-PD-L1 antagonist antibody is significantly improved relative to the individual's responsiveness to treatment with the therapy that does not comprise the anti-PD-L1 antagonist antibody; and/or
(ii) the reference immune-score expression level is a median of the expression level of each of the genes in the reference population.

84. The method of claim 83, wherein the benefit comprises an extension in the individual's OS as compared to treatment without the anti-PD-L1 antagonist antibody.

85. A method of treating NSCLC in an individual that has been determined to have the presence of a tertiary lymphoid structure (TLS) in a tumor tissue sample from the individual, and has thereby been identified as one who may benefit from a treatment comprising an anti-PD-L1 antagonist antibody, the method comprising administering to the individual an effective amount of an anti-PD-L1 antagonist antibody, wherein the anti-PD-L1 antagonist antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

86. The method of claim 85, wherein the presence of a TLS is determined by histological staining, immunohistochemistry (IHC), immunofluorescence, or gene expression analysis, wherein:
(i) the histological staining comprises hematoxylin and eosin (H&E) staining;
(ii) the IHC or immunofluorescence comprises detecting CD62L, L-selectin, CD40, or CD8; or
(iii) the gene expression analysis comprises determining the expression level of a TLS gene signature in the sample.

87. The method of claim 78, wherein:
(i) the immune-score expression level of the genes is an mRNA expression level or a protein expression level; and/or
(ii) the immune-score expression level of the genes is detected in tumor cells, tumor-infiltrating immune cells, stromal cells, normal adjacent tissue (NAT) cells, or a combination thereof.

88. The method of claim 87, wherein the immune-score expression level of the genes is the calculated Z-score of the genes.

89. A method of treating NSCLC in a human individual that has been determined to have (i) a number of B cells in a tumor tissue sample from the individual that is above a reference number of B cells; or (ii) clonally expanded B cells in a tumor tissue sample from the individual, and wherein the individual has thereby been identified as one who may benefit from a treatment comprising an anti-PD-L1 antagonist antibody, the method comprising administering to the individual an effective amount of an anti-PD-L1 antagonist antibody, wherein the anti-PD-L1 antagonist antibody comprises a heavy chain comprising HVR-H1 sequence of SEQ ID NO: 1, HVR-H2 sequence of SEQ ID NO: 2, and HVR-H3 sequence of SEQ ID NO: 3; and a light chain comprising HVR-L1 sequence of SEQ ID NO: 4, HVR-L2 sequence of SEQ ID NO: 5, and HVR-L3 sequence of SEQ ID NO: 6.

90. The method of claim 89, wherein the B cells or clonally expanded B cells comprise CD79+ B cells, IgG+ B cells, and/or plasma cells.

91. The method of claim 78, wherein:
(i) the tumor tissue sample comprises tumor cells, tumor-infiltrating immune cells, stromal cells, NAT cells, or a combination thereof; and/or
(ii) the tumor tissue sample is a formalin-fixed and paraffin-embedded (FFPE) sample, an archival sample, a fresh sample, or a frozen sample.

92. The method of claim 78, wherein the NSCLC is a locally advanced or metastatic NSCLC.

93. The method of claim 78, wherein the NSCLC is (i) a non-squamous NSCLC; or (ii) a squamous NSCLC.

94. The method of claim 78, wherein:
(i) the anti-PD-L1 antagonist antibody comprises a heavy chain variable region comprising the amino acid sequence of SEQ ID NO: 7 and a light chain variable region comprising the amino acid sequence of SEQ ID NO: 8; and/or
(ii) the anti-PD-L1 antagonist antibody comprises a heavy chain comprising the amino acid sequence of SEQ ID NO: 9 and a light chain comprising the amino acid sequence of SEQ ID NO: 10.

95. The method of claim 78, wherein the anti-PD-L1 antagonist antibody is atezolizumab.

96. The method of claim 78, wherein the individual has not been previously (i) treated for the NSCLC; or (ii) administered a PD-L1 axis binding antagonist.

97. The method of claim 78, wherein the individual has no EGFR or ALK genomic tumor aberrations.

98. The method of claim 78, wherein the individual has previously been treated for the NSCLC.

99. The method of claim 78, wherein the anti-PD-L1 antagonist antibody is administered as a monotherapy.

100. The method of claim 78, wherein the method further comprises administering an effective amount of one or more additional therapeutic agents, wherein the one or more additional therapeutic agents comprise an anti-neoplastic agent, a chemotherapeutic agent, a growth inhibitory agent, an anti-angiogenic agent, a radiation therapy, a cytotoxic agent, an immunomodulatory agent, or a combination thereof.


SEQUENCE LISTING (83 sequences)

SEQ ID NO: 1 - protein, 10 aa, synthetic construct
GFTFSDSWIH

SEQ ID NO: 2 - protein, 18 aa, synthetic construct
AWISPYGGSTYYADSVKG

SEQ ID NO: 3 - protein, 9 aa, synthetic construct
RHWPGGFDY

SEQ ID NO: 4 - protein, 11 aa, synthetic construct
RASQDVSTAVA

SEQ ID NO: 5 - protein, 7 aa, synthetic construct
SASFLYS

SEQ ID NO: 6 - protein, 9 aa, synthetic construct
QQYLYHPAT

SEQ ID NO: 7 - protein, 118 aa, synthetic construct [sequence omitted]

SEQ ID NO: 8 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1099 bp, Homo sapiens
  source 1..1099: mol_type = unassigned DNA
caaactaaccaacccactgggagaagatgcctgggggtccaggagtcctccaagctctgcctgccaccatcttcctcctcttcctgctgtctgctgtctacctgggccctgggtgccaggccctgtggatgcacaaggtcccagcatcattgatggtgagcctgggggaagacgcccacttccaatgcccgcacaatagcagcaacaacgccaacgtcacctggtggcgcgtcctccatggcaactacacgtggccccctgagttcttgggcccgggcgaggaccccaatggtacgctgatcatccagaatgtgaacaagagccatgggggcatatacgtgtgccgggtccaggagggcaacgagtcataccagcagtcctgcggcacctacctccgcgtgcgccagccgccccccaggcccttcctggacatgggggagggcaccaagaaccgaatcatcacagccgaggggatcatcctcctgttctgcgcggtggtgcctgggacgctgctgctgttcaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtgacacccctactcctgccaggctgcccccgcctgctgtgcacccagctccagtgtctcagctcacttccctgggacattctcctttcagcccttctgggggcttccttagtcatattcccccagtggggggtgggagggtaacctcactcttctccaggccaggcctccttggactcccctgggggtgtcccactcttcttccctctaaactgccccacctcctaacctaatccccccgccccgctgcctttcccaggctcccctcaccccagcgggtaatgagcccttaatcgctgcctctaggggagctgattgtagcagcctcgttagtgtcaccccctcctccctgatctgtcagggccacttagtgataataaattcttcccaactgca

SEQ ID NO: 14 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 15 - DNA, 2707 bp, Homo sapiens
  source 1..2707: mol_type = unassigned DNA
gtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaa

SEQ ID NO: 16 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 17 - DNA, 2611 bp, Homo sapiens
  source 1..2611: mol_type = unassigned DNA
aactgagtggctgtgaaagggtggggtttgctcagactgtccttcctctctggactgtaagaatatgtctccagggccagtgtctgctgcgatcgagtcccaccttccaagtcctggcatctcaatgcatctgggaagctacctgcattaagtcaggactgagcacacaggtgaactccagaaagaagaagctatggccgcagtgattctggagagcatctttctgaagcgatcccaacagaaaaagaaaacatcacctctaaacttcaagaagcgcctgtttctcttgaccgtgcacaaactctcctactatgagtatgactttgaacgtgggagaagaggcagtaagaagggttcaatagatgttgagaagatcacttgtgttgaaacagtggttcctgaaaaaaatcctcctccagaaagacagattccgagaagaggtgaagagtccagtgaaatggagcaaatttcaatcattgaaaggttcccttatcccttccaggttgtatatgatgaagggcctctctacgtcttctccccaactgaagaactaaggaagcggtggattcaccagctcaaaaacgtaatccggtacaacagtgatctggttcagaaatatcacccttgcttctggatcgatgggcagtatctctgctgctctcagacagccaaaaatgctatgggctgccaaattttggagaacaggaatggaagcttaaaacctgggagttctcaccggaagacaaaaaagcctcttcccccaacgcctgaggaggaccagatcttgaaaaagccactaccgcctgagccagcagcagcaccagtctccacaagtgagctgaaaaaggttgtggccctttatgattacatgccaatgaatgcaaatgatctacagctgcggaagggtgatgaatattttatcttggaggaaagcaacttaccatggtggagagcacgagataaaaatgggcaggaaggctacattcctagtaactatgtcactgaagcagaagactccatagaaatgtatgagtggtattccaaacacatgactcggagtcaggctgagcaactgctaaagcaagaggggaaagaaggaggtttcattgtcagagactccagcaaagctggcaaatatacagtgtctgtgtttgctaaatccacaggggaccctcaaggggtgatacgtcattatgttgtgtgttccacacctcagagccagtattacctggctgagaagcaccttttcagcaccatccctgagctcattaactaccatcagcacaactctgcaggactcatatccaggctcaaatatccagtgtctcaacaaaacaagaatgcaccttccactgcaggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgctggcatgagaaagcagatgagcgtcccactttcaaaattcttctgagcaatattctagatgtcatggatgaagaatcctgagctcgccaataagcttcttggttctacttctcttctccacaagccccaatttcactttctcagaggaaatcccaagcttaggagccctggagcctttgtgctcccactcaatacaaaaaggcccctctctacatctgggaatgcacctcttctttgattccctgggatagtggcttctgagcaaaggccaagaaattattgtgcctgaaatttcccgagagaattaagacagactgaatttgcgatgaaaatattttttaggagggaggatgtaaatagccgcacaaaggggtccaacagctctttgagtaggcatttggtagagcttgggggtgtgtgtgtgggggtggaccgaatttggcaagaatgaaatggtgtcataaagatgggaggggagggtgttttgataaaataaaattactagaaagcttgaaagtc

SEQ ID NO: 18 - protein, 659 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 19 - DNA, 891 bp, Homo sapiens
  source 1..891: mol_type = unassigned DNA
attgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctga

SEQ ID NO: 20 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 21 - DNA, 1286 bp, Homo sapiens
  source 1..1286: mol_type = unassigned DNA
agaagaagtgaagtcaagatgaagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactaatttaagtcattgctgactgcatagctctttttcttgagaggctctccattttgattcagaaagttagcatatttattaccaatgaatttgaaaccagggcttttttttttttttgggtgatgtaaaaccaactccctgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggactgaaaatcaggaggtgtaataaaatgttcctcgcattcccccccgcttttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagacttggaagcagatgatacagacttctttttttcataatcaggttagtgtaagaaattgccatttgaaacaatccattttgtaactgaaccttatgaaatatatgtatttcatggtacgtattctctagcacagtctgagcaattaaatagattcataagcata

SEQ ID NO: 22 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 23 - DNA, 1300 bp, Homo sapiens
  source 1..1300: mol_type = unassigned DNA
gtgaccgcttcagggcagttggtagatgcccctctgggagagatccccaggggtgacagccatggaccctggaagggcctgggctagggacagggaccagagccagtccagggagaggacagagccaatggactggggtgtactgtaacagccctgctggcgagagggaccagggcaccgtcctccagggagcccatgctgcaagtcgggccagaggtgcccctgaacctgaaggccaatgagacccaagacaggccaagtgggttgtgagacccctgaggagctgggccctggtcccaggcagcgctggcccctgctgctgctgggtctggccatggtcgcccatggcctgctgcgcccaatggttgcaccgcaaagcggggacccagaccctggagcctcagttggaagcagccgatccagcctgcggagcctgtggggcaggctcctgctccagcccagcccccagagagcagaccccaggtgctggccccgggggttttggtctgagcctcagtcactgtgttatgtcttcggaactgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataggttcccaactctaaccccacccacgggagcctggagctgcaggatcccaggggaggggtctctctccccatcccaagtcatccagcccttctccctgcactcatgaaaccccaataaatatcctcattgacaaccagaaatcttgttttatctcattttttttctcacataaattgctagcctccccggggttctcagtgtggggtacagggaattctgcacccagtgtgaaaatcacccaagggaggaggctcacagcctccctgagtcatctccccagagggtccttcctctcccagtcaccccttctccaactctccactgtacccctgagctaccagtctggcatcagttcagaccagtcccacaccctcctaaattttacttctcaataaatacctgatcatgtaaaacgca

SEQ ID NO: 24 - protein, 214 aa, Homo sapiens
MRPKTGQVGCETPEELGPGPRQRWPLLLLGLAMVAHGLLRPMVAPQSGDPDPGASVGSSRSSLRSLWGRLLLQPSPQRADPRCWPRGFWSEPQSLCYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - DNA, 5878 bp, Homo sapiens
  source 1..5878: mol_type = unassigned DNA
attgggtacatgcgcagtgccgctcgcgcgggccgcgccaatcctgcagcgccttcaacaggtttcgggcggcgaattccagttctccgggttttggggctccctgcgggaggcagtgctggatctgggaatctctaggaaaggaaagagcaaaggaggggaaagatggggggctgcagtctccacgtacgtccctcaaagcgcgtcctaaaacccggataaccggagcgctccccatggaccacacggagggctcgcccgcggaggagccgcctgcgcatgctccatcgcctgggaaatttggtgagcggcctccacctaaacgacttactagggaagctatgcgaaattatttaaaagagcgaggggatcaaacagtacttattcttcatgcaaaagttgcacagaagtcatatggaaatgaaaaaaggtttttttgcccacctccttgtgtatatcttatgggcagtggatggaagaaaaaaaaagaacaaatggaacgcgatggttgttctgaacaagagtctcaaccgtgtgcatttattgggataggaaatagtgaccaagaaatgcagcagctaaacttggaaggaaagaactattgcacagccaaaacattgtatatatctgactcagacaagcgaaagcacttcatgttgtctgtaaagatgttctatggcaacagtgatgacattggtgtgttcctcagcaagcggataaaagtcatctccaaaccttccaaaaagaagcagtcattgaaaaatgctgacttatgcattgcctcaggaacaaaggtggctctgtttaatcgactacgatcccagacagttagtaccagatacttgcatgtagaaggaggtaattttcatgccagttcacagcagtggggagccttttttattcatctcttggatgatgatgaatcagaaggagaagaattcacagtccgagatggctacatccattatggacaaacagtcaaacttgtgtgctcagttactggcatggcactcccaagattgataattaggaaagttgataagcagaccgcattattggatgcagatgatcctgtgtcacaactccataaatgtgcattttaccttaaggatacagaaagaatgtatttgtgcctttctcaagaaagaataattcaatttcaggccactccatgtccaaaagaaccaaataaagagatgataaatgatggcgcttcctggacaatcattagcacagataaggcagagtatacattttatgagggaatgggccctgtccttgccccagtcactcctgtgcctgtggtagagagccttcagttgaatggcggtggggacgtagcaatgcttgaacttacaggacagaatttcactccaaatttacgagtgtggtttggggatgtagaagctgaaactatgtacaggtgtggagagagtatgctctgtgtcgtcccagacatttctgcattccgagaaggttggagatgggtccggcaaccagtccaggttccagtaactttggtccgaaatgatggaatcatttattccaccagccttacctttacctacacaccagaaccagggccgcggccacattgcagtgcagcaggagcaatccttcgagccaattcaagccaggtgccccctaacgaatcaaacacaaacagcgagggaagttacacaaacgccagcacaaattcaaccagtgtcacatcatctacagccacagtggtatcctaactaccgtctttttgctaggacttaaactgacttgagtgtggcaaaaagttaacaaaaaaggagaaaaaatgaacaatcgtttgtggtttcttgggaaaacttttcataccaggtgatactattcaaaaaccccgttgtctccctgcaagtgctgatttgaaatgcagaagccacagtaaaaaaaaaaaaaaaaaaaaaaaaaaagaaaaaaaaatcaaaatgtataaatattggaaatcaagtttttcagctgttttgttggttggttggttggtttttgtttggttttgtttaaatgggcaagaagtaaataatgtggctggaatacaagttgaacaaactagaagacacaaatctaacatagtttttatggaccaaggaacttgtatattgtataagctttagtaaaaggtacattttcaccatacctttttttatatcacggtattatagtacaccttgttaccaaataggttgttctcttccccacccacctttgagcttttgctctaaaatacattcaggttccaagcctgaccatccttgtttaatctatcatactcttccaggttttttttttttggtctaaggctggaacttttttcttttttttcagctgaagtcttatgacttttcatgagtcaaaattgtttggatttcagcaagtcaaatcttgcaaaggcctgcatattttttttaagattatatgaagtctgtgcaaaagctttaaaaaaatgcctctgccttgcctgcaatacatgcaatgtatgttaacttagtctctcttctcagacactgttggtagttatttctgtgttttcctttttttaaaaaaaaatatggacttattgtggttatctgagaggttctaacattcacatgcaatttggtgtggccatttagctattaatgagttaatggcgcagaacttgttgatatttgaagtgttctctccccttttcccatgacgtaaatacataggtgtgttccaggatttgttcaggtttttcccccctcctaatcttgtacataacttgtattatgtgtaagttaaacattttattttgaacttggaatgttcccagtgatttcattcagcagggtattttctgccttgttggcaagtgacaaaaaatatgggaagtatttgctaccagttggtagatggtgcccttaatggtagaatgaggaaaatgtccgcaaaagcatgttttattatctttacttttttggggggttggagggggtagcctagccagaacatcattgtaatcttaaaacataagatgcttttattagatgatcaactaaaatagctggaagacagtactttagaaacagatagttgtaagattataaaatgcaaatgtaacttatgttttcatttttttctctgccttttttgtttgtttgttttctcttttccagtactgagcatctccacaaatgtctcctaactcagaaaatgtttcttttcttttcagttgagatttggttgcattcagggttgtaggttggccttgcttgctaaccccgccggttttaccgtgctttcattcctgaactttgtttatgcctttgtttggtttcttcgaaattgcagcagactcattgggctacatttagtacaggaaccacgtgtgtaatgttatacaacacagtctagtaatacaatcatccctcttagagtaaaaactacctctagattgtggtaagcttttactgtcccataaaacaggagccacagtaccttatgaatgcaaaactgtaacttcctacagtgtttccctacagaacattgtctttctggtgtcctgggctgttttgaaaaagtttccattaatagactttttagaaattattattagtagcattttttttccagctttgctgtcttcatcactcactctatgctcagactatgccactgtaaatattcttcctaacatctttaaatcgccttttcctcagttttcaaggggaaggtcatttgtaaagcacgttaggtggttaaatcagttattgcggttttctcttactgcaagcctttttaatcacccccaggctgcattttattctatatcgccttttttcttcaaatctgctccaatcactcacttctctcttataagctaatcctgcctcacaccttaaatctgtttcagtgatcaagggcagaactcattgtggccttatctttctttgttgtaattgttcactgtctctttcttacagaccacttatttctgagtagtagttattcctctctatggagtcatggcaggaatcattacacagtgcttttgttcagagcatggacatgttcctagtgctgctttgctttaacggccacaagtttcctccacttcctaggtttggtatttagttaaggaatcatattaaattaaccaataacaaaagagatacttttgaagaacaaactattccttacccatttttgtagctcaaaaataatttttcaagttcatgaccttattaaaatgaacttgtgtttttttaacaaacatgtatgttttattttgatagtttctttccgtaagataattgaaatattatactgtaaacccttttcttttctttttttgaaaagtccaagaatgtacttatacaggcatttttccccacctatttttggccattctcataccacagactaaagagtgaaatgatttgtccattgtagcttattgtttatcagtagttcttttgtcagctgcttacattttttctttcatggttttgtgaatcattttcagtatgtaatttataggaaccttgtcctctggttatagtagactgtgtgccctcctccagtgatggcattattagacatgctggtcatttaccctcagaaagactctcttattagaatggtgagtgcttcagttatagtatgtttgaatttttaaaaaattctgttttagaaatgtatcttatgctctcatgactatgcagtttctaaacatacacatagaagctgagtctctgatccaatatgtttttatttgttccatttaatttatcacatagattgggaaggcaagctaaaagccttaaaaatgccctttatattttgagtgatttcagcgttgaacactagtatactatctaaatttgctgctcactttctttaaactgtggcaattaaaggcatgtttatacatgacttaatcgtgaaatgtttgtcactcttactgcacagacttatctgcaatcataactggttagtttttttgttttgttttgttttattgtttttaatgaaactggtaccatctgtgctttcacaaaaaacttccaatgccatttttgagaactaacctaactagtcatgctaaccagaaaatccactggggaggaggttccttttgaaacaaaatgctgttcagttagtaaccaagttactttgattgcaaaagcagctgtgtttctgataagtactgaacaaatgtgtgtaattttctgtgccagacttatgactttgttttcaagcactgtaatgtgggatggatggttagaaacaataatatattagggtttctgtttaaccctttcaggactgaactgtatctccttttgttaattttcccctgtgttgtgataaatgtttgccagcattcagtactgtgttggtccagatgtaggtttatatgctcatttttagcttatttcttgtaccttgcagcatgctctacgcattcagtccttaaggggtttattttacaaactgtgcgcctgtaaggtttattagcaataagatagaaaattgagcaagtttataccataattttgtagaaaaaaagaatctgctcagttccatatttcatccgtgaaaaacttgcaatacgagcagtttcaaggaataaataaaaaggaaatgtaaaccattgtaaaagtcttctgtcgaatgtgcctgatgcatgtattatcgtcttttatttcagaatacttcataaagataaaattaaattctatattatagttggtgtatttacaatcttaccatgtacatcacatcaaagtgatagctctactaatttaatttccttgtcaatgtttttaactatatagtgctttaaagagattttttttccctgtgtaaaaaaaaaaaaaaaaaa

SEQ ID NO: 26 - protein, 500 aa, Homo sapiens
MDHTEGSPAEEPPAHAPSPGKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 27 - DNA, 925 bp, Homo sapiens
  source 1..925: mol_type = unassigned DNA
acacacacatctgcacctcaaccacagactacacttgctgaactggctcctggggccatgaggctgtcactgccactgctgctgctgctgctgggagcctgggccatcccagggggcctcggggacagggcgccactcacagccacagccccacaactggatgatgaggagatgtactcagcccacatgcccgctcacctgcgctgtgatgcctgcagagctgtggcttaccagatgtggcaaaatctggcaaaggcagagaccaaacttcatacctcaaactctggggggcggcgggagctgagcgagttggtctacacggatgtcctggaccggagctgctcccggaactggcaggactacggagttcgagaagtggaccaagtgaaacgtctcacaggcccaggacttagcgaggggccagagccaagcatcagcgtgatggtcacagggggcccctggcctaccaggctctccaggacatgtttgcactacttgggggagtttggagaagaccagatctatgaagcccaccaacaaggccgaggggctctggaggcattgctatgtgggggaccccagggggcctgctcagagaaggtgtcagccacaagagaagagctctagtcctggactctaccctcctctgaaagaagctggggcttgctctgacggtctccactcccgtctgcaggcagccaggagggcaggaagcccttgctctgtgctgccatcctgcctccctcctccagcctcagggcactcgggcctgggtgggagtcaacgccttcccctctggactcaaataaaacccagtgacctcacttcttttctctgcaaaaggtgcttgtggggctgggagtgcagacattggtgtttctgctgatgtcccttgtgaaaaaaaaaaaaaaactctcaaaatcaa

SEQ ID NO: 28 - protein, 189 aa, Homo sapiens
MRLSLPLLLLLLGAWAIPGGLGDRAPLTATAPQLDDEEMYSAHMPAHLRCDACRAVAYQMWQNLAKAETKLHTSNSGGRRELSELVYTDVLDRSCSRNWQDYGVREVDQVKRLTGPGLSEGPEPSISVMVTGGPWPTRLSRTCLHYLGEFGEDQIYEAHQQGRGALEALLCGGPQGACSEKVSATREEL

SEQ ID NO: 29 - DNA, 741 bp, Homo sapiens
  source 1..741: mol_type = unassigned DNA
gagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaa

SEQ ID NO: 30 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 31 - DNA, 780 bp, Homo sapiens
  source 1..780: mol_type = unassigned DNA
agaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaa

SEQ ID NO: 32 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 33 - DNA, 660 bp, Homo sapiens
  source 1..660: mol_type = unassigned DNA
agcacaggacacagctgggttctgaagcttctgagttctgcagcctcacctctgagaaaacctcttttccaccaataccatgaagctctgcgtgactgtcctgtctctcctcatgctagtagctgccttctgctctccagcgctctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactgagctgctcagagacaggaagtcttcagggaaggtcacctgagcccggatgcttctccatgagacacatctcctccatactcaggactcctctccgcagttcctgtcccttctcttaatttaatcttttttatgtgccgtgttattgtattaggtgtcatttccattatttatattagtttagccaaaggataagtgtcccctatggggatggtccactgtcactgtttctctgctgttgcaaatacatggataacacatttgattctgtgtgttttcataataaaactttaaaataaaatgca

SEQ ID NO: 34 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 35 - DNA, 309 bp, Homo sapiens
  source 1..309: mol_type = unassigned DNA
accatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacac

SEQ ID NO: 36 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 37 - DNA, 862 bp, Homo sapiens
  source 1..862: mol_type = unassigned DNA
agagaggttgagaacaacccagaaaccttcacctctcatgctgaagctcacacccttgccctccaagatgaaggtttctgcagcgcttctgtgcctgctgctcatggcagccactttcagccctcagggacttgctcagccagattcagtttccattccaatcacctgctgctttaacgtgatcaataggaaaattcctatccagaggctggagagctacacaagaatcaccaacatccaatgtcccaaggaagctgtgatcttcaagaccaaacggggcaaggaggtctgtgctgaccccaaggagagatgggtcagggattccatgaagcatctggaccaaatatttcaaaatctgaagccatgagccttcatacatggactgagagtcagagcttgaagaaaagcttatttattttccccaacctcccccaggtgcagtgtgacattattttattataacatccacaaagagattatttttaaataatttaaagcataatatttcttaaaaagtatttaattatatttaagttgttgatgttttaactctatctgtcatacatcctagtgaatgtaaaatgcaaaatcctggtgatgtgttttttgtttttgttttcctgtgagctcaactaagttcacggcaaaatgtcattgttctccctcctacctgtctgtagtgttgtggggtcctcccatggatcatcaaggtgaaacactttggtattctttggcaatcagtgctcctgtaagtcaaatgtgtgctttgtactgctgttgttgaaattgatgttactgtatataactatggaattttgaaaaaaaatttcaaaaagaaaaaaatatatataatttaaaacta

SEQ ID NO: 38 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 39 - DNA, 1332 bp, Homo sapiens
  source 1..1332: mol_type = unassigned DNA
agaaggaggccaggagttgtgagtttccaagccccagctcactctgaccacttctctgcctgcccagcatcatgaagggccttgcagctgccctccttgtcctcgtctgcaccatggccctctgctcctgtgcacaagttggtaccaacaaagagctctgctgcctcgtctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggctacctcttctaagagtcccatctgctatgcccagccacattaactaactttaatcttagtttatgcatcatatttcattttgaaattgatttctattgttgagctgcattatgaaattagtattttctctgacatctcatgacattgtctttatcatcctttcccctttcccttcaactcttcgtacattcaatgcatggatcaatcagtgtgattagctttctcagcagacattgtgccatatgtatcaaatgacaaatctttattgaatggttttgctcagcaccaccttttaatatattggcagtacttattatataaaaggtaaaccagcattctcactgtgacgactctgttgattttgtttcactaatcggaatcacagactgagaggaattctgggggaggagtagggaaatatgaaaaggggcagaaacagcaagatagggaccaagtacttctacagtcataccagacatttccctggagatactttcctgaaaagttgaaacagacaccattagtttataaaccatattgtaactgaaatgtgatagaaaaattttctacttaaatgaatatcaagatgacgctgcaatgcatatttacgcacacaagctcattttcataaatgaagctttcctcagaatgatggctaacatctgttgaggtcttaccaagtgctgggttttgggctaagtacttatattagtaaaagtctagcaaatactgcccatagtctagcaaggactccttacctggaagttgctgaaagccttggtaatgttatctttgttctccactctgcttttggggaggatgttttccatgactatacgagatgaggcttggggctgggtggccagagttagcaaataaaaatacagaatggatacta

SEQ ID NO: 40 - protein, 89 aa, Homo sapiens
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 41 - DNA, 683 bp, Homo sapiens
  source 1..683: mol_type = unassigned DNA
attcccagcctcacatcactcacaccttgcatttcacccctgcatcccagtcgccctgcagcctcacacagatcctgcacacacccagacagctggcgctcacacattcaccgttggcctgcctctgttcaccctccatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaacctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaactaa

SEQ ID NO: 42 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 43 - DNA, 864 bp, Homo sapiens
  source 1..864: mol_type = unassigned DNA
acagacccccaacttgcagctgcccacctcaccctcagctctggcctcttactcaccctctaccacagacatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatagcccagtgagcagcctggagccctggagaccccaccagcctcaccagcgcttgaagcctgaacccaagatgcaagaaggaggctatgctcaggggccctggagcagccaccccatgctggccttgccacactctttctcctgctttaaccaccccatctgcattcccagctctaccctgcatggctgagctgcccacagcaggccaggtccagagagaccgaggagggagagtctcccagggagcatgagaggaggcagcaggactgtccccttgaaggagaatcatcaggaccctggacctgatacggctccccagtacaccccacctcttccttgtaaatatgatttatacctaactgaataaaaagctgttctgtcttcccacccaa

SEQ ID NO: 44 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 45 - DNA, 2761 bp, Homo sapiens
  source 1..2761: mol_type = unassigned DNA
aaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataacacatgaaagctctttaaagggtcgatcgggaacaggaaaaaaaacctatggaaaatatgacaacac

SEQ ID NO: 46 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 47 - DNA, 1175 bp, Homo sapiens
  source 1..1175: mol_type = unassigned DNA
gagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 48 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 49 - DNA, 1479 bp, Homo sapiens
  source 1..1479: mol_type = unassigned DNA
gttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaa

SEQ ID NO: 50 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 51 - DNA, 1219 bp, Homo sapiens
  source 1..1219: mol_type = unassigned DNA
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 52 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 53 - DNA, 1060 bp, Homo sapiens
  source 1..1060: mol_type = unassigned DNA
cggctcccgcgccgcctcccctcgcgcccgagcttcgagccaagcagcgtcctggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatca

SEQ ID NO: 54 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 55 - DNA, 3207 bp, Homo sapiens
  source 1..3207: mol_type = unassigned DNA
agccagctgccgtctgcgctttgcaaagcatgcagttaggggagcagctcttggtgagctcagtgaacctgcctggcgcgcacttctacccgctggagagtgcgcgaggcggcagcggcgggagcgctggccacctccccagcgcggccccctctcctcagaagttggacttagacaaagcgtccaagaagttttccggcagtctctcctgcgaggcggtgagcggggagcccgcagccgccagcgcaggggcccccgcggccatgcttagtgacaccgacgccggggacgcatttgccagcgctgcggcagtggccaagccggggcccccggacggccgcaagggctccccctgcggggaggaggagctgccctccgccgctgcagccgccgccgccgccgccgccgcggctgcggccactgcgcgctactccatggacagcctgagctccgagcggtactacctccagtcccccggtcctcaggggtcggagctggctgcgccctgctcactcttcccgtaccaggcggcggctggggcgccccacggacctgtgtacccggctcctaacggggcgcgctacccctacggctccatgctgccccccggcggcttccccgcggctgtgtgcccacccgggagggcgcagttcggcccaggagccggtgcgggcagtggcgcgggcggtagcagcggcgggggcggcggcccgggcacctatcagtacagccagggggctccgctctacgggccgtaccctggagccgcagcggcgggatcttgcggaggactggggggcctgggggttccaggttctggcttccgtgcccacgtctacctgtgcaaccggcctctgtggctcaaattccaccgccaccaaactgagatgatcattacgaaacagggcaggcgcatgtttcctttcttgagcttcaacataaacggactcaatcccactgcccactacaatgtgttcgtagaggtggtgctggcggaccccaaccactggcgcttccaggggggcaaatgggtgacctgtggcaaagccgacaataacatgcagggcaacaaaatgtatgttcacccagagtctcctaatactggttcccactggatgagacaggagatttcattcgggaaattaaaactcaccaataacaaaggcgcaaataacaacaacacccagatgatagtcttacaatccttacacaaataccaaccccgactgcatattgttgaagttacagaggatggcgtggaggacttgaatgagccctcaaagacccagacttttaccttctcagaaacgcaattcattgcagtgactgcctaccaaaacaccgatattactcaactaaagattgatcataacccctttgcaaaaggcttcagagacaactatgattcatcccatcagattgtccctggaggtcggtacggcgttcaatccttcttcccggagccctttgtcaacactttacctcaagcccgctattataatggcgagagaaccgtgccacagaccaacggcctcctttcaccccaacagagcgaagaggtggccaaccctccccagcggtggcttgtcacgcctgtccagcaacctgggaccaacaaactagacatcagttcctatgaatctgaatatacttctagcacattgctcccatatggcattaaatccttgccccttcagacatcccatgccctggggtattacccagacccaacctttcctgcaatggcagggtggggaggtcgaggttcttaccagaggaagatggcagctggactaccatggacctccagaacaagccccactgtgttctctgaagatcagctctccaaggagaaagtgaaagaggaaattggctcttcttggatagagacacccccttccatcaaatctctagattccaatgattcaggagtatacaccagtgcttgtaagcgaaggcggctgtctcctagcaactccagtaatgaaaattcaccctccataaagtgtgaggacattaatgctgaagagtatagtaaagacacctcaaaaggcatgggagggtattatgctttttacacaactccctaaagagttattttaacctcaaaaattagctaactttttgcagatggacttggtggtgttttttgttgtcttctttgcctaggttgccaaaaagatgtttgccttccaccttgatgcatcctgttttgtgcaattctctaaaagaaggtgccaaagctttttgattgctgcaggtaactgaaacaaacctagcatttttaaaaaataagattaatggaagactttaaggtattttaaaattcgaagggtatccaaggttctgtatttatttattggggagacactaacccttcaaagaagcaggctgtgaacattgggtgcccagtgctatcagatgagttaaaacctttgattctcatttctatttgtaaattcttaagcaaatagaagccgagtgttaaggtgttttgcttctgaaagagggctgtgccttccgtttcagaaggagacattttgctgttacattctgccaggggcaaaagatactaggcccaggagtcaagaaaagcttttgtgaaagtgatagtttcacctgactttgattccttaacccccggcttttggaacaagccatgtttgccctagtccaggattgcctcacttgagacttgctaggcctctgctgtgtgctggggtggccagtgggactcaggagagagcaagctaaggagtcaccaaaaaaaaaaaaaaaaaaaagggagaatttaaaagtgtacagttgtgtgtttagatacactatagaataatgtggtatatattgtacaaatagtctacataggtgtctgggataatgtaaaactggtgctttggctttgtaaagaatttgcaaatcacttaacagctgcaggggcaaggggagagtttcatcatccccatgatatttgggaatattctgtttacttcttagatagttaagaatgtattcagctactatgtactaacttgaaccgtgtttaaggaaaactcctatttcatcctcttcttgcgccatcccctctccctaacttggtaatgtgaagaaactaaaacctgataccacagctcctataggcattttagagatcttggatttttatgtacagtcttagtcatttttaataaatgtggttcagtaagggaa

SEQ ID NO: 56 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 57 - DNA, 884 bp, Homo sapiens
  source 1..884: mol_type = unassigned DNA
cagccacaatgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtggggcaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaaataaccgtttcctttcatttactgtggcttcttaatcttttcacaaataaaatcaatttgcatgactgtaaaaaaaaaaaaaaaaaa

SEQ ID NO: 58 - protein, 262 aa, Homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 59 - DNA, 2583 bp, Homo sapiens
  source 1..2583: mol_type = unassigned DNA
agtgacagcggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttgaa

SEQ ID NO: 60 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 61 - DNA, 1211 bp, Homo sapiens
  source 1..1211: mol_type = unassigned DNA
acattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacta

SEQ ID NO: 62 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 63 - DNA, 884 bp, Homo sapiens; identical to SEQ ID NO: 57
  source 1..884: mol_type = unassigned DNA

SEQ ID NO: 64 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 65 - DNA, 3394 bp, Homo sapiens
  source 1..3394: mol_type = unassigned DNA
gtgcttgagaaggttcaatggcgtggcagggactagcggccgagttcctgcaggtgccggcggtgacgcgggcttacaccgcagcctgtgtcctcaccaccgccgcggtggtaagcggccgggcggaccggacgtcgccttggttacagcctctggcgggaggggtgagggtcgccatggttacggcgtggctcccgggcagctcttggctggcccctggttccacagcagctggagctcctcagcccctttcaactctacttcaacccgcaccttgtgttccggaagttccaggtctggaggctcgtcaccaacttcctcttcttcgggcccctgggattcagcttcttcttcaacatgctcttcgtgttccgctactgccgcatgctggaagagggctccttccgcggccgcacggccgacttcgtcttcatgtttctcttcgggggcgtccttatgaccgtatccttcccgcaggctctggaacctcgggctagggcgcctcggcgtccagcctgtgttggtcctggggccaacacagccatgccagaaagggacacagtcgctgtctccagcttagtatgtgttgagggcccactctgtgctcagctgcaggggtcagggctagatcttcagtgctgtatgcaaaatacaaagccacgcacaaaagagccaggcaccgttcctgccttgggcgctcatgggcttctcgctgctgctgggcaactccatcctcgtggacctgctggggattgcggtgggccatatctactacttcctggaggacgtcttccccaaccagcctggaggcaagaggctcctgcagacccctggcttcctgtgagtgttgagagccctccctccctctccccaccctcagaaggatccccaccgatggggacctgtgctggcctgtgctcaacacgggcccctccccacagaaagctgctcctggatgtccctgcagaagaccccaattacctgcccctccctgaggaacagccaggaccccatctgccacccccgcagcagtgacccccacccagggccaggcctaagaggcttctggcagcttccatcctacccatgacccctacttggggcagaaaaaacccatcctaaaggctgggcccatgcaagggcccacctgaataaacagaatgagctgcagtctcttggcccacagcactggcttccccatctcacctggccacatccttctatgcctgccccgtcctcactcagtgtggcctctcagcccaactgcaggtggtaggataggggtgcccacagagggcaaagaaactgcccatggttgcctggcagagctttgagctcacaggttgccaggcagagcttttgagctcacaggtgacaggctcagggttctcatcctggccccaccagggccttgggcaagtcctgcccaccataggcctctgctacctgccagccagcggggaagttcaccagatttcggctgctggggccaggacaggcctctcctaggttgtgccaaaccagcctacagatgttcctgccagtggtgccttcaggctgatgccaatctagcctctcctgtctctcatcagccaccctgacaggtgggcgtatgcctcatttttcatctggtgatgccaaagccccatggattcaggtgcagaagagggccaggactaggtcttctgccctttctatgacctcagagcctaagttttcaccttagcagagttctgagactgggtgaggcagggacttctggaaggttctgttcctgccctttttagctgaggacgtgtgtgagccttatccgacccctgtggctcatttttctcttctgacctggcagctttccttgttgttctaagcctgtccatgttgtggtttatttctggatgctcagtggcacggggcctcctccaaagacaggttgtcattttcatggtaacaacactgttctctgttgagtctgccctccgtgttgtagccagaccttgtggagatggctttgcggcggtgtgagctggcggtcaggagtacccagccttcccggcacctcccagccaggtggccctgcccgacctgtggggtgaggcagccaaggttccttccatccccccagttgtggagacacagggctgcctcaccctttcattgccgaggttcctacctcatggacagaacaaacacctcagcaatgaaacctgttcatgtctaagagcagctgggctgggaatcttcccctttgttcaaggccttccagtaaggcccagctgtccccttgctgtgcatggggctctggggagttccactctttgatggagggcagaggccctgagtgcaaactccctgggaagagtcccatgctaacatgtgctcaaaggagcccccctctcacatctcagcgaccaagaaccccaatccctaatcagagctctgcctctgccccatatggggccctaccccacttccagggagcacagcagcctctgaccctagccctgccctggcagcatgggacctgccaacagctgagggtggcagcagtctgtgctgggatctgtgcccggatctgtgcccattctcttcagtaaggctggagctgcgagccagtttgctgccctcccagatcctgtgtccattctgaagaatggggacacctccctattacagatgagaacagaggggacatgaactccttggaggcagggctgggaagggaccctgggctgtgtctcctccctgtaccgtgtcaactccaagagctggcaccaggccgggagggctattcccatattcctcacagctggcttgtaaggcaggagctggcccaggccacagcacctgctggggtgggaaagggccccagctgacctggctatgggtgccactggagctagaacagccctcctgcacccaggctggcgacagcccagcagtgcccacacaggactgggccctccgcaggggacttcagagcagcaaggccccagctggcagtagcctgaccatctggacacagcagagccagggcggcccagggacggcaggagagctcgcggcaccttcctgaggccaagcaaggggagcagggttagggctgttcctgaaaggcagagagcctctgccctgagcctcacagctactcttctcagctctctgggtctggaaggagaacaggctgaggggagctgagaggagctgaggtgctacccggagccccattcacccccacctgcccacttgggaatctgaggcagaggagggtgaggcctgtgtgccaaccttgttcacataccaccttcgtccccccaggccccggccccactcctggctctcattatttttatgttaaaactttgaagaaattgaacatgacctgttgaagaaattg

SEQ ID NO: 66 - protein, 235 aa, Homo sapiens
MAWQGLAAEFLQVPAVTRAYTAACVLTTAAVQLELLSPFQLYFNPHLVFRKFQVWRLVTNFLFFGPLGFSFFFNMLFVFRYCRMLEEGSFRGRTADFVFMFLFGGVLMTLLGLLGSLFFLGQALMAMLVYVWSRRSPRVRVNFFGLLTFQAPFLPWALMGFSLLLGNSILVDLLGIAVGHIYYFLEDVFPNQPGGKRLLQTPGFLKLLLDAPAEDPNYLPLPEEQPGPHLPPPQQ

SEQ ID NO: 67 - DNA, 1173 bp, Homo sapiens
  source 1..1173: mol_type = unassigned DNA
ggcggcctggcccgggacctgctgctgctccagccatgtccatgacaaccagagcctgggaggagctggatggcggcctgggcagctgccaggccctggaggaccactctgcgctggccgagacccaggaggacagggcttcagcgacacccaggctggccgactccggcagcgtgccccacgactctcaggtggctgaaggccccagtgtggacaccaggcccaagaagatggaaaaagagcctgccgccagggggaccccaggaacggggaaggagaggctgaaagccggagcgagccctcggagcgtccccgcgcgcaagaaggcgcagaccgcgccgcccctgcagccgccgccgccgcccccggccctgagcgaggagctgccctggggagacctgtcgctcaacaagtgcctggtgctcgcctcgctggtggcgctgctgggctcggctttccagctgtgccgcgacgccgtccctggggaggcagcactccaagcacgtgtgcccgagccctgggtcccgccaagctcagccccgagggagccatcgtcgcccctgcctaagttcgaggcccaggcgcctccatcagcgccgcctgcgccccgggccgaggcagaggtcagacccaagattcccgggagtcgggaggctgcagagaacgacgaagaggagcccggcgaggccaccggagaggccgtccgggaggaccgtgtgaccctcgcagaccggggacccaaggagaggcctcggagagaggggaagccgcggaaggagaagccgcggaaggaggagagacctaagaaagagaggccgcggaaagaggagaggccacgggccgccagggagccccgggaagccctaccccagcgctgggagtcacgcgaagggggccaccggccgtgggcacgggactccagggacgccgagcccaggaagaagcaggcctgggtgtccccgaggcgtcccgacgaggagcagcggcctgggagtcgccagaagctccgcgcaggcaaggggcgggactgagccggccccgcgccggagtccaggggccccttctggacgccccgcgactctggcgaaataaagcgagtgctgcggcaaaaaaaaaaaaaaaaaaaaaaaaaaaaaaaaaaaaaaaaaaaaaaaaaaaaaaaaaaaaaaaaaa

SEQ ID NO: 68 - protein, 331 aa, Homo sapiens
MSMTTRAWEELDGGLGSCQALEDHSALAETQEDRASATPRLADSGSVPHDSQVAEGPSVDTRPKKMEKEPAARGTPGTGKERLKAGASPRSVPARKKAQTAPPLQPPPPPPALSEELPWGDLSLNKCLVLASLVALLGSAFQLCRDAVPGEAALQARVPEPWVPPSSAPREPSSPLPKFEAQAPPSAPPAPRAEAEVRPKIPGSREAAENDEEEPGEATGEAVREDRVTLADRGPKERPRREGKPRKEKPRKEERPKKERPRKEERPRAAREPREALPQRWESREGGHRPWARDSRDAEPRKKQAWVSPRRPDEEQRPGSRQKLRAGKGRD

SEQ ID NO: 69 - DNA, 32230 bp, Homo sapiens
  source 1..32230: mol_type = unassigned DNA
gaattcactcatcaccaaggggaagatgctcaatcattcatgagggatctgcccccttgatctaatcacctcctagtggccccacttccaacactgggaatcatattacaacatgagatttggaggggacaaatatataaaccatatccttccacccctggtcccccaaatctcatgtccttctcatgttgcaaaatacaatcatgccttctcaacagtcccccaaagtcttacctcattccagaatcaacaaaaaattcccaagtcccaagtctcatctgaagatgagtcccttccacctatcagcctaggaaatcaaagacaaattgactcccaaaatacaatgagagtgcagacattgggtaaacattcccattccacaagggagaagttggcaaaaagaaaggatctacatgccccatacacgtcagaaatccagcagggcagttattaaatcttaaagctccaaggtaatctcctttgacttcatgtcccatatccagggcacactggtgcagggggtgggctcccaaagccttggacaactctacccctgtggctttggaggatgtcgcccctgtggcttctctcacacgatagagttgagtgcctgtggcttttccagattcagggtgcaaactgccagtggatctaccattctggggtctggaggacagtggcccccttcccacagctccactaggcagtgccctggtggggacattatatgggggttcaaccccacattctctttggcactgctctagtagaggttctctgtgagggctccatgctggcaggaggcttctctctgggcacctagggtttctcatacatttctgaaatctagagtgaagatgccaagcctccttcactcttgcattctgggggcctacaggcttaacaccagatggaagttcccaaggcttatagtggcttgcactctccaaagaagcagcctaagctgtacttggggccctttgagccaaggctggagccagaggaaccaggatgtggagagcagtgtcccaaggctgcacaggcagcagtggccatgggcatggcccacaaaaccatcctttccctcttggcctctgggcctgttatgggaaagaccacctcagagatttccgaaaggccttcaaggcctttttccctttgttctggatatgagcacttagctcccttttagttatgctaatctctctagcaactggctgctccatagcctacttatatttgtctcctgaaaataccttttcttttctaccacatggccaggctgtgaattttccacattttttatgctctgcttccctttgttgatgaaaaaagccaaactccataaaacacttgacgagattgattctgagccacatgtgagagccatgaactgtggcacagcctcaaaaggtcctgagaacacctgtgcaaggtggttgggttgcggcctgcttttatgttttagggagacatatcaatcaatacatgtaaagtatacattggtttggtttggtttggaaaagcaggacaattcaaagtggggaattccaaatcataggtggatttgaagattttctgattggcaattggctgaaaaagttaaattatctaaaaagttgaagtcagcaaaaagcaatgcttaagataagggggttgtggaagccaagtttcttgttatgtagatgaagcctccacgttccagagagaagagatggtcaatgtctcttatcagaacctaaaaggtgccagactcttggataaatctctcctgaatcaggaagagacctggaaagggaaaaagattctcaacagaatatacatttcctccacaagagacggctttgcagggcccttccaaaatgtgtcagagaaatatattctggcataaaatactttaatttccttccatgcctgccacctgtcatgtgatgctataccagaatcaggttgggatttgatatcttattgctacaaagagtctgctttgtcagtcttaagctccctgttttaatgttaacactggtcagctgagcctaagctccaacagggagagggtatagtgagggagtccaaaccaccctccccttcctgtcatggcctgaactggttttccaggtttctttggaatccccttggattagcattttattttgtcgtttacaccttttaaatataagttccaactttaagtcatttctttgctcccatatcttaccatagactgttagaagcaaccaggtcacaccttagaaacatctgcttagaaatgtcttccacagatgccctaagtcatcactcttaaggtcaaccttccacagatccctagggcatgaacacaatgcagccaagttgtttcttaggatttaacaagcgtgacctttacaccagttcccaataagtttctcatttccatctgagatttcatcagcctggctttcacagtccatatgtctatcagcattttggtgacaaccatttaatagtctctaagaaattccaaactttccctcatcttcctgtcttcttctgagtcctccaaattcttctaacctctctgcctgttacctgattccaaagctgcttccacattttcaggtatccttatagcaatgttccactcctcaataccaatgttctgagttagtctatttgtgttactagaaaaaaaaaaaaaacatgaatctggtaatttataaagaaaagaggtttgcaccaggcactgtggcaaggaagggaggattaactagctcacagttctgcagccattccggaaacacggccctggcatctgcttctggcgaggcctcagggagattgcaatcacggcagaaggcgaagggggagcaggcatgtcacatggcaagagggagcaagagaagagaagtggcacgtcctagacttttaaacaaccagctgtcacagggactaactgagggagaactcactcatcacctgggagatggtgctaggccattcatgaggggtccagccccatgatctaatcacctcccaccaggcccacctccaacactgggaatcacatctcaacatgaaatttggagagacacacatccaaactattgcaccacaggaagctggaagaggtggacggtcctcccctggagcactcagagacagccaccaacaccttgatctcagattcctgactcccagaactctgaaaaaatttttgttgttttaagccacatttgtgatattttgttacggcagccacagcacggatatagctggtaacagagttctttagcctccaaaatacaagcctggggtaaataactattgaatttggccacatggaaatcaggaagaatgtaataagcatcatcgatggacgggatgacctgggagatcgtggcaagtttaaagctaattggaagttggcaaccaaaaaatcctgtcttgcaaattgccaggaaattgccaagaaaacttttagaaaacatggttaaagatggagaaacctggatgaatccctggtttctgaagaggtgcttaggcattgaagtgagactgatgagacttcacactgtaccttcagaagggcagacactcacgagatcatcaaccacggctccctcgctgggaggcagacacagagctggaggcggcggccacaaactaggtgggcagagaggcgtcagcatcgggacttgcgggaggccctgggaaggaacaggcagcggtggggcctggacagccctggggaaaggaagttacttggaggagaccaggggcttgcatttgggccaaaaccaaggacaagagatcagctggtcccagggaactctggagcccagcaccccaccacgggtgcagggtcctgacaacccgagagggctgtgggcaccgggcaccagcctgtggggattctgaaggctgctgcccagccacacgtcccggttctgaacaaagggttcaagagacacctgcaaacctacctttctgagagcagcctcttagcctcagctggtattggcctttggtcaccccacatgccccgacagtggggcctggcctcagaaaggggcccctccatttgtactttctatctgatccttgacacagtgctaacaccaaagaccaaacccgaatcttggttcacatactctgcaaagaaatgactaagactagttcttccggaggtcagttttagaacgtttattctgacttaattctgccttatctctgtgcaccaagaaaactatctgcacatctgtagtaaaatgtgaaggtaccttcattttgtttgtatactaaacttttaagaaatacctcactttttaaaacttggttttgtgttttgtatgcttataatattttccataatagatcattttaatgatgaactctgagtcttctgacagaatgcagatcaatggattcctagccatcagggaggcgttgattgcaaaggggcctggtggaatttttggagagattctacatcttcttgcaactgttcatctggattacggtggtggttgcatgactgtgcatttgttgaaacttacatgtcacaggatggattatactgaatgtaaactacacttgagtaaatatgacttttaaaactttaaaaagttgggggatggttcactgccccggctcgaagccccctggccacgctgcctggccagcccacccccatccctgccagcgcttgcctccagcgtcccaacagcctcctgaccccctgggctgctctaaaccctcaggagcgcagccatccgggatcagctggatggagatggggagcccgagactcgtgccacaccacgtcctcccgcccacaccagccacacgcagacgtcaaagcagcactgtcctcgcacgcactgctcccacccaccctggcacggccattccaggcctggggcaggaaggcagatgctccccctgcccccagacacaagcattcctgcacacacccccagcacacacacgcactcccatgcgcacactgacacacacaggtgtgtgcagctgagacacagcccgttcccaggaagcccagcccccatcactgagggaaaggggcagcaccgtagggccacaggagtggcagctggacacagagccaggagctggtgaaggcccaggccactgaggcgggctgccggcatggctgggcgtgaaggccagaagagggcaggaggggctgggggcactatgcctattgggcctaggtgggcacacgccgggcaggagaggaacagcccagcccctcagacaggaaggggtgggggcaggggccatttgtggaggccagggcagggccagcaccccaaggaaaagcagagcagggtgagaacggacatggggctcagagctgagcaggcctgctgggccccaggagggagacacagatgaccggagatctcaaggctggcagaggccagagatggagccccagctgggaagccatcctccttcctgggggcccacgctgcccggcccctccagcccagcaagcttggggcattggatagaaccgggagagagccgaccaggcactgaggcccctgccccaaatgcccacagcctggggaaaatgagcaggtacatgggaggggcaagtggagccccaggcacacccacacagtgcacacggcctcacctgggccggagggggcaggaggctcgccaccccgctgtggtttctctcctaatctcaccctgggtttctctcacacttgatgcagatgatgtttctctgacattgtggactaagagttggtgctggaaggggttagccatcttggagatgttgctatgggatgcagggattttgcgtgtgagaaggacatgattatggggggaacggagggcaaactgtcatgggttaaaatgtgtcccctataaattcatgtgttgaagtcctaacccccaggaccgcagaatgtgaccttgtctggaaacagtctttgcagctgcaatcaagttcagatgaggtcaccctggagtagggcaagcctctgatccaatatgactgctgtcctcatgaaaagggggaatctgggcacagacagcacgtggggagaacgccctgtgaagatggtgctgcttccataagccaagagcaccagagacggccggcaaagcccagcagcaaggagagagcctgggacagagtctcccatgacacagaggagccagccccaccgaggcctccatcccagatgcccggcctccagaaccaggacggaataaacgtctgttgtttaagccacgcagtctggggtgcagtgttgccagggccacagttaacggatacgagtgttgtcctgagctgccagccccacaggctgcacgaggcctccctgccccagcccagtgcagactccccagccccctgggtgtgccatgggcagtggggggcccctcactccgtcctcccccagcctgggaggttgagcccattatgagctccatggggtgaagctggaacgagaggctgggagccgactgggagcctgcggctggaggatggatttccccagggacccacacgtgcacctccacctgtctcctggacattctctctgagggcagggctggtgccagctcagggatccagcagggtcacaagggcaggccgggtccttgtggagagcacatttagtgggagggacatgatttcccttcaaagtgcccattctggacgcttcccgttccatgctggacgcttcctcttccacgctggatgcttcctgttccacgctggatgcttcctgttccacgctggatgtttcctgttacactctggatgcttcctgttccacactggatgcttcctgttccatcctggatgcttcctgttccatgctggacatttcctgttccactctggatgctccctgttccatgctggatgcttcctgttccatgctggatgcttcctgttccatgctggacatttcctgttccactctgcatgcttcctgttccactctggatgcttcctgttccacactggacgcttcctgctccacgctggacgcttcctgttccatgctggatgcttcccgttacattctggatgcttcccgttccatgctggacgcttcctgttccacgctggacgtttcttgttccactctggatgcttcctgttccacgctggatgcttccttttccacgctggacacttcctgttccgcgctggacacttcctgctccacactggacgcttcctgctccaggctggacgcttcctgttccatgctggatgcttcctgttacattctggatgcttcccgttccatgctggacgcttcctgttccacgctggacgtttcttgttccactctggatgcttcctgttccacgctggacgcttcccattccactctggatgcttcctgttccatgctggacatttcttgttccactctggatgcttcctgttccatgctggatgcttcctgttccatgctggatgcttcctgttccatgctggacgtttcttgttccactctggatgcttcctgttacatgctggatgcttcctgttccatgctggacgtttcttgttccactctggatgcttcctgttccatgctggatgcttcctgttacattctggatgcttcctgttccatgctggacatttcctgttccactctggatgcttcctgttacattcttgatgcttcctgttccatgctggacatttcctgttccactctggatgcttcctgttacattctggatgcttcctgttccatgctggacatttcctgttccactctggatgcttcctgttacattctggatgcttcctgttccatgctggacatttcctgttccactctggatgcttcctgttacattcttgatgcttcctgttccatgctggacatttcctgttccactctggatgcttcctgttacattctggatgcttcctgttccactctggacgcttcccattccactctggatgcttccttttccatgctggacctttcttgttccactctggatgcttcctgttccatgctggatgcttccttttccattccggacacttcctattccattctggacacttcctgtgcgacacctcctcgggcttttggtctgcccagtccctctggcctcataccatccccccttacctcccacttccacgttcgtccttcctcagctcctccctctctctagagcttcggcctggcaaggtccctcctgatctcagtccaggctcccccagcacaggtaggagacttgcacctgcccttggacctccccaccctgcatgatgccagcatcccccaggccccagggaggccccatttctctctctgcttgtagtccagtggccctggagtgccactgcaactcgggtgtgcccctcgcctctgaggaagctaagtgccctaagctaagcagaggccatcccctctgctcagccccagggccctgccccctaccccttcccctcacctgcaccacaggctctggccaactctgcccaggctctgaatgggcccctctggctcccctctgctgctacactgccctgcaccacctccactcagcttcagtgtgttcatccacctgtcccacgtcccctcggcccccaggagcacagctggtggccctggctcctggcagcccatcttgttccttctggagcaccagcctcagaagccttcctgtgcagggtccactcggccagccctgggaccctcctggtctcaagcacacacattctccctgcagccagacctgcccctgcctgtgagctcagacctgagccttggaacgccttcccttctccatcccagctcgcctttgccagctgctcagcgggatgaactcacactcccctccctgcaccatgagtgagagccagctggagagacgcccaggccaaagcagccaccagggcccagtgggggtcagaagcttcaggtgagaggcccaggtattgagaggctgagaccacgggcagaatggtcataatcactgccagtatcagtccagccccagggactcagagacagagaaaagagcagtgaacaaggtccgggctccccaccttctcccacgagtatgggggcagccaccacccccatccccacacacccatgaggcagcctcggctgtgtctggactcccccttaccctgtgacacagaaaccaccagaagaaaagggaacttcaggaagtaagcggtgccgccggtttcaatcctgttcttagtctttgcagcgtggagttcacacccctggggacctgagggccgagctgtgatttcctaggaagacaaatagcagctgacggcgtgggcaagtctgcccacatgtaccgcgccaaaacaggaagggctgagacccccacctcggtgagtagggtcagcacagggcaagggcacaggctcgggaggagaaggacagagcctgggtgcagccgtgggcgctcctggacctcagctgctgaacaggctacaagaggctggggagacgtgggggcaaggccagccccacatggagacccaagcggagccagcacgggggaggtgggcagccttcaggcaccaacgcccacccagtgcaagatgacggggaccgtgggcaggggcttccaagccaacagggcaggacacaccagaggctgactgaggcctccatgacgaccaggctgggagcacgaggaacctgacgggatgcggcagagccggccgtggggtgatgccagcatgggcaggacccacctgagctgaggaggcagtagaacgagggaggaggagaggccccaggtgaacggaggggcttgtccaggccagcagcatcactggagcccagggcagggtcagcagtgctggccgtggggccctctctcagccaggaccaaggacagcaggtgagccgggagcagagcagggagggtgagtgtggcagcaggacaggagggtggaagccaaggagcccagaggcagaggcagggacaggggaggcacaggggctaggctcagagccacctgatggcgctggggcacctgctggcggggagcagggctgtggtcagcagcggagtggaggggagagctgtgctgagtgcacagatgggaggagggaagagtccagggaggcccagaaaggcccagagtgcagcaggcctggggcgaggggaggggtgaggctccgtgcgttcagggagctgacccagcagagcagaggccactgaggagctgaggttctggagaggcttccagagcaggagcagtgcagggacgggaggatctgggagctcacccaggaggggcacatgggcaagggcaaggggctctgttggggagacctgactggacactggggctgctccacagcatagggaacaagccaagtgctgcaaaaacaaaaatgaggccagaaaaacagcccaaacctggacagagggtgccaggacaggcaggggggcaacagtgacctgagtgacattgctgcccgggttgagggagggcagagtgagcagggggcaggcattggagttcagggtaccaggaccgagcagccacaggtgagcagggcaggtgggggcagaaggagcagggggcacctcctggagctcagcagaccagggcagagcaactgaaggtgaacaagggcaggtgggaggcaggatgagcagggggaagaccctggagctcaggggaccagggcagagcagcctcaggtgcctcaggtgagcaggggctggtgggtggcaggacgagtaggggacagctcctggagctcaggggaccaggacagagcatcaagagctgagcatggctagtgggaggtgggcgaacagggtgcagcccctggaactcgggaccagggcagagcagcggcaggtgagcacgggctggtgggaggcaggaggaacagggggcagctctgggacttcaggggaccaggggagggcatctgaaggtgaacaggggctggtgggggcaggaagagcagggggaagcccctggagctcaggggaccagggcagagcagccacaggtgagcaggggctggtaggaagcaggaggagcaggggacagcccctggagctcagagcaccagggcagagcaccctcaggtaagcaggggcaggtaggaggcaggacgagcaggggacagcccctggagctcaggggacagaggagagcatcagaaggtgagcaggactgaggcttagcctcagggaatcagagcagagcagccacaggtgagcagggccggtgggaggcaggacgagcaggggacaggcactagagctcagggcaaggcaaccacaggtgagcagggccggtgggaggcatcactcagctcctagattttggcaggagctgggtagttgctggcagcagacagctgagggctggtgaaagtgcagtgcagcctcctggtgccaggaagggagtgtgagcccatcccactgagcagttggcaagggtgagctgggatggagaagggaaggcattccagggctcggggctgagctctcaggcaggggcaggtgtggctgcagggggaatgtgtgcttgagaccaggagggtcccagggctggccccagcggaccctaggcaggaaggcctctgaggctggcgccccagaaggagcaagatgggctgccaggagccagggccaccagcacaatgaagctgagtggaggtggtgcagggcagtgtagcagcagagggctgccagaggggcccattcagggcctgggcagagtcagccagagcctgtggtgcaggtgaggggaaggggtggtgagcggggccctggggctgagcagaggggatggcctggctgagggcagggcacttagcctcctcagaggtcaggggcacaccccacctgcagtgggactccagggccactgggccagcggcagagagaaatggggcctccctgtggcctgggggtcctggcaccacgcagggtggggagggccaagggcaggtgcaaggctcctacctgtgctggggggcctgggttgagcccagcagggaccttgccgggggaagctctggagagagggaggaggtgggctggtggccgagaaggccaggccagggctgggagggtgaggttgtggtgactgagcctccagaagtaatgcaggacactgggaggcagggggcatccaggcactcagggccctgacctgggctgctgcacactggggctaaggggaaaggaggggagaggctgaggaggaggctccaggaggctattccaaggcagggggttccggggccctggggctgaagggcgccgaccctatgcagtgtctggcccctctgctgcacagaagaaaagggccttggagggcagagggcaggctatgaccagggccctgggcaagtcaggcccactcactagcggagggccacgctggggcggcagggtcaggagcttcaggggactcgggggacccacgagaagccatctgagaacagtgtccactggtcaagccaggcacccataaaaggctggagtggggccaatgggcatgagccgtccctgaggtggcaccgatggccagagctgaggccaagctagaggccctggactgtgctgactcccggcaggcacagagcgctgacctggctgccgagccccgcctcctaggctgcaggggtgcctgcagaagggcaccacagggccaccggtcctgcaagctttctggggcaggccgggcctgactttggctttggggcagggagggggctaaggtgacgcaggtggcgccagccaggtgcacacccaatgcccgtgagcccagacactggaccctgcctggaccctcgcagatagacaagaaccgaggggcctctgcgccctgggcccagctctgtcccacaccgcggtcacatggcaccacctctcttgcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacagaccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtctacatacttcccgggcacccagcatggaaataaagcacccagcgctgccctgggcccctgcgagactgtgatggttctttccgtgggtcaggccgagtctgaggcctgagtggcatgagggaggcagagcgggttccactgtccccacactggcccaggctgtgcaggtgtgcctgggccgcctagggtggggctcagccaggggctgccctcggcagggtgggggatttgccagcgtggccctccctccagcagcagctgccctgggctgggccacgggaagccctaggagcccctggggacagacacacagcccctgcctctgtaggagactgtcctgtcctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtccagatgcccacacacacactcagcccagacccgttcaacaaaccccgcgctgaggttggccggccacacggccaccacacacacacgtgcacgcctcacacacggagcctcacccgggcgaaccgcacagcacccagaccagagcaaggtcctcgcacacgtgaacactcctcagacacaggcccccacgagccccacgcggcacctcaaggcccacgagccgctcggcagcttctccacatgctgacctgctcagacaaacccagccctcctctcacaaggtgcccctgcagccgccacacacacacaggcccccacacacaggggaacacacgccacgtcgcgtccctggcactggcccacttcccaatgccgcccttccctgcagctgaggtcacatgaggtgtgggcttcaccatcctcctgccctctgggcctcagggagggacacaggagatggggagcgggtcctgctgagggccaggtcgctatctagggctgggtgtctggctgagtcccggggccaaagctggtgcccagggcaggcagctgtggggagctgacctcaggacactgttggcccatcccggccgggccctacatcctgggtcctgccacagagggaatcacccccagaggcccgagcccagcaggacacagtattgaccacccacttcctgtccagagctgcaactggaggagagctgtgcggaggcgcaggacggggagctggacgggctgtggaccaccatcaccatcttcatcacactcttcctgctaagcgtgtgctacagtgccaccatcaccttcttcaaggttggccgcacgttgtccccagctgtccttgacattgtcccccatgctgtcacacactgtccccatgctgtccccacatgtccctgacactgtcccccatgctgtccccacctgtcccggacactctcctccgcgctgtcttgacctgtgcccaacactgtcccccacgctatccccccatccccaacaatgtcccccacagtttcctcctgtcccctatccccgacactgtcctccacactgtccccacctctccctgtcactgtcgcccatgctgcccccacctgtcccaacactttcctccaagctgtcctcacctgtccccaacactctcccccacactctctccacctgtccctgacactctcccccatgctgtccccacctgtccctgatgctgtcctccacactgtccccacctctccctgtcactgtccccatgctgtcccctgtccctcacactttcctccatgctgtcctcacctgtccccaacactctcccccactgtctccacctgtccctgacactgtcccccacactgtccccacctgtccctgatgctgtcgtctgtgctgtccacatactgttggtgacctggctctgttctccaagttcaagcctcagagcaggcagtggtgaggccgtggcacctgggtggcctgaggggtgggcgggccttgggggcagggctgtggcctcgctcacccctgtgctgtgccttgcctacaggtgaagtggatcttctcctcagtggtggacctgaagcagaccatcgtccccgactacaggaacatgatcaggcagggggcctagggccaccctctgtggggtgtccagggccgcccagaccccacacaggagccgtgggccatgctcagccatcacccaggccacacctgcccccgacctcaccgccctcaaccccatggctctctggcctcgcagtcgccctctgaccctgacacgccccccttccagaccctgtgcatagcaggtctaccccagacctccgctgcttggtgcatgcagggcgctgggggccaagtgtcccctcagcaggacgtccctgccctccggcccgccaggtgctcacacaaaaggaggtagtgaccagcatcccaggcccccactcaggcaggacctcgccctggagccaaccctgtccacgccagcctcctgaacacaggcgtggtttccagatggtgagtgggagcatcagtcgccaaggtagggaagtcacagcaccatcaggccctgttggggaggcttccgagagctgcgaaggctcactcagacggccttcctcccagcccgcagccagccagcctccattccaggcactcccgtgaactcctgacatgaggaatgaggttgttctgatttcaagcaaagaacgctgctctctggctcctgggaacagtctcagtgccagcaccaccccttggctgcctgcccacactgctggattctcgggtggaactcgacccgcagggacagccagccccagagtccgcactggggagagaaggggccaggcccaggacactgccacctaccacccactccagtccaccgagatcactcggagaagagcctgggccatgtggccgctgcaggagccccacggtgcaagggtgaggatagcccaaggaagggctgggcatctgcccagacaggcctcccagagaaggctggtgaccaggtcccaggcgggcaagactcagccttggtggggcctgaggacagaggaggcccaggagcatcggggagagaggtggagggacaccgggagagccaggagcgtggacacagccagaactcatcacagaggctggcgtccagccccgggtcacgtgcagcaggaacaagcagccactctgggggcaccaggtggagaggcaagacgacaaagagggtgcccgtgttcttgtgaaagcggggctgctggccacgagtgctggacagaggcccccacgctctgctgcccccatcacgccgttccgtgactgtcacgcagaatccgcagacagggagactcgagcgggagtgcggccagcgcctgcctcagctgtcagggaggactcccgggctcactcgaaggaggtgccaccatttcagctttggtagcttttcttcttcttttaaattttctaaagctcattaattgtctttgatgtttcttttgtgatgacaataaaatatcctttttaagtcttgtacttcgtgatgggagccgccttcctgtgtccacgcgcctcctgcccccggtgggaagcacggtcaggaggaggctggtccagctgcacctcgggggctccctgcatacgccccccgcctcctgcagccacacgcattgcccgagcgaccctccctggcccctgtcgctacatggacccccggggtttctcctcttttctacatggatgcagtttctcctcctgctgggcacggtgctgcctgccctggtcactctgcgggggacagggcctccagggaaagctgggtcgaggctgggagctggctcaggctgcccaggcagagccacagggagggccttccagaaccaaccatggtccgaagcgagaggtgggtgtcagatctgtgtgagtcagctcaggaccacagcggggcggctcccacagcagacatggatcctcccaggcctagagaccaggaatctgagatcaggatgcaggcagggctggtttctctcaagccctctctccttggcttgtagacaccgtctcctccctggtcctcacatggccatccctctgtgtgcccgtgtcctaagcttctcttcttataagaacacacatcggattagattagtgaccccctatgaacttaatgacctctgtaaagaccccatctccaaatagtcacattgtgaggccagggattaagacttgaatatatgaatttgtaggggccacgatttaacccatcacagtccagactctggcccccaaaattcatgttcttctcacatgcaaaacacattcatcctgtctcagcatccccctgggcactaggtcatgtagcaaggacggattttcaacagaaataactattgcaacagaagaaagagtccggcatgacctggactcaccttcatctgtgcagaggccacagccttgtaaagggaggtggtagggggagcagggagggtgctcggggctcagtcgtcggggaagggaaaagttgcccagcgctggtcagcgtccccgggatgggacccgctgtgtctgtgccggccactgttgaggtcaggattctgtcctcccagagcctggagacacaggccccatccttcccaatggggacacttcagggagtggctctcaggtcccgagaaagaccctcctgggtcacaggaaatgcacagacatcgggaacggatagaaggtcgtgtggttgcggccctctcagcagataccctgagaaagggaggtcggggttggtccaaacggtgagttctggtgcacggagctttctcaggcaggtgttgacggggcaggggtcggcctaggggtacggccagaagctgttagaaactgttagtgtctgctcaagtctttacaagccaaggttgaggccgagtggagaggctccgaggagcctggctggaactcagtcaaggacagggtcttgttactgcagtggctgcggtggctgcggtggctgcgaaatgccgtcggagttgcctgtggcaggagagagaccatctcacccaggaaggaggagtggttggattcgtttgtgtggcatcgagcagctggagcttcaccaaacacagagttggggactaaatccccagactccaggccctgccatgccgtgggaaggctcgccactggagggtgcgctccagggggcctggcctgaactgggtgctgaagcccagccctttaactctcaggacacgctgctgcagccccgcggggggtgagggagagagcacctggggtgcagggcgggcagctgctgcatcaccggctctatcccaagcccaaggatggcgtcccagagatgcaggagagctttgtccagagaaggtgccagccctcagggaccctgctggagagatctccaccctctgcccttcaaggggccctacgggcctccaggtgctctggtggggtgggctccagtccactgtctgaggatggacggcctggccaggataaggaaaggaaacccaggacggtgccgggctccgggtcattccgtgcactgagcaggctgagttgggaagaagcagatgcttcctgcagctgctgccccctgcagggcctggcgcctggaccaggttcccctggggaaattgggcccctccctgagccacccggggcccacctcccactttctacctgggaccgagcatcctccagagggtcagccctcctgcgggaacaccatgcccagccccaggaccctccctcaactctccagcaaggctgcccctgcacaccccccagcagcccatgctgtgatgtaacatgacatcgtgtgacatggtgtgatgtcctatcacagtgtgacatccctggtgtgatgtggtgtgacatggtgggatgtggtgtgacatggtgtgatgtccctggtgtgaggtggtgttggacatagtgtgatgtgatgcgacagatgtaacatccctggtgtgatttggtgtgacgtagtgtgatgggtatgacatccctggcttgatgaggtataatatgaaatgatgtggaatggtgtgacatgatgtgatgtgatgggacagggtgtgacgtccctggtgtgatgtgatgtaacaggctgtgacatagtgtgatgtgggtggtgtgacatgatgtgatgtggtgtgatggtgtgggacatccctggtgtgatggggtgtgattaaaccttgattccatgcagcacatgtttctgtgagcacagggttggggctaaagttacaggttaacagcatctcaaagcagaacaatttttctttgtacagatcaaaatggagtttcttatgtgttccttttctacataggcacagtaacagtctgatctttcttttccccacagtgtgacatggtgtgacatttctggtgtgactcttgtgttgtgacatttgtggtcaccccaggatacagaggtctctgtggccaagggaagggggagaatggaaccatctgagcatgttgacctggaggaattggtggcccttgagtccacgaagcccacccttccaggtgcccctgccccacgtgacccaagtgggcttgcagagcagcaagcaggactctggttagacaggaggaaggacctgccaccacgtggccttgtgaggagacagagcgaggctgtgacctcggcgtccgcccagcacagggtgctgctgaagctcctccggtcctctcagcagcggtctcagagcaaggcccaaggcaggctgaagagaggggcagagggaggatgctggggaggcaggtgtgaggggactgagagcccaggtttcagctgagcccctccacagggaaggagcctagctgaacacccatctccccacacgctcccaaccctgcctctgcccgaccacctcccagagggcacctcgaaccctctgctacccacactcagcaaggggtatggtgcccccaccaagcccagccagcaaagcctggcacagccacgcctgtgcccaccactcccatggccaaggtcactgctaacatggcaggacagagccaggcctggaggagacagaacatcagtcccatggggaagctccctgctcacacggcagggccaggcctggagaagacagaacaccccatctggcatggtactcaggctgcacatgcctgccacgaacgggggccacgcgacaatgcctgccacacatgggggccacgcaacgatgcctgccacacatgggggccacgcgacgatgcctgccacacatgggggccacgcgacgatgcctgccacgcatgggggacacgtgacacacacatacacacacgggcctcacaggcacacaaatgcttgcgaacccagcacccacccagcacactcaggcataggctccctgggcaggtcacaacctcacgccttgagctagtccatgtgccaggcccgtcacccacgtactcgtccccggtgctagctattgagccacaccgtcttctctgtggatccctcccagcccactcagcacaatggacatgctctctcccgtccagtgactgcaccggcctcttcccctcagcacccaagtctggccatctcccaggaaacccggaccaccacgggcagggaccacgttcctcactgtccacgtggaccaccccacacctgaccccagatgcagtcacactacgtcctgcttcaatattgaaaaggggaaaagctggaggagggtaaagatgaaagagaaaaaagcaagaggggagggtcacattcttctgaggctttgattacatctcactgagcccccacgttgcatgaaaaggaggggtggagggagcaattacacatttgccttgtgctcagtaaatctgcactttataagcaaataaacagagtagaggaagaagtcaaatatgcattcgtctcaggggcaggagggatgatttcttgtctcattttgtcccatgtcatgaagaccgggctgttaatttatattgtcagggtgagggaggccacctgggtagacctggcctatctgctgctgctctcagtttggaaacaaaaggaaacgcatgactttttttttttttttcatgactcagcttcccacctcaactgttctttttggcatagtgactttagggtcctgagattttattttcctttcacaatggtcaagatcacaccctcagtattcaggagagctggtccaacccagacccctgccgtccctggacgcttttcaaatacgtgctgcagtctctgacactctctcaccatggtagaaactgaggtcttggtgagtctgcagcccctgctcatggggacaatgcaggctgaaacccgcatttctgaccccaaagctcctgttcctttcactcacccccacaccagctctttgagtccagagctttgtccttgcctgagtcctaccctcagggacaggggcccaacccagccaccaacacatcataccctgagagggtgccaggagcccagagatgtttggagagcacagtagccctggaagctctgtggagatgctgcacatttctctattcaacagatactcgccaggtggccagcagcagagatgccacatggcatggagctcgccttggccaacaggacaggtgtggggttgggaggccttcccagggcactccttgaagcagagctgtggggaaaagttggggccaatctcagatctccccgtcaacaccgggtctcctgccctcctgggccacagaaaactaagctccgtggatactgcggctgggtggggccgtgggggagaagaaatcacaacgagttaaaagatcattttctaaaactgttatgatcaggactcacataaccatatgacgacacatttcagagatgctctttatctcattaattaaggtgtcgtaaccagttcaaagtggaattctaagtactacacttacataattgattcaggaatgctaaaaggagttcatagatagatgcaaaactggccttttccctggaagatgaggagcaattcattgtccttccaaagatgagaacttgaatttctaccaactcaaagagcttttgcattgctatcaattatgtacaacttagagcagtggtccccaacatttttggcaccaggaaccagttccatggaagacaatttttccacagaccaggatcgggggatggcttggggacaaagctgttccacctcagatcattaggcattagggtgtcataaggagtgtgcaacttagatcccgggaatgtgcggctcgcaatagggttcgctcctgtgagaatctaatgctgccactgatctgacaggaggtggagctcgggcaggaatgctcacacacccctcacctcctgctctgtggcccagttcctaacaggccatgaaccggttccagtgcatgacccagggattggggacccctggcttatagaggtgtaaaatagttcaaaggaaataaaagatgcagagctccacagaatgaaataacctggaagagtgtacaagacgatgccttgctttccatggaaggcacctactaatttttctcaatgtttcctataaacatatataactgactgacagaaacagatccataatataaagaagacccctgtaaaccaatgagaaaaaaaatcaaataatccaacgaggaataggcaagagaattgaacagatgttttacagaagatatccaaatagccactaaacatatgaaaaggtgttgaaccacactagtcaacagggaaatgaaaatgaaaaaccacatgagagaaagtagttctgattccagtaatgctagagcagctaatatcagactagccctttggcagatggcaattataaacactggaaacactgtaagcacacacaacacccacacacaccaattgcaggcactggaacatgaccagaagtaggcaaacactagtaaggattattccgtgaaatattcgtctgaagtcacaccccagtgcatgtaatgggtgcagctagagtttaagcaggaaactgcagccctcctggtgaggagtgggatgcagggctgcattttcagagcagctggaaatgaagagaagatgtccataaaggagaagctcaccgaagggaaaccacacaatctgcaagtaaactccactgaaacctctggccgatcccttaggtgtgcatgggtagggaaaactccaaagggcccagcagaaagcaacacctgtaagtcgagagaactgagattccagctactgccaactgccaggcagacagacttgggagtttgagtcaactcaagctaactgctaacattgaaaaaaacaattaatgctctgctaagaaagaatgcaaaccccatagcctgtacgcatgttatcaacatcaggtgcacatccaaaattaccatggatgcaaagaaacatgaaaatgtgatccatagtcagaagaaaaagggatcaatggagatcaactccaagatgacctagatgcagatacagttatcagacaaggactttaaagaagttatgttaaacatgttcaaagacttaaaggaaaatactattataatgagtgactagatggggaatctctataagagacggaaaatatttacaagaaccaaatgaaaattctagaactgaaagtatgattctgaaatgaaaaacatcatttttcagagcagggtgagaatggacatgggactcagagctgagcaggcctggtgggccccaggagggagacacagaggactgggggatttcaaggctggcagaggccagagatggatccccagctgggactggacctgggcttatgggagcaacaggtgacccatcctccttcctgggggcccaccctgcccggcccctccagcccagcacaggcattggatagaaccgggagagagcaggccaggcactgaggcctctgccccaaatgcccacagcctggggaaaatgagcagatagatgggggggcaagtggatccccaggcacacccacacagtgcacacagccccacctgggccagagggggcaggaggctcgccacccctgctgtggtttctcccacacttgatgcaggtgatattcctctgagattgtggactaagagttggtgctggaaggggttagccatcttggagatgttgctatggggtgcagggattttgcatgtgagaaggacatgattatggggggagcggagggcaaactgttgtgggttaaaatgtgtcccctataaattcatgtgttgaagtcctaacccccaggaccacagaatgtgaccttgtttggaaacagtctttgcaactgcaatcaagttcagatgaggtcaccctggagtagggcaagcctctgatccaatatgactgctgtcctcatgaaaagggggaatctgggtacagacagcacgtggggagaacaccctgtgaagatggtgctgcttccataagccaagagcagcagagacggccggcaaagcccagcagcaaggagagagcctgggacagagtctcccatgacacagaggtgccagccccgccgaggcctccatcccagatgcccggcctccagaaccaggacggaataaacgtctgttgtttaagccacgcagtctggggtgctgtgttgccagggccacagttaacggatacgagtgttgtcctgagctgccagccccacaggctgcacgaggcctccctgccccagcccagtgcagactccccagccccctgggtgtgccatgggcagtgcggggcccctcactgcatcctcccccagcctgggaggttgagcccattatgagctccatggggtgaagccggagccagaagctgggagccgactgggagcctgcggctggaggatggatttccccagggacccacacgtgcacctccacctgtctcctggacattctctctgagggcagggctggtgtcagctcagggatccagcagggacacaagggtgggccgggtccttgtggagagcacatttagtgggagggacatgatttcccttcaaagtgcccattctggatgcttcctggtccacgctggacacttcctgttccacgctggacgcttcctgttccacgctggacgcttcctgttccacgcttgatgtttcctgttccatgctggatgcttcctgttccatgctggacatttcctgttccactctggatgctccctgttccattctggatgcttcctgttccatgctggacatttcctgttccactctgcatgcttcctgttccactctggatgcttcctgtgcgaaacctcctcgggcttttggtctgcccagtccctctggctgcatctcgtcccccgctacctcccacctccacatccgtccttgcccagctcctctctctctccagagtttccacctggcaaggtccctgatgagctcagtccaggctcccccagcacaggtaggagcctagcacctgcccttggacctccccaccctgcatgatgccagcatccccaggccccagggaggccccatttctctctctactgctggcccagtggccctggagtcccactgcaactcgggtgtgcccctgacctctgaggaagttaagtgtcctgtccctagccaggctatcccctctgctcagccccagggccctgccccttaccccttcccctcacctgcacgataggctctggccaactctgcccaggccctgaatgggcccctctggctcccctctgctgctacactgccctgcaccacctccactcagcttcagtgtgttcatccacctgtcccaagtcccctcggcccccaggagcacagctggtggccctggttcctggcagcccatcttgttccttctggagcaccagcctcagaggccttcctgtgcagggtccactcggccagccctgggaccctcctggtctcaagcacacgttctccctgcagccagacctgcccctgcctgtgagctcagacctgagccttggaacgtcttcccttctccatcccagctcgcctttgccagctgctcagtgggatgaactcacactcccctccctccaccatgagtgagagtcagctggagagatgcccaggccaaagcagccaccagggcccagtggggggccagaagcttcaggtgagaggcccaggtattgagaggctgagaccatgggcagaatggtcataatcgctgccagtctcagtccagccccagggactcagagacagagaaaagagcagcacacaaggtccgggctccccaccttctcccgtgagtatgggggagtatgggggcagccaccacccccatccccacacacccatgaggcagcctcggctctgtgtggactccccctcgccctctgacacagaaaccaccagaagaaaagggaacttcaggaagtaaggggtgccgctggtttcaatcctgttcttagtctttgcagcgtggagttcacacccctggggacctgggacctgagctgtgatttcctaggaagacaaatagcggctgacggcgggggcggggccgcccacatgtacctcgccagaacaggaagggttgagacccccacctcggtgagtggggtcagcacagggcaggggcacaggctcgggaggaggacagcctgggcgcagccgtcggcgctcctagacctgagctgctgaacaggctgcaagaggctggggagacgcgggcgcgaggccagccccacatggaagcccaagcggagccagcacgggggaggtgggcagccttcaggcactgatgcccacccagtgcgagacgacggggaccgtgggcaggggcttccaagccaacagggcaggacacaccagaggctgactgaggcctccaggacgaccgggctgggagcacgaggaacatgacgggatgcggcagaaccggctgtggggtgatgccaggatgggcacgaccgacctgagctcaggaggcagcagagcgagggaggaggagaggccccaggtgaacggaggggcttgtccaggccggcagcatcaccagagcccagggcagggtcagcagagctggccgtagggccctcctctcagccaggaccaaggacagcaggtgagccgggagcagagcagcgagggtgagtgtggcagcaggacagaagggtggaagccaaggagcccagaggcagaggcagggacaggggagggacaggggctgggctcagagccagctgatggggctggggcacctgctggcggggagcagggctgtggtcagcagcggagaggaggggagagctgtgctgagtgcacgggcgggaggagggaagagtccagggaggcccagaaaggcccagagtgcagcaggcctggggcgaggggaggggctgaggcccagcagagcagaggccactgaggagctgaggttccggagaggcttccagagcaggagcagtgcagggacgggaggatccgggagctcatccaggaggggcacataggcaaggggctctgttggggagacctgactggacactggggctgctccacagcatagggaacaagccaagtgctgcaaaaacaaaaatgaggccagaaaaacagcccaaacctggacagagggtgccaggacaggcaggggggcaacagtggcctgagtgacattgctgccccgggttgagggaggacagagtgagcagggggcaggcattggagttcagggtaccaggaccgagcagccacaggtgagcagggcaggtgggggtagaaggagcagggggcagctcctggaactcaggggaccagggcagagcagccacaggcaaacaggagaggggagggggggcaggaggagcagggggcagctcttggagctcaggggaccagggcagagacgccgcaggtgagcaggggcaggtgggggggcaggaggagcagggggcagctcttggagctcaggggaccagggcagagcagccacaggtgagcaggggcaggtggggggcagaaggagtagggggcagctcttggagctcagaggaccagggcagagcagccacaggggaggaggggcaggtgggaggcaggatgaacagggggcggctcctggaactcaggaaacaggggagagcatcagaaggtgagcagggccagtgggaggttgcagagcaggggacagctcctggagctcaggggaccagggcagagccgccgcaggtgagcaggggcaggtggggggcaggaggagcagggggcacctcctggagctcaggcgaccggggcagagcagcctcaggtgaaaagggccggtggggggcaggaggagcaaggggcagctcctggaggtcaggggaccagggcagagccgccgcaggtcagcagggccggtgggaggcaggacgagcaggggacaggcactagagctcagggcaaggcagccacaggtgagcagggctggtgggaggcatcactcagctcctagactttggcaggagctgggtagttgccggcagcagacagctgagagctggtgaaagtgcagtgcagcctcctggtgccgggaagggagtgtgagtccatcccactgagcagttggcaagggcgagctgggatggagaagggaaggcattccagggctcagggctgggctctcaggcaggggcaggtgtggctgcagggggaacgtgtgcttgagaccaggagggtcccagggctggccccagcggaccctgggcaggaaggcctctgaggctggcgccccagaaggagcaagatgggctgccaggagccaggaccatcagcacaatgaagctgagtggaggtggtgcagggcagtgtagcagcagagggctgccagaggggcccattcagggcctgggcagagtcagccagagcctgtggtgcaggtgaggggaaggggtggtgagcggggccctggggccgagcagaggggatggcctggctgagggcagggcgcttagcctcctcagaggtcaggggcacaccccacctgcagtgggactccagggccactgggccagcggcagagagaaatggggcctccctgtggcctgggggtcctggcaccatgcagggtggggagggccaagggcaggtgcaaggctcctacctgtgctggggggcctgggttgagcccagcagggaccttgccgggggaagctctggagagagggaggaggtgggctggtggccgagaaggccaggccagggctgggagggtgaggttgtggtgactgagcctccagaagtaatgcaggacactgggaggcagggggcatccaggcactcagggccctgacctgggctgctgcacactggggctaaggggaaaggaggggagaggctgaggaggaggctccaggaggctattccaaggcagggggttccggggccctggggctgaagggcgccgaccctatgcagtgtctggcccctctgctgcacagaagaaaagggccttggagggcagagggcaggctatgaccagggccctgggcaagtcaggcccactcactagcggagggccacgctggggcggcagggtcaggagcttcaggggactcgggggacccacgagaagccatctgagaacagtgtccactggtcaagccaggcacccataaaaggctggagtggggccaatgggcatgagccgtccctgaggtggcaccgatggccagagctgaggccaagctagagacactggactgtgctgactcccggcaggcacagagcgctgacctggctgccgagccccgccccctaggctgcaggggtgcctgcagaagggcaccacagggccaccggtcctgcaagctttctggggcaggccgggcctgactttggctgggggcagggagggggctaaggtgacgcaggtggcgccagccaggcgcacacccaatgcccgtgagcccagacactggaccctgcatggaccatcgcagatagacaagaaccgaggggcctctgcgccctgggcccagctctgtcccacaccgcggtcacatggcaccacctctcttgcagcttccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcaggccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacct

SEQ ID NO: 70 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 71 - DNA, 1901 bp, Homo sapiens
  source 1..1901: mol_type = unassigned DNA
gcctgactttggctttggggcagggagggggctaaggtgaggcaggtggcaccagccaggtgcacactcaatgcccgtgagcccagacactggaccctgcctggaccctcgcggatagacaagaaccgaggggcctctgcaccctgggcccagctctgtcccacaccgcggtcacatggcaccacctctcttgcagcctccaccaagggcccatcggtcttccccctggtgccctcctccaggagcgtctctgagggcacagcggccctgggctgcctggtcaaggactacttccccgaaccggtgacggtgtcgtggaactcaggggccctgaccagaagcgtgcacaccttcccggctgtcctacagtcctcaggactctactccctcagcagcgtggtgaccgtgccctccagcagcttgggcacccagacctacacctgcaacgtagatcacaagcccagcaacaccaaggtggacaagacagttggtgagaggccagcacagggagggagggtgtctgctggaagccaggctcagccctcttgcctggacgtaccccggctgtgcagccccagtccagggcagcaaggcaggccccatctgtctcctcacccggaggcctctgcccgccccactcatgctcagggagagggtcttctggctttttccaccaggctccaggcagccacaggctggaagcccctaccccaggccctgcgcacaaaggggcaggtgctgcacttagactggccaagagccatatccgggaagaccctgcccctgacctaagcccaccccaaaggccaagatctccactccctcagctcagacacctctcctcccagatctgagtaactcccaatcttctctctgcagagcccaaaaccccatgttgtgacacaactcacacatgcccaccatgtgcaagtaagccagcccaggcctcgccctccagctcaaggcgggacaggtgccctagagtagcctgcgtccagggacaggccccaaccgggtgctgacacgtccgcctccatctcttcctcagcaactgaacccctggggggaccgtcagtcttcctcttccccccaaaacccaaggataccctcatgatctcccggacccctgaggtcacgtgcgtggtggtggacgtgagccacgaagaccctgaggtcaagttcaactggtacgtggacggcgtggaggtgcataatgccaagacaaagccgtgggaggagcagtacaacagcacgtaccatgtggtcagcgtcctcaccgtcgtgcaccagaactggctgaacggcaaggagtacaagtgcaaggtctccaacaaaggcctcccagcccccatcgagaaaaccatctccaaaaccaaaggtgggacccacggagcgcgaaggccacgtggacagaggccggcttggcccaccctctgccctgggagtgaccgctgtaccaacctctgtccctacagggcagccccgagaaccacaggtgtacaccctgcccccatcccagaagatgaccaagaaccaggtcaccctgacctgcctggtcaaaggcttctaccccagcgacatcgccgtggagtgggagagcaatgggcagccggagaacaactacaagaccacgcctcccatgctggactccaacggctccttcttcctctatagcaagctcaccgtggacaagagcaggtggcagcaggggaacgtcttctcatgctccgtgatgcatgagggtctgcagaaccactacacgcagaagagcctctccctgtccccggggtaaatgagtgcgacggccggcaagcccccgctccccgggctctcgcggtcgcacgaggatgcttggcacgtaccccgtctacatacttcccaggcacccagcatggaaataa

SEQ ID NO: 72 - DNA, 32230 bp, Homo sapiens
  source 1..32230: mol_type = unassigned DNA
gaattcaccattttaaagtgtacaattgagtggcatttagtacattcacgatgttgtgcaatcatcactcctattattactctgtaaactttacaaattcatcgtatttatttatttaatggttgttacagtttgcaactaaaatagaaggaaatacatcaaaatattagcagtggctcttctgggtgaagtacaaggggcaaattgttttctcctgtttgtttttctgtacacgttgtttactgtgtcacattgtttactgtgaatagttactatttgaaactaaagatcaagggcgataaactgtattttaagaggaagtcagaaaacaaacaaggtggccctctcgtatgctgtgctgacacctgtgtccctgtagcctcaactacgcctgttgcccccacctgcgcctgaagtttgaggtcgctggtggcacctctgtgccagggggccctggaacctctcctaggatggtcccagagaaaggagagagatctgcttcctggaatctcagctctagatgctttggtcaactccatgtcatctgtgtttgataagcttgatttccatttgttttggcccctcttttagtgtgctttttgttagcagaaactcacgaactagagaactgcaggtcccccaggggagtgggttgagatgtttttgacatcagccacctctttctcccacagttttgttattaaaaccccattttaacaaccttgaaagacaggttttaaaagaaagttgcaattttcttacaattctactgcttaattaaaagagtaggccctgtggttccctttccccatcctggtggtgttctccctgcccctgtggtggtgttctccctgcccctgcctctcaccagtgtatgctgttcccaggtggcagtaatcctatctcaggtctggcattccaaggcaggctcctgctcctgggtggggaggggcctccatgctgggttgaatgtctgtgctttgctgtcccctgacccacagagtgcccttggacaggacataggtctcagtttcctaagtgagagtcccggattagatggactgtggttaaccctttatgtgtcatggcctctttctgtataaagccacagatctttccctgcaatgagcatttatacagacacttttcagggttttgtgaacccgtgagctggacatcctagagctctccttcaatcccatggcctctagggtaccctgctctggaaaggcaagactgggcctcttttttgggaccccggagggcagaccttgggtttcgtcgtggcacccctgctgggtgatgtgcccagggctttccacgatgatcccactgaatcctcacatagccctgaggggaagctgccaccatcatcacctccacatcacagaggagtacacaggcaggacagtcacactcggacaagtggtggagccaggacacaaacccaggtctgtctggactctaagcttaggctcttaccatgaactgtgatccaaccatcgggagagtcagcccaatgaccttcaagttccctccaatgctaaggtcacacctgagaagcttgctagggtttctaaagcatcttcgtgcacatatgtccttccagggttcttcccagcagtttgtgagatgggcagggcagatgaagggaaaaggcttcaagacattaaggaaagggcccaagatcacacccgagtcaggatcagagcaggaattggaatctaatctctacacagagttgccagataccaatctgtgcttctcatggttagaatttacctacttatccaactattcacccatccaactacccacccacacatgtacccaccagtccacgcattcatctatccagccatccactcacccacccaactatccactcatccattcacctaccaatccacccactcatccacccatcaatcactcacccatctatacatccatccacctacccctttctccctccctccttctttccctccctccctttctttcctcattcagactgtttattgagagccttgaaggtcaggcacttggatggctgctggggagatgaagcttgaagatgtgtgaacgggctgctcctcttggagacatcatcttttccctcatcagtgagggttctggaaggggaatgtgggttgggaggcaggcaggatggagaggtggctgttgagttcctgccctttgggttttgggtcacagtactagactcatagaattgggttaaaaacttgtatttttttttctaaggagaagctggaaagtgaacaagtaagatagggcgtctggctccttgctgtgtatagaacagatacccagcaatgaccataggtgcagagtttatttatgtagagactttttgaatataagaaaagaaatgacaaatatatatgagttactatataatgaatataagtataattaaaataaaccaactataggtagtgcagggtgagtttccaataataaattctaaaccaacttccatcattcatggaaccagatattctattcaggaattaatatgcattctcccatacatttcctattatacacatgggaggtagctactcatattaaatcaatatcaaatagatgggaacatgaggagagagattagctaccaaggtaaattgtcatctctcagtggcagagaaatttgtcaaccgaggtcttcctagaaatatgctctgacctaccatacaattcttcctgcctgaggaactggtaaaaactctccaagtccatcctggcttccttctttccttcccaacttctgctcatcctgacctgaccttgtcccaagctgctctatggcttttattgctctttgcagaaatcgcttaaactttttgtttgcagttgctccctcctctgtgtttaactgtattttaagggggggttcattttccaatcctctttgctaaatgtcggctgctaggtactcatcccccctggaatgccctctctttccatttttatgactcagcaactttccctgggcttcctgcctctgagctggctggagctgtgagacaatttcgggctttgggcagcacctaagacatacaggatgcgaatccatgttaggatcgtagtgacctcttccatataatcccccattgtactactcctcaagagagaacactctgtcttctcatcagtgtaatctcaaaccctgcaccctgactgttgtgggtgtagcagacacacaagaaatatttgctgaatgttaagtgaattggaataaacatgttgatttaagtgacaaaaccttctatttttcataataataaatgtataatgattgtcaaaaaatgaaaaaagttagatgtacaaaatgaaaaccaatattacctacagtttacccccgcaaccggtagccccggggaatagatgtggatacggatgtgcatctaggtatggataaaaatgcagggattcatgcacacacaggtcacgtcttatctggtggcctctttgggataatatgttcaaggcttttcagatgccattcgcatcatcgaatacagcatttttataggatcttacatcttaaattggcttaatgttttgttctccgattataaaatctgaaaacaggaaaaattaaataagaatacaaagcaagtctctgatcctgcactgcccaagaggccctgttagcgcgttggtatggcgccttccagatctttttctgcatgaaatcatgggcttatcacaaagatgggaacttctttcctcacttcttcttgccctgttgtgaacatctgtctatggcccttctccaaggccatctggggatgtggatgtgaggctggcatctgctctcgggctcttttctgtcacgttttgctgttagctccctggctaacatccacatgcactggctctctggcatctccatgataaattcttatgagcaggatttctgggtcaaaagagagtgcattcccaggctgtggagacaggggacacgttcaggagggcctaaaaggctgaaccgtgtctcagccttgaggcctctcagaatcgccagcttgggaagttaggaggcctgtggttgctcatcactccctcaccaactcgttctgcccagggaaggtcctggggtgggggcttcagagccctgtcctggccacccccctccctggggtcctgctgacaatcagggccactgctggctggttctgagatgccttctggtggaccctgggacccaggatggagctcagggtgtggggaggcagccagggggcagcatcaggcccaggggctggactaggagggggtggggtggggcgcaggctctactgcccccacctgtgagctgcacaaacatccagcagcagccctgaaactgccccatgctcctcctgggccacacctgggcctgtttgtcactcatcccatgcccgggtggccatgagctcagtttctcttcctcttatttttctccttttgtcactctgagttctggtttcagccaacttggggttaaatttagcctggggatttccaggggtggccagctgcaggcagggccaccagagctgggaaagcgcatcccccaaccccatcccttggcctggcccccaccctactcccatctcaggcctccctggccaaccccttcacccaggaatctgctggtttcctgaatggctggcactcggggtccctgacccagttcctccttgaccagagggaaggctgggcccaggaaggcaaaggcatttgcccaggttcacacagtgtcacccgcaggcccaggcaagagcaatgactgagggtaggtgagggtccccataggccaggcacagcttaggcatcatctcaatccgcaaacctaccttgtggggagggactcttatcacccccttgggcagatgaggaaactgagggctacagaagagaagcagattgctcaaggtcaagggttgatgagggctgagcatggtggggggaccccttctctgtctgacactgaggtctgggctcccagggcagatgcccctttgggaagaacagtgctctgggctcccacaggcctttgcagccaccctccttccttgcatctcccaaggcacagggaaggaatggccatcagggcctgtagccctaactctcatccatgctgctttcaggcctcttttctgcacccctgaaatggggctggagctgtggaatggtggaggaagtgaacaatctggtctctggcagagggcagtggtccacacagccaccctccatacaggtcttctccatgcaggctcctccacacgggtcccaccacacagcgccctccacacgggcccctccacacaggtcccctccacacaggccccctccacacaggcaccctccaacagatcccctccacatgggcccctctacctggcaccctaccgacaggcccctccatgcgggccccttcacacatgatccctccacgcgggcccctccacgcaggcccctccacatgggcccctccacacatgatttctccacacaggcccctccacatgggcccctccacacatgatccctccacacgggcccctccacacatgatccctccacacgggcccctccacacatgggcccctccacacgtgggcccctccacatgccaccctccatacaggtcccctccacatgggcccctctacatgggcccctccacacaggggcgcctatgtgagctcgggcagatccctttctctcctggtctgtttctccatttgtcagatgggaggccctgggctggaagtcaggacaccacagagctggctctggctttttgggatctaactgctctgggcctcagttttcctacctgtgaaatgggaccatgattctggctgtggcctcacagaatccctgagtggctcaggtggataacatgtgtccacgtgttttataaatggtacgggacgataaggccactcaatagcgtctcaacacaggatgcagctacctgtagagttgcagatgtggtcagacctgaggtttagggcctctactctcggccttgctctctctgggccctgcccctgcccctctgcagagctctggggcttggggcactctggtccactggcctggaccagcatgaatgttcatggccttggtccaccgtccaacccagagaactgtgcaccctgcagggccctgccttgggcctggctcccagcatcctctgctcattccctctcctccaccgggtggtcactacctgtgtcctatgcagggcccagacctcttttctcccttgttccctgcccccctggaagcctggtccatcttggagcccatccatgtggcacagactccaaggacttcctgggtattaagtagggtggcaggccaggtgacgtgggggacagagtatggggcctttgaggtgagctttcagggtgcttccatgagggcccttgtgcactactgtggccctgtgggaagaagggatgctgtggtcactcctatacctttgctcacactcacccctaacacacccgtcctctctctgcccttcagtccttgatccagctcctaggcctgtttgaggttgctgctgccaccaggctacctttgatgaccacatggaatccccagtatctcctgccccttgtcttcttcaagcaagggggtctcatctgcaatctgagctctgggctgggcggggacaagtacccccccttgcttgctttacttcaggaactggcctgggtggctggcatcctgtgcatgttgaggggacggtggtgggggatgccagtggatgagctgattgaagatggtgctgggtctagggccaccctgttcagcctcgtgggtgggcctgtggggaggaaggcagaggtgggggcactcatccctggcctgcccaccctacagttcgcatcttatgtgtcaggtgacatgtgcaatggctacgtgtggcgggcctggctcctggctttcctgggccaagggagctccaggctggtgtcctctaagaatgagctggtgaagtcccacaggtggatgggatttgccgtctgcaggggtgaacccaggccctgaggctgggtacttggtctcctcccatactctcatcagaacctggagccctggaatatcctgggaacgtttcctccacatggagaaagacagggaggtctgtaacgggggcagtgggtaggatctggtcatcggacagggggcattaagcctcaccacagtggcctgttcctcacacctgtcaacctctctttgacctcagcatctagacccagcatggggaagactggacagggaatggggcaccccaaggccacagccccctcctcccatgagaaggcacactccaggtctcacctgggggtgggctggggcagtcctgccttgcctgggacaccatgtgtcaagagccctgcacaggccaggcctcggggcttgtggaaggatgagtgtgtgtgtgtgtgtgtgtgtgcgcgcgtgtgtgtgtgtgtgcatgttcagatatatggatggacagacacacaaataagtgactgaccagtctgaggtcctgcccactgggtagggaccccggaagtcctcttggcagctgtgcttccaggagcttcgttactgcctcaggctctgggctcccagggaagctcttggaagggttcttgtttcccttcccatccccttacttggcccagctcccttgaagggtccaagacctgggggcagcaggtggagtgtgatcagcagggtcccatgtccagctctggccagccctgcccaccccaggcccctctgagctccaagtgtctggctgtgccccccaattgggcacagctctgaacctggcatttagaacggttcctggggagctgtgaggagggggtagggcctgcatgaatagaagcttctggagcagaaggtgctacttctgatcattgattgaccatgatctccagaccctgtctaactcaagcctgggcacggggcttgggtgtcttggtgcggggacacaggaccattctccagctgggctggtggaagctttggaaactgaatcgcccccctgcctcccaccctggggagaaggaggcacctgctgccagggctgaggcttggtggggtttcccagctatttcgggcctcaggcactcttggtttcaccaggccagctgcactggggtgagggttccagggccggtggggagcccagcatgggcagggggctggcctgaggctgtctggggctggaacagcccaaggatgaaaacaaccaacggcagagacaaacaaaacatctattttccatctgattgtccctccccccacccagagagtcagtttccattattatcaacccagctgcacagaggggaaactgaggctgagctcgagccactaactccagccacaggggcctggcgctagtcagggctcctcagtcgctggctgcctccagcccctgggacgtcgccccccgtgagaccccagccaggtgctccctaccccccatcctctgccctatttctccccagcacaaatcccgcctcctacatgacattgttccttgtctgttgacttgatttccctctcccgcaaacatgagctgtctgaacctggctgtgcctgccttaaccctgctgggtttccaggagcagcaggtcctcagaagagagctgaacatgtgacttcgctctgggcagcccctgagaagggggcttacttacctggccggtctggggtctcaccttcttcagctactctgcccagcagcccctggggaccagcaccccttccttctgggagctgctctctgctgcccagacctttgcaaactttctgtcccctgtgggagcccaggccaagtctgaccaccacttcccatcctcctccccagacaggtcaggagaggatgtgtctaggggtctgagtgggaggctcccctttggcctgaaactgtgaagatgaggcccctttctcctctatcatttccatagtcagaaagtcccaacatgtccctgcacaggctgggaactgggtgggcagggagggcccatcctagtgacacctgtgaggggtccaggctttggtgcctggcacggcaactggcacattggatgctcagtgaacatcagacctgatgtgagaagcaggagggacccgtggccagatcaagggtgttggtgttgctataggaagagagagtcaccatgaaggatggcctgtatggaaagaggcacactgcgggtgccctggtgggtcgtcctggctgtgggctactctttcctggagctgacacgggggctctgtgttgccagccacagaagtcacccagcccctccctggctcctgtcccaaagccaggaggcattctccctgctgcctgtggtctccccaggaactagggctgcccagaggagctgtcccttgccggtctctggacgtggtgtgctgttgctgagtgggagatggtattagggccacccctggttgtgttgtgagctgggcctgagtgagctcatttctaggggctccaggcaggggaccaggaatgtcatggtgactgcctggatgtggcccctagtgaggctctcctggcccctggcccccattgtaatgtccagcactgcttaccatagtgctaggtggctgggcttggcttcctccccctcattattagccattatgaagaacgtcattcagtcatttagcaaacagttactctgagcttgggcaaccaaaatttgttgtttaaactgggatgttcctgggagtggaaggggcactattaataacttcacgagtctctctgatagatgtgggtgggggtcctggaggcagcccaggcctgggtctctgggcagagcccaccattactgctacaatgggccaggcacggccaggcctgggtcccgagctgaggctgggtacacataggcacctgggaaactgcagctgattggtggccagtggtctggggacctggatggggctaagggtcaccaggggctggacactgctctagcatccacctcagcttccagagtcagtggcacaaactgggaatgtcctggaaaatgcaggatgcaggtcatccccctcccctcagaccttgcccagcccacgtgtttcctggggagaccacccttgtgaaattactggccaaacagggtcaggtttgatgtggtcccaagtgaccaagtgacaaagactgaccctgatcctgaccctactgcccctgctgatgtcactgtgttgacatttccgtggcccatctgcaatcctcaagatgaccatgtaggagagcagtttccccagatcaccccagaaagagggagtcctcacgcttgggttgttacaagaggtacgggtgccttttgaggtgtttgcataactaagcattttgggaaaagacctagtcttcccggcctccgttgatgccagctagggatgcttctgaatccccgagggaactggagcatctcacgtgtggcgcaggggatcctgggcagatttggggtcatttttccttcacgtctcattcagcacagggaagaggaccagtgtcaagtcatgacacacgactctttcagggccgtagaagctggtccttaggtcagggatggagcccagggtgtggggtcaacatcaaggactcgtgtcctgcaggcggggtgggtggggaatccagaacctggccctggtgtgacagagactctggagaagaggatgacatgtcgggagggaggagcaacatcctcagcattgtgctgtgtcattagcagtgggcattgcgagtggatggtgattatttcctctatgtccccaaaattcgcatgttctagttggctggaaacttgattttggacctttgtgttgaggaatgggggcttgcctgggatctggtggcctcgttatggggcatctgagctggtttgcttgttcccaggtgagtgtcgatgtcccccttcttgcccctggattcagtgaatatgggggttgaagggtaatggtgcctttgtccaggcctggctggggacactctcttggtggctttgcaggccccacaggaggctgggcctgatgggagagtcacccactcagttcatagtagctcccagtcctttcctcccgcagggccctgagcagccacccccctctcctccagcagggcccctgcaccccctcccaggctattctgggctctggctttgctgtagaggaagccacagggcccctccctgatctgactcagaacacctccctcagcccaggctgcagctaagcagggccagggccaggacgagttcctctcaccccatcccccactgtgacactcttccctataccttgaggggttcccccagcacagaccaaccctccttcggtgtcatgaggccatcactcgtgaggggctggccccggtctgagctagacacagtggcatgtgaagacaggcgtgggctaaggcccaactcggctactcctgaactgggtggagcctgggcagcgctggtgcatccatcatggttcacgtcccctctgtgaggtgggcaatggcagctttaggcctcagggtcactgtgagggtcagtgagggactgtgatgttcccactgtggcccaaggatcacagctggggctctctcagatggttctttcagttaaatcttttcctgagtgtggggagatgctcctgctgccccagacactccctgggcagagtgccaaccttggagacttgacagggtgggggcagtggtgtctccatgtcgttctccaagcttgtgagctcctctagggcaggtggtcctgggctgcatacaccagcccctggcccacagctgagagtttgttgaatgaatgaatgagtgagtctgaacaaatgaatggatgaatgaatgaatgaaaagcccttagggccgaggagtcatgaaaagaggcgggcatgggtcagccggtaacctactgtgtgaccctaggtgggtcatcacccctcccatcggagcctcagtcctctcatctgtgaaatgggcacatgaacacctgtcctggctctgctttgtagactcagagggcacccccatggctgccctcctgcctgcctctttcacccactgagtcaacagagaatgaggagcccaggtctgcccatcccacagagccacaagggggttcatgcactcagcaccatctcttggccttgcccaggcacggcctcaggatggaagggtggcctaggtcaggggcctgggcaaccactgtgctgccctgaacacctgcaggacctgggcaggctcagcctcctctgcacgccttagggaaggggctggctctgggttgaccttggatcagagtcccagtgcccaccatctgtgcagccctgggcagctgtgggactcgggcaggctgctggtctccgaatctccgtctcctcatctgtaaggtgtgcgggacatggacacgtgcctcctggggtggtttgggatcccatgacatagagaggacagacgccagtgcagggtttccctggaagtggccctgggccaggtggcaggtgaggtagcatcttcagcagagccgggagcagttccactgtcgagcagtggccatcgaggggctcagtgatacccagcccagcacctggctgaacatcggaggacgtgaggggtgcagcccctgcttccaggctcccgtggtgtcccagatcggtgctgggcagggcggcaccaggagagcgtggtcagcagccctgggcagccatcaagcctgaggcaccactgggctgagggctggggccggggaaggagcaaggaacccttttacattccggtagttcatgaatcacacagtgaaagaaagagtcaccaagggtgctagctgccggtggccctgatggggcagcagcagaacaaagtgggaggggaccatagaagagaggaccttggcggggggtgggggtgggggtgggtgggtgaggggcttcaagatgacgagaatgatgtgagttgccaagcccatcctgaggtcaacagaaggaggacagtgaggggaccattggattgagccacgtgcaggccactggggctgtgatggggtggtggtgagactgctgggcttgaggcaggaagggaggggaccgtggaggccaggagcccttgattaccacttgtaaaaggcaggagagagggaggggcagccagaggaaggcagcattcacattttattacttcttcctggcaggaccacatttgtgctcctaaggtgaccatcaggagggtctctggcccttcatactgagatgttgaggcagagagactgtggtccaggttctctccaggaagagcccaccattactgctgcagtgggccaggcaggactaggtctgggtcccaagcccaggctgggcacatatgggtgcttgggaaaccatagttgattggtggccagtggcctagtacctggatggggccggggtcaccatgggctggtcactgctccagccccagcatcaaccccagctcccagagtcagtggggaaggggactggagtggggtccgattacagtgaccacagggaaatgggggccacagggaaatgggtgcggggagcaggggagattcgggaggtgggtggaagaggaccacgtgctcccatcctgacagcccagagcctgcgtgtgcctggggcggctgctctctcaaagggcatgcgtctgtcttggggactgagggtctctgaagtcaggtcgcctcttgctcgaaagccccctgccaccttgctcttgactttagcacctcctcacagggcagcacgtgttatgttatccgggaggaaatggaaaccctgggaggttctctggtgggggaagggaggagcggaggtagactaagagggggtcagcagggggagaagggggaaccagcccagagttctccagggacgggtgcgtgttgtcctagcctgtgccctgggagtgacaccaggtgagttgagatttcagtacccagcatttttgtgactgaagtaaagaaaattctgatttggcccttgcctgcttgtacaagttaactgcttgctttttgcccagcatcccttgttcacacaatgtgatcataagctgcagatgtcctgtttctttgtcaagaaggaggagataacttcagagtcacaaaagtattttgcaggagtgaatgcctttagggacgtagagaccagccgctgtcgcccgcaagtctagttgcttaagatgttacctgagactgaataaacacagacttttcccctgaaatgccacctccctcactccttagccacataaaaactccctgcttcatccttttgttaacatggagtggaggaatttgctttcttgtcttcttactttagccaaatgaaataaatctttttctatctccaagcacttgtgtcagtttttgacctccgctgcatatcaggtacacgaacctgaatctggggttctacaaagagtctcacatgtcaccactcagcccccctcgaacacctactgcgctcggtgctgggccccgagaggaccggggttcctctttcccacagccccagacccagagccccatgtgctcttcccttgggagagcagtccctgactgaagatgtcaggaatttttggatgttgagggagcaactgggaccaggaatggaggggattgggcttggggagccacctgccagggactgactctcctggaggtgggggacaccgtaagccctggatccagaaccaggactgagatggggtctctaggaaaaggggactctcatggtccctccctccgagtgccctgaccctgatatttgacccggaggaaggagaaggggcatgaggaatctggctactccaacctgaggccccgtccccttccatcccttgttggtccaataattctgtcacttgaatcccgggtcccgggatgggggcctgggtccagtgggctttgagctctccagggaggaagctggatgaagtgggtgggcgtggccaggtgggctgagggcagccaggcacctggggtccagagctgagatctgcagttgggtggaccaggtttgagtccctactgtgacctgtggcaggtcactcaggctctctgggctgtgatgtcaccatcctagagtgaggatcacaataggatcttcctccaagggtgatttggcctggagggcatttagtctcagggggcctgctggaagacagtgccctagagctgtggctaaactcccagtgccttcctggaaccccaggaggagaaggggccacaagagaccagggatagggagggcttgggctgggctgggacggccccccaggagtggctcagcctggactgggcagggcatggagaaagggagaggaactcgggaaaggggctgtggtcggctaaattaggccccgcggggacagtcgtgtcccaatcctggacctgtctatgtgtcatgtgtggccacagggactttgcagatgtcatgtcaattaggagcctgaggtgggaaatgctcctgggttatccaggtgggtccaagctggggtctttgtagaagaaagagggaggcaggagggtcagagtgagaggacaagatgtggggatggagggagacggagagagagagaaggaagagagggagagaagaaggtagggagggagagaagagcaagagagagatttgaagatgctgagctcaggctctgaagttggaggaaggggccaggagctgaggttgtcctctagaagctggaaaagtcctggaaacgaattctccccagagtccccagaaggagccggccctgctgggaccctgattttagcccccaaggactcattttggacttctgacctccagacctgggagagagtcagtgtgtgatgttttaaggcactaaattaggggtcatttgtgataacagccagcagccacaggaaagtcaaacaggagccgtgggagagactccccaggatgccacaagggcccctcccaggccccacccacagtaacccaggcctgacccctgcaggtgcccccactctacagaggaggggccatgcaggaggggctgggtggggctgggtcgtgttctcttcccccaccccttcctctcagagagcagttggagccccagactcagggaacctcacagagaaagtcctgatgagggacctggttttccagtgaaggcccgggatccaatggagaagtgttcccaaaaatgatgttcccaaagacggccagtgactttctgttcacgcacttgagagaatcaccatgtctaagggctgccagatagttacctgtgcgtgtgtcaaggttcacatcccattactactgaggccttggatagaacatgttcccacttttagttgtgacgagatgactggccccagcattgagaacaccaccttcccttcagcaaagggtttgtttaaatttacagaagtggtgaagtgcttattaccctccttgcactggttcctagggagctcagagcctatcttttccaagtgtcccgagcctcctgtaggcctttcgtgtgagtctccttcctggcttcctcccattcctcttaagtgtctgaacctgctgtcaggtgaggaatcgaggtgggtccctcattgaacagcttgagaagtcagtggtgctggagtgtcctggacagtctggctctcagggatgacagggagggggtgagggtgggtgggagctgagctgggcacagagggaggagggcattccgggggcaagggccaggggcaaagaggctccaggcttggagcagggttttcaggctgggaaaagagatggtgggtgaagggtgtctctgttagggcagagatagagggtgtaactggagtcccagccagagacaactccatccaggcaggaggcagggaggtggggggctatgctttgaggagaagggggaggaatgaaagggcagagggactggtgttcagtgatcagacttctcctatccgggcctctttcaccaggagacaccccgggaggctatgagcctctccctcagggccactttctcactgggtgccaggattcccccacccaacactgagaacccaggggaggcctcaaattatgtgtgagataagaaaagaagagagggagcaggatttctcgttgacgatgaggatatttattgagaagggctggatgacttgggatgggggcagagacccctcccctgggaacctgcagctccaggcccctgtgggtggggtgagggtcgggggcctaagagcattctgcaggggccactgtcttctccacggtgctcccttcatgcgtgacccggcagctgtagcttctgtgggacttccactgctcgggcgtcaggctcaggtagctgctggccgcatacttgttgttgctttgtttggagggtttggtggtctccactcccaccttgacggggctgccatctgccttccaggccactgtcacggctcccgggttgaagtcacttacgagacacaccagtgtggccttgttggcttgaagctcctcagaggagggtgggaacagagtgaccgagggggcagccttgggctgacctgcggggtggatgaggggcagggggtcagagtcctggtgtccacctggggagcccctgacctcagtcttgaagtgggtatgggactaaggcccagggcagggacctcaggccggacccttgctggactgaggtcaggtcagcggtgagtggagacacttgctgtcagatgggagttgcccccactcttgggggtcacagaccccctcttggatctgtctctttttcatattttccatcacctatagacccccaccggtgctggctccacactggatgggatggagtccaggaccacatctcaccctgggtatttgtctatcttgggggtgtctgtctgaacacctcctaccagttccccttgttctcatctcttcatgcagggcagctgttggtcctgggttcaggctcttctctctcacagggagtgggtttcttacccacacatctccctggtgccatcattgcagcccccaccccacctccctgcagagacccctctgggctgcctggtgagacagggtgtcagtcctgggggctggattccagtagatcaaaggcctccatttgaggcattttttttttactccctgagctgaacgtggagctttgtctgttcgaaagagtctccatgtcgtgagaagttatctgggggatctgactctccagtgggttttccctgacaaacaggcacccgtcccagtcaccgtccctaccagaagtccctgtcctgggcatctgctctgaggtcttcctccagtggaggaattggtaagcatccacgtttagggcctgagttttctgagacccaccaccctgtctgtggtgtcctgcctgtcttgggggatggcgtgtgacccagcgctgtggggccaccttctcacggaatattcttcctctatctttgtcctcctgccatttcctcccccatctctccaagtcctcagtcaaaggcctcctgtcactgtgtttaatgcagccccgtaactgggacccacagaggacagaatcacagaagagggcaggagcccaggcaaggaggggcaaagattccagacctccagacaggcttagaccttagccttcgacagacaggaaagggaaaaactccccgtgtttaacttgggatctcaaagaggagagaagcggggagacttaccgaggacggtcagctgggtgcctcctccgaacacagcacacagtgacacagccccacacacaaacccctcctgacacacccgacccgttccccgctgcagcagcttgggcgcccaggtccagtggccctgtcatgaagcaggtctggccgataggagggtgagggggatggcccctccatgcatggatgcacactttactggaagtcagggatcccttcacccccagagggagaggggagagggaggaggcatgtctgcacatcacatctgggtcccctgctactctctgtcctggaggaagccatgtgtcccggctcagctcctttggagactgtcaccatggaagaagtgagaatcctgtttatgccacagggaaggtggagctggtcccctatggagatccgagttccctgggagggtcctggatgcccagcacagcccctagtgggatgcccatgtggtaatgattctaaatagcacaggagagtgtgtgtgtatgtatttgcatgcaggcatgtttgtttgtgaatacctgtgtgcctatgaggtgttcatgtgtgtttatgtttgtgaacatgtatgtatgtgtgtttgtctcatagcccaggccccccatcctgtgctacctcctgggacaatcatcaacccggttcttagatcccacatacagctcttaaaaagaccaattggtttacaggcacgtctatggcaccactctggaatctcatgattgactttgacctccccagccccactgaccatccgtggataactggacattaggtacttgaggtgtggtcagttttgggctggttacttaggggtgagagagagtagagttgacccttatcccccaggggacttgagtataactgggacactggtgacacatgtgggaccaggggtacccagctggcctgtggggtgagtgacctgggtccagagggtcagggtccagtctgtgcctgcttcagagatagatgtgggggtagagggcagggtcctgggagtcaggaacagggtgggcccctaaggagtaggggagaccaagggcccatggactggagaggctccttccgcctcagaaggagggaccccttgagagagggaggctgtccaggggtgaggcctgatgatccgtggagggtactcgttcttctgggccatggggaaccaaggagcggtcgccccaggccgctggaggtggacatgtccacacaaacacagcacgtatgggaaaggggcagtcctggggcctgctgcccaaggctctgttctggaatattctctgggcctttctaggtgatggtggaactacccctcaaaaacaatccagacttcattcccacaaggctaaaagaattttgttcatcttgcaggttggaccaatttagggtagaaaatgtataaacagaagcaaaacacaaaacccagggtttctggttgactgaggatatttattgagggtttattgagtgtaggaagaagggctggatgacttgggatggggagagagaccccctcccctgggaacctgcagctccaggcccctgtgggtggggtgagggtcgggggcctaagagcattctgcaggggccactgtcttctccacggtgctcccttcatgcgtgacctggcaactgtagcttctgtgggacttccactgctcaggcgtcaggctcaggtagctagctgctggccgcgtacttgttgttgctctgtttggagggtgtggtggtctccactcccgtgttgacggggctgccatctgccttccaggccactttcacagctcccgggtagaagtcactgatcaggcacaccagtgtggccttgttggcttgaagctcctcagaggagggcgggaacagagtgaccgatggggcagccttgggctgacctgcggggtggatgaggggcagggggtcagagtcctggtgtccacctggggagcccctggcctcagttttcaggtgggtgaaggactaaggcccaggatagggacctcaggctgggcccttgctgtactgaggtcaacttggcggtggcggggatgcttacctcctgatgggaaactcctcagcatctgaggactgaggagaatcaggcctcttctgctccagcctcctctccagattctcccctccttatcagccagctctcacctcacctatgctccccgacgctggctgggttcagccctgggccctcgtctttccttgggggacgtctctgtctgaacatcccctgccaggtccctcctctgggtctgttcacgagggcatctgtgtcctgtttttaggttctcctcccttttgacaagtgaatttcttcctcacatatcccccagcaccatcattgcagtctctgccccactttcccacagaggccccttggtggggaggggatgtcagtcctgggagccgggtcctgggagtccaggagatggagggcttgcatgtgaggtatattttctgttctgagcctggcgctgtgtctgctctatggggtctccctgtcgttgggagatctggctgtcctaggacaaatgtctgtcctgacaggtccctgtcacctgcgcccaccaggagtgcctccctcagcatctgctttgggggtctctctttttgggtgggatggggaggctctaaccttggactgagtgtcctgagaaccaccctgtccatggcagcctgcctgtcccgggggaggaggagggacccagcaccctggcgccacctcacaggacattcttcctccatccttgtcccccttcatttccccaagtccccagtcaaaggcctcccttcttggtgtttaaagcagccccatagttgggacccagaaccctgggaagctgaactttttccagcatccttcccctgtcccatggccctggtgccccagcctgagcccctgctctgctggtctttggggactggagactctgtgcgagggagcagaacatcccagagcccaggggagaatgccccctgagtgacagcggcaggttcaggcctgagcagggagcattaagaggtcactgggatcttcccatcatgccccaccaggtgcctaagaagggacaggctggaggaatttggggccaggaaaaaaggcaatggtggcagaccccagagagattgagacctctaccctagagagaacagaaaggacagaaaagtctccccagatctcaccctagacccaaaagaatagaaaaggggactcaccgaggacggtcaccttggtgccactgccgaacacattacactgtgatataaccctgcacacaaaccctcctgatacacccgacccaggccacccccccccaaccccctgcagctgctcacaaggcccagggtccagcccccttttctcatgggtgggtgagccataggtggggtgaagggggtcaccttctactgacctgaatatcacttccttgggggcttaggggttcatccacctcagtcatgggaagggttgacaggtctgtgtgtggaattgtctcccaggtaattgcatttctgaggagttgggtggcctggatcagctggtgtcaggaatgccagggaggaaggggaggtgagcggctgggtgacgccactgacatggtgcagctgggacctctttgagaagttgggaatggtggggagggtccttgatgccccccacatcctcagcactggaggttatgatgggatgggcttggaatgggaattgggggttgtgggaagaatgacgttgaccacagggacacagaccccactgcaaggtctgagctccagcatccttggcccagctgtcctgaaggagcagctcacagatcactgcccaagtccgcctcctgaacccatggggacagcttcctacctataggccatgtgtgctggattagagctgcaatggcccagaggagagcttctcaagggaggtcataggcctcactggcagtgtggtttcctccaggagctctacgtgtgggggatctctgctgtccacttcagctcccccggcttacacacacacccacatgtgtgtgctctgcagtcccctacatgcacgggtgacccacacagcctgtgctctggagtgtcaccggcacggggggcacccacagtggatgggcatgtgcagtgtggggcctggacacacacagcctggacctgcatcctggtctgggcccatcacctgaagagctcacacctgggacacactcctggcaccatgcagtccccaacagtattccccacagcaagcgcctcccctgactgctgagatgtggcctgctgctccctgatcagaatcccgtccacatcttctcctgtctccctcttggcctggacagaaaaccctgccccaccctcccatatccaggggcgaccccctagtcccaaagcctggagacagcaggtctcagctttgctttgctcatgtcctgacctgggcctgactgcagccccttctaccttcctgatcctcccagaaggatgcccagcatggacatgtgtgtttatgtgcattgtgtgtgtgtacacgtgtgtgtgtattatagctcagatcccatgctgagttcttcagaagaagaatcagcgtcacccaagctcctggcataaccccagaatctgactttgaactcaccagtcaattgacaacttgtgcacagcaggacattaggcattcataatggtgtcagtgaggagcagagccgacccttgtcccctaggaatgttgaatacagctggggcatccggtgacacatgtgggatgagaggcacccagagggggatgtggggtgagtgaccagggtccagggggtaagcatcccatcagtgattattatagggagggcctgtgggggctgagggcagggtccctgggatgacgggaacagagtgtgcccatggagaaggggagatgctctaggctttgatgaggggaaatacaggtggggtgaccacatggccttggagggggatggacaaacaccttgagagagggatggcatctggacagtgagggccgaggacaacaaagaagggagctgagatgttctcccggggccatagggatgtaaggagggtgtgtccccgggcagccagaggtggacgtgctcacagggcagcaggaagcacaacagagacgtgggtaagaatgaagatgtctggttccttctgaagactatttccttgttctggaacattctatggggataaggtgatggaaaaagtatggctcagagaaacacgaagatggtcccaggctgcattgtcacaagtccaaaggattctgttgatctttggagataaaaaaatttaggatggaaagtggataaacaaaaatgagcagacagcatgttttctgattgacaaggaggacatttattgagggtttattgggtgtcggcagaaggactggatgacttggggtgggagggaacacctctgccctgggatcctgcagctccaggcccctgtgggtggggtgagggtcgggggcctaagaacattctgcatgggccattgtcttctccatggtactttctttgtgcgtgacctggcagctgtagcttctgtgggacttccactgctcgggcgtcaggctcaggtagctgctggccgcatagttgttgttgctctgtgtggagggtgtggttgtctctatgcccttggtgactagggtgccattttccttctatggctcctgggtagaagtcatttatgagacacaccagcatggccttgttggcttggagctcctcagaggagggcgggaacagagtgaccaagggggtggccttgagcggacctgcagaatggatgaggggcaggaggtcagagtcctggtgtccacctggggagcccctgacctcagtgcatgaggagtgttcaggccccccagtaccagtgcaggacctctcagatgtggtccacactgtctcagaagggtgtgacatcatgttcctaaaagaactctgtcagtcagaaaacctctgagcttggcccctgcccatcgcctgtttggaaatcttccaaggacttgggcttttgtatagacagtggaccttcaattgcctggcgctgcctcaccctgtcatcctgacgtgatccggctctggctgtgttctctgagctctgtgagcctcttgttagattcactctggaaacttttctcacctgacacagctgctctgggttgggggagagtgggtctctcacgtaagagctcttgtttgtacccacatccgtctctcctggatcttctgcagtggagtcagggcctttgctcacccaagtcccactgtctgtctgtcctgggctctttgtctgtggagtctgtgttagagtctctctgtgtttagggccaatctacagtttttctgccttgggatatgcctgtcggtgaactcaggggttcctgcttctaggggtgcctcccacagagacccatcagccacctccctgaatgccatggcctttcgtgacacttcacctggctccttcatgtccagagccttcccgtccctcctgacagggcttcatgggtcccaggccagctcagggctctctccctgggagcccacagaaacctggagaggcccctgcacctcctaccttgttcccagcctggatccagagtcccacagtccaggggactgggccctgagactctgagttttcattcccctatcttggctctgagggtttcaggtccttctaaagtccaccaggtggtcactccttcgagccccttgttccctctccctcatgagatggggcctcagtttcccttgcacaaagttcgttagacagaagcccaggtgggggaggacagaactgagcaagttttagggaagcagcctggctctggcagacagacagacagatgagtctcagtaggtcatccagtctagtgccctacatctgaggtcagaggtgaaggggtcttggtgacccactgaggtaagggtctaaacagggaggggagaggtccttgaccagggcctaggctccagagagaaagcatcaccacaggtcaacacaagatacagaaaagaacaaaagcagcagaaaatttcccagtaccttggggagaccagggaaggagggggaaaagactcatctaggacggtcagctcggtcccctcaccaaacacccagtgctgtgacacaggctcatacaaaaaccctctctggacgcctctacccccaacccccgctgcagctgtgatggaggaagctgaggccacagcccctggtggcatctgctcatgagggagttcacacggctgccacactcacccccacagagagaaggggacagctgacatcctgtgaccagcaggatccctagaaatcctggttcctggaggacagtggatttggttttcccaccaaccatcatgcagccatggggatgagtttattggagggcactgtgtcactaggagtgaactcccatggtcagggataaatggtctccctggtggagctgactcgacactgactgcttggtgacgccctggggtgatgcagagagtgccctctccctccaggaagaagctggttaccctagagtgcggtgtcttgggaagcttcccatgaacctatctgtgactccaggagcctcagcccagccttccctgggcttgtcctagttgggatgggcctggccagtgaggcctggctggtccccactggaaatgccagccccatcctccagggtaaaatggaaggatctcctgcacccctcaacctggtctcattttggtgggtgaggtggggtagtggctgccactgccttattcaaatgtgagggacataaaggagctcccgagtgtggtctccaaatatcaccttctgctaagggaggtgaggtcagtgcgggatcccaggcctggggcaggaaagggacatgatgagacagaacctctcatcctccgagaaatcaggagccatggaagctcctggggcaatgggagaaaatcagatgccacctgaggaggcgcccagtgggtaaatgtttatgttttgtagccgcattgtcattgcaattcagtatatctgcagtggagcccacatgtcagatgtcctcacacgctagagctcactccggtactacattgtgctgtgcgccctggaggatggctgggcaccagatcatcagttatcaaagtggtgaacaaaagggaagaatgtacattttttcttcttcccaaagcatgaagcacactcctgagcgcacggtgcagctgctgtgccctcccttctggaagccctcatgagaaggaggtggaccgaagtcttgaccatctgccttccttctcatctgaggcgcgaagttattgatcatcaaatgcagctaatgtcactgaaagaatgacaacacaacatgggtggctttgctggaagtagccacgtgtcctgggaagcgagtttgccaatgtccagcctgaacccccttgagcctcttgatgctagaaccatttttcaggaaaagagaggttgagggaagcactgggttcctgctcagggaggtggcgcctgcatcccgactgtgggaggtcccgggaccagctctctgctcctgcagctcctgccttgccagggaaagctagagcaatttgggagacacctgcagatggaatgggtctaaaaacacattcaccaatgataacgtcaagtattaatccacatagaaatgaaattaaatcacaaatatgcaacagcaccacaaagtcgacaaaaatagacacaatgatattcacaagacatttgataagactctcattaactatttttgatgtcaagaaatccttgatattctctccagttgtgtgtgtgatgactagcatttgcttcacacccttgcttgaaggagtagtttagttattctgctgtgggtgggtccgcattatctgaaaatggatgatgctgcggactggctgatgggaaccttggagtcattactattcttctgtctccctatttttgtgttcagagatcttcatcataaaaatgtacaacttatagagagggtgaagcttgggacagtgtctcccatgggcttgaattttcatagctgtgaaatgggtcaaacagaggctgagggactctgggcacctgaaattcaattcagctcacggctgtgatttggggcaggggtggctgggactgagctccaaaggggcccctctctttgtgtatcattgtggaaaaagcccccaggccttgcagctctgttcctgtcctggctggccatccagcagctgctgccatgtccctgatgaaccagaaacagtgggggtcccagcccgggcactatggattaaccgagcctgtcaaggccttcaagggaccaaattgcccactgtcagggaccccgggatgagggtgtctctgagtgaggctgccggtgcaccatggtgcagggggtgaggggacggaacaacgtgagtgacagagggagtggggcctggtgggatgggggtgtttggggcagttgagatcccgtgaaagcctgactttgcctatagatagcccctaggtcatgttcaccctgtgctgaccttgcgtgacccagccttgactctagtccagaatgagactctgacacagactctccccgacaaccatgggatgcccgttctgtgcctgggatggtgtctttgctcatttgtctcagtccacaggagtcctcccctcacacgagctgaccatcacaggctcagccacacaccaagcacatactcactccaccccagagcctgactttagaattgggaacttcatggttgagatctcagccacgtgctggttaactttgaacctcaccctaagcccatctgatcataactgatcacccgtgtgccccagcctcaccccactttccagagctagcgacgtgaacaccagtatcttgcagagcatcctagggctcactaagttggctgggatggtgagcaggattctggacttcctgtttccctattgcaggacttgtaagtgtctttacttagccaccctgtattgtgaaaatctgtgaaattcacctgtgagtttccattcccagacctttccacaagttcctggtgatggcaccaccatggacttctcacccgataaactgggcacctggattttcctcatccttgcttagaatttaaggaggttgcaaactaaatatgagataagaaatgaagacagacagcaggatttctggttgacaatgaggatatttattgagggttactgggtgcagggagaagggctggatgacttggagtgggaggggagacacatgccctgatcctgcagctctaggcccccgtgggtaggtgagggtcggggacctaagaacattctgcaggggccactgtcttctccacagtgttcctttcatgcgtgacctggcagctgtagaggttgtgggacttccactgtcgggtgccagtcttaggtagctgctggccgcatacttgttgctctgtttggagggtgtggtggtctccacaccctgggtgatggtgatgccatctatcttccaggccactgtcatggctctcgggtagaagtcactcatgagacacaccagtgtggccttgtcttggagctccctgcctctggggtcagaggagatgaaggtgtctcagtgaccagctgaggcatacaggggtctgaatagggaggagagagatcagcctcccaggaccgaggtcccaaaggaaaccctgaccacaggctgtggcaagtcacagacaaagatcgaaatcagaataaaattgtcaccaacttggcagggaaagaaagggaagaagagactcatctaaaatgatcagctgggttcctccaccaaatacaaaatgcggtgacacaggttcatacaaaaactcttccctggggcccctaactccccctccccatcccccacctgaagctatggtgcaggaagccagaccactgcccctggaaggcatttgctcagcagagtccacacacctgcctgcccttctcaagctgagatgggacagtcgaccaagcgtatgcagtaacgggacgtctggaaatggcgaggcctgaggaggttggtgtctggatcaggttttccaggtgactgtgacccaggaatgggaatgagctccttgagggcatttgcttcacgaaggagtgaagtgacatggaaggggacagggattctctatagggttgttgtaaccctgatctcttggtgtgactacagggtaagagcaggaggcgtcctcaggtgtccccagtgagtgccttagaggaggggcagcttgccccagagtcagcagtcatgggaagctccaacgtgaccacaaccacaactccagggcgtctcacttccgtcctccttgtgttgtcttagtttgggatgggtctcactaggttttagtatgaaatgccagcctcaacctccagggagaagttggaagcaagcccctgttaccctttcagggtcttgtctcatttctgggtatgtctggagtgtctgactcaaaagggctgtgtgagaagcaccccaagtgtggtcactaaatcccacctcccagttatggggactagtccacttggggttaatgggcaattccaggcacaaggcaggagaaagatgtgcccaggatagattgtgaacagagagcggaaagccatcgaggtctcctgggttccttcaggaggatcctggggccaccagaagaggcactgagtgggccacggtgggtcattgtcatcatcaatgaaatagaaccatggtggactcaagtgagtcacatacagtaacataactttgagctcaccatgaaataattgttcatcttcacgagatggcaggcaacatggtcatcattattaagatgctaaatgaaaggaaagactcaacatgtctatgctatttcttactgaaataagaatcacactcccagaggagcaaagaacatatgagggcacaccttttacagacagtcagtagaaggaaaaggtggtcgaactgaaatgcagtcaccttgcatccatataccatctaatggcactggatacaaagtgtgaagcccagctaacgtcacaaatagatgaccccacatgggagcccagtagccaagcggtcttgctgaaaagtcacactgagccttgttaaacctttctggccaagaaccaattttcaggaaacgcagaagcagagaggagcgcttgtttccagcttggggaggccacaggctgtatcctgaccatgggaaggtcctgggaccaacccccagctcctgtagctcctgccttgcaaggaaattaaaaatcaatgaggaagatacctgcagacagaacaggtctaaaaacacagtcaccagtcacaatttcaaaccatattgaatcctcatagaaatgaaatggaaaaccacagaataacaacaacacaaacacaggtctagtgaaagaagcataggatatttgtgaagtatttgatgggaaacattctttttttgatatgaaagaattagatatttatatttaggtttgtaagaatgtagttttttggggggggtccatatttttagagacacaaatggaatatttagactaatggacatgatgtctacaatttgcttcaaacaactgtgtaaaagaggttacttgattgtgggtgggtcaacatgagctgtattatcctgtttttttattttcttgctgtcagtttccgtaatatcttacaaggcagtaattagagagattagtcttgggacagcaccaggggacatccagaggggatcctacccagcagggccattgagaaggtggagatcaaagcggtcctctttgctataggggtctcagcgttcacatctatgaaatggggccagacagggtggtgggaatcaggacccttaagtttccttctaacttgtaggttgtgacttgaggcaggggttctatgattaggctccaagaggaaccccttctctgccccactatggccctagagttctctgcccttgcaggctctgtctcaggcctggcctggccacccagcagctctggtgaaggcacctgccacaccgctgatgtgctggaggcaccaagaaggggcctgacagtgcccaagactgatgaggagggctgggaagtgcatctcccaaggccacctcactcgctgtcagggaccctgggatgattgtgtctctgacatcagggtgtaggtgcacatggaggggctgaaccacatgattgagggagggagtaggccctggtggggaggggtctaaggtagctgagtcccatgaaaacttgacctgcctgtagcccttaagtcacacttaccctgccctgacctagtgtgacctggccctgattcttgggcgagtatgattttgacacagcctccatcagacaaccaatggatgccgattctatgcttgggatggtgcctttctcatttgacc

SEQ ID NO: 73 - protein, 115 aa, Homo sapiens
MAWIPLFLGVLAYCTGSVASYELTQPPSVSVSPGQTASITCSGDKLGDKYACWYQQKPGQSPVLVIYQDSKRPSGIPERFSGSNSGNTATLTISGTQAMDEADYYCQAWDSSTAH

SEQ ID NO: 74 - DNA, 32230 bp, Homo sapiens
  source 1..32230: mol_type = unassigned DNA
gaattcctgacctcgtgatccacccacttcagcctcccaaagtgctgggattacaggcagccactatgcccggctgtgaacatgaatttttaaactgcatggtgctttgggtcccagcttctccatctgtacaatggggacagtactaaggttttctttttcttctcagttgactgaattatttccatagtctgtctgtcttgccactcttgttacccacacgaaaggacctaaggtaatttctgacagcctgggactccttgtgcaaaacagaaggtgccacagacctcattttaggagaaacctttgttttcctcttggaaccccaagaactgtaagcagacaggtccatctcaaaatccaaggctctgctctgttttgctttgctttacctgactttttttttgatttgggtgggcatcaaaaattagtaggggagagctaaagaaagttgtggatatgaacatgtatttatggtaaggtaagttatgaaggagagaaatcttaggatcttgtatggcaaattcttgcactaaagtagataatgactaattaggaaagcaggaaatataggacaagtcagaaagtcataagatgggagagaaaagcttacgcctggtagatcctctcctgtctagaagtgtcgtatgtgtgatgtttatatgaaggagctctaattaattggattaaaagataatgaaagctctaaaattgtcagaaaaatagaagctctaatgccttttatttcacgtgacttcagcaatctttgggaaatgagagttttaaaattattggtggtaaaaagtcttcaaaaagtagacatttggtctaaattaagtcagaggttaaattggctaagtgctttaatgtcataaactgcttctttgactttggaaaattgttcagtttacctaatttggaactgttagatttctaggtaaggcctggggagaggtggagttagccatgtctcctagctatgctggaagagttagactttatctgcagttctgtcatgtatcctggactctgcacctggtatgtaattgaaactgcttacattgaaaagaaaaattatgtgtttttggttttaaaaggggtgggaatatggttttttccaaaaagtgagttttggcaaatttagaaagtttaagaattatttttagttgtaagaaaataaagtttaagcaagttatagaaggtttatgaaaattaactttgtaaagaattgtgtgtgtgagcaagttgactaaagggacagtaatgttagatgtaataaatgcaggtacccttccactttatcttctgtttcttaggttataaatattcactacctacgtttctaaaaactctttcactgtaatattaacacataactccattctccttgtattgcctgctttttcatagtatcctgaaattgcattagtagatttagattcagaagatggtggggaagcatcttatgagtgttttaagtaaatttaaggcaaagggttttctgtacatgggtggttatcacagcatcacttgttgcagcaaaacactgggtaacaacaatgtttatcaatgagaaattgttcggataaatgatgatttgtgccttgcgaggctcaactattcaaaagcaccctcttttcctcacttgctttacctttggttctgggcctggagcctttcagaggttactcaggacagtggactcctgtttcctgtgactaatctggacagcggcttcctatttcattcttcagcatcttccaaaaatttctgaagataatttcttcctttgtgaccacccatactcttggtttactttgcagttaacaatttcatgccttcactggattatctgtgtgaaggtgtggggtgtatgtgttcatactctggagggagcagaggaaaatagccacagccaaaggagtaatcttgcactaaatactcattataagaatgttgatacgttcatagatggtaaatctgtcagtaatttttacttgtatgattcttggttaagactgcactcactggtgataagcgtgctgaagggatcataggtgctatacttatgtgtgggaattgcattcaaaccaccaatgcctgtctactcaagttttgttgccttttgattaaaaactcttgggaatgtttccctggcatgcaaatatgggaactgcagtttgattctaggtagaaactctttggtagttgaaatgccataataagagacacagctgtcaatgggcaacaattcactgttggtaaggaatttacagtgctacttgccatattcttctttatccagactttgtgccaagaaaatgaaattctttttaaaaatttttttctagagtaatcctacaatattgctgttatcaagcctgatgctgtaattagtagaaaatgtctagaaattaaaagaaaagtagtattttccaactatggttaaattaattgcaaaataacttcctcttaattgtcagattggagacaagcttattaagagtgaaataaaacataatgtgttgctggtgctaggtacctctaaagcctttgagcactttcttctttataaaggctcaatctactcatgtgtcctgtaaataatcctgctctaccagcatcctatgtaaattttaaaaattatagttcatatattagattgtaacaaatgttgttcttctttaattatatgcaatgcaatcttgttatagactcacattagcttgccttattccctgtatgtttgctgtaaaatgtatagtccttttcaatatatgatagcttaaagatggacttaatattttgttttagaaccaaaatcttttatcttatatacaggaaaattacattggaattcagagatcagcaaacatttcctgtaaaggatgaaatagtaactattttcaggtttgctcagcatgtggtctctgacaactaaactctgaagtcataccaaggcagcatcagtaatgtttaaatgagtgaatggcgctgtgttccaataaactttatttacagaaacaagtggtgggccacattttgactgcagggtatagtttgtggacccctgaacaatggctttttctgaaacaacacaattgaattgagatttgaaagaatgttgaggcagcattgtttgtctggggtaatacctgaggtttgtcatctcacaccagggaaatcagggatgtggacacacaagaagtgagttcaagagtggatgattaataggtgaaagaaagagaaaagagaacagctctctctcctgcagaaagagagaggggcacctgagtgggtcttccggttttgtggtgaaaagcacaaggttttataaaggagcttgaggaggtggtgtcttgatttacacagggctctagagactggttggaccaggtgtgaacgtttgcatagcacagggagaagctggccattgcaccctaatcttttattatgcagatgggttctttaccttaccaggaccatattgtctgttccttactgtacctgtggttgacagagaaaagggaagatggagccaccatgttgaacatgcctggcccacaggtagccttttcctattggcacagctgctggcattcctatgagagcttatcaatgtctgcagcccaattttacaagatgctctttgttagaaaataaatgatatgggttttttttttcattagtaggaaaactttacactaggacttccttactttcagtatctgcctaaataatttctttttaattcctacatcaatgttgcatgtgcaatagagaaggtgacatgtagggagctgtgtcagcaggggttgcatatttcttgatgagcatcccatgatgcattcttcttcacttgtggcaacattagcattaactaatctacagtaaagtagatgggtccacaggtcatgggacatacactagctctgccaattacatgttgatttaaataatatcattctctaaggacagctttttttcccccagatcctggtatgtatgtggagttggggagagacaccaaactgtaccccatttcctcatacattgcttatacttttagaagtgtttgaaagataatatcacatactagtagtttactgtttcttattcaagttagttttaatagtcgctagggtgaatgttttcaaaattgtatagatattcgggttgaggcaatactataaattatgagttcatctcagtttttttgtgctagcgtaatgacatttcggagacataatgcatatgtgcaaaatacttcacctctctggaaaacagtaaagggctacataataggacagacaaaaataaaggcttggcgtaatggctcacacctatgatcccagcactttgggaggacaaggcgggtggattgcttgagctcaacagttcaagaccagcatgggcaacatggcgaaacccatgtttacaaaaacgataaaaataagccgggcgtggtggcatgtgtttgcagtctgagatacttgggggactgaggcaggagaatcgcttgagcctgggaggtcaagggtatagtgagccgagatcgtgccactgcactccatccagcctggataacagagtgagaccctgtctcaaaataagtaaaaatttataaaacaaagaatgtttctaactgtaataaaggagaagtgatcaagcttaagtgtagcttagatttaatctttgatttccgggtactatattacttaaaaatatgatttggatatttagaagttgttcaaattagaaactcaggagtggggtcactggtcactaaggtgctgaatatttttaataccttagtggatatgtaatcaacaaagccaacggaaccaatagagcatttgctttcataagaatttaagtgttgctgggtactcttaaatcgagctcacattattggtgtcatttaatataattactgaatttctttgaacccagtttccactttttaaaaattatttagctcattaggctgaaaacttctctttgttacattttattacacttcagtgtaatatgtgtaagatgtacattgcttctctctagaaaggtgagacaagttgaagcagggttgggggtgggtgcttccagttcataggtaaataggagacaaacggttgcattcttttgagtttctgattatcctttcactgaatacacaatttacatgcaagatgccatagaggaatagtcacttatgcctgagtctggcttagtgaatctgcatttttacataaaaaactgggcagagaaagccaatcagatacacatttgtctcaggtgaggagagggatgactttgggttctgtcctttgtcccccacctgtaaagataatctatcaattgccaatttatattgccagggtcaaattcaacagaactgttttagtgtaaagatcttgaggcccacaagaaatttcctttctgacaaattatgagggaggtatatagctttcgaaatctttgtagttatttttttattttattatttgggttttttttttctttattgacaaataattgtatatactcacagggacatagtgatgtttcaatacagaaaagttatagtgagcagagcaggataatttcatatcctttgttaaaggaaaataaatctccgggacaccccactaagccaaataatagttatcttatttagaaataaaatgggaggcaggtttgtttgatggagttcctagctgaggtgggagaattgcttgaacctgggaggccgaggttgcagtgagccgagatcgcgccattgcactccagcctgggcaacaagagcgaaactctgtctcaaacaaacaaagactatagaaaagctatttgtgaaaatgctttttgatgtgctgttttatgtcacagaatgcaacctgtgttttgattcagcaagttcaaaacactctttttgtggaatctaagaattgattattttgatcctattgagctttcataagaaaatatgaatatccagttgtaaaaactagaaataagctataagctatctgtggaatgctttgtgaggtgctgttttatatcataaaattgaacttgtgttttgattgaacaagttgaaattactcattttgtagaatctaagaagtaacgttttggaacttcttgagcccttattaaaaaatagaaatattcagtcctaaaaactagaaacaagccatccgtgcaaatgcattgtgatgttctgttttgtatcataaagttgaacctgtgtgttgattaaacaggttctgaacacttttttttgtagaatttatgaagtggcatttctgagcctattgagcccttatacaaacatacagttttaaaagctagaaacaagctatctgtcaaaatgctttgggatgtgctattttatgtcacggaatgaaacctgtgttttaattcaataggttcaacacattcttttggtagaatctgagaagtgacattcccaaacctctgaggcctttaaaagaaaatacgaatattttcctctaaaaactagaaacaagcaatgcgtggaaacgctttctgatctgttagtgtcacagattggaaactttgttttgattgagcaagttcaacgcactctttgtaggatctaagtagtgacatttccaaacctattgagcccttataagaaaatatgaatatttagtcctaacatgctatctgtgaaaatgctttttttttttttttttggcacagagtcttgctgtgttgccaagctggagtgcagtggtgtgatctcggctcactgcaacttctgcctcctgggttcaagcaagtctcctacgttagcctccctagcagctgggactacaggcgtgcaccaccacgcccagctaatttttgtatttttagtagagatgtggtttcaccatgttggccaggatggtcttgatctgttgacctcgtgatccgcctgcctcagcctcccaaagtgctgggattacagacgtgagccaccatgcctgactgaaaatactttttgctatgctgtttcataacacagaattaaacctgtgtttttgtgagacaggttccaaacactctttttgtagaatctaaaaaagtgatgtatccaaaccttttgagccataataagaaaacatgaatatttagccctaactgctggaaacaagctatctgtcaacacactttgtgatgtgatgctttatatcacagaaatgaacttatgttttgatgaaacagattcaaaacactattgtagaatcttaagaagtgacatttctgagcctattgtgaccttataggaacatacaaacatccagtcctaaagtctacaaacaagctatcttggaaaacactttttgacgtgcagttttataacacagaattgaacctgcattttgataaaaacacgttcccaactctctttttgtagaatctaagaagtgacatttcgaatctattgagcatttacagtaacgtatgaatatcttgtaataaaaactagaaacaagctatctgtaaaaacactttgtgatgtgctattttatgtcacagaatggaacctgtgttttgtttcagcaagttctgaactctttgtagaatctaagaagtgacgtttttgaccttattgagcccttataagaaaatacaaatatccagctataaaaactagaaacaagctatcagcgaaaacactttttgctgtttcatatcacaaaattgaacctgtatactgattaaacagtttccaaacactttttttgtagactcttaagaagtgatgtttctaaggcagtgaggccttatgggaacatatacataatccagtcctaaaaactagaaacaagctatctgtgataaccatttgtgatatgctgttttatgtcagagaatggtacctgtgttttgattcaacaactgtgagacactctttttgtataatctaagaagtgatgtcaccaaacctattgagttttataaaaaatatgaataaccctaaaaacgagaaacaagctatctgtcgaaaatgctttgtgatgtatttttttatatcaaataatggaaccagagttttgatgaaacagcttccaaacactctttctgtagaatctaagaagtgacatttccaagtcaattgagttcttacaaaaaaaatgcaaaatccagtcccaaaaaactagaaacaagctacctgtaaaaatgctttgggacatgcagttttatgtcacagaacggttcctgtgttgattcaacaagttcaaaacactcttgatgtagaatctaagaagtgctgtttccaaacctattgaacttttattaaaaaagtaaaaatatccatccccaaaaactagaaacaagttatctctgaagatgctttgtgacgtgatgttttatgtcacaaaatggaaactacgttttgatttagcaagttctaaacactttttctagaatataaaaagtgacatttctgagcttattgagctcttagacgaacatacaagtatccactcctaaaaatgagaagaaaaacctatctctgaaaatgcaatgttatgtgctattttacatcacagaatgaattttcaaacactctttctgtagaatctaagaagtgaaatttccaaacctgttaagcccttataggaacatatggatattcagtcttaaaaactagaaacaagctatatgtgaaaatgcattgtgaagtgctgttttatgtcacagactggtccctgtgttttgattcaccaaagttgaaacactcttttggtagaatgtaagaagtgatgtttccaaacctattgagcttctataagaaaatatgaatatccagccctaaaaactagaaataagatatctgtgaaaacactttgtgatgtgctgttttatgtcacagaatggaacctgtgttttgagttgacaagttccaaacactctttttgtagaatctaacaaagtgacatttccaatcttactgagcccttataggaagatgtgaatatccagtcctaaaaatgagaaaaaaagctatgaaattgcattgtgacatgttgctttatgtcacaaaatggaatctttgtttttattcaacactttcaaaactttttgtagaatgtaagaagtgacattttcaaacctattgagcccttataagaaaatgcaaatatttagtcataaatattagaagctctctgtgaaaatgctttgtgatgtgctgttttatatcacagaattgaacctgcattttgatgaaacagcttccactcacttttttatacaatctaaaaggtgacatttcctagcctcttgagcccttattggagcatatggatatcctgtcttaagaactagaaacaagctagctgagaacatgctttatgacatgctgttttatgtaacagaatggaacctgtgtttttagtcaacaagttccagacactctttttgtagaatcttaggaagtgacattttctatttttattgagcccttataataaaatattaatatttagtcctaaatattaggaaaaaacctacctgtgaaaacgctttgtgatgtggtttcatatcacagaattgaacctgtgttttgacaaagcaactttcaaacactgtagaatctaagaagtgacattactgagcctattgagcttttataggaacatacaaatatccagttccaaaaactagaaacaagctcttggtgaaaacgctttgtgacatgctgttttatgtcataaaaatggaacctgccttttgatgaaacagcttccaaacaggtttttttttagaatctaatagttgacatttctggccctattgagcccttatagaaacatatgaatatccagttctaaaaactagaagcaagccatctgtgaaaatgctttgtgatgtgctgctttttgtcacggaataatacatatgttttgattcaacaagttcaagacactctgtagaatctaaaaagtgaagtttttgaacctattgagacttgattaaaaaatacgaatattcagccctaaaagctagaagcaagcaatctgtgaaaacactttgtggtatgctgttgtatgttatggaatggaacctgcattttgatgaaacagcttccaaaacacttttttgtagaatctaagaagtgacattttggagcctattgaacccttacaggaaaatacgaatatccaatcctaaaaactacaaacaagctatctggtaaaacgctttctgatatgctgttttatgtctcagaatggtactggggtttcaattcagcaagtttgaaactttctttttatggaatctaaaaagtgacatttgagaacctattgagccgttataaaaaattcaaatattagccataaaaattagaaacaagctagctgtgaaaacgctttgtggtgtgctgttttatgtcaaagaataatacctgtgttttgatttggcaagttcaaagcacttttttttctagaacctacgtagtgacatttacaaatctgttgatcttttataagaaaatatgaatatccagccacaaaaactaggaaaaatctgtgaaaatactttgttatttgcattttatgtcacagaatgaaacctgtgttttgatgaaacaacttccaaaccctctttttgtaaaatctaacagtgacacttatgagcctatggagcccttatagaagcactcaaatacccagttttaaaactggaaactgtctcaaaaacactttgtgaggtgcttttttatgtcacagaatgaaacgtgtgttttgagttgaacaacacacaagtttccaacactcttgaatctaagaagtgacattttcaaacttgagcccttatagaaacatacaaatatccagtcctaaaaacaagcaaaggctatctgtgaaaatgctttgtgatatgctgctttgtgtcatggaatggaatctgtgttttgattcaagtttgaaacactctgtagaatctaacaagtgatgttttgaacctattgagtcattataagaaaatgcaaatatttagccataaaaatgagaaacaagctttttgtgaaaatcctttgtgatgtgttttctatcacagaattgaacctttgttttgttgaaatagtttctaaacatacttattgcagaatctatgaagttatatttctgagcatttgagcccttataggtacacatgaatatcagattctaaaaactagaaaggagctatctttgaaaactatttgtgatgtgctgttttatgtcacaaaatgataaccatgttttgattcaaaaagtttgaaacactctgtagaatctaagaagtgacatttctgagcctattgagcccttagtaaaaaaacaaacaaaaaacgaatatccactcctaaaaaccagaaaaaagctatctggtaaaatgctttgtgttgtgctgtttcatgtgacagaatggtgcctgtgttttgattaaacaagtttgaaacactttttttttgtagaatataagtagtgatgttttagaatgaattgagcccttataagaaaatgcaaatatttaaccataaaaataagaaacaaacaacctgtgaaatcactttgtgatatcctttttttatgtcacagaatggtacctgtgttttgatttaacaaatttgaaacacactttttgtagaatctgagatgtgacatttacaaaccaatcttaagccagttagatactctgatgagccagctgtgtgtgtatgtgtgtatgtgtgtgtgatctttgattattaaaactgaaaaaataatcgtatattcatgactagtagacacagtgttttaaaactgattctgaaagatttgagggttaggcttgttttaaaaacaattgcataggtcctggaaacaccaagaattagtggcttctcattcctagtcacatgaaaaatattttactactcttgtgaatggccataaataatatgcagaattttttctgtgtcctaaccttttatataattggttctacagtgtacatcactgtgtgcaactttgttttgtgtgctcagacttacattttccagttttctccactttgatacaattggtctgtgtttcatacattttgtattttctatagcgttcctttatatgactctaccacattttattgagtctcctcatgctgacaggtaagtagatgaattaataaatgaatgaataaataaaaatgagagcaaatttcaaacaggtgattaatttagtgattgtaaccatggactcagcaggttagaaataaaaactgaaagatttagaatgcaagcaggcacatcataccttctaatagccattggaactatgacccacaaaccattagcctagaatgatgggacatatcttgtctctaaaaaaaggtcactgttcagttctcaaagaatggatataaaaatggcagataccattccacattattctacttcctttaacctctacgcataaaacttgagctgcagggaatgttacctcctgttcttaaaaagacttgtagttcagtgaggaactcactcctttcttcattcatgtgttcaaataatatagggaaatgaatggaaaagtgaagagaagttgaagactattatggggatagcacaggattagtcagcctcatggtattattgatctgccttattccacaaatatttaccaagcacctgctacatgcaatttgtatactaggtgctggggatacaggagataacaaagtccctgctgtcaatgaaaggggtcagtcctttgattcatctttagttaattttaagcacacaacaactatctcaaactttcttcacactttccaagctcccgatccttcatgcactcttggtagatgcctgaccttacctcttaccttactagaacctaagaccacctggtataaaatcccttaagcacactcagatttgcgtgattttacaatgatgtctgttcccaaagcatcctcacttctcttggagctgagggtgaagagctcctctttcttcccatccgtgatcttggaacatgtacgactacacttgtgacagtaaatgcttaatacaggcagaaggtttgttccttatttcctccctggggcctccctccagtgaatatctcttctagcattgtcagttgtatccaacagaaaagcagagatctctccattggtccaaaaacctaagatggactctcactgctaaacttctcaaatgaaaattcctccactttagtcctgattaccactcaaacactgttagactgggcaggtcactacccctgcctagttctgtttccccatctacataacagagaaataattgcgccttatctatcctacagaagtgtgaagaggtttgatgtgacagcagtgaaagcaatttaaaaagcacagagtgctatacataagaagacattttcataattattccgctctaacatgatatctgtattcatacgatctcaaagtacttacccaagcatggattctaactcttgtgtatgtaagcaaaggtgtaagtctgtcaccctccatgcaaatagaatctcccctcccttggcccctgcatctcaccatatacaggaattaactcaagatgaatcaaagttttaaatgcaagaccgcaaactatgaaaatcctttaagataacctaggccatacccttcttgacaaaggctttggcaaagcatgtgtatgtctaagtccctaaaagcaatagaaataaaaacaattattgacaagtgagacctaatacatgaaagagtggctgcacagtagaagatattaccaacagagtaaacagacagccttcacaatggaagcaaatgttcccaaactacatctgacaaaggtctaatatgtacgatctaccttaagtccttaaatcagatcaaaaagtccaattaatggttagagacatgaatacacactgctgaaaagatgtaacagccaccagtaaacatgaaaacatgctcaacctcgttaatagagaaatgcaaatcaaaagcacactggtcaaaatggtgattataacacagtccacaagaacagataccagcaggacagaggagggcactggggcaattgatcatgattcttatggatagtcatttagtgttattaatgaatttcataacaaaatgatcaccatacaaaaaatacaaaatgtgttttcttccctgagaaaggaaaataaaaacttggaatgccgattcactatgccaaaaggaaaagttaagctgaaagctgagtcatgcaagaaactgcctttctttttttttcctgagcagacagctactgataaatagttaaatttctccacaggtagctactctatgttcatctcatcttacttaaagtgctgatttactgagcaggagaaaaatatataactgactattcctttacctgtttgttttctcttgcaacatgtggattcagcaacgtgaccatactctccctcttttccccgcagcctgcttttctcttctaaacactgaaaccctaaaatatttttggagaaaggcacagacctctgtcgcccagccatgtctttaaccttggaagaagaaacttctaaattgataagagatctgtctcagacactttttcgtttacatcccgttatctcagaaagcaaaacaggctttgttccaacccaaagtaatttgtgaatattcaagcggtatcattctgatgtaaaatttacataaattcttgggtttaccaaaatgcaaatgggcaagcagatcactatttacagataatggcctaaaaaatggtaagagtaaatgctgtggagctaaatgtttctgagtctgccatttcctagttttgtagtttctctcgggcaagttacccaaagttgctgtatgttattatcttgacatttaaaatagaagtaataactttattataaataatagttaataccatagggtcattaggaaagtgtctggcatatatagtcacaacccagtgaatgccattattgtttttgctattatcagtataataattgatactaattttgatgctgaggtaaaataatatctggcagcttttaatttgctaatgttaaataactgaaaatagctttaaaaagtgaaaatatccaaatcaatgacaatatttgatttatataaaataagcttcattttttttagtttttttgctcctaattattacttcactcatcacactgatatctgagcactttttaaaacatatccacccttctcagttgtactacttattcccttttcttgtaatcccagccttcaaagatcctttctcatttttctaactcatttcacgactccctcaccttgacccccagttatatctctcttgaagtcaattccattgaattgactacttggcccaaacttcaccccaactcagtctctcttcaggaaggaaagtccccacacaaaaaggaatgtgtctttcaactcatgcgccctttttgaagaagctttccagacagcttccttacagtgatctccgttatccatcctttagatcgctgtggagtcctacaggggcgcaatgtggacagtcattcaaatgaagcacacctaagtgcttgcattttctaaaggcccattgattcatgtaactcccgtacttgcacagataagatgttcctgcctgaatgcagcagatggaaatgaaaattgtattctctgtcaaggctgattgtattacagcttaatgatttgcattttaaaacttggaagttctcataaatattaaagtgaaagaacaattggctacattattaggtaggttcggctgttcgtataacctggtgctaactatctaacctgcctgagtgagcctcagtcttcattactgcagcatttgtaagatatcaatgcctgcttgaactcatgtcttactaagattatggccaggattgagtaacatatggatataaaagcacttgacaaatcagagtatttcataaatgtaataataatatgctacatttataacatctatactcttttcctgtgtctatcttcatttaaaccttgcagtttctatcataatcaaagattttgtaatcaggttacaatgagtaggatttaacccataagcaattttcaaatttcctagatatgttttcagttttcacatttatgttggtgacaaaattctgttttcagttttggtgtttgcatcttctaaaacagtgtcttgggtacttgctctttgatgagacactgtgcagacataaagagtgtaagaaaacagaacagtttatctcagctttttgtggtctagataacctgcacactgctttcttggctcactgcaacctcctcttcctgggttcaagtgattctcctgccttggcctcccgagtagctgagactacaggcatgtgccaccacgcccagctaatttttgtatttttagtagagatggggtttcaccatgttggcccggatggtctcgatctcttgaccttgtgatccccctgccttggcctcccaaagtgctgggattacaggtgtgagccaccgtgcccggcctgcacactgctttttaataagctgatctgtcttcttgtttacataggctgtgctatagtcatttaaagttcaaaattactgctaaaaagtaattaatcctttactgaaatattcctgctatttcatgagctgtcttacaatttggaggaatctatttgtcactgatgtttaaaaaagaaagtcacagcagggaggctttagcttcattgaggaacattattataaaataaaatgatcgagggtaatttttttttgtccttgtggtactgtttctgcagatctacattaatctgagcatttaaagtaataataaatttgttatatcctctttattattcacttcaccatggtagcccctagtggtaaaaggagatgtaaattgctcagtgctcacattgcagggaattttttcctcagttgaataaaaaggagttaagcctctttggaaatgagggctatagcctgtcaataactacatatattcctgtttttaagaaatcagaattctggtgtccagtaaatgtagatgccagcattattaaaaagtgattgagcagagaaatgaattgaatttttgacccactctggcttccagctttcatgggagcctatacagggggatgatgaagagggatataagagaataaatgattgctctattaatctactttaattgatactaaaatctgacagcgaattacaagttccaggcagtaaggcttaccaattcattggtaatttcaaggttctactttgctttctttttgccttcagctccttgcaaaggacacctttttttttttttttttttttgagatggagtattgctcttgttgcccaaattggagtgtaatggcccttaacatccgcctcctgggttcaaatgattctcctgcctcagcctcctaagaagctgggattacaggcatacatcaccacgcctggctaattttgtatttttagtagagatggggtttctctatgttggtcaggctggtcttgaactcctgaccccaggtgatttgcctgcctcagcatcccccgcccaaagtgctgggattacaggtaggagccaccacacctggccaaggacagccttttatagttcagtttctcagtttatctttgactattcttaattaagcttgacctggttctccaccactgacgctattggagttgcaaagcattttgctgttataatgatagctcccttgatggctgttcattattaactggttttcaaaagtgctgcttttaactttcattttataaatatttaactcaatttttataaaatgtgcatagactatagaggttctgcttacctgtttaatacagtaaaaaaatcccttttgtaatcttatataatattaagaatgtgtccttaattcatgcttaacataaattcttacacaactagcagtatctcaaaaaataaatgatttaatagtttatgacccttatacatataattaattgtagtaaaatatcttagaaaatagaatgagtattttttataaagataaaaaatcctgatttttttttctggtcttaatataaaattaaatgtgttaaagggaacttataaattacatagtagttcattgctcaggaaaagaaatgacaaagtaattaagagtgtaaaattaaaaggattaatttttcttaaaggccgatgagttaacaacacgcatcaatctctcttccttctccaaacttcagacaaatgatggcaaagtaatttttaagaagtcaatttatagaggcaaaaaacctgaagtacagagagtaggaaagacagtaatagaattgagagcagttgacatccataacattcctattattacctccattttctggataagaatatggagaagaggaatcgttaagcaatttgctgattgtcacatattcagtaagtggcaaagttagtattgaaacccaagtagtcttgttttaaagtcatacactcaatcactgtgccataattcaataaattgtgaaggatgggaagcagatggagaagtgattgatgaagcaggactgagaaagtgctgcctatgtgcccacagtgtgaagaccctacaagacatcagaacatcatccagtgaagctgtgagcagctcaccacttggaaaaactgagtgttttggaaggcagggataaagtgcagttctgaaaagagaaaaatccatcaagttctaaacggtcgcgctctcttaccccacagtgggcaatggtctctccattcatcattttcttttccctgagtcccttgaatcttaaatatactattctgtgaattaaacatggtaaacattagactttgaccagcgctattatcagaagttcacatacctaatcaaaggacctgtaaccttcttcccctccttagtttctacaccaccagtagccaattatactatcccagctcccatcctaaaatccccttctaaaatagaaggcaaaagatcatcagtcatttgagcaaaatttcagcatgacagcatgagtagttgagacctatagtaacaactaaaatggactgggataaaacaaataatgtttggaatcaaagaacaggaaaaatacaaccgatattggagcaagaaaaaataaaaaagaatatgaaaaatagataagaaatagctattggaaaggtacaagaaatacaattttaaaaccagtggaacataagacaaaccacaaaatgagaaaattctattaataaaaggtcgggggtcggggaactgtattctttttttttttttttttttttttaagacagagtttcgttcttgttgcccaggctggagttgcaatggcgcaatctcggctcactgggcaacctccaccttctgggtttaagctattctcctgcctcagcctcccaaatagctgggattacaggcatgcgccaccacgcctggctaatttttgtatttttagtagagattgggtttcaccatgttgatcaggctggatggtcttgaacccctgacctcgggtgatcccaaagtgcagggattatgggtgtgagccaccgtgcccggccactgtatcttttaaaaatgtaattgtcatggaaggcaagaaaggtttaggaggctttccatattgaagaggactgaggagacatggcaactaattgcaatacatgattctagactgcatgcaatactggaggaaaatcatgctataaagaattgactcaattgataaaatcagaatactagcagtagattacatcaaagtatcgtatcaatgttacatttactgaatctgactaccatactatagttaagtaagagaatattcctatttttaggaaatacagaagtgtttagaggtaaagggtcatgatatatacaacttaacaaaagacttagacaaatatggggtatttaaataaaggagaatgacgtgcaatctgagaacaagaagtccaacagagaaaagcaaaaatagggagtcagcgagcaaccagtgagatgggagcagaaggatggaagggtggaggagtgacatctaaacatattaagaactcaaatatgttgattattttgtatatctgaagtttgaaattatcactgacaatcttctaaacttcaggaaaaatgaggatactgtgctacaaatgtaaacttaagaacaatatttacatagtcatcataatgtaattatgaaatactgatttcaccaaaaccaatttgatttaacacatcgagagaacagtgggaatagagtcctgtaattggtgcacctgtcctaattcctatgtaggtattataatagaaagttaataggtaatgtttaaaattgataaatcaagataacaatatcagcatgttaggttaaaaacaaggtaattaccaaaagcaccagataaaagagccaaacattgctttctctattgaacgggactacagcatagagaagtgttaagcaagctgccattattcttttgctcttattataagcctcttaatactatttgattactagctcatgagctcctatttataaaaataaaagcaaattttcgaagtcagaacaattcatgctgctacaaaatccctaaagtcatttcaacaatacagcacactgatgattatatagacatttttcttgtctgagttctttgtctagaaactttttaaaagaaacgtggtcctgtattacatgtagcatgctttggaagaaaaactatctccaatcaaaaagtttttaaaagagcccagatgttatcgtcctttgtatctctaaaagcaattgcttttataaaattaggatctactcttttttgaaatttgaatgctgaagttttgtgggtttcagcaaaagataggcataacatacatctctacattttattaaatatgatggtgcagatgttaaaaatatgtttgaaatgcagtaccttctgttcttgctttcaactcctaccctagagtaaatgaaatgctaatcacctcttgtgttagtgatagaatttgtgagtttccacagataaaatatgtttactgtaatttccaaatagttgatagccagtagcaatatcttgacttcagcaactggtatgtaagggtctatattaaagtgtttatattatgactagtataacagatggtattatttgttacagcaggtcataacactaaaaaataaaactattacacatctacaatttgatcacaaattttagtggcagtgatcacttattttatgaaagagaggcagtaattgccgaagtactgcctaaaaatgtaagacaattctcattacagggtaaggtaaaattattgacttattaaccagaaattcacatctatcccgctacaggaaaaaaagtgtacatttaatatctactacagtcagttgagttttcaaagtaatctgtcatctctaccatgaatatggggaacaggagaataactgctggatttttggcatttgacacctaatatatagaactttaaaggaggttatctattatataaataatctttgaagaagagcaatttatgtttttggacaaccaaaaaattacttgtttagcttcacaggaattttaaagcatgttttcacttattattaaacagtaacattgaatatactgctcttttatcagttctcatttttttcaacattcagtagatgactttaaagaactctcaccttcctggttttctttctaaactgagattatttcttctctcacttatgcacctattttctaatggccatgtctcaaaaaatttttattctagcgtcttttttccttttatcttggttttgctgtagtgctagatagatggttacttgtgagactttcaaatctatataccaggctccaacttctctccctctccctctctttttgagtaagggtttggctccatcacccaggctggaaggtcatggctgattgcagctttggactcctgggctcaagggatcctcccacctcagcttcctgattagctgggactccaggtgtacagcatcatgactagcaattccattgtttttagaaatggggtcttgctctgtcgaccaggctggtctagaactcccagcctcaagcaatcctcctgcctcagcctcctaacgtgctaagattacaggcgtgagccaccacacctggccaacttctaagtttcagtgctgtagttttaaattgtttctcatacttatctaatattttttcccaccatgtcaaacccttaaaaactcagtatggacaaaataaatattaacttttttgagattgataattgtatgatagtgcttattacctcaaaagaattactggggcagcagtataatccagtagatgggggaaatagtggatattaagaccgaaatgggatcataactttttagttgtatttgcccacattaattgttttaaagggggtaaaggcctggaatatgaaaacaggaggatataaaatatatggaggttgtgaaagatgactcagaacttgaaacaattgtttcactgcagtaaatacatgttgaatgcatagaacagagaaaaacagaagaaaagaagaaggaaaaacaaactccatgatttaaaatattgatgaatgtgacaataattgtatctttaaaataactgagaaatggtagaaataaaaattataagcttcaactgtatattacttattcttgacaaagatgatagttgcaagctgaagccaaaatttctaactaggactaatgcaaagatttcaagagaaactttaataaagaattattgatgcaagcgctttcagaaagcaaaggtagggactaaccattagctcttgtttagaaagataaaggtcacaaataaaatttgtgttttaatctatacattttcagcattatgtcatcataaatttgcatgttgtttattacaaatgggaaaaggtttcctgtaatatatcaagatgagttttaatagtttcaaggaccattgatttgtaaacttaagactattatagtgttgtggaaaattaaatataattggtgttttaggaaaagttgttcattgaagtttccattttatgtgccttaacagattaattttgcttattaattctgtgatatttaagcactgctgtaagcttcacagatttggcaatagccactatatacttaatgagtacaaactatggagtatttccatattaaggtagaagaaatatttaaaagcacttctacttgtgatgtcattgggggtgcaacacaatataaagtacgcacattgctggaggagaagcataaaataaagaactcacggggcagaaacttaagacacacctaaatcaaatctttgttttcagcattatctttcattactattctttgttacaaaaaaacattgctatctgtccttttctatatgttctctacattaaaacttattatttgtatacatactgttccctacatctgcagtgccattcccagctcacatatctgccctcttaaatttatatcaaattcaactcccttcaaaaagccttctcagttgctttaacccaagccagtagtctgtcttctagaaaacagttgatgccgatgcctccttttcctttagcaactgaaaataatattatagttatttaatgtgtgtgtgtgtgtgtgtgtgtgcataacatcatactatttagtagtatctatatctatcaagtatctgctacagtggatgatagatgacttaaagacagaaatatgttattcatatctacttctcacaggtctagtgctgttgcaggatattttttaaggaatcagagagactgatggggttcaggaggatatttattatttaggtgcaccagcccagtcggattaacatccaaaggactgagccctgaacaaagagttaagttaccttttaagcattttgtagggtgggaggagatctgtgcagggggaagcatgttagagaagtgagaaacaaagacagttattcaattaattgagaaatgcgttacatcatttcttacttttcaaggaaaaacatgttttgcgacttttgtttatgtgtctagtgactttgcagctgcccagctagggaatcagggtcttcacaatgcctgggaatggaggggagagaaagctcattagccacagaaaaataggccgttagttttttaaaggactctagctctttctctttctcagggggaattggattttcttacatacaactgaggttctgcttacacattctttaatttcttttaattcctgttccagtgcagtgcaatgttcatagtagatgcttaatattttttgaattattgtatctttagtcattgcgagccatcattttgagagtcagtctcattcttttagccctagattatttttattttcttgagatacgtgaataaagggcacaggaaaaaaacacagttgagagttttggataatagtaacctaattcacccagattttacttcatattggttggaactctaagttgggtgcaactaacatgagacttgttttgatttattgtaaacatattcttgaatgactaatgaacagtgcttcatgtgaacaaattttcaccttctgataatttccaatattacactttgaaaaacattcccattgtagtgctctagaaaatataatttcaattgttaaaattcaatattactgtaattgaggatattaaataacttgttgttacatgatgctagctgcagcttaaatttcagatcatgagtgttgttaatattatatagttggaattagttctgagatcaatgaatcaatgctgtcttactgttttgtctttaatttttataccacatttattttgtgagtggatttcataaaaggaagttgctttatgttgccaagctcattatcttgggtttttgtccagagctgaattccctttttcatcaaaggacagagagagcccattataaactattgagtaatgctgatttgatgggtaggccacttacctttaagtggaaaggaggccaggcgcggtggctcacgcctgtaatcccagcactttgggatgctgaggagggcggatcatgaggtcaggagatcgagaccagtctggccaacttggagaaaccctgtctctactaaaaatacaaaaattagccgggcgtggtggcgggtgcctgtagtcccagctactcgggaggccgaggcaggagaatggcgtgaacctgggaggcggagcttgcaatgagtcaagatcacaccgctgaactccagcctgggtgacagagcgagactccttctcaaaaaaaaattacacaaaaatgttagactaataaatattaataaacactaaaggaagaaatttagtggaacatttatcagaggcaacagaccaggaagttatagttaccctgtgaaaaggcatcatctaggattcatagaaaacatttgcaaattggcaacaaccaccaccacaacaaactaaaacaaatgaaagaaatagacatttatacagggaaaatataaatagataataagtgtaataaattttgtgactttccacttgaaaacaaagaaatggaaataaaacctatgtaaagaagtggtggagtgagccgagctggggtgggaggggtgggggcatcccaggcagagggaacagcagatgcaaaggtctggaggctggagcaggctgtgaggggtgagagcagcaagtacatgaagtctagaccacaaatcgatgcttcaccctcaaaaatagcctctcactggccaccactcaggcccagatacatgtgtaaggtgggcagccttcaggaaaagtgaccgcaaaagtgcaccacattgggaattctgaggtgtttgagaaagttatggatgaagggtgaggtagactctagtggggaatgtcctcttgccacttggacttctctgtctatttcatgggctattatcaacttctaaagagccagtggtttgggtgcacatttaggtgtaagtacctcttgcctcaagccttaactctctttccaatgagtcaagtgagctgttgtgtttaagtaacttctctttattgtgaaatctttcctatcactttaagaaccttcttcttcagacccaagtaatcccaaggcacctataatgtccctcctggagaagacaaaagggtgtcttctatttctgtgtggcccgatatttcagggcccctaaccaggttagttcaaggtgctcagcataaccatgaatctgctacccatgtttctcaggtgggtcataggccctggagggattgtggctgtggaccaagcagggggtgctgtgggctgttctgagaaggagggttttggagtgtccaagctgagaacatagaggccccagatgttgtatgagtaaattttcatctgacagaattatattttcctaactgaaaacatgaaacagagatgtaacaactgtgcctctttggctctgtctttgcatgctgtcatcagccactgaacaggagctgagagggaagacataaagaacagaaaacccatgagggggatggtccatgtgggtgtggaagtgagggtgagtggggatttggccaaagaggcctaatccacaggagtagagtctcatgtgtgactttcagaatgagcaggagtgagataggtggagggggagtgttggaacatctcatcagaggtgagaatccatagaaggagacactggtcattacagtcagtgagaaaagtgactttagcctgagcctaaggtcattggatcaaggaaggggccaggaggagctccttgaagggaagctgtgagcttgagaggcaggctgaggcagatcagtagaatgagcccccgagaagggctcacagagtcactgaaggactgtgaggtggcagggacaagctcacatggcctttggtcagtgagtctccagctgtaatgagaatgaaatggggaagatctgggacaggaagacaagctgggggcgctggtaccatcatggggagatgatggtgggtggctctaggttagttagagttggaatgaagagcagaaagcagcttcgtgatacctttatgtcataaaatacatttggcatggagaggacttggactttgtgcatgaaggatatggatgtttctaggctgactcccaaatgtttctaggctgactcccagaaactaggatgtttctaggctgttcctggctgagaatttggatggtaactatagtccaaagaaacaaagaacactgaaatcatatcttattttttttaactcacaaaatagaggcaggtagatatttatatgtgttttgttggctcaatgattttttttagtaataaacatatttacttagaaactactgggtgtctgtatgctctgggatttttttctcctgtcctgatgacatctctttcctttctctgggttcaggagttacttcccaggttgtggtgacacaggaacattcactttccacaactcctggaggggcagtcaccttcacctatggctccaaaattggggctgtcatcagagcatctcgacagctgggcccaacagaacatctggcaatgtccccagggcaatggtgggcacaagcagtcaggtcctgggacccctgcacaatcccctggttctcttcatgaagaaaaagccactctggttataatgaggactagaccaagaatgaggcccagtgttacatgctcggtgtggtgatctgaaatatcaccacagggacaaatacaaaaatacaagtgggaaactacactcaaacccttggatcaagcctcagtcctatgcttctgaagcaaatttcttgtttttggagtctggttttcaaccactgtagactcagatcatttattcttcaggcccccaatgggatcatcacaagcactttttctatcaatcatggcagtcacttgagatcaaaggtgaatttcttcttcctcaagttataagcaacactttggcatatttggtgaacttcaaagaaaatggctaagatagaggatgagagacataagaggcttcaggaatatttcagcagatgcaaagcagactacagggtaactcatttcccatatgatttgattgattgcaaaatgtgaaattaaatgcatacaaaggggaatgtgtgtcatttttcctcttaatgacaaagtaaaaaaaaagcaataaaacaaaaacaagcactatttcatgtgtcttgataaacttgtagaaagtcttataatatccacaatatctaggatacagtaaaataactttccatatgaagaaccagaagaacacaacttaaatgagaaaagacaattgatagacaccaatactgaaataatttaggtgctgatgttatgtaactgagattaattaaggagtaattatacaattccttcagtgagcaattttagacattattttttgaagttttaggttcaggggttcatgtgcaggtatgttacgtaggtaaacttgtgtcagaggggacattcttgaaacaagtggaaaagaagcaaatcccgaaaagaaaaataagtcatagaaacaaaccaaattaaaattagaaaacagaaaaatgcaataaattaaaaagaacagtctggagggactctggaacaggtaacagtgaaattaaagttcaggcattagaggaattatttaccgttaagtagggaagaaagtacaaacaatagccatctaagcccagcagagggagctgtggatcttctcagaggtgagcaaggccggaaatgaactcccggggctctcatttatggctcctgggtccatactcactggcccgaattggggtttagagcaggtgctttctcccaaagggcaaactcagagttcccagcagcccatcctccctgacagagcggctgctgcctggctgagcactcagaacttagggagccggtggttttggtagaggccccatgtttgcataatgtcctaaccaaacacacatctacccggggaggtgacgaggagttagaggaactgtcccagggtccagaagtagccgggggttctggggatccttggaaagtccacaccatagcttggatgtctatcctcccctctctccctcctcactgcccaggttcatgtagatttcaaagacaggactttggagggatctgtctgctgtgattcctcacacataacatgtatctgttttagtttcttggttcagttctcaggctgtggtgactcaggagccctcactgactgtgtcctaaagagagagacttattctcatgtgtggcttcagcaccacagcactgaattcagcagaagcctggtcaagtcctcaggacattgatgtgtaacacagataacaaacacccctggatgcccacttgattctctggctccctcctgggggaaagctgccctgacccctcaaggggcccagcctgaggatgaggctgagtatactgtgggctacaccacagtggtccttagcacagtgagagacccagatggggaagcaggacatgaacgagcttttggctgatccggggtacaaacatgaacgtaggatatcaggatcacgtggccagacacctgagctctcaaggacacctaaattttaactatccgtagagagaaaggtgggtggaggggtgtaaattgattcctgtatctgtttgaattttaaagtggaaaatatttgtgaatcatttgggagatgcagattcccaaaaagaagaatttatgtttatattccaaacagaacagtaatagtgagatcaactctaaagactaaggtcaaacacacttcatccagaaagatggatctcaaaagattttgttgttgttttgcctattaaaagctaagagaggaaattatattaataaagtttatttcatgttctatgttaatagtgtgtatttcatagctttttaagcattaaaaattataaaagtgttaagagggattctcttctctaatgttatataagaaagtatccacctaggaaaatattttgaaggtctaacccccagtacttctaaatgtgaccttatttttaaaatagtgttattgcttatgtaattcttaggatgaagccatcctagagcgaggtggcctcttgatccaatgtgcctggtgtccttataagatgatggtagtaaggagattggaagacacagagagaatgccgtgtgacgacgaaggcagagattgaatttacacagctgcaagataaggagcactaaagattgctggcaaaccaccagcagccagaaagagggaaggaaggatctcccctcaggtatatatggaaacattgccctttctatgaattttgggaggctagcctccagaactgtgagacaatacatttgtgttatcttaaccaaacttgtgtttaatacattctgacagcagttctaggaaaccaatacaccagacaataaattacacaatacttctgaaattgattgtacttctttcattttggcaaccagtttttccatgaatatgattatatctgtgctcatatttctaactttataaatatgcttgtaatttaaggttaagataagaaatccttgtgtcttaaaaatccctaaatatatgagaactgaataataatattaacattatgagatctaatggaaaattgtagcttttacataattccacttgttcactagtatcaagatgtgaaggtgggaagatcaccttgttattaacaaaacactcctggtcatctctgtggatttgagagtgcagggggagacacctacatattgtttgggaagtcacaaatgcagcatgcccagagcatcatgtacaacagtgtgagtttgctggacatgtgggcaggacgcagaacagactggggcatgaggaaccacctatcaccaaacccaccagcgccttcctccctggatcagcccagagctgctcagacttgaatgtgcacatggatcatgtgggatcctggataatgtggattctgatcagtgggtctggactgtggactgaaattctgcgtttctagaaggccccttcttccaaagatccacatacatgttttaatattaccatattcataagaatggtgggaaataagggaagccactttcagacagaaaccaaatagaaatttatctgagagggagctaaaggattatgatcaaagagaaatccccaattccaggcagggagctgatcacagagggcagatgggaagttgggagaggttctatttttttttttttaagaaatagaaatgaatgcaaacacaactcaaacctcctcttgatccaacatccctgctggctcctgcccaatttctgcactggcttcttctccagtcttttcccaagcatggaccctcctcaccctctctgcttccccatctcaaactcacaacttcccttctcagttctcctgtttctctccctttcattccacttccttcttctctgtgacctccttagacaatcaccaaaatatttctagaagaaaagccatgggtatttttcatcttttcttgaaagttgtcactcacattccctcttcgttcttcagcctctcttaccccttactacaaaagatgcctgaggccactgtttctcttttccatcccctcaaatagcttctccttccaacccgtttctctcctcagttactagagcattcagtgaagcactcagtcaggttaaaggatcctcccaaatgctcaccctgctctctatgtgtgatctcctgaagaatcaagtagggagagaaacatggcttcatgagtttgtggggaagtgggtgcatggctgaactgtccttgttctcagcaaatgtactttatttcatgcacagtttgtatatgagacaaccttctcctgcggactccttataaaatgggaagaattatgtgttgtgttttcacagaggaagcccaaatattccatgctcatgggtaggaagaatcaatatcatgaaaatggccatactgcccaaggtaatttatagattcaatgccatccccatcaagctaccaatgactttcttcacagaattggaaaaaactattttaaagttcatatggaaccaaaaaagagcccgcatcgccaagtcaatcctaagccaaaagaacaaagctggaggcatcacactacctgacttcaaactatactacaaggctacagtaaccaaaacagtatggtactggtaccaaaac

SEQ ID NO: 75 - protein, 117 aa, Homo sapiens
MAWAPLLLTLLAHCTGSWANFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSN

SEQ ID NO: 76 - DNA, 32230 bp, Homo sapiens; identical to SEQ ID NO: 69
  source 1..32230: mol_type = unassigned DNA

SEQ ID NO: 77 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 78 - DNA, 427 bp, Homo sapiens
  source 1..427: mol_type = unassigned DNA
acaacaggcaggcaggggcagcaagatggtgttgcagacccaggtcttcatttctctgttgctctggatctctggcgcctacggggacatcgtgatgacccagtctccagactccctggctgtgtctctgggcgagagggccaccatcaactgcaagtccagccagagtattttatacagctccgacaataagaactacttagcttggtaccagcagaaaccaggacagcctcctaagttgctcatttactgggcatctacccgggaatccggggtccctgaccgattcagtggcagcgggtctgggacagatttcactctcaccatcagcagcctgcaggctgaagatgtggcagtttattactgtcaacaatattataatttaccgtggacgttcggccaagggaccaaggtggaaatcaaacga

SEQ ID NO: 79 - protein, 121 aa, Homo sapiens
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 80 - DNA, 32230 bp, Homo sapiens
  source 1..32230: mol_type = unassigned DNA
gaattcctgtttctagtagggacagaaacggaacccaggctgttctggccaatcatgccctatctcagaatgttacatttccagaacattctacagttattccagaaaactacaatcaagaaagggaaggggactgggttgattcaagacaaaatgaaaactattctgcaaataccatagtgaaatcacagctcagtaatctttttgtgactggcttatttcatttagcgtaatgtcctctagtttcatccatgttatagcatgtgtgagaatttccctttttaaagctaaataatatgctattgtatgtatatatcacatttggattaccagttcactcctttgtgaacatttgagttgctctacctgttggctactatgaataattcggttctgaatgtgggtatacaaatatctcctcaagttaatgtcttcaattacttgggtatatgtccaaaagtggaattgctggattatatagtatttccattattaattcttcgaggaattgccatctggtttcccacagcaggtgggccatttacatcaccacgacagtgtccacaggagttccagttccctaagttctcaccatgactggtcattttctgttggaaaaaaaaatcctaataggtgtgaggtgggttttgtttttatttttctaaggattaataatattaagcatcttttcctatgctagttatctaattatctctaaagaatattctccagagaaatgtcgattcatatacttttctcgtttttaatcaggtattttattttaatgttgacatgtaggacttatttttatgtactagatattattaaccccttatcagatatacgatttacaaatattttcttctattccacatgttgcattttcactgtgttggttatgtcttttgatgcccattttacatttttatgtagtccaatttatcttcttttctacttttgcctgtattatggtattaaaggtgttagtattcaaatgtctataaatactgacttactatgctgagaaggtcactctagaccatctctctgatggtggagctaagaattttacactctctcattttgtaccagggcacagtgcagctaggtgagaacacagtggctttctcgagcttatttgtcttgcatttttggcgacaaggattgttgttcacattggcctcctctggcaggtccacctggaaagcattatatttagcaagaaaaaaggaggctgtgaatgatgtcaactgtgtatattccctgttgcaagtgtcaaatccatgaagcataaaaggatttactagaggatattaaattccttgcaaagtgttgaaaaacctgaaggaataggctccaggcaaagcctctagaacaattccaagaatggcactgctggcgcaggctggggaggagctcctgctgcctgagactccacattcaagctgtctcctgcaggaaagagagcagctcttctcactactgccccctgaaggacagcagctctgctatcaactactagagacctgacccctttccctgcagccctgcctgtgttgattatatcttcatgtcagactcatgtagattcatctgttttcaagggtacaggggttatttctgcccaagtccacactgtggctttctatcataaatacacgtttctacacttgaatttccaggaattactagtatcaacagcaaaatgctacaacccaacataaagatttcctttacaaagaacatcatgctccccatgtagcattgtgctccccaaaatgtggggaaactccaatctgttcatcccaacaccatctaggaaaaaatgttcttctctcatgagccattaatctgtctacttattgtttaatttttggcctaaattgtaaaggtggctaaaaatagcactttgaatttttttaaagaaagggtatatagaaaatattaaattatttaaaatacatatatgcatgacacatttagttattgtcaccagcttctttcacatttattttattctgtagtttctggcaatactttctctatggtagagtctggaaatgctactcctcatgtccttggcttcctaagtcccagatgtggtgaagaatgcacatcagctgtgttactacagaatgcttttggattgagatgggggaggggacgtgcttgatctcactcaaatgtgctcaatttcctggtctgggtcacagccaggcagcccagtcctatgggcacaatatcttcactcgccaggtttctatcaaaatgagagtgtccttcttgactgtggaaaacacggggcagggagaggatcaagaaatagcaaaaggagtatttggacttgcctgagctgccactctggtttccccaaatgttttcattccatttcattcatgataataaacacctttctgcctaaccagccataatcacttttcttagatccagctgattcagtgataaaactaaagggaaaaaataaatacagttttcaacgtttaagctgaccaggtggctgtctctggtgtttatacctccatatcccactatgcatttcatcttctctttgcctcactggagatccaagtgcagggaatgcttcacgtgactgtgatccctagaacttcatcttcatgactgttgaaatcttctctgagttttaccagtggaaatggcaataaatagaaagattccagaaggtcccctagggtccatattttctgtacttcttctctaaaataagtaaaatcaattccatttgctcttgctttatattggctacatctatgtgcccaggacacaccctaaagccctgtgttgacaacttatccagtattgtattaagtctaaatggtcacatgcttatcatatcatttcttccaatttcatggaataattgtcatgtcaactctttggattaagaaccatcaaccagataaccacagccctgaataaatagaaacagaatatttcaagctgttcagttggcataacagtgatgagtctatagctcattttcaagatataaatattctatttatgggagtaaaagcacgaatcatggttagtgtttcagagcctccaccacatctgacagcatagcaattcaacagcacaaggtttgtgtgccagctgtgcctgactaataatgtttttttcccctgtctatagggaaatctgcacctgtgcacatgacaagagcatgagctacacttttttcactttttctgaggtaaatacattgcttgatccaaaaccgtattatggaataatttcctgactctgatgaaagcatttggaaaatcctcaaataatatttgtgcagaaatacaaccaaaaagaaaggcaaattcatatataggaaaataaccagtgctctcctcatgttacatgaggtccactagtatcacctgaccccagctgttcatctgatccctgatgtgttgtacaatgttaggggttcaggaggggtcattcttcttggcaaatgaggtgttcagaagagccagtagccctttaagtctcagttagttgaaattcataattgagtccccaaagggacctccataacattggttcatgagactccctggtaaagctgggaaaggtgactgactgaagtccatgtgttgaatcatcctcattattaaaagcccctgctcattaatagcattttgattaatattcactgggaaagcattttttttttattttggctcatctttgaaaagtctagtcatagcttttcaatcactttgtctgcaatcgttttgttgcatttctttgaaggcctgatgatctgtcaaaaccaacagccaatattattacattgccgttatgttgggaaaagctgaatgttgggagaagctgaggcagggcttgcatgtctgacgtaatgtaaaagagtcttggaacacgtccggggtccagggtctaaaaccccttgtggcctttagtacaccaagctctgtgctaaagggtggaaggctaccctgacgcaccataatctaagcccagggcataaaatcccttgtgacttggatagaatccaggactcgtggctctggagtgtgtctacacttgctggctcctttctctttgctctccaaggatcgattgtatcttgagttaaaagaacctgctctccattatctcaagtagtagagcaaatgctaaaccatcacaggtgtaaatcatgtgcttaatgcaatgcatccttttggcctccacattcttaccacgtgtttctttgttggattaccaataaatagcatgggatcccagggctcagggccttcttagcctccatacactacgatggtccctggtgcccacatttctctctcaaactgtctttttctcaatctttgactccaccagactttgtcacccccacgacctggtgttgggtctgatcaccccaaaactgttatcgttgcaatgtgatcatcagatataacccttgcagtttactcattgttatgatttttacatttgcagggcttccttgacaatggcactaatgtgacgccatccagcatccattattgtgagaactctatttggaatatcaccattgtgggcatgaacagaatggagactttcataggatgcaatgtttaatattcaacattagtcaaaatttgttctagtcctacccttggagctccattgagttggaaaaatataaataaatttattttaaatataatataaatgtaaatataaacaaatttaaatataatataaatataatataaatatacataaatttaaacataatataaatataaatataaataaataaataaacaaaaaaattttctgtagcaagagtaagaactacatgtggtccagaataccattattttatggaaggttacaaaaaattttaaatgtatttaattcagataagaattttataaatatgcatatattgcatatatgatagaataaatgtacttaaatatgaatactatatatatttgtagccaacattttatcaaaaacatatatattcacttctatgtatcttatatacatgtatgcatacttttccgtaagataaattttaattttttattgaaatactaattagttttaattttgtctttaattttcttttaataacttcataatttgattactggatattcacatttaaatcaccaatttaagaaaaaatacatgtgtgtacatataggtagaggtgtaaatactgtatcaggaagcttttatgcattactgattggtaactggttaaatacacaactcagtgactgataacagtaaacatttgttttcatgttcacggatgctcatgttcacaatcctctgactgatcaaggaagggctcagctgagtggatcctctgaaggacacagacctcatcttcagcctatagatatcaattgtctgaggactaagctgaacaccagtgactacacatgatacaagattcttgtggcaggtcacaggagtgaacatcccaaaccaaactggacagttgaatttaagtccaataatttctaacatagcttcagatatttaaaatatattcctttatttcagtgagtacaaattttcaagaaaatgtttactccatttaattatagaggtgtttgatcattccatggacaaataattatgttttcatcctttacaatcttgaaaatatttgcaaatgtaaatttgcattaataagaaaataaagctggatgtgttttcaacatgtggctttaaatataattttttaaaatggcctcattgggggaaaatcattttaacttatatgaatatccttttttgcctctcttgtgttctatagagtggcccaataagaggtcctcccatgagatttggaatcaggaaaggatggctcaatattctccattggtacctaggacagacacagggacagagatgaggactggagaaacacctggaaagatgctgtaggaagctgagagcatcagcacccccacccctaagcttccagacaggactgaggaccacatggttagatagcccatacttcaggggaagatgcattcagttttctgagggagcaacagagattcctgcttctaatatcaactttcctgactactatatccttggctttgaaaggttgtagtgggaaagttaatcgtaggaattgggtcattcttgtcatacccaacagagccaagaaaccaggagggaaagacactcagggtgcaaaatattgtctgaagaatgtaattgaaataggccctattatcccatggaactaatgtttatggttttttgaaggaacatagaaattgactcctccagtcttaaaaactcaagatagttatatttttcttatctgaggagttcttttgtcaggaaaccaaccatcaggcctccagatactatcaaaaggagctgaaacttacatatcactgaatcgggacagtgagacatcagactcttcacccattgtgattgcctaactgacctcctgcttcctgttgaccaaatcatcttccttacccctccctaattcctgttttcccacatttcttccctgatatatatacccctcattttagtagttcagggagatacatttgagaatggtgtcccatctcctcggctgcagcacctgattaaagcctgttccttggcaatacttgtcttagtgattgtttttctgtgtggtgagcagcaggatctacactgaatccctggcatttcagtaacaaaattctctgcaagcttcactgcctttggcttattgtaacctgaaatcaaatttatccacaacttctgagataacttgatataactgtaggattcactttgtccaccactgcttcccagtctgagcttgccagctcccaacccttcctagtgcccatgaactttctcaaagagccataggtaacatgttccctttttcgtaaaactctaaccttctctttgttcttccaacatattgaagaccactgagttttcctgtatgccccatttggcaaatatttctttgcacgtaaaacattaaatttagagattcatctctacattttatttagacttcagtagtttagactctaattgtctgtattaagacaattcctgctttgaatatctatagtggcctcttctctgttatataaagtccagctgaagccataaactagactcttcaggtgtcatgatctctgtctttattaaatcaggagaggcattgctagatctgtgcagttggggctgagaaagagaaaagaattagggtgcagaggtgactccatgtccccctctaccaacaccatcagagtgtggctgcatctgaggaccactctcagctgatagaggcatcaggaggagcagctggggcagccctgcctcacacatctgcttccctgggggtttatgttcgggtgtgtaacactgtgggagaataactattatactgttggcagtaataagttgcaaaatcatcaggctgcaggctgctgatggtgagagtgaattctgtcccagatccactgccgctgaaccttgatgggaccccactttctaaactagacgccttatagatcaggagcttaggggctttccctggtttctgctgataccaggccaaccagctactaatactctgactggcccggcaagtgatggtgactctgtctcctacagatgcagacagggtggaaggagactgggtcatctggatgtcacatttggcacctgagattggaaatagaaacacaaatattcatactattgatcatattataggaagacttccctgaataaccaggcagtactgagcacactgggctgagtaaattcctagtgttctccttccttacctgggagccagagcagcaggagccccaggagctgagcggggaccctcatgtccatgctgtgtcctgactgggtctgactcctgcacaaagtgtgaccagcctattaataaggcttcagggcaggaggttgtgctctgggaacatgcaaatgagcaggggatggggcaggctgggcacagctgcagagctggctcatctcagtaactcagcaccagctcagtgtccccaggtgtcccaggtaagaccagggtagcacaaatttgtctgcagagaatgtgtttctactggggactattttattatgagaaacaatttttaggtatttttttgagaattttaaatattcctcaggagccgatagagtaatgtatttcattggtgtatcaggattatttaggagaatattcttgtttgtaggaaacacatagtaaaatgttagatggtaggattctcaagtcttcaaaagactctcataagattccgggtagggaagggggtaattgtgctatacctgcaacatttctgtgagtttaacattgttcctttctaaaaaaaattaaaaataaaatgtatcggcatgatgctatatatttgtaagtattaggtaatggtgttatgcctttgttcttactagtattagatcaagcaatttattacagatatacaaagatgataccgtgttgtctccatgcatgcagcactcacagatccaccactatcaagaactgcaggtctctttaatacccagagactaaatgaggtgcaccttattcttgttttgggtaccttcatagtctaccttcttttctgccattgggtattatttcccaaagttcatctgtcttagtgagggtggccactgcacggagcatgtccctgccatgcaccatcaatgacactttcttcttatactttttatcagtgcatggggacatcatcctgacccagacaccagcctccctgttaacacctttaggaaaaacatactcaatctcttatcaagcaattgtctatgtacatggagaaatcagttggatccagatgaaactggacatggatttgcattcattatatctcatatctctaatgtaccctgaacgtcccagcctgactcagtagcaggggaagtggatgtaaccacatcagcatcagtgggctgcagcctcgggctccacaaaattttactgatgcctgactaggggagcaaaatcacagtgctgcagcccatgcacaaacatttctgctgctttgtaagcagcctgaattttaagggaacttgcttatattggaagaaaggaagaaagttccatttgtcctctaaatgtttgctgaaaatgaaccgacaaaagaataatgaataagagaaaaggcaaacaaaattcacttagagtgcggtgggatatcatagtggggtgattacccagataactcaatgagatccagttgttcatatttcctttctagggaagagggaattgggaagtgtaggcaacctggagagaatagatgaaaaaagaaatgcatcctcaaaagaacaggcaatagcctgcctggataaagcatcaacttgcagtctcttctatttttgattcatgttttgtgttaatcttccctgatataaaatttcccaggaagaatttccttgacaattgtttttcttctggagaatttgcttttaggcagataagggatatttaggaaaagacttcttgtgcatttgctgctttctaaatgcctttggctttacataatcatcataccaatgcagcatagtttgagatgttattttctggattcctttacttgcagccacctgccaagatcctgtttcagagagatgcagctgcagattgagtgagcagttgacccctgaacaacatggaggttggggcactgaccacaggtgcagatgaaaacctgtgtagaagttttgcatttctaacttaagtactaatagcttacttttgactggaagccttagtgataaaataaatagttgattacacttttttacattttatatatattttatactctattcttccaataaagtatggtaaagaaaaaaatgttcttaagaaaaccataaaaatgagaaaatatatttactactgattaagtacttgctttcaggtgacacagaagaaaatataagtgtatctgcaaacttcaaacccaagttattcaagggttaactgtaccatgatgaatgtagcagtccccatctgtagtctagggctttctcctttgctgtacctctgctcacttccaatggccatatatatgtcttatgttctttatgatcttgggcagagaggtctgcctacatgcattgctggccagatgtcctggaatgtgtatctctgaggaaagtgctatggtttgactgtgtcctccaaaatccatctgttgtaaagttcattctcagtgaaatggtttttgccaggtgggccctattgggatgtgtttaggtcatgagggtggagccctctagtggaatacattaatgccagtataaacagggtttatagggctggaatctttctctctcttctgctggtctgtcctgataagacatggccttccttccattgaaggactcaatgccccaggcatcgtcttgaaagcagagaaagctgaccttaacctgcccatgccttgatctcaaactttccgtactccagaagtgtgagaaaatatatttctgttttttatgaattacacagcgacaaggaacctgttatagcagcttgaaagagaacaggagagacagctcacaatcagtgaggataagatgaggtatatacatatcccagctttctcatctctcaggtggaatagcccagaggaatttagtccatgtttccacatgtggttgatcttcagttatcctgagtcaggtgggtggttgatgtgtcttttaccattcatcttctgctcctgcctcactttcttcctttcctcccagtgtaaatttgctgcctaaacaggaatactcattgctggtggacccaaaataagacagaaaaaaaaaatcattgtacttttgtatgagggatatttctcatctgaattcttatcacttctctttctttgacatctaggaatattcagaaaacacttttttttaaccaatctattttagattgaatttattgattttttttcctgtgtgttttgtgaaccaaaaattaagttgtaagccaccaacgagctaaatggactccccttttggcagagagaacttcaaagaaatctgaaaaactaggttaggccatgactggcaggtgggtttggatgtgtctcattatgctctcttccctttggagttcaggcacaactgaccagcattatcattataacagagatctttggactgaggaaacagatgcttgtagcaataagataccatactccaacatgacagataataggccctgaagaaaatctaaaaattttactctaaaaatatttctttttctttttcttttttttgtttttttttgagatggagtcgcgctctgtgcccaggctggagagcagtggcgcaatcttggctcactgcaagctccgcctcctgagttcatgccattctcctgcctcagccttccaagcagctggaactacaggtgcccaccaccatgcccgactaattttttttgtatttttagtagagatggggtttcaccctgttagccaggatggtctcgatctcctgacctcgtgatctacccacctcggcctcccaaagtgctgagattacaggagtgagccaccgcacccaacctatttctctgaaatattctgaagtggccctgcaaagctgcgcgttgtgggagaaatttgcattctgtagagaatctcctatgcttactaatcattttccaaaggtctgactttttttttttaaggtctgacaagcaacgtctactttttctgctacccataggattcatctacacgaaaagaaccttggcttatctaaactcaagcacaccccgttatctaaactcaagcatttctttatggtgaattcaactctttaggcagagcttaactctttcaaccagttgccaatcaggaaaactttgaagccacctgtgacctggaagcccctgcttcaagatatcccacctttccagtacaaactaatgtatatcttatatgtattgatttatgtctttgcctgtaatttctgtgtctccctaaaatgtataaaaccaacgtgtaatccaaccaccttgggcacatgtttacaggaccccctaaggctgtgtcacaggccataatccttatctttggcaaaataaatctataacttgattgagacctgtctcagatactgttttgcttacactgggtcacaaaatttaaaaatcctctaaaactatctacacatccataaatctacattagaggcagtggagtgagcacactccaacagtcaaaactcacaagttagaggaacctgagtctaagattttgtccagctctcttgcttcataaaaataatttgtgatttttttaaattttactctagagaaggacaattttggggaatatgtttatggtcagtggaatgaataatgtccccaccccaaaagatgtccatatcctcgtctctggaacccatgttatacgagttattaagaaactattttaggcatatagagaggaaacagggtccttgggaaggttttgtttcttttaaagcagctccagaaatgtttcttgtttagcaggaaagccctggctcttagagctggtccggcaagcttttttttcttttttagagagggagtctcgctctgtagcccaggctggagtgcagtggcacgatctcggctcactgcaagctccgccctctggtttcctcatgcctcagcctcccaagtagctgggactacaggcgcccgccaccacggccggctaattttttgtatttttcagtagagacggggtttcactgtgttagccaggatggtctcgatctcctcacctcctgatctgcccgcctcggcctcccaaagtgctgggattacaggcggcaagctttgatatacaaatactggccattagaaactgggtccagccaaacatggagattcccacattcttcttctttttttttttttttttttgagatggagtctcgctctgtcgcccaggctggagtgcagtggaacgatctcggctgactgcaacctctgcctcctgggttcaagcgattcttctgcctcagtctcctgagtagctgggattacaggcacatgccaccacgcccagctactttttgtgtttttagtagagacggggtttcaccatattggccaggatggtctccatctcttgacctcatgatccaccttccttggcctcccaaagtgctgagattacaggtgtgagccacggcgtccagctcccacggtcttcttccttgccccgacatttgcctgacaacatggctgcccccacatatccccatgtgtgtagaactttatggcaccctgcatttgcatattaaaacactagggtgggagggccagttttttctcaagctacatgaatgacatgcttggtaaaaccaatcccctaagccctatgcaaatcagacaccacctccttcagcctcctcatataagcagccacttttccaccgcacatggagttttctcttagttctaatctcccctctctctgtctctgtacggtggagctgttttcttcttccttccttcttgcgtattaaacttttcgctccttaaaacaaccccacgcatgtccgtttctttttaaacaaacccgcgtgagactaagaacggtggtggtcctccagtcatcagagccctatcataaggattctaccttacatagcaaaagaagaacttgacaggtgtaagttaacgactttgagaataagcaacctggttatccagatgaaaccaatataatcacaaaggtcattaaaatagaggaggaggcttgcagagaggagctgggacaatatggaaggatgtggtttgaggagggaaggggccatagagccagaaatgtgggagcacctggaagataaaagcagaggattcagttctttcctctggagcctccagaaggaatgcagccctactgacaccttaacattagctcagtgagaatgctgacctccagaactacagggtaatacatttatgttgtgtgaagccaataagactgtggcgatttaaacagcagcattggaaactaatgcaaggggaagaaatgtctttcagctcactgagcgcgtgcttgtcttctgttcttggaaatatttccaccttgttttctggtgtcagttatgacaagagacagagaaaatttttccgagagaagagctaccacgagaattcgtttttagctagaaaatctcctgggtaaaatctcatgattatctgttatacgatctgggtatcacagagtttggggtcagatctcacaacatgtgataggaggaagagggttttgttagtttgttttcatattgcgagggaaaattagattttcaggacacaatctgagaaggacagacagagtcagagatattgtaagagaaagaggagatgtggaaggagtcagggcaatgaaacacgtgtcccagctcccaaatctaaaataaagtcattgattttacaaggtgaagaaaagctcttcaccttccatcttcttaggaaggatcaaattcttcccagagtccctgcccctccttcacccccctgacattgcattgtggagctaatcacatgtgcccttaatccctgctcctgtcccaggctgggagaggctcactgtcctcaccatgcccagcaggctagagctggtgccctaagcaccaaaggagaagaggatgatttctgtgcaggcaataagagctttaccgcattttacatttccctaaaattctaagcaaagttctgtggagagaagagaacatggggcaactttatctggtggggctggaagagaccaggctctgagaatgacaattaattcacatttctacttttccagatgccaaatgctagctttatcaattgtggaaatgtggactttgagtttaagaacaggtttcaggtactgcaataattgaggagacatttggaatagagtccttagtctagggctaaagtcagactggcagtgcctgagagcttccaggcgctggccctgggctgtgagggaagcagcagttctcctgagccacagggctgaggacctgggagcaaccacaggccttggccacagggctgcctggcaggggcttgagggaaggaaactgctcacaaattcacaggagctgcattaagcatatatcccgcagcctggcaagagtgaaagtctcagagacccagaccttagggctggggctgggattctgggctggctgctgtcggctctgccctcccttgtcctgaatgactggcaccctgctggagccaaaaatggagggtcggccaatgtcctcaaagtctttactcagccagaccctgttctgcttggaaagaaaatgaacacatggtatacaaataaacataaaatcatgtgtatctgtaataagaatttctaatataaattttacatattgaatacatatattttagaaataagttgtacttatacatttgcctatactatgtgtctatttatggataaatatgtggtacatgtttatatataaagatatataactttggttgctagtataaggtatagttttaaactttaataaagtaaacacgaacattgaaaatgagcactgggtgcaccacttcatggccctcctcactccagggcctaagggtgataaagctcaggaccctcttccatgtcccccggcatggacagcagtgaactacccattgctgaggatcatgagtgacctgagggggatctgcagaatcacagagcagcagatgcccagaagattgatagtttggaccctgaatggagggacattttgatcacagggctcagcatagacaagtactcttctggtcatttctgtcaaacaaaacagcacatgtagctcagaggctgcatgatcacaggatgaagcacctccatgagtcattgccagcttcccctctgactggggtgatgtgggatctctctgccccactttcatggctcaccagctgctgggactctgttgacagtggtctacatctgtcccacgcacaaacttctcaatagttgtcattttacttactggtctccaataaccacaaattgccagccatgacccttccacaaaaattgatggagtggggttggaactatgggttcagcaatgtgccagggcatgtatataagagaaacaaggtgtggccagcccatgtctgaagtgaggatgaattttcaccatgaattcggagtaggaggtggaagaaaacatcctgacatgctccaggcagtgatgcccacaagatacactagaagcgcccacagaggaggtgggaagggggtgtgagaaaaagagggagtgtgaatttacaggtgtctccagactgttcacctgaaaagagatcagacccttaacaacacctgggaccccaggcagagctgttgctatggaagatttgtgcaggacagggataggaggagggagcacctacctgtgatggcatcttggttgctgttggccagaatgacatcttttcaggcctactgtccctgacccaggcagggatcatgtccaggagggcagcaggagctcagagccaggccctggttctgctagtgccaggctaggatgttcttcatactctggccagctctacaggtgacagtgaccctctcccctgagacacaaggaggaggacaacgatggcatccacataacatgctcactgtcatccaagtgggggaacaaacatgcagatccccaaatattaataccgagtttgtagttcctccaacttggtgcaattctgatcagaaggaaaaacaggctaacaacttcatcagcagggaaagttcatgtttaatacaaactatctgaggctgaagcctgagtttcccttcctcaccaggcagtcaggacagcaggagcaaaaggagaaaagctgggtcccaaagtccacaaggtgcctcctgagactgatcctgcttagagaaggtggtgacagtggatgagtcttctgggactcccacaccaacataccctgagctggatggcttgagccatagacatttattgctaatttattaaacgtttattgtgaatgtattagaaccctggaaagttcaagatcaaagtccagaaggatttgctttctggtgagaaccctgcttcttgttttcagatgtcacccctggatacatcctcacgtgtgttcagcaatgtgccgggcatgtgtaagtaggagaagcaagtacatgctttcttcagtgcatgcttggaggctggtgaggggttaaggaacagatcgatgatgtttatttttactaggacacaaatcctattaaatcagagccccaaggttttcacttcatttcaccctaattaccccttcataacctctatgtctaaatacagtaattttgcaggttggagattcaaaatatgaatttgtgaaacacaattcagttcatagcaggtgtgctttgaggacatgcccaaggcagtaggaagggcaaggcgaggcttccagtctcagagcacagaaggcgttttcccaccactcagcacactggcagctcctcttaggtgatccaggtcacacatgagacgccattcccgccttgggggctgcctgctgataatggaacaatatcacccttaattttcactatttctaagatcagacttctattttatgtttattatggagtttgtccaatggccaggtttgtaacataacctccaatgtttggtttgtgacataacatcttatagaattttagatgctggtttccagtgagacttgatggagttttttcatgggttttcatggagttgatcttattacatccaccattatgctgcattctgtagggaacacaaaatgattcctctcctcaataaaccagtcacctagttgaagataaagggtgttcagaaaagatggtcacaaaacaattaggggctagattctgtgatccatgtgagatgccaatgatgtgattctggaaagcagaaggtgaggttgtgagataccctgcagcacataatgcccaaggctagggtggcgagtagagggcccctggtctggatctgtctacccagcttcattcctcccattcgtgaaacagtggctgcaccagccaggaagcatctgagaccacctgattctctggaataatagctgctgaggtttgtgctgtgagctataacactgtgagtctctaactgtaaccatttagattagcactaatgaaaataatttttaagatggtgaaaatgtctatatctatgctttgcaagtgtagccgttatatgtcatatgtgactcctgaacacttgaaatgtagtgaaaacaactaaggaactatattgttaattttaattaattccaatatacatgtttgtggctgggtactgctaatgttggccatgtgagtcagggaagttttagatggtaataaacagcaatgtcatcaggacaaccaatctgctaaatgttaagcaaagatgatcccaggaccattgcaccaagctggagtggacaaggtggaaagagcacgtaggtggatctttggaacccagattctgcttcctccaggtgaatgatgtaaactctctgtcttgaagacactgacattgaagctagcgactggggagctgagtgtttctcctggaagctgcagtggaaacagaacatagatcttccttagtattactcatgtccctctccataggcttcccagcagagaggtgctgtctgatcaagtgggaggtgaatgctgtgctccctaatcaatagtctttctcagtcccatgggacatatgaaccctgtcacatgcagcccacgcagttgaagccagagccccatgtccagtgtccagtgttgcctctgccttcggtgatcccaccctacaggacccagtgatgaaggcttgagtggcagggaagagctttggggagcctgccaactagcaataagggtgcagacttgggcttccagcttctgagcaagggaacattgtatgtcctaggtgtgatccaggtcaaggtgtttgataaaagatattacaataaattattttcttaagaaaataaaaacacagaaggcatctgattattataggactgtttgggttttataccactaaagtggtaaaaataaatttttcaatgcgcagtggtttatcaggaatctcccagcccctgcccttcccgtgatgattctgtagtctttgcaggttgtatggtcacttggtggcctccttaccgatcagatgcatctacagcacaaccctaagatcttccccgattaacatagatgagcaacttcaacctccagataccacataccattggctgatttttatgcaatttatcaacactgaaagtattctattccatttcattatgtagttattattctgtgcatggctcattcccctactgagtatcagtatttgcgatcaaaaattgttttatttacttacatgcactaaaccttaaatgatagcatttattagttctgaaaactctgacattaagttaaaatcccttaaaagcaactatatgcttctttttttaattagaataaatatggtgcaatcacctcaactaaggatactaaactacttttacagttgaaaatgattaattggacatggggaaacatattgacaaaagtaacaagcattattatgatgttgaataaacacagtttgcatgtaaagtgtacagttatgattaattgtattaaaacacaggaggaagaaaggtggggattgctggattggttggtgattcagtccttagaagcaagggggagctgatgagtgatctcaggcaggcatccaatataaggtccaaatatgattaattacaatgttcttttccttaaggtttgatattttaagctggtcctgctggggaagttttgagcacattcatggaatatgtggaataaagattaaaaacagtatatttcaaaatactcttaaaaagctcagaaacacccaaagaaaactattttataaaatatcatgccttcctcgcccattcttgatatttgtgtttaaccaagcatgactcacaattcaacttttctccacattgttgcctagagtgttgaagaaattcctctaataggacagaaatggagtcaatcttgcaatatctggccaatcgtgttctggccagtcttgctttatctcagaatgttacatttccagaaacttctacagttattcctgaaaactacaagcaagaaagggaaggagactgggatcattcaaggcaaaatggaacctgttctgcaaatactgtagtgaaatcataactcagtaatcgttttgtgactggcttatttcacttagtgtaatgtcctctagtttcatccaagttgtagcatgtgtcagaatttccctttttaaagctaattttgcctttttaaagctacaatatgctactgtatgtatataccacatttggattaccagttccctcctttgtgaacatttgagttgcttctaccttttggctattgtgaataattcggttctgaatgtggatatacaaatatatcctcaagttaatgtcttcagttacttgggtatatgtccaaaagtggaattgctgaattatatagtatttctatttttaattcttcgaggaattgccatctggtttcccacagcaggtgggccatttacatcaccacgacagtgtccacaggagttccagttccctaagttctcaccaagactggtcattttctgttggaaaaaaaatcctaataggtgtgaggtgggttttgtttttatttttctaaggattaataatgttgaccatgttttcctatgctagttttctaattatctctatagaatcttctttagagaaatgtcgattcatgtactttcctcatttttaatcaggtattttattttaaggttcacatataagacttatttttgtatagtagatattattaaccccttatcaaatataagatttacaaatgttttcttctattccacatgttgcattttcactgtgttgattatgtcttttgatgcccattttacatttttatgaagtccaatttatcttcttttctacttttgcctgtattttggtattaaagttgttagtatttaaatgtctataaatactgacttactatgctgagaaggtcacactgcaccatctctctgatggtggagctaagagttttacactctcccattttgtaccgggggacagtgcagctatgtgagaacccagtggctttacccagcttatttgtcttgcatttttggtgacatggattgttattcacattggcttcctctggcaggtccacctggaaagcattatatttagcaagaaaaaaggaggcagtgaatgatgtcactgtggactgtgtatattccctgttgcaaatgtcaaattcgtgaagcataaagggatttactagaggatattaaattccttgcaaattgttgaaaagccttaaggaacaggctccaggcaaagcctctagaacaattccaagaatggcactgctggcacaggctggggaggagctcctcctgcctgagactccacaacattcaagctgtctcctgcagaaaagagagcagctcttctcactactgcccccagaaggacagccgctctgctatcaactacgagagatctgacccctttctctgactgcccatcagatgtgttactacagagtgcttttggattgagatgagggagggaaaatgcctgatctcactcaaatgtgctcattttcctggtctgggtctcagccaggcagcacagtcctgtgggcacagtatcttcacttgccaggttcctataaaatgagagtatctttcttgactgtggaaaatatggggatggtaagggtcaaggaataggaaaaggagtacttggagttgcctgagctgccactctggtttccccaaatgttttcaatccattaaattcatgataataaacacctttctgcctaaccagccataatcgcttttcttagatccaaatgattctgtgataaaactaaagagaaaaaataaatacacttttcaacgtttaagctgaccaggaggctgtctctggtgtttacatgtccatatcccactatgcatttcgtcttccctttgcctcactggagatccaagtgcagggaatgcttcatatgactgtgatccctagaacttcatcctcatgactgttgaaatcttctctgagttttaccagtggaaatggcaataaatagaaagattccagaaggtcccctagggtccatattttctgtacttcctctctaaaatatgtaaaatcaattatatttgctcttgctttatatgggctacatctgtgtccccagcacacaccctaacgccctgcgttgacaacttatccagtattgtattaagtctaaataggcacatgtttatcatattatttcttccaatttaatgagataattgtcatgtcaaatctttggattaagaaccatcagccagaaaaccacagccctgagaaaatagaaatagagtatttcaagctgttcagtttgcacaacaatgatgattctatagctcatttacaagatataaatattctgtttatgggagtaagagcaacattcacggtgagtgttgtagaccctccgccacatctgacagcatagcaattcaatagcacaagggtttgtgtgccagcagtgcttgactaataatgggttttttttctatagggaaagctgcacctgtgcacatgacaggatcatgagctacactttacttcactttttttgaagtcaattaccttcttgatccaaagccacagtatggaagaacttcctgactctgatgaaggcatttggaaaatcctcaaataatattttatgcagtaacataaccaaagacaaaggaaaattcatatataggataataaccactgctctcctcatgttacatgaagtgcacttgaatcacctgacatcagctgttagtatgagccctgatgtgtggtagaatgttaggggctcaggaagggtcaattttcttggcaaattagtgttcaagagagccagtagccctgtacatctcagttagttgaaactcatattattgagtccccacagggacctccgtaacagtggttcatgaggttacctgggaaagtgggcaaaggtgactgactgaagtccatgtgttcaatcatcctcattatgaaaagccccctgctcattaatggcatttggattaatattcactgagaaagcatgtttttcgattatggctcatttttgaaacatctagtcatagctcttccaaaatgactttgtctgcaatcatcctgttgtgtttcttttaaggcctgatgatctgtcaaaaccaacaaccaatattattaaattgctgttctctttgcaatgtggcaatgtgatcatcagatgtaggccttgcagtttacccacggttaggatttttacatttccagggattccttgacaatggcactaatgtgacgccatctagcatccattgttgtgagaactctatttggaatatcatcattgtgggcatgaacataatggagactttcataggatgcaatgattaatattcaacattagtcaaaatctgctgtagtcctacccttggagatctactggtttggaaaaatattttaggtaaggacaacaatttggtaaagttttaaaaataaaggctaaaaagaaattttctttagcaagagtatgaactatctgtggtccagaataccatttttttacagatgcaagaaaaaattatttaaatgtatataatttagataataattttgtaaatttgtatatattgcatatataatagaataaacataattaaatatgaatactatatatacttatagccaacattttatcaaataaatatatattaacttctatgtatcttatatatatgcacatttttccataaataaggtaaattttaattttttaatgaaatactaattagttttaatttcatctttaattttctcttaataacttcataatttgattacttaatgttcacatttataccaccaatttaagaaaaaacacatgtatctatatataggcagaggtgtaaatactgtatcagtaagcttttatgcattaatgattgataatcggttaaatatacaactcagtggctgataactgtaaatatttgttttcatcttcatggatgctcatgttcacaatcatctggctgattaaggaagggctcagctgattgattcctccgcagggcactgagcttgtctttagcctacagataccaattttctgaggacgaggctgaacagcagtgactacacgtgacacaagattcttgtgacaggtcacaggagtgaacaacatcccaaaccgaactgcacagttgaatttaagtccaataatttctaacatagcttcacacattttaaatatattttttgatttcagtgagtacaaattttcaagaaaatgtttactccaattaattatagaggtgtttgatcattccatggacaagtaattatgttttcaacctttacaatcctgaaaatatttgcaaacgtaaatttgcattaataagaaaataaagctggatgtgttttcaacatgtgcttttaaatataatttttgaaatgacctcaaagggggaaaatcattttaacttatgtaactatccctttttacctctcttgtgtcctatagagttgcccaataagaggtcctcccatgagatttggaatcagaaaaggatgactcaatattctccattggtacctaaggcagacacaggaacagaggtgaggactagagaaacacctgaaaagatgctgtaggaagctgagagcatcagcacccccacccctaagcttccagacaggactgaggaccacatggttagaaagcccttacttcagggacagatgcattctgttttctgagggagcaccagagattcctgcttctaatatcagctttcctgactactatatccttggctttgaaaggtcatagtgggaaagttaatcataggaattcggtcattcttgtgatacccgacagagccaagaaaccaggagggaaaggcactcagggtgaaaaatactgtttctagaatgcaattgaaataggccctattatcccatggaactaaagtttatggttttttgaataaacagaaatcgactcctccagtcttaaaactcaagatagcaacatttatcttacctttcttttttttttttttttttttttaaatttcaggaaagcaaccatcaggcctcccagatactatcaatttgctgaaacttatatatcactgaatcgggacagtgagacatcagacccttcacccattatgattgcctaactgacctcctgcttcctgttgacccaattatctttcttaaccctccctaactcctgtttttccacatttcttccttgttatataaacccctaatttcagttggtcagagagatacatttgagaatggcatcccatctcctcagctgcagcacctgattaaagcctgttccttgaaaatacttgtcttagtgattggctttctgtgtgacgagctgcaggatctacaccgaatccctggcatttcagtaacaaaattctctgcaagcttcaccgttttggtttattgtaacctgaaatcaaatttatccaaaacttctgagataacttgatataattctaggattcactttgtccaccactgcttaccagtctgagcttgccagctcccaacccttcctagtgccaatgagctttctcaaaagagccataggtaacattttccctttttcataaaatgctaaatttctctttgttcttccaacatattgaagaccactgagttttcctgtatgccccatttggcaaatatttctttgcaaataaaacattaaatttagagattcatctctacattttatttagacttcagtagtttactctaattctctgtattaagactatatctgcttcaaatatctatagtggcttcttctctgttatacgaattccaactgaagccataaactagactcttcaggtgtcatgatctctgtctttattaaatcaagagaggcattgctagaactgtgcagttggggctgagaaagagaaaagaattagggtgcagaggtgacttcgtgtccccctctaccaacaccatcagagtgtggctgcatctgaggaacaatctcagccaatggaggcatcaggaggagcagctggggcagcccagtctcacacatctgcttccctgggggtttctgttcgggtgtgtaacactgtgggagggtaattgtaatcttgtagacagtaataagttgcaaaatcttcaggctgcaggctgctgatggtgagagtgaaatctgtgccagatccactgccgctgaaccttgatgggaccccactttgtaaactggatgcagcatagatcaggagcttaggggctttccctggtttctgctgataccagcctaaatcatttctaatgccctgacttgcccggcaagtgatggtgactctgtctccaacagatgcagacagggaggatggagactgggtcatctggatggcacatctggcacctgagattggaaacacaaaaacaaatggtccacacaattaatcatgtagtaagagaatttccctgaatagccaggctgtgctgagcaccctgggctgagtaaactgccagtgttctccatccttacctgggagccagagcagcaggagccccaggagctgagcggggaccctcatgtccatgctgtgtcctgagtgggtctgactcctgcacagggtgtgatcagcctgttaataagtcttcaggtcaggagactgtgctctgggaacatgcaaatgagcaggggaaggggcaggctgggcacagctgcagggctggctcatctcagtaactcagcaccggctcagtgtccccaggtgtcccaggtaagaccagggtagcacaaatttgtctgcagagaatgtgtttctactggggactattttgttatgagaaacattttaaagatattttttgacaatattcctcgagagtcaatggggtaatatatttcattggtgtatggggattattttggagaatattcttgtttgtaggaaacacagtacatattagatggtacgattctcaggtcttcaaaagactgttataagattccatttagggaagggggtaattgtgctatacttgaaacatttctgtgagtttaacattgttcctttctaaaaaattaaaaataaaatttattgacatgatgctatatatatttgtaagtattaggtaatggtgttatgccattgttcttaccagtataagatcaaacaatttactacagatacacaaagatgatgccgtgcttcttcaatgcatgtggcactaacagccccaccattatcaagagctacaggtctttttaatacccagagactaaatgggctgcacctgtgtagaatttttgcatccctaacttaagtactaatagattgcttttgattggaagccttagcaataaaataaatagttgattaacatgttttttaatgttatatatattatatactctattattccaataaagtatgctaaagaaaaaaatgttattaaggaaaccataaaatagagaaaatatatttactacttattaagcatttgcttacaggtgacacacacagaacaaaatataagtgtatctgcaaatttcaaacccaagttattcaagggttaactgtaccatgatgaacgtagcagtccccatctgtaatctagggctttcccctttgctgtacctctactcatttccaatgaccatatatatgtcttatgttctttatgatcttgggcagagaactctgcctggatgcattgctggccagatggcctggaatgtgtatctcttaggaaagtgctatggtttgactgtgtcctccagaatccatctattgtaaagttaattctcagtgtaatggtttttgccaggtggggcctattgggatgtgtttaggtcatgaggctggagccctctagcggaatacattaatgccactataaacaggaattacagggctgagatctctctcctgctcctctgacatgttaagacatggccttccttcctttgaagaactcaacgctccaggcatcatcttgaaagcggagaaagcagaccatgtggttgatcttcagttatcctgagtcaggtaggttgttgatgtgtcttttaccattcattttctgttccctccctcactttcctcctttcctcccagtataaatttgctacctaacaggaatccttattgctggtggatccaaactgagagagtaaaaaagaaaagtcattaatcttttgtatgaggggtatttctcatctgaaatcttaccatttctctttctttgatatgtacaaatattcaggaagcacctaatttttttttttttaccaatgtatcttagattgaatttacgctgtgattttttttctgtgtgttaaaacaaaaaatcaagttgtaagccaccaacctactcaatgggcttctcttttggcagagagaacgtcaaagaaatctgaaaaactaggttaggccatgactggcaggtgggtttagatgtaactcattatactctcctccctttggagttcagacaaaactgaccagtgttatcattacaacagagatctttagactgacaaatcaaatgctttgtagcaataagataccatactccaacatgacagataataggccctgaagaaaatctaaatattgtaccctaaaaatattttttttgatgtattctgaagtggccctgcaaagctgcctgttatgggggaaatttgcattctgcagagcatctcctcctcttactatgtcttttccaaagagttggacatttctttaaaggtctgataagcaacattcaccatctactttactgttacctgcaaggttcatctaagtgacaagaaccttgacttccacacccccttatctaaactcaaggatttctttatgatgaattcaactctttaggcagagcttaactctttcaaccagttgccaatcaggaaaactttgaagccacctgtgacctggaagcccctgcttcaagatatcccacctttccaggacaaactaatgtatatcttatatgtactgacttaggtctttccctgtaatttttgtgtctccctaaaatgtataaaaccaacgtgtaatccagccaccttgggcacatatttgcagggcctcctaaagctgtgtcacaggccataagccttatctttggcaaaataaacctatacattgattgagacctgtctcagatactgttttgtttacactgggtcacaaaagttaaaaatcctctaaaactctctacacatctataaatctacattagagacagtggagtgagtatacctcaactcaagtcatactttgagtttaagaaccagacagattttcaggtacttcgataaatgaggagacatttggaatagggtccttagcccagagctaaggtcaggctggcagtgcctgagcgcttccaggccctggccctggtctgtggaggaagccactgttctcctgagccacagggctgagaacctgggatgagccacaggccttggccgcagggctgcctggcagggtcttcagggagggaaactgctcacaaattcgcgggagctgcattaagcatatatccccccagcctggcaagagtgaaagtctcaggacacagaccttagggctggggctgggatcctgggctggctgctgtcagctgtgtcctcccttgtcctgaatgactgggaccctgctggagccaaagagggagggtcagcaaatgtcctcaaggtcttcactcagccagactctgttctgcttggaaagaaaaggaacacatgctacaaaaataaacataaaattatgtgtatttgtaatatgaatttctagtataaactttacatattaaatacatacattttagaaataagttatatttatatatttgtatatactatgtatctgtggataaatatgtgatgcatgtttatgtataaagagatgcaagtttcattgctagtataaggtatagttttaaactttaataaattgaacgtgaacattgaaaacaagcactgggtgcaccacctcatggcccctcctcactccagggcctgagggtcataaagctcgggactctcttccatgtgccccctgggccgatagcagtgagctacccattgctgaggatcatgagtgacctgagggggatctgcagaatcacagggcagcagatgcccaggagatggatagtgaatttggaccctgaatggagggacgttttgtccacagggctcagcacagacaagtccttttctggtcatttctgtcaagcaaagcagcacacatggctcagaggctgcatggtcagaggaataagcccctccatggctcattcccagcttcccctctgactggggtgatgtgggatctctctgcccagctttcatgactgaccagctgctgggaccctgttaacaatggcctatgtcagtgccatgcacaagcttcttgatagttaccattttactttctggtctccaataaccacaaattgccaaccatgaccgtttcacaaagattcatggagaggggtcagaattatgggttgagcaacgtgccggggcatgtatgtaagagaaacaaggtgtggccagcccatgtggaagtgaggatgaattttcatcatgaattcagagaaggaggtgggagaaagcatctgacatgcttcaggcagtgatggccacaagatacattagagatgttcatagaagaggtggcaagtgggtttgagatgaaaagggaatgtgaattcagaggtgtcccccagcctgttcccctgagaagatttcagacccttagcaacccctgggcccccaggtagagctgttgctagggaagatttgtacatgataggggacaggaagagggatcacctacctctgaaggcatcttggttggtagtggccagagtgacatctgttcaggcccattattatccctgacccaggcagggatcatgtccaggagggcagtaggagccagcaggagctcagagccatgccccagttctgctggtgccaggctaggatgttcttcatactctggccagccctgcaggtgacagtgaccctctaacctgagagacaaggaagaggatgaagatggcatccacataacatgctcactgtcatccaagtgggggaacaagcatgcagattcccaaatattaataccaagcttttatttcatccaacttgatgaaattctgatcagaaggagaaacagtctaacaaattcatcatcaaggaaagttcatgtttaatacaaactatctgaggctgaaacctgacttccccttcctcaccaggcagtcaggacagcaggagcaaaaggagaaaagctgggtcccaaagtccacaaggtgactcctggggctgatcctgctcagagaacgtggcaacagtggataagtctgccgagaatgccacaccatcatactctgagctggagggcttcaaccacagacatttattgctaatttattagacatttattgcgaatatattagaaccatggaaggttcaagatcaaagtccagaaggatttgctttctggtgagaaccctgtttgtttccagatgtcctctctggatacatcctcatgtgtgttcagcaatgtgccaggcatgtgtaagagaagcaagtgcatgctttactcagtacacgcttggaggctggtgaggggttcagtaacagctcaatgatatttctttttattaggacacaaatcctattaaatcagaaccccgatgttttcacctcatttcaccctaattacctcttcataacctctatgtctaaatgcagtaatattggaggttggagattcaaaatatgaatttgtgaaacaccattcagttcatagcaggtgcactttgaggatatggccaaggcagtaggaagggcaaggcaaggcttccagtctcagagcacagagggcattttcccaccactcagcacactggcagctc

SEQ ID NO: 81 - protein, 116 aa, Homo sapiens
MDMRVPAQLLGLLLLWFPGSRCDIQMTQSPSSVSASVGDRVTITCRASQGISSWLAWYQQKPGKAPKLLIYAASSLQSGVPSRFSGSGSGTDFTLTISSLQPEDFATYYCQQANSF

SEQ ID NO: 82 - DNA, 32230 bp, Homo sapiens; identical to SEQ ID NO: 72
  source 1..32230: mol_type = unassigned DNA

SEQ ID NO: 83 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS